SEQUENCE LISTING (29 sequences)

SEQ ID NO: 1 - RNA, 23 nt, synthetic construct
  source 1..23: mol_type = other RNA
  modified_base 1..2: mod_base = OTHER; note = Phosphorothioate linkage
  modified_base 1: mod_base = um
  modified_base 2: mod_base = OTHER; note = 2-Fluororibose nucleotide
  modified_base 3: mod_base = um
  modified_base 4: mod_base = OTHER; note = 2-Fluororibose nucleotide
  modified_base 5: mod_base = gm
  modified_base 6: mod_base = OTHER; note = 2-Fluororibose nucleotide
  modified_base 7: mod_base = OTHER; note = 2'-O-methyladenosine
  modified_base 8: mod_base = OTHER; note = 2-Fluororibose nucleotide
  modified_base 9: mod_base = cm
  modified_base 10: mod_base = OTHER; note = 2-Fluororibose nucleotide
  modified_base 11: mod_base = OTHER; note = 2'-O-methyladenosine
  modified_base 12: mod_base = um
  modified_base 13: mod_base = cm
  modified_base 14: mod_base = OTHER; note = 2-Fluororibose nucleotide
  modified_base 15: mod_base = OTHER; note = 2'-O-methyladenosine
  modified_base 16: mod_base = OTHER; note = 2-Fluororibose nucleotide
  modified_base 17: mod_base = cm
  modified_base 18: mod_base = OTHER; note = 2-Fluororibose nucleotide
  modified_base 19: mod_base = gm
  modified_base 20: mod_base = OTHER; note = 2-Fluororibose nucleotide
  modified_base 21: mod_base = OTHER; note = 2'-O-methyladenosine
  modified_base 22: mod_base = gm
  modified_base 23: mod_base = cm
  modified_base 21..22: mod_base = OTHER; note = Phosphorothioate linkage
tttcgaatcaatccaacagtagc

SEQ ID NO: 2 - RNA, 21 nt, synthetic construct
  source 1..21: mol_type = other RNA
  modified_base 1..2: mod_base = OTHER; note = Phosphorothioate linkage
  modified_base 1: mod_base = OTHER; note = 2-Fluororibose nucleotide
  modified_base 2: mod_base = OTHER; note = 2'-O-methyladenosine
  modified_base 3: mod_base = OTHER; note = 2-Fluororibose nucleotide
  modified_base 4: mod_base = um
  modified_base 5: mod_base = OTHER; note = 2-Fluororibose nucleotide
  modified_base 6: mod_base = um
  modified_base 7: mod_base = OTHER; note = 2-Fluororibose nucleotide
  modified_base 8: mod_base = gm
  modified_base 9: mod_base = OTHER; note = 2-Fluororibose nucleotide
  modified_base 10: mod_base = OTHER; note = 2-Fluororibose nucleotide
  modified_base 11: mod_base = OTHER; note = 2-Fluororibose nucleotide
  modified_base 12: mod_base = um
  modified_base 13: mod_base = OTHER; note = 2-Fluororibose nucleotide
  modified_base 14: mod_base = OTHER; note = 2'-O-methyladenosine
  modified_base 15: mod_base = OTHER; note = 2-Fluororibose nucleotide
  modified_base 16: mod_base = um
  modified_base 17: mod_base = OTHER; note = 2-Fluororibose nucleotide
  modified_base 18: mod_base = gm
  modified_base 19: mod_base = OTHER; note = 2-Fluororibose nucleotide
  modified_base 20: mod_base = OTHER; note = 2'-O-methyladenosine
  modified_base 21: mod_base = OTHER; note = 2-Fluororibose nucleotide
tactgttggattgattcgaaa

SEQ ID NO: 3 - RNA, 21 nt, synthetic construct
  source 1..21: mol_type = other RNA
  modified_base 1..2: mod_base = OTHER; note = Phosphorothioate linkage
  modified_base 1: mod_base = OTHER; note = 2-Fluororibose nucleotide
  modified_base 2: mod_base = OTHER; note = 2'-O-methyladenosine
  modified_base 3: mod_base = OTHER; note = 2-Fluororibose nucleotide
  modified_base 4: mod_base = um
  modified_base 5: mod_base = OTHER; note = 2-Fluororibose nucleotide
  modified_base 6: mod_base = um
  modified_base 7: mod_base = OTHER; note = 2-Fluororibose nucleotide
  modified_base 8: mod_base = gm
  modified_base 9: mod_base = OTHER; note = 2-Fluororibose nucleotide
  modified_base 10: mod_base = OTHER; note = 2-Fluororibose nucleotide
  modified_base 11: mod_base = OTHER; note = 2-Fluororibose nucleotide
  modified_base 12: mod_base = um
  modified_base 13: mod_base = OTHER; note = 2-Fluororibose nucleotide
  modified_base 14: mod_base = OTHER; note = 2'-O-methyladenosine
  modified_base 15: mod_base = OTHER; note = 2-Fluororibose nucleotide
  modified_base 16: mod_base = um
  modified_base 17: mod_base = OTHER; note = 2-Fluororibose nucleotide
  modified_base 18: mod_base = gm
  modified_base 19: mod_base = OTHER; note = 2-Fluororibose nucleotide
  modified_base 20: mod_base = OTHER; note = 2'-O-methyladenosine
  modified_base 21: mod_base = OTHER; note = 2-Fluororibose nucleotide
tactgttggattgattcgaaa

SEQ ID NO: 4 - RNA, 22 nt, synthetic construct
  source 1..22: mol_type = other RNA
  modified_base 1: mod_base = OTHER; note = Phosphorothioate linkage
  modified_base 1: mod_base = OTHER; note = 2-Fluororibose nucleotide
  modified_base 2: mod_base = um
  modified_base 3: mod_base = OTHER; note = 2-Fluororibose nucleotide
  modified_base 4: mod_base = gm
  modified_base 5: mod_base = OTHER; note = 2-Fluororibose nucleotide
  modified_base 6: mod_base = OTHER; note = 2'-O-methyladenosine
  modified_base 7: mod_base = OTHER; note = 2-Fluororibose nucleotide
  modified_base 8: mod_base = cm
  modified_base 9: mod_base = OTHER; note = 2-Fluororibose nucleotide
  modified_base 10: mod_base = OTHER; note = 2'-O-methyladenosine
  modified_base 11: mod_base = um
  modified_base 12: mod_base = cm
  modified_base 13: mod_base = OTHER; note = 2-Fluororibose nucleotide
  modified_base 14: mod_base = OTHER; note = 2'-O-methyladenosine
  modified_base 15: mod_base = OTHER; note = 2-Fluororibose nucleotide
  modified_base 16: mod_base = cm
  modified_base 17: mod_base = OTHER; note = 2-Fluororibose nucleotide
  modified_base 18: mod_base = gm
  modified_base 19: mod_base = OTHER; note = 2-Fluororibose nucleotide
  modified_base 20: mod_base = OTHER; note = 2'-O-methyladenosine
  modified_base 21: mod_base = gm
  modified_base 22: mod_base = cm
  modified_base 20..21: mod_base = OTHER; note = Phosphorothioate linkage
ttcgaatcaatccaacagtagc

SEQ ID NO: 5 - RNA, 21 nt, synthetic construct
  source 1..21: mol_type = other RNA
  modified_base 1..2: mod_base = OTHER; note = Phosphorothioate linkage
  modified_base 1: mod_base = OTHER; note = 2-Fluororibose nucleotide
  modified_base 2: mod_base = OTHER; note = 2'-O-methyladenosine
  modified_base 3: mod_base = OTHER; note = 2-Fluororibose nucleotide
  modified_base 4: mod_base = um
  modified_base 5: mod_base = OTHER; note = 2-Fluororibose nucleotide
  modified_base 6: mod_base = um
  modified_base 7: mod_base = OTHER; note = 2-Fluororibose nucleotide
  modified_base 8: mod_base = gm
  modified_base 9: mod_base = OTHER; note = 2-Fluororibose nucleotide
  modified_base 10: mod_base = OTHER; note = 2-Fluororibose nucleotide
  modified_base 11: mod_base = OTHER; note = 2-Fluororibose nucleotide
  modified_base 12: mod_base = um
  modified_base 13: mod_base = OTHER; note = 2-Fluororibose nucleotide
  modified_base 14: mod_base = OTHER; note = 2'-O-methyladenosine
  modified_base 15: mod_base = OTHER; note = 2-Fluororibose nucleotide
  modified_base 16: mod_base = um
  modified_base 17: mod_base = OTHER; note = 2-Fluororibose nucleotide
  modified_base 18: mod_base = gm
  modified_base 19: mod_base = OTHER; note = 2-Fluororibose nucleotide
  modified_base 20: mod_base = OTHER; note = 2'-O-methyladenosine
  modified_base 21: mod_base = OTHER; note = 2-Fluororibose nucleotide
tactgttggattgattcgaaa

SEQ ID NO: 6 - RNA, 21 nt, synthetic construct
  source 1..21: mol_type = other RNA
  modified_base 1..2: mod_base = OTHER; note = Phosphorothioate linkage
  modified_base 1: mod_base = OTHER; note = 2-Fluororibose nucleotide
  modified_base 2: mod_base = OTHER; note = 2'-O-methyladenosine
  modified_base 3: mod_base = OTHER; note = 2-Fluororibose nucleotide
  modified_base 4: mod_base = um
  modified_base 5: mod_base = OTHER; note = 2-Fluororibose nucleotide
  modified_base 6: mod_base = um
  modified_base 7: mod_base = OTHER; note = 2-Fluororibose nucleotide
  modified_base 8: mod_base = gm
  modified_base 9: mod_base = OTHER; note = 2-Fluororibose nucleotide
  modified_base 10: mod_base = OTHER; note = 2-Fluororibose nucleotide
  modified_base 11: mod_base = OTHER; note = 2-Fluororibose nucleotide
  modified_base 12: mod_base = um
  modified_base 13: mod_base = OTHER; note = 2-Fluororibose nucleotide
  modified_base 14: mod_base = OTHER; note = 2'-O-methyladenosine
  modified_base 15: mod_base = OTHER; note = 2-Fluororibose nucleotide
  modified_base 16: mod_base = um
  modified_base 17: mod_base = OTHER; note = 2-Fluororibose nucleotide
  modified_base 18: mod_base = gm
  modified_base 19: mod_base = OTHER; note = 2-Fluororibose nucleotide
  modified_base 20: mod_base = OTHER; note = 2'-O-methyladenosine
  modified_base 21: mod_base = OTHER; note = 2-Fluororibose nucleotide
tactgttggattgattcgaaa

SEQ ID NO: 7 - RNA, 21 nt, synthetic construct
  source 1..21: mol_type = other RNA
  modified_base 1..2: mod_base = OTHER; note = Phosphorothioate linkage
  modified_base 1: mod_base = OTHER; note = 2-Fluororibose nucleotide
  modified_base 2: mod_base = OTHER; note = 2'-O-methyladenosine
  modified_base 3: mod_base = OTHER; note = 2-Fluororibose nucleotide
  modified_base 4: mod_base = um
  modified_base 5: mod_base = OTHER; note = 2-Fluororibose nucleotide
  modified_base 6: mod_base = um
  modified_base 7: mod_base = OTHER; note = 2-Fluororibose nucleotide
  modified_base 8: mod_base = gm
  modified_base 9: mod_base = OTHER; note = 2-Fluororibose nucleotide
  modified_base 10: mod_base = OTHER; note = 2-Fluororibose nucleotide
  modified_base 11: mod_base = OTHER; note = 2-Fluororibose nucleotide
  modified_base 12: mod_base = um
  modified_base 13: mod_base = OTHER; note = 2-Fluororibose nucleotide
  modified_base 14: mod_base = OTHER; note = 2'-O-methyladenosine
  modified_base 15: mod_base = OTHER; note = 2-Fluororibose nucleotide
  modified_base 16: mod_base = um
  modified_base 17: mod_base = OTHER; note = 2-Fluororibose nucleotide
  modified_base 18: mod_base = gm
  modified_base 19: mod_base = OTHER; note = 2-Fluororibose nucleotide
  modified_base 20: mod_base = OTHER; note = 2'-O-methyladenosine
  modified_base 21: mod_base = OTHER; note = 2-Fluororibose nucleotide
tactgttggattgattcgaaa

SEQ ID NO: 8 - RNA, 21 nt, synthetic construct
  source 1..21: mol_type = other RNA
  modified_base 1..2: mod_base = OTHER; note = Phosphorothioate linkage
  modified_base 1: mod_base = um
  modified_base 2..3: mod_base = cm
  modified_base 4: mod_base = um
  modified_base 5: mod_base = OTHER; note = 2'-O-methyladenosine
  modified_base 6: mod_base = um
  modified_base 7: mod_base = OTHER; note = 2-Fluororibose nucleotide
  modified_base 8: mod_base = OTHER; note = 2'-O-methyladenosine
  modified_base 9: mod_base = OTHER; note = 2-Fluororibose nucleotide
  modified_base 10: mod_base = OTHER; note = 2-Fluororibose nucleotide
  modified_base 11: mod_base = OTHER; note = 2-Fluororibose nucleotide
  modified_base 12: mod_base = um
  modified_base 13: mod_base = OTHER; note = 2'-O-methyladenosine
  modified_base 14: mod_base = gm
  modified_base 15: mod_base = OTHER; note = 2'-O-methyladenosine
  modified_base 16..19: mod_base = um
  modified_base 20: mod_base = OTHER; note = 2'-O-methyladenosine
  modified_base 21: mod_base = um
tcctatgactgtagattttat

SEQ ID NO: 9 - RNA, 23 nt, synthetic construct
  source 1..23: mol_type = other RNA
  modified_base 1..2: mod_base = OTHER; note = Phosphorothioate linkage
  modified_base 1: mod_base = OTHER; note = 2'-O-methyladenosine
  modified_base 2: mod_base = OTHER; note = 2-Fluororibose nucleotide
  modified_base 3..5: mod_base = OTHER; note = 2'-O-methyladenosine
  modified_base 6: mod_base = OTHER; note = 2-Fluororibose nucleotide
  modified_base 7: mod_base = um
  modified_base 8: mod_base = cm
  modified_base 9: mod_base = um
  modified_base 10: mod_base = OTHER; note = 2'-O-methyladenosine
  modified_base 11: mod_base = cm
  modified_base 12: mod_base = OTHER; note = 2'-O-methyladenosine
  modified_base 13: mod_base = gm
  modified_base 14: mod_base = OTHER; note = 2-Fluororibose nucleotide
  modified_base 15: mod_base = cm
  modified_base 16: mod_base = OTHER; note = 2-Fluororibose nucleotide
  modified_base 17: mod_base = um
  modified_base 18: mod_base = OTHER; note = 2'-O-methyladenosine
  modified_base 19..20: mod_base = gm
  modified_base 21..22: mod_base = OTHER; note = 2'-O-methyladenosine
  modified_base 21..22: mod_base = OTHER; note = Phosphorothioate linkage
  modified_base 23: mod_base = um
ataaaatctacagtcataggaat

SEQ ID NO: 10 - protein, 10 aa, synthetic construct
EQKLISEEDL

SEQ ID NO: 11 - protein, 8 aa, synthetic construct
DYKDDDDK

SEQ ID NO: 12 - DNA, 54 bp, synthetic construct
  source 1..54: mol_type = other DNA
gtcgtatccagtgcagggtccgaggtattcgcactggatacgaccagaaactca

SEQ ID NO: 13 - DNA, 54 bp, synthetic construct
  source 1..54: mol_type = other DNA
gtcgtatccagtgcagggtccgaggtattcgcactggatacgacattcctatga

SEQ ID NO: 14 - DNA, 20 bp, synthetic construct
  source 1..20: mol_type = other DNA
gccgcgctaaagcactttat

SEQ ID NO: 15 - DNA, 20 bp, synthetic construct
  source 1..20: mol_type = other DNA
gccgcgcataaaatctacag

SEQ ID NO: 16 - DNA, 16 bp, synthetic construct
  source 1..16: mol_type = other DNA
gtgcagggtccgaggt

SEQ ID NO: 17 - DNA, 20 bp, synthetic construct
  source 1..20: mol_type = other DNA
gccgcgctaaagcactttat

SEQ ID NO: 18 - DNA, 19 bp, synthetic construct
  source 1..19: mol_type = other DNA
ctggatacgacattcctat

SEQ ID NO: 19 - RNA, 22 nt, synthetic construct
  source 1..22: mol_type = other RNA
tagcttatcagactgatgttga

SEQ ID NO: 20 - RNA, 24 nt, synthetic construct
  source 1..24: mol_type = other RNA
ttaatgctaatcgtgataggggtt

SEQ ID NO: 21 - RNA, 51 nt, synthetic construct
  source 1..51: mol_type = other RNA
gtcgtatccagtgcagggtccgaggtattcgcactggatacgacctgttgg

SEQ ID NO: 22 - DNA, 18 bp, synthetic construct
  source 1..18: mol_type = other DNA
gccgcgctactgttggat

SEQ ID NO: 23 - DNA, 22 bp, synthetic construct
  source 1..22: mol_type = other DNA
gccgcgctagcttatcagactg

SEQ ID NO: 24 - DNA, 24 bp, synthetic construct
  source 1..24: mol_type = other DNA
gccgcgcttaatgctaatcgtgat

SEQ ID NO: 25 - DNA, 16 bp, synthetic construct
  source 1..16: mol_type = other DNA
gtgcagggtccgaggt

SEQ ID NO: 26 - DNA, 19 bp, synthetic construct
  source 1..19: mol_type = other DNA
ctggatacgactttcgaat

SEQ ID NO: 27 - DNA, 17 bp, synthetic construct
  source 1..17: mol_type = other DNA
ctggatacgactcaaca

SEQ ID NO: 28 - DNA, 17 bp, synthetic construct
  source 1..17: mol_type = other DNA
ctggatacgacaacccc

SEQ ID NO: 29 - RNA, 21 nt, synthetic construct
  source 1..21: mol_type = other RNA
  modified_base 1..2: mod_base = OTHER; note = Phosphorothioate linkage
  modified_base 1: mod_base = OTHER; note = 2'-O-methyladenosine
  modified_base 2: mod_base = OTHER; note = 2-Fluororibose nucleotide
  modified_base 3..6: mod_base = OTHER; note = 2'-O-methyladenosine
  modified_base 7: mod_base = um
  modified_base 8: mod_base = cm
  modified_base 9: mod_base = um
  modified_base 10: mod_base = OTHER; note = 2'-O-methyladenosine
  modified_base 11: mod_base = cm
  modified_base 12: mod_base = OTHER; note = 2'-O-methyladenosine
  modified_base 13: mod_base = OTHER; note = 2-Fluororibose nucleotide
  modified_base 14: mod_base = um
  modified_base 15: mod_base = OTHER; note = 2-Fluororibose nucleotide
  modified_base 16: mod_base = OTHER; note = 2'-O-methyladenosine
  modified_base 17: mod_base = um
  modified_base 18: mod_base = OTHER; note = 2'-O-methyladenosine
  modified_base 19..20: mod_base = gm
  modified_base 21: mod_base = OTHER; note = 2'-O-methyladenosine
ataaaatctacagtcatagga

(19) United States
(12) Patent Application Publication
(10) Pub. No.: US 2025/0242035 A1
(43) Pub. Date: Jul. 31, 2025

(54) GLYCAN CONJUGATE COMPOSITIONS AND METHODS

(71) Applicant: GANNA BIO, INC., Watertown, MA (US)

(72) Inventors: Namita Bisaria, Somerville, MA (US); Mohui Wei, Boston, MA (US)

(21) Appl. No.: 19/182,831

(22) Filed: Apr. 18, 2025

Related U.S. Application Data
(63) Continuation of application No. PCT/US2023/077372, filed on Oct. 20, 2023.
(60) Provisional application No. 63/380,433, filed on Oct. 21, 2022.
(60) Provisional application No. 63/490,453, filed on Mar. 15, 2023.
(60) Provisional application No. 63/499,060, filed on Apr. 28, 2023.
(60) Provisional application No. 63/579,355, filed on Aug. 29, 2023.

(51) Int. Cl. A61K 47/54; C12N 15/113

(52) U.S. Cl. CPC A61K 47/549; C12N 15/113; C12N 2310/14; C12N 2310/351

(57) ABSTRACT

The present disclosure provides methods and compositions for modulating cell surface proteins and receptor complexes using a novel class of glycan conjugates that can be used to engage the signaling pathways within desired cell types. Such defined cell-targeting bioactive glyco-ligands are directed for cell engagement and activation in therapeutic applications.

### RELATED APPLICATIONS

The present application claims the benefit of U.S. Provisional Application No. 63/380,433, filed on Oct. 21, 2022, U.S. Provisional Application No. 63/490,453, filed on Mar. 15, 2023, U.S. Provisional Application No. 63/499,060, filed on Apr. 28, 2023, and U.S. Provisional Application No. 63/579,355, filed on Aug. 29, 2023, each of which are incorporated by reference in their entireties.

### FIELD OF THE INVENTION

The disclosure herein generally relates to the field of RNA therapeutics and glycobiology. More specifically, the embodiments described herein relate to cell signaling molecules, e.g., glycosylated ligand compositions, production, and therapeutic administration in subjects.

### BACKGROUND

Glycans have many applications in nanomedicine, including generating biomaterials, coating nanoparticles to evade the immune system or initiating cell signaling on specific cell types. Sampaolesi et al., (2019), Future Med. Chem. 11(1): 43-60. For example, glycans can be used to target macrophages, B cells, or hepatocytes, among others. Sampaolesi et al., “Glycans in nanomedicine, impact and perspectives” (2019), Future Med. Chem. 11(1): 43-60.

In one study, the signaling capacity of certain glycan residues was demonstrated in dendritic cells, which increased cell surface expression of MHC II, CD86, CD40, the C-type lectin receptor CLRE, and the mannose receptor CD206 following exposure to nanoparticles functionalized by covalent linkage to dimannose and lactose residues. Brenda et al. “Mannose-functionalized “pathogen-like” polyanhydride nanoparticles target C-type lectin receptors on dendritic cells.” Molecular pharmaceutics vol. 8, 5 (2011): 1877-86. The expression of these cell surface markers was not increased following exposure to nonfunctionalized nanoparticles. Brenda et al. 2011. Both functionalized and nonfunctionalized nanoparticles were internalized into the dendritic cells, and blocking the mannose and CIRE receptors prior to exposure to the functionalized nanoparticles prevented the increase of MHC II, CD40, and CD86 at the cell surface. Brenda et al. 2011. Thus, interaction with the mannose and CIRE receptors, as well as internalization into dendritic cells, was necessary for the functionalized nanoparticles to upregulate cell surface expression of MHC II, CD40, and CD86. Brenda et al. 2011.

More recently, the ability of a specific glycan, N-acetylgalactosamine (GalNAc), to bind the asialoglycoprotein receptor (ASGPR) has been exploited to target RNA therapeutics to hepatocytes. Hu, B., Zhong, L., Weng, Y. et al., (2020), Sig. Transduct. Target Ther. 5(101). Unlike other cell types, hepatocytes contain roughly 500,000 ASGPR receptors per cell, allowing GalNAc-containing ligands to target them with high specificity. Hu, B., Zhong, L., Weng, Y. et al., (2020), Sig. Transduct. Target Ther. 5(101). Alnylam Pharmaceuticals, Inc., for example, has targeted its siRNA therapies to hepatocytes by conjugating them to tetravalent and trivalent GalNAc ligands. Hu, B., Zhong, L., Weng, Y. et al., (2020), Sig. Transduct. Target Ther. 5(101). In 2019, Alnylam received its first-ever approval a GalNAc-conjugated RNAi therapeutic GIVLAARI® (givosiran) by the US FDA for the treatment of adults with acute hepatic porphyria (AHP). The approved drug is a double-stranded siRNA that causes degradation of aminolevulinate synthase 1 (ALAS1) mRNA in hepatocytes through RNA interference, which leads to reduced circulating levels of neurotoxic intermediates aminolevulinic acid (ALA) and porphobilinogen (PBG), factors associated with attacks and other disease manifestations of AHP (product insert December 2020).

Recent developments expanded the repertoire of endogenous scaffolds for glycans beyond the canonical proteins and lipids to include RNA as well. Flynn et al., (2019), bioRxiv: 787614. Sialylated glycans attached to RNA were found to be displayed at the cell surface and interact with members of the Siglec receptor family. Flynn et al., (2021), Cell 184(12): 3109-3124. Such evidence of glycosylation on RNA implicates the potential importance of glycosylation in cell signaling.

What is needed, therefore, is a new class of cell signaling molecules that can be used to target specific cell types and mediate a desired biological function.

### SUMMARY

Described herein are methods and compositions for producing pharmaceutical compositions comprising one or more glycans operably linked to one or more sites on a synthetic scaffold domain. The invention provides methods to enable development of directed therapeutics to drug a number of targets mediated by glycan mediated interactions. Such compositions demonstrating a desired biophysical and pharmacodynamic properties are used for the treatment of various conditions including cancer, inflammatory conditions and autoimmune diseases. Accordingly, the glycan mediated compositions and methods of the present invention provide a novel class of therapeutics and a new therapeutic modality.

In various aspects, the invention provides one or more glycans operably linked to one or more modified sites on a synthetic scaffold domain. Such synthetic scaffold domains include but are not limited to one or more nucleic acid sequences wherein at least one nucleobase site is modified, e.g., modified sequences on a scaffold (e.g., a synthetic scaffold domain) to operably link a signaling molecule, e.g., one or more glycans. Methods to covalently conjugate glycans to RNA have been recently demonstrated using click chemistry. Dong et al., Nature 2020 demonstrated that converting a terminal amine to an azide provides a chemical handle on a glycan, which can react with an alkyne on the nucleic acid to lead to a covalent conjugation. Preferably, such methods are employed to operably link one or more desired glycans on an RNA, which can result in various combinations of therapeutic glycosylated RNA molecules.

Preferred synthetic scaffold domains include one or more nucleic acids selected from DNA, RNA, Y RNA, miRNA, mRNA, siRNAs, antisense oligonucleotides (ASOs), circRNA, ribosomal RNA, small RNA fragments (e.g., transfer-RNA fragments), and related RNA types.

In other aspects of the invention, the glycans conjugated to synthetic scaffold domains comprise one or more N-linked type or O-linked type glycans. Such glycans include but are not limited to, one or more glycans selected from, for example, Tables 1A-1F and FIG. 1. Preferably, the glycans conjugated to the synthetic scaffold domains comprise self-antigens that are not readily recognized by the host immune system as foreign antigens or does not elicit an undesirable immune response. Exemplary embodiments of the invention demonstrate glycan-conjugated synthetic scaffold domains, e.g., glyco-ligands mediating desired cell signaling, receptor-mediated signaling cascade to target cells of interest or interaction with specific carbohydrate receptors.

Provided also are methods and compositions for site-specific modification of a target region of a synthetic scaffold domain, the method further comprising contacting one or more glycans with defined areas of the target nucleic acid molecule whereby one or more desired glycan is stably attached to the synthetic scaffold domain.

In various aspects, the pharmaceutical composition comprising the synthetic scaffold domain is characterized by its glycan site occupancy on a specified scaffold target greater than 10%, greater than 20%, greater than 30%, greater than 40%, greater than 50%, greater than 60%, 3 greater than 70%, greater than 80%, greater than 90% or higher. Preferably, described herein are glyco-ligand compositions comprising: one or more glycans; and a ribonucleic acid sequence operably linked via covalent bond to the one or more glycans. More preferably, such a pharmaceutical composition comprising a glyco-ligand is characterized as having a glycan site occupancy greater than 10%, greater than 20%, greater than 30%, greater than 40%, greater than 50%, greater than 60%, greater than 70%, greater than 80%, greater than 90% or higher.

In related aspects, a pharmaceutical composition comprising a desired glyco-ligand modulates a cell surface protein on a target cell. In other aspects, a pharmaceutical composition exhibiting a desired glyco-ligand modulates the activity of a target cell through a cell surface protein. Preferably, the pharmaceutical composition exhibits characteristics associated with one or more the following:
stable glyco-ligands that mediate a desired biological function;
configurable and programmable glyco-ligand for modulating biology; and
specific cell-targeting glyco-ligands to deliver or enhance other bioactive molecules to particular target cells. (See, for example, FIGS. 2A-2D, 3, 4A and 4B).

In yet other aspects, the glyco-ligand composition exhibits improved stability properties. For instance, the glycans conjugated to RNA modulate physicochemical properties, such as conformational stability and interactions with cell surface proteins.

Also described herein is a method for modulating activation or inhibition of a cell surface protein on the surface of a cell population present in a subject comprising contacting the pharmaceutical composition comprising glycosylated synthetic scaffolds (e.g., synthetic scaffold domains). Accordingly, provided herein are methods and compositions for contacting glyco-ligands on a cell surface protein to transduce cellular signaling. Preferably, the glycans on the glyco-ligand elicit a receptor-mediated signaling cascade to target cells of interest or through interaction with specific carbohydrate receptors, such as lectins. Lectins have carbohydrate binding affinities ranging from mM to nM. [Cummings R D, Darvill A G, Etzler M E, et al. Glycan-Recognizing Probes as Tools. 2017. In: Varki A, Cummings R D, Esko J D, et al., editors. Essentials of Glycobiology [Internet]. 3rd edition. Cold Spring Harbor (NY): Cold Spring Harbor Laboratory Press; 2015-2017. Chapter 48]. For example, lectins bind monosaccharides with binding affinities in the mM range, complex glycans in the μM, and complex glycoconjugates with multivalency in the nM range. [Cummings R D, Darvill A G, Etzler M E, et al. Glycan-Recognizing Probes as Tools. 2017. In: Varki A, Cummings R D, Esko J D, et al., editors. Essentials of Glycobiology [Internet]. 3rd edition. Cold Spring Harbor (NY): Cold Spring Harbor Laboratory Press; 2015-2017. Chapter 48]. More preferably, the carbohydrate receptors recognize certain glycan structures or a limited number of sugars residues (e.g., even a terminal sugar residue), and the receptor glyco-ligand interaction induces a much more robust response with multiple presentation (e.g., cluster effect). In various aspects, the glyco-ligands of the invention may be characterized as either positive or negative regulators of target receptors.

Also described herein is a method for formulating a glyco-ligand composition. In some aspects, the method further comprises lipid formulation. In other aspects, the method comprises non-lipid formulation. In preferred aspects, the glyco-ligand composition does not include either lipid or non-lipid formulation. In some aspects, stabilizers and excipients are included in the formulation.

Provided also are methods for administering the pharmaceutical composition. Preferably, the pharmaceutical composition is administered subcutaneously or intradermally via microneedles.

In various aspects, the disclosure provides methods and compositions for administering to a subject one or more pharmaceutical composition comprising one or more glycans linked to one or more sites on a synthetic scaffold domain. Preferably, the pharmaceutical compositions are used to ameliorate certain diseases including, for instance, cancer, inflammatory conditions and autoimmune diseases.

Provided herein are pharmaceutical compositions comprising a glyco-ligand. The glyco-ligand can comprise one or more glycan moieties. The glycan moieties can be operably linked to one or more sites on the synthetic scaffold domain (e.g., an RNA such as siRNA). The one or more glycan moieties can include at least one of any glycan disclosed herein. The one or more glycan moieties can include at least one of glycans M-5, M-6, or M-7 (the structures of which are shown in Table 1E). The one or more glycan moieties can include at least one of glycans M-2, M-3, or M-4 (the structures of which are shown in Table 1E). The one or more glycan moieties can include at least one of glycans H-65, H-14, H-10 or H-40 (the structures of which are shown in Table 1B). The one or more glycan moieties can include at least one of glycans H-33 or M-1 (the structures of which are shown in Table 1B and Table 1E). The one or 5 more glycan moieties can include at least one of glycans K-4, K-10, K-12, or K-13 (the structures of which are shown in Table 1D). The one or more glycan moieties can include at least one of glycans H-3, K-22, K-23, K-24, K-27, K-28, K-29, K-30, or K-31 (the structures of which are shown in Table 1B and Table 1D). The one or more glycan moieties can include at least one of glycans K-51, K-52, K-53, H-7 or H-8 (the structures of which are shown in Table 1B and Table 1D). The one or more glycan moieties can include glycan H-9 (the structure of which is shown in Table 1B). The one or more glycan moieties can include glycan H-6 (the structure of which is shown in Table 1B). The one or more glycan moieties can include at least one of glycans H-23 or H-64 (the structure of which is shown in Table 1B).

The glyco-ligand can include a linker between the glycan and the synthetic scaffold domain of the glyco-ligand. The linker can be a linker of any structure. The chemical structure of the linker can be

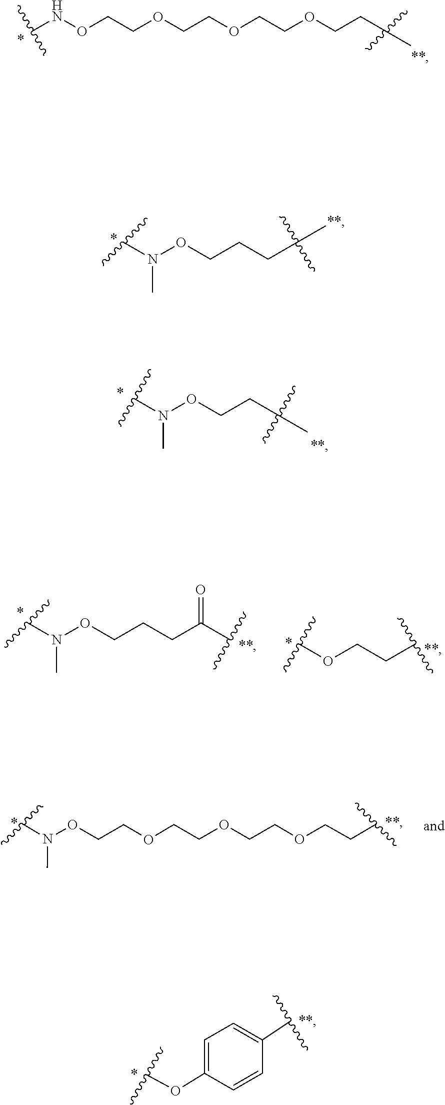

where * indicates the point of attachment to the glycan (e.g., at the non-reducing end terminal monosaccharide of the glycan) and ** indicates the point of attachment to the synthetic scaffold domain (e.g., RNA, siRNA). The linker can be formed by any chemical reaction. The linker can be formed by a click chemistry reaction (e.g., a bioorthogonal click-chemistry reaction).

The synthetic scaffold domain of the glyco-ligand can be any type of RNA, such as siRNA. The siRNA can include one or more of any known ribose modification, modified base, or phosphate linkage modification, of which many are known in the art. The phosphate linkage modification can be a phosphorothioate linkage (PS), phosphorodithioate linkage (PS2), phosphoramidate linkage, phosphorodiamidate linkage, thiophosphoramidate linkage, mesyl phosphoramidate linkage, methylphosphonate linkage (MP), methoxypropylphosphonate linkage (MOP), 5′-(E)-vinylphosphonate linkage (5′-(E)-VP), 5′-Methyl Phosphonate linkage (5′-MP), (S)-5′-C-methyl with phosphate linkage, 5′-phosphorothioate linkage (5′-PS), or a peptide nucleic acid linkage (PNA). The ribose modification can be a 2′-O-methyl (2′-OMe), 2′-O-methoxyethyl (2′-O-MOE), 2′-deoxy, 2′-deoxy-2′-fluoro (2′-F), 2′-arabino-fluoro (2′-Ara-F), 2′-O-benzyl, 2′-O-methyl-4-pyridine (2′-O—CH2Py(4)), Locked nucleic acid (LNA), (S)-cET-BNA, tricyclo-DNA (tcDNA), phosphorodiamidate morpholino oligomer (PMO), hexose nucleic acid (HNA), Unlocked Nucleic Acid (UNA), threose nucleic acid (TNA), 4′-deoxy-4′thioribonucleic acid, or glycol nucleic acid (GNA). The modified base can be pseudouridine (ψ), 2′thiouridine (s2U), N6′-methyladenosine (m6A), 5′methylcytidine (m5C), 5′-fluoro-2′-deoxyuridine, N-ethylpiperidine 7′-EAA triazole modified adenine, N-ethylpiperidine 6′-triazole modified adenine, 6′-phenylpyrrolo-cytosine (PhpC), 2′,4′-difluorotoluyl ribonucleoside (rF), or 5′-nitroindole. The siRNA may contain one or more modifications to one or more nucleotides that include 2-OMe modification, fluoride modification, a phosphorothioate modification or a combination thereof.

Described herein are also methods of treating a disease or condition. The method can include administering a therapeutically effective amount of a pharmaceutical composition (e.g., a pharmaceutical composition containing a glyco-ligand) to a subject in need thereof. The pharmaceutical compositions described herein can be used to manufacture a medicament for the treatment of a disease or a condition. The pharmaceutical compositions can be used for the treatment of a disease or condition in a subject in need thereof.

### BRIEF DESCRIPTION OF THE DRAWINGS

FIG. 1 are diagrams of exemplary glycans of the present disclosure.

FIG. 2A is a diagram of various scaffold domains (e.g., synthetic scaffold domains) including, a phospholipid, an antibody, a peptide, and RNA types suitable for glycan conjugation and glycans operably linked to the RNA types (glycoRNA). FIG. 2B is an illustration showing protein-based and nucleic acid-based glyco-ligands where the nucleic acid-based glyco-ligand is configured to be a desired structure and orientation for glycan-receptor engagement. FIG. 2C is an illustration showing a bioactive molecule conjugated to a glyco-ligand (radio-ligand conjugated to glycoRNA). FIG. 2D is an illustration showing additional select molecules such as toxins, enzymes and proteins/peptides conjugated to glyco-ligands.

FIG. 3 is a diagram of an exemplary glyco-ligand conjugation chemistry.

FIG. 4A is an illustration of an exemplary configurable glyco-ligand presented in an orientation leading to clustering of signaling proteins and binding to one or more receptors. FIG. 4B is an illustration showing a sialylated glycoRNA mediated binding to a sialic acid binding-immunoglobulin lectin-type (Siglec) receptor.

FIG. 5 is an illustration showing receptor-mediated internalization of exemplary glyco-ligands of the present disclosure.

### DETAILED DESCRIPTION

The present disclosure provides methods and compositions for modulating cell surface proteins and receptor complexes using a novel class of glycan conjugates that can be used to engage the signaling pathways within desired cell types. Such defined cell-targeting bioactive glyco-ligands are directed for cell engagement and activation in therapeutic applications.

### Definitions

Unless otherwise defined herein, scientific and technical terms used in connection with the present invention shall have the meanings that are commonly understood by those of ordinary skill in the art. Further, unless otherwise required by context, singular terms shall include the plural and plural terms shall include the singular. Generally, nomenclatures used in connection with, and techniques of, biochemistry, enzymology, molecular and cellular biology, microbiology, genetics and protein and nucleic acid chemistry and hybridization described herein are those well-known and commonly used in the art.

The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999).

All publications, patents and other references mentioned herein are hereby incorporated by reference in their entireties.

The following terms, unless otherwise indicated, shall be understood to have the following meanings:

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Throughout this specification and claims, the word “comprise” or variations such as “comprises” or “comprising”, will be understood to imply the inclusion of a stated integer or group of integers but not the exclusion of any other integer or group of integers.

As used herein, the term “synthetic scaffold domain” refers to without limitation DNA, RNA, cellulose, chitosan, glycosaminoglycan (GAG), hyaluronic acid, chondroitin sulfate, alginates, polycaprolactone, collagen, including nanoparticles or nanostructures. In certain embodiments, the term “synthetic scaffold domain” refers to an RNA or DNA, preferably an RNA, more preferably an siRNA.

As used herein, the term “modified sites” refers to one or more sites on a scaffold or a position on the scaffold, e.g., polymer containing reactive functional groups suitable for glycan conjugation or more specifically the conjugation site of one or more glycans.

As used herein, the term “polymer” refers to a substance composed of natural or synthetic monomers, such as ribonucleotides.

As used herein, the term “bioactive” refers to a biologically active molecule. For example, in the context of an assay with respect to a “bioactive polymer”, receptor binding as demonstrated by SPR can detect biomolecular interactions, including those between a saccharide and protein, to indicate a biologically active molecule [Nguyen H H, Park J, Kang S, Kim M. Surface plasmon resonance: a versatile technique for biosensor applications. Sensors (Basel). 2015 May 5; 15(5):10481-510.].

As used herein, the term “moiety” refers to a molecule. For instance, a “carbohydrate moiety” or an “oligosaccharide moiety” generally refers to a glycan composition.

A “modified sequence” is a nucleic acid molecule that includes at least one difference from a naturally-occurring nucleic acid molecule. A modified sequence includes all exogenous modified and unmodified heterologous sequences (i.e., sequences derived from an organism or cell other than that harboring the modified sequence) as well as endogenous genes, operons, coding sequences, or non-coding sequences, that have been modified, mutated, or that include deletions or insertions as compared to a naturally-occurring sequence. Such sequences also include all sequences, regardless of origin, that are linked to an inducible promoter or to another control sequence with which they are not naturally associated. Such sequences further include all sequences that can be used to down-regulate or knock out expression of an endogenous gene. These include anti-sense molecules, RNAi molecules, constructs for producing homologous recombination, cre-lox constructs, and the like.

The term “polynucleotide” or “nucleic acid molecule” or “nucleotide sequence” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

An “isolated” RNA, DNA or a mixed polymer is one which is substantially separated from other cellular components that naturally accompany the native polynucleotide in its natural 10 host cell, e.g., ribosomes, polymerases and genomic sequences with which it is naturally associated.

As used herein, an “isolated” composition (e.g., glyco-ligand) is one which is substantially separated from the cellular components (membrane lipids, chromosomes, proteins) of the host cell from which it originated, or from the medium in which the host cell was cultured. The term does not require that the biomolecule has been separated from all other chemicals, although certain isolated biomolecules may be purified to near homogeneity.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

As used herein, an endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

As used herein, the phrase “degenerate variant” of a reference nucleic acid sequence encompasses nucleic acid sequences that can be translated, according to the standard genetic code, to provide an amino acid sequence identical to that translated from the reference nucleic acid sequence. The term “degenerate oligonucleotide” or “degenerate primer” is used to signify an oligonucleotide capable of hybridizing with target nucleic acid sequences that are not necessarily identical in sequence but that are homologous to one another within one or more particular segments.

The term “percent sequence identity” or “identical” in the context of nucleic acid sequences refers to the residues in the two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson, Methods Enzymol. 183:63-98 (1990) (hereby incorporated by reference in its entirety). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Sequences can be compared using the computer program, BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 76%, at least about 80%, at least about 85%, preferably at least about 90%, and more preferably at least about 95%, at least about 96%, at 12 least about 97%, at least about 98% or at least about 99% of the nucleotide bases, as measured by any well-known algorithm of sequence identity, such as FASTA, BLAST or Gap, as discussed above.

Substantial homology or similarity exists when a nucleic acid or fragment thereof hybridizes to another nucleic acid, to a strand of another nucleic acid, or to the complementary strand thereof, under stringent hybridization conditions. “Stringent hybridization conditions” and “stringent wash conditions” in the context of nucleic acid hybridization experiments depend upon a number of different physical parameters. Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. One having ordinary skill in the art knows how to vary these parameters to achieve a particular stringency of hybridization.

In general, “stringent hybridization” is performed at about 25° C. below the thermal melting point (Tₘ) for the specific DNA hybrid under a particular set of conditions. “Stringent washing” is performed at temperatures about 5° C. lower than the Tₘ for the specific DNA hybrid under a particular set of conditions. The Tₘ is the temperature at which 50% of the target sequence hybridizes to a perfectly matched probe. See Sambrook et al., Molecular Cloning: A Laboratory Manual, 2d ed., Cold Spring Harbor Laboratory Press, Cold Spring Harbor, N.Y. (1989), page 9.51, hereby incorporated by reference. For purposes herein, “stringent conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for 8-12 hours, followed by two washes in 0.2×SSC, 0.1% SDS at 65° C. for 20 minutes. It will be appreciated by the skilled worker that hybridization at 65° C. will occur at different rates depending on a number of factors including the length and percent identity of the sequences which are hybridizing.

The nucleic acids (also referred to as polynucleotides) of this present invention may include both sense and antisense strands of RNA, cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. Other modifications can include, for example, analogs in which the ribose ring contains a bridging moiety or other structure such as the modifications found in “locked” nucleic acids.

The term “mutated” when applied to nucleic acid sequences means that nucleotides in a nucleic acid sequence may be inserted, deleted or changed compared to a reference nucleic acid sequence. A single alteration may be made at a locus (a point mutation) or multiple nucleotides may be inserted, deleted or changed at a single locus. In addition, one or more alterations may be made at any number of loci within a nucleic acid sequence. A nucleic acid sequence may be mutated by any method known in the art including but not limited to mutagenesis techniques such as “error-prone PCR” (a process for performing PCR under conditions where the copying fidelity of the DNA polymerase is low, such that a high rate of point mutations is obtained along the entire length of the PCR product; see, e.g., Leung et al., Technique, 1:11-15 (1989) and Caldwell and Joyce, PCR Methods Applic. 2:28-33 (1992)); and “oligonucleotide-directed mutagenesis” (a process which enables the generation of site-specific mutations in any cloned DNA segment of interest; see, e.g., Reidhaar-Olson and Sauer, Science 241:53-57 (1988)).

The term “downregulate,” as in “downregulating a signal,” means the process whereby the level of target gene expression prior to and following contact with the glyco-ligand can be compared, e.g., on an mRNA or protein level. If it is determined that the amount of RNA or protein expressed from the target gene is lower following contact with the glyco-ligand, then it can be concluded that the glyco-ligand downregulates target gene expression. The level of target RNA or protein in the cell can be determined by any method desired. For example, the level of target RNA can be determined by Northern blot analysis, reverse transcription coupled with polymerase chain reaction (RT-PCR), or RNAse protection assay. The level of protein can be determined, for example, by Western blot analysis.

The term “silence,” as in “silencing a target gene,” means the process whereby a cell containing and/or secreting a certain product of the target gene when not in contact with the glyco-ligand, will contain and/or secrete at least 10%, at least 20%, at least 30%, at least 40%, at least 50%, at least 60%, at least 70%, at least 80%, or at least 90% less of such gene product when contacted with the glyco-ligand, as compared to a similar cell which has not been contacted with the glyco-ligand. Such product of the target gene can, for example, be a messenger RNA (mRNA), a protein, or a regulatory element.

The term “attenuate” as used herein generally refers to a functional deletion, including a mutation, partial or complete deletion, insertion, or other variation made to a gene sequence or a sequence controlling the transcription of a gene sequence, which reduces or inhibits production of the gene product, or renders the gene product non-functional. In some instances, a functional deletion is described as a knockout mutation. Attenuation also includes amino acid sequence changes by altering the nucleic acid sequence, placing the gene under the control of a less active promoter, down-regulation, expressing interfering RNA, ribozymes or antisense sequences that target the gene of interest, or through any other technique known in the art. In one example, the sensitivity of a particular enzyme to feedback inhibition or inhibition caused by a composition that is not a product or a reactant (non-pathway specific feedback) is lessened such that the enzyme activity is not impacted by the presence of a compound. In other instances, an enzyme that has been altered to be less active can be referred to as attenuated. The term “deletion” as used herein with respect to gene sequences generally refers to the removal of one or more nucleotides from a nucleic acid molecule or one or more amino acids from a protein, the regions on either side being joined together. The term “knock-out” as used herein with respect to gene sequences generally refers to a gene whose level of expression or activity has been reduced to zero. In some examples, a gene is knocked-out via deletion of some or all of its coding sequence. In other examples, a gene is knocked-out via introduction of one or more nucleotides into its open reading frame, which results in translation of a non-sense or otherwise non-functional protein product.

The term “vector” as used herein is intended to refer to a nucleic acid molecule capable of transporting another nucleic acid to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply “expression vectors”).

“Operatively linked” or “operably linked” expression control sequences refers to a linkage in which the expression control sequence is contiguous with the gene of interest to control the gene of interest, as well as expression control sequences that act in trans or at a distance to control the gene of interest. The term is also used herein with respect to a glycan moiety conjugated to a synthetic scaffold domain as described herein.

The term “expression control sequence” as used herein refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “recombinant host cell” (or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

The term “polypeptide fragment” as used herein refers to a polypeptide that has a deletion, e.g., an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, at least 6, at least 7, at least 8, at least 9 or at least 10 amino acids long, preferably at least 12, at least 14, at least 16 or at least 18 amino acids long, more preferably at least 20 amino acids long, more preferably at least 25, at least 30, at least 35, at least 40 or at least 45, amino acids, even more preferably at least 50 or at least 60 amino acids long, and even more preferably at least 70 amino acids long.

A “modified derivative” refers to polypeptides or fragments thereof that are substantially homologous in primary structural sequence but which include, e.g., in vivo or in vitro chemical and biochemical modifications or which incorporate amino acids that are not found in the native polypeptide. Such modifications include, for example, acetylation, carboxylation, phosphorylation, glycosylation, ubiquitination, labeling, e.g., with radionuclides, and various enzymatic modifications, as will be readily appreciated by those skilled in the art. A variety of methods for labeling polypeptides and of substituents or labels useful for such purposes are well known in the art, and include radioactive isotopes such as ¹²⁵I, ³²P, ³⁵S, and ³H, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods for labeling polypeptides are well known in the art. See, e.g., Ausubel et al., Current Protocols in Molecular Biology, Greene Publishing Associates (1992, and Supplements to 2002) (hereby incorporated by reference).

The term “fusion protein” refers to a polypeptide comprising a polypeptide or fragment coupled to heterologous amino acid sequences. Fusion proteins are useful because they can be constructed to contain two or more desired functional elements from two or more different proteins. A fusion protein comprises at least 10 contiguous amino acids from a polypeptide of interest, more preferably at least 20 or at least 30 amino acids, even more preferably at least 40, at least 50 or at least 60 amino acids, yet more preferably at least 75, at least 100 or at least 125 amino acids. Fusions that include the entirety of the proteins of the present invention have particular utility. The heterologous polypeptide included within the fusion protein of the present invention is at least 6 amino acids in length, often at least 8 amino acids in length, and usefully at least 15, at least 20, and at least 25 amino acids in length. Fusions that include larger polypeptides, such as an IgG Fc region, and even entire proteins, such as the green fluorescent protein (“GFP”) chromophore-containing proteins, have particular utility. Fusion proteins can be produced recombinantly by constructing a nucleic acid sequence which encodes the polypeptide or a fragment thereof in frame with a nucleic acid sequence encoding a different protein or peptide and then expressing the fusion protein. A fusion protein can be produced chemically by crosslinking the polypeptide or a fragment thereof to another protein.

The term “non-peptide analog” refers to a compound with properties that are analogous to those of a reference polypeptide. A non-peptide compound may also be termed a “peptide mimetic” or a “peptidomimetic.” See, e.g., Jones, Amino Acid and Peptide Synthesis, Oxford University Press (1992); Jung, Combinatorial Peptide and Nonpeptide Libraries: A Handbook, John Wiley (1997); Bodanszky et al., Peptide Chemistry—A Practical Textbook, Springer Verlag (1993); Synthetic Peptides: A Users Guide, (Grant, ed., W. H. Freeman and Co., 1992); Evans et al., J. Med. Chem. 30:1229 (1987); Fauchere, J. Adv. Drug Res. 15:29 (1986); Veber and Freidinger, Trends Neurosci., 8:392-396 (1985); and references sited in each of the above, which are incorporated herein by reference. Such compounds are often developed with the aid of computerized molecular modeling. Peptide mimetics that are structurally similar to useful peptides of the present invention may be used to produce an equivalent effect and are therefore envisioned to be part of the present invention.

A “polypeptide mutant” or “mutein” refers to a polypeptide whose sequence contains an insertion, duplication, deletion, rearrangement or substitution of one or more amino acids compared to the amino acid sequence of a native or wild-type protein. A mutein may have one or more amino acid point substitutions, in which a single amino acid at a position has been changed to another amino acid, one or more insertions and/or deletions, in which one or more amino acids are inserted or deleted, respectively, in the sequence of the naturally-occurring protein, and/or truncations of the amino acid sequence at either or both the amino or carboxy termini. A mutein may have the same but preferably has a different biological activity compared to the naturally-occurring protein.

A mutein has at least 85% overall sequence homology to its wild-type counterpart. Even more preferred are muteins having at least 90% overall sequence homology to the wild-type protein.

In an even more preferred embodiment, a mutein exhibits at least 95% sequence identity, even more preferably at least 98%, even more preferably at least 99% and even more preferably at least 99.9% overall sequence identity.

Sequence homology may be measured by any common sequence analysis algorithm, such as Gap or Bestfit.

Amino acid substitutions can include those which: (1) reduce susceptibility to proteolysis, (2) reduce susceptibility to oxidation, (3) alter binding affinity for forming protein complexes, (4) alter binding affinity or enzymatic activity, and (5) confer or modify other physicochemical or functional properties of such analogs.

As used herein, the twenty conventional amino acids and their abbreviations follow conventional usage. See Immunology—A Synthesis (Golub and Gren eds., Sinauer Associates, Sunderland, Mass., 2^{nd} ed. 1991), which is incorporated herein by reference. Stereoisomers (e.g., D-amino acids) of the twenty conventional amino acids, unnatural amino acids such as α-, α-disubstituted amino acids, N-alkyl amino acids, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). In the polypeptide notation used herein, the left-hand end corresponds to the amino terminal end and the right-hand end corresponds to the carboxy-terminal end, in accordance with standard usage and convention.

A protein has “homology” or is “homologous” to a second protein if the nucleic acid sequence that encodes the protein has a similar sequence to the nucleic acid sequence that encodes the second protein. In embodiments, a protein has homology to a second protein if the two proteins have “similar” amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89 (herein incorporated by reference).

The following six groups each contain amino acids that are conservative substitutions for one another: 1) Serine(S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

A preferred algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990) (incorporated by reference herein). For example, percent sequence identity between amino acid sequences can be determined using FASTA with its default parameters (a word size of 2 and the PAM250 scoring matrix), as provided in GCG Version 6.1, herein incorporated by reference.

“Specific binding” refers to the ability of two molecules to bind to each other in preference to binding to other molecules in the environment. Typically, “specific binding” discriminates over adventitious binding in a reaction by at least two-fold, more typically by at least 10-fold, often at least 100-fold. Typically, the affinity or avidity of a specific binding reaction, as quantified by a dissociation constant, is about 10⁻⁷ M or stronger (e.g., about 10⁻⁸ M, 10⁻⁹ M or even stronger).

The term “region” as used herein refers to a physically contiguous portion of the primary structure of a biomolecule. In the case of proteins, a region is defined by a contiguous portion of the amino acid sequence of that protein.

The term “domain” as used herein refers to a structure of a biomolecule that contributes to a known or suspected function of the biomolecule. Domains may be co-extensive with regions or portions thereof; domains may also include distinct, non-contiguous regions of a biomolecule. Examples of protein domains include, but are not limited to, an Ig domain, an extracellular domain, a transmembrane domain, and a cytoplasmic domain.

As used herein, the term “molecule” means any compound, including, but not limited to, a small molecule, peptide, protein, sugar, nucleotide, nucleic acid, lipid, etc., and such a compound can be natural or synthetic.

The term “N-linked glycan” or “N-glycans” refers to a N-linked oligosaccharide structures, that are covalently bound to a nitrogen atom, optionally via an amide bond, optionally as an N-glycan conjugated at an asparagine or arginine residue via an N-acetylglucosamine residue on the glycan generally via glycosyltransferase. These “N-linked glycosylation sites” occur in the peptide primary structure containing, for example, the canonical amino acid sequence asparagine-X-serine/threonine, where X is any amino acid residue except proline and aspartic acid. “N-linked glycans” refer to N-linked oligosaccharide structures. The N-glycans can be attached to proteins or synthetic scaffold domains, which can be manipulated further in vitro or in vivo. Common N-linked glycans typically include complex, hybrid, high-mannose, branched, and multiple antennary structures. The term “N-linked type” with respect to a glycan can refer to a scaffold having an attached N-acetylglucosamine (GlcNAc) residue linked to the amide nitrogen of an asparagine residue (N-linked) on the protein or synthetic scaffold domain, that is similar or even identical to those produced in humans.

“O-glycans” or “O-linked glycans” refer to O-linked oligosaccharide structures. The O-glycans can be attached to proteins or synthetic scaffold domains, which can be manipulated further in vitro or in vivo. Common O-GalNAc core structures typically include Core 1, Core 2 and poly-N-acetyllactosamine (LacNAc) structures. In some embodiments, the O-linked oligosaccharide are covalently bound via an oxygen atom on a serine residue. The term “O-linked type” with respect to glycans can refer to conjugates having an attached N-acetylgalactosamine (GalNAc) residue linked to the oxygen atom of a serine or threonine residue on the protein or scaffold, that is similar or even identical to those produced in humans.

As used herein, the term “monosaccharide” refers to a carbohydrate molecule that cannot be hydrolyzed into two or more simpler carbohydrates. Examples of monosaccharides include, but are not limited to, GlcNAc, mannose, fucose, glucose, fructose and galactose.

The term “glycan” refers to oligosaccharide structures—the predominant oligosaccharide structures found on glycoproteins include glucose (Glu), galactose (Gal), mannose (Man), fucose (Fuc), N-acetylglucosamine (GlcNAc), N-acetylgalactosamine (GalNAc), glucosamine (GlcN), galactosamine (GalN), glucuronic acid (GlcA), iduronic acid (IdoA), and sialic acid (e.g., N-acetyl-neuraminic acid (NeuNAc or NANA)). Hexoses (Hex), categorized as monosaccharides with 6 carbon atoms, such as glucose, galactose, mannose, are not readily discernable via mass spectrometry and may also be present. N-glycans differ with respect to the number of branches (“antennae” or “arms”) comprising peripheral sugars (e.g., GlcNAc, galactose, fucose and sialic acid) that are added to the “trimannosyl core.” The term “trimannosyl core”, also referred to as “M3”, “M3GN2”, the “trimannose core”, the “pentasaccharide core” or the “paucimannose core” reflects Man₃GlcNAc₂ oligosaccharide structure where the Manα1,3 arm and the Manα1,6 arm extends from the di-GlcNAc structure (GlcNAc₂): β1,4GlcNAc-β1,4GlcNAc. N-glycans are classified according to their branched constituents (e.g., high-mannose, complex or hybrid).

A “high-mannose” type N-glycan comprises four or more mannose residues on the di-GlcNAc oligosaccharide structure. “M9” reflects Man₉GlcNAc₂. “M5” reflects Man₅GlcNAc₂.

A “hybrid” type N-glycan has at least one GlcNAc residue on the terminal end of the α1,3 mannose (Man α1,3) arm of the trimannose core and zero or more mannoses on the α1,6 mannose (Man α1,6) arm of the trimannose core. An example of a hybrid glycan is GlcNAcMan₃GlcNAc₂.

A “complex” type N-glycan typically has at least one GlcNAc residue attached to the Manα1,3 arm and at least one GlcNAc attached to the Manα1,6 arm of the trimannose core (sometimes referred to as “G0” or “G0F” fucosylated). Complex N-glycans may also have galactose or N-acetylgalactosamine residues (“G2” or “G2F” fucosylated) that are optionally modified with sialic acid (“G2S2” or “G2FS2” fucosylated) or derivatives (e.g., “Neu” refers to neuraminic acid and “Ac” refers to acetyl). Complex N-glycans may also have intrachain substitutions comprising “bisecting” GlcNAc and core fucose. Complex N-glycans may also have multiple antennae on the trimannose core, often referred to as “multiple antennary glycans” or also termed “multi-branched glycans,” which can be tri-antennary, tetra-antennary, or penta-antennary glycans.

The term “glycoform” generally refers to an isoform of an oligosaccharide attached to a protein or scaffold, e.g., a RNA molecule, that differs only with respect to the number and/or type of attached glycan(s). Glyco-ligands can comprise one or more different or the same glycoforms. Glycoforms can be referred to as homogenous, predominant or heterogeneous based on the presence or absence of one or more isoforms of an oligosaccharide attached or conjugated on a protein or a synthetic scaffold domain measured typically through analytical techniques.

As used herein, the term “predominantly” or variations such as “the predominant” or “which is predominant” will be understood to mean the glycan species as measured that has the highest mole percent (%) of total N-glycans after the glyco-ligand has been removed (e.g., treated with PNGase and the glycans released) and are analyzed by mass spectroscopy, for example, MALDI-TOF MS. In other words, the phrase “predominantly” is defined as an individual entity, such as a specific glycoform, present in greater mole percent than any other individual entity. For example, if a composition consists of species A in 40 mole percent, species B in 35 mole percent and species C in 25 mole percent, the composition comprises predominantly species A. The term “enriched”, “uniform”, “homogenous” and “consisting essentially of” are also synonymous with “predominant” in reference to one or more glycans.

The mole % of N-glycans as measured by MALDI-TOF-MS in positive mode refers to mole % saccharide transfer with respect to mole % total N-glycans. Certain cation adducts such as K+ and Na+ are normally associated with the peaks eluted increasing the mass of the N-glycans by the molecular mass of the respective adducts.

The term “effective amount” or “therapeutically effective amount” means a dosage sufficient to produce a desired result, e.g., an amount sufficient to effect beneficial or desired (including preventative and/or therapeutic) results, such as a reduction in a symptom of a medical condition (e.g., cancer, an infectious disease, an immune-mediated disorder (e.g., an autoimmune disorder, an inflammatory disorder), etc.) as compared to a control. With respect to cancer, in some embodiments, the therapeutically effective amount is sufficient to slow the growth of a tumor, reduce the size of a tumor, and/or the like. An effective amount can be administered in one or more administrations.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), or unsubstituted isobutyl (i-Bu)). In certain embodiments, the alkyl group is a substituted C₁₋₁₀ alkyl (such as substituted C₁₋₆ alkyl, e.g., —CF₃, Bn).

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In some embodiments, a heteroalkyl group is a saturated group having 1 to 18 carbon atoms and 1 or more heteroatoms within the parent chain (“heteroC₁₋₁₈ alkyl”). In some embodiments, a heteroalkyl group is a saturated group having 1 to 16 carbon atoms and 1 or more heteroatoms within the parent chain (“heteroC₁₋₁₆ alkyl”). In some embodiments, a heteroalkyl group is a saturated group having 1 to 14 carbon atoms and 1 or more heteroatoms within the parent chain (“heteroC₁₋₁₄ alkyl”). In some embodiments, a heteroalkyl group is a saturated group having 1 to 12 carbon atoms and 1 or more heteroatoms within the parent chain (“heteroC₁₋₁₂ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and I heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀ alkyl. In certain embodiments, the heteroalkyl group is an unsubstituted heteroC₁₋₁₀ alkyl. In certain embodiments, the heteroalkyl group is a substituted heteroC₁₋₂₀ alkyl. In certain embodiments, the heteroalkyl group is an unsubstituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

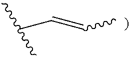

may be an (E)- or (Z)-double bond.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered 31 heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo [3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro [3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not intended to be limited in any manner by the exemplary substituents described herein.

When substituted, exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR)₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb}) OR^{aa}, —OC(═NR^{bb})R^{aa}, —OC(═NR^{bb}) OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O) OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O) R^{aa}, —C(═O)N(R^{cc})₂, —CO₂R^{aa}, —SO₂R^{aa}, —C(═NR^{cc}) OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), OC(═NH) OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂ (C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, OSi(C₁₋₆ alkyl)₃-C(═S)N(C₁₋₆ alkyl)₂, C(═S)NH(C₁₋₆ alkyl), C(═S)NH₂, C(═O)S(C₁₋₆ alkyl), C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphor sulfonate, citrate, cyclopentanepropionate, digluconate, dodecyl sulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N+(C1-4 alkyl)4 salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal. A “patient” refers to a human subject in need of treatment of a disease.

The terms “administer,” “administering,” or “administration,” refers to implanting, absorbing, ingesting, injecting, inhaling, or otherwise introducing an inventive compound, or a pharmaceutical composition thereof.

The terms “treatment,” “treat,” and “treating” refer to reversing, alleviating, delaying the onset of, or inhibiting the progress of a “pathological condition” (e.g., a disease, disorder, or condition, or one or more signs or symptoms thereof) described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The terms “valency” or “multivalency” as used herein generally refer to one (monovalent) or more (multivalent) glycans on one scaffold capable of binding to the receptors or the carbohydrate recognition domains of a target. Relatedly, the term “heteromultivalency” as used herein generally refers to different or a mixture of heterogeneous glycans on one scaffold capable of binding to the receptors or the carbohydrate recognition domain of a target.

As used herein, the term “selective” or “selectively binds” refers to a ligand-receptor relationship wherein the ligand binds to the receptor with at least 90% specificity, such that there is less than 10% off-target binding. In some embodiments, the ligand binds the receptor with at least 95%, at least 98%, at least 99% or at least 99.9% specificity.

Unless otherwise defined, all technical and scientific terms used herein have the same meaning as commonly understood by one of ordinary skill in the art to which this present invention pertains. Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting.

### Nucleic Acid Sequences

In various aspects, the methods and compositions comprise one or more glycans operably linked to one or more sites on a synthetic scaffold domain comprising a synthetic nucleic acid polymer wherein the nucleic acid polymer comprises RNA. The methods provide synthesizing one or more glyco-ligand to modulate a desired receptor to mediate a biological effect.

In preferred aspects, the method for synthesizing a glyco-ligand of the invention includes conjugating a glycan to one or more short hairpin RNAs, double-stranded RNAs, long noncoding RNAs, circular RNAs (circRNA), small cell nuclear RNAs (Y NRA), short interfering RNAs (siRNA), antisense oligonucleotide (ASO), messenger RNA (mRNA), guide RNA on a ribonucleoprotein (RNP), aptamer and other such nucleic acid molecules.

Preferably, the synthetic nucleic acid polymer comprises at least one nucleobase modification. More preferably, the methods and compositions comprise modification of one or more nucleic acid sequence by an insertion, deletion or alteration of one or more base pairs at the target region for conjugation. In various embodiments, Y RNAs, small nuclear RNAs, and small nucleolar RNAs are modified at guanosine residues.

The nucleic acid polymer can be an siRNA or ASO described by Hu, et al., Sig Transduct Target Ther 5, 101 (2020). The siRNA or ASO can comprise one or more phosphate modifications. Exemplary phosphate linkage modifications include, but are not limited to, phosphorothioate linkage (PS), phosphorodithioate linkage (PS2), phosphoramidate linkage, phosphorodiamidate linkage, thiophosphoramidate linkage, mesyl phosphoramidate linkage, methylphosphonate linkage (MP), methoxypropylphosphonate linkage (MOP), 5′-(E)-vinylphosphonate linkage (5′-(E)-VP), 5′-Methyl Phosphonate linkage (5′-MP), (S)-5′-C-methyl with phosphate linkage, 5′-phosphorothioate linkage (5′-PS), a peptide nucleic acid linkage (PNA), or variations thereof. The siRNA or ASO can comprise one or more ribose modifications. Exemplary ribose modifications include, but are not limited to 2′-O-methyl (2′-OMe), 2′-O-methoxyethyl (2′-O-MOE), 2′-deoxy, 2′-deoxy-2′-fluoro (2′-F), 2′-arabino-fluoro (2′-Ara-F), 2′-O-benzyl, 2′-O-methyl-4-pyridine (2′-O—CH2Py(4)), Locked nucleic acid (LNA), (S)-cET-BNA, tricyclo-DNA (tcDNA), phosphorodiamidate morpholino oligomer (PMO), hexose nucleic acid (HNA), Unlocked Nucleic Acid (UNA), threose nucleic acid (TNA), 4′-deoxy-4′thioribonucleic acid, and glycol nucleic acid (GNA). The siRNA can comprise a Locked Nucleic Acid (LNA) comprising a methyl bridge, an ethyl bridge, a propyl bridge, a butyl bridge or an optionally substituted variant of any of the aforementioned. The siRNA or ASO of the present disclosure can comprise one or more modified bases. Exemplary modified bases include, but are not limited to, pseudouridine (ψ), 2′thiouridine (s2U), N6′-methyladenosine (m⁶A), 5′methylcytidine (m⁵C), 5′-fluoro-2′-deoxyuridine, N-ethylpiperidine 7′-EAA triazole modified adenine, N-ethylpiperidine 6′-triazole modified adenine, 6′-phenylpyrrolo-cytosine (PhpC), 2′,4′-difluorotoluyl ribonucleoside (rF), and 5′-nitroindole. Further exemplary modified nucleic acids are shown in Table 5A.

Phosphate linkage modifications, ribose modifications, modified bases, and combinations thereof can increase the stability and/or permeability of a nucleic acid molecule (e.g., siRNA, ASO) in comparison to unmodified nucleic acid molecules. Modifications can increase the pH stability of a nucleic acid molecule (e.g., the stability to acid or base, the resistance of a molecule to chemical hydrolysis). Modifications can increase the enzymatic stability of a nucleic acid molecule to any nuclease enzyme including, but not limited to, extracellular endonucleases, extracellular exonucleases, intracellular exonucleases, intracellular endonucleases, or combinations thereof. Modifications to nucleic acid molecules can increase permeability across a cell membrane (e.g., increase permeation into a cell), across a layer of cells (e.g., across an endothelial layer of cells, across an epithelial layer of cells), or a combination thereof. Phosphate linkage modifications, ribose modifications, modified bases, and combinations thereof can decrease the immunogenicity of a nucleic acid molecule (e.g., siRNA, ASO) in comparison to unmodified nucleic acid molecules.

In other aspects, one or more nucleobase on a synthetic scaffold domain is modified, for example as a target site to which one or more glycans can be operably linked in the assembly. Preferably, the nucleobase modification provides a covalent linkage to one or more desired glycans resulting in a glyco-ligand composition. In certain embodiments, the glyco-ligand composition comprises a plurality of modifications to the nucleic acids suitable for better industrial suitability and applicability.

Certain modified sequences are made to alter the functionality of the nucleic acid sequence that are undesirable, counterproductive, interfere with, detrimental to, or are less suitable as a glyco-ligand composition.

In such embodiments wherein the synthetic scaffold domain comprises RNA, one or more nucleobase is modified at one or more guanosine sites. As the case may be for specific types of RNA, for instance siRNAs, specific patterns of alternating 2′-O-methyl and 2′-O-fluoro nucleotides are made with insertion of phosphorothioate bonds (PS) at the extremities of the strands to enhance pharmacokinetics properties. In such embodiments wherein the synthetic scaffold domain comprises ASO, modifications on the 2′ position of the furanose sugar can enhance metabolic stability and binding affinity for the biological target, as well as improve toxicology and pharmacokinetic properties. (Prakash, TP. An overview of sugar-modified oligonucleotides for antisense therapeutics. Chem Biodivers. 2011 September; 8(9):1616-41). In more preferred embodiments, the synthetic scaffold domain comprising RNA comprises from about 5 to about 10 ribonucleotides, from about 10 to about 20 ribonucleotides, from about 20 to about 30 ribonucleotides, from about 30 to about 40 ribonucleotides, from about 40 to about 50 ribonucleotides, from about 50 to about 100 ribonucleotides, from about 100 to about 500 ribonucleotides, from about 500 to about 5,000 ribonucleotides or greater.

Accordingly, the present invention provides an isolated glyco-ligand composition comprising nucleic acid molecules and variants thereof conjugated to one or more desired glycans. Exemplary nucleic acid sequences are non-encoding sequences. The modified sequences can be selected from nucleic acid sequence that are greater than 50%, greater than 60%, greater than 70%, greater than 80%, greater than 85%, greater than 90%, greater than 95%, greater than 98%, greater than 99%, greater than 99.9% or even higher identity to the wild-type non-encoding sequences. In other embodiments, the nucleic acid molecule of the present invention is partially noncoding.

In some embodiments, the nucleic acid polymer is an siRNA. In some embodiments, the nucleic acid polymer is an siRNA comprising a modification to one or more nucleotides, including, but not limited to, a 2′-OMe modification, a fluorine modification (such as a 2-fluororibose modification), and a phosphorothioate modification. In some embodiments, the nucleic acid is an siRNA comprising a modified backbone.

In some embodiments, the nucleic acid is a circular RNA, wherein the circular RNA is modified as compared to a naturally occurring RNA by being self-ligated, thereby lacking a cap or tail. In some embodiments, the nucleic acid is a circular RNA comprising an IRES sequence selected from IRES is from Taura syndrome virus, Triatoma virus, Theiler's encephalomyelitis virus, Simian Virus 40, Solenopsis invicta virus 1, Rhopalosiphum padi virus, Reticuloendotheliosis virus, Human poliovirus 1, Plautia stall intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus, Hepatitis A virus, Hepatitis GB virus, Foot and mouth disease virus, Human enterovirus 71, Equine rhinitis virus, Ectropis obliqua picoma-like virus, Encephalomyocarditis virus, Drosophila C Virus, Human coxsackievirus B3, Crucifer tobamovirus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human ATIR, Human BAG-1, Human BCL2, Human BiP, Human c-IAPl, Human c-myc, Human eIF4G, Mouse NDST4L, Human LEF1, Mouse HIFI alpha, Human n.myc, Mouse Gtx, Human 43 p27kip1, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, S. cerevisiae YAP1, tobacco etch virus, turnip crinkle virus, EMCV-A, EMCV-B, EMCV-Bf, EMCV-Cf, EMCV pEC9, Picobirnavirus, HCV QC64, Human Cosavirus E/D, Human Cosavirus F, Human Cosavirus JMY, Rhinovirus NAT001, HRV14, HRV89, HRVC-02, HRV-A21, Salivirus A SHI, Salivirus FHB, Salivirus NG-J1, Human Parechovirus 1, Crohivirus B, Yc-3, Rosavirus M-7, Shanbavirus A, Pasivirus A, Pasivirus A 2, Echovirus E14, Human Parechovirus 5, Aichi Virus, Hepatitis A Virus HA 16, Phopivirus, CVA10, Enterovirus C, Enterovirus D, Enterovirus J, Human Pegivirus 2, GBV-C GT110, GBV-C K1737, GBV-C Iowa, Pegivirus A 1220, Pasivirus A 3, Sapelovirus, Rosavirus B, Bakunsa Virus, Tremovirus A, Swine Pasivirus 1, PLV-CHN, Pasivirus A, Sicinivirus, Hepacivirus K, Hepacivirus A, BVDV1, Border Disease Virus, BVDV2, CSFV-PK15C, SF573 Dicistrovirus, Hubei Picoma-like Virus, CRPV, Salivirus A BN5, Salivirus A BN2, Salivirus A 02394, Salivirus A GUT, Salivirus A CH, Salivirus A SZ1, Salivirus FHB, CVB3, CVB1, Echovirus 7, CVB5, EVA71, CVA3, CVA12, EV24 or an aptamer to eIF4G (see PCT App. Publs. WO2020237227A1 and WO2021113777A2, both of which are incorporated by reference herein in their entirety). In some embodiments, the circular RNA comprises, in the following order, a) a post-splicing intron fragment of a 3′ group I intron fragment, b) an IRES, c) an expression sequence, and d) a post-splicing intron fragment of a 5′ group I intron fragment. In some embodiments, the circular RNA polynucleotide is made via circularization of a RNA polynucleotide comprising, in the following order: a) a 3′ group I intron fragment, b) an IRES, c) an expression sequence, and d) a 5′ group I intron fragment. In some embodiments, the circular RNA comprises a first spacer before the post-splicing intron fragment of the 3′ group I intron fragment, and a second spacer after the post-splicing intron fragment of the 5′ group I intron fragment. In some embodiments, the first and second spacers each have a length of about 10 to about 60 nucleotides. In some embodiments, the circular RNA polynucleotide is made via circularization of a RNA polynucleotide comprising, in the following order: a) a 5′ external duplex forming region, b) a 3′ group I intron fragment, c) a 5′ internal spacer optionally comprising a 5′ internal duplex forming region, d) an IRES, e) an expression sequence, f) a 3′ internal spacer optionally comprising a 3′ internal duplex forming region, g) a 5′ group I intron fragment, and h) a 3′ external duplex forming region.

In some embodiments, the circular RNA polynucleotide is made via circularization of a RNA polynucleotide comprising, in the following order: a) a 5′ external duplex forming region, b) a 5′ external spacer, c) a 3′ group I intron fragment, d) a 5′ internal spacer optionally comprising a 5′ internal duplex forming region, e) an IRES, f) an expression sequence, g) a 3′ internal spacer optionally comprising a 3′ internal duplex forming region, h) a 5′ group I intron fragment, i) a 3′ external spacer, and j) a 3′ external duplex forming region. In some embodiments, the circular RNA polynucleotide is made via circularization of a RNA polynucleotide comprising, in the following order: a) a 3′ group I intron fragment, b) a 5′ internal spacer comprising a 5′ internal duplex forming region, c) an IRES, d) an expression sequence, e) a 3′ internal spacer comprising a 3′ internal duplex forming region, and f) a 5′ group I intron fragment. In some embodiments, the circular RNA polynucleotide is made via circularization of a RNA polynucleotide comprising, in the following order: a) a 5′ external duplex forming region, b) a 5′ external spacer, c) a 3′ group I intron fragment, d) a 5′ internal spacer comprising a 5′ internal duplex forming region, e) an IRES, f) an expression sequence, g) a 3′ internal spacer comprising a 3′ internal duplex forming region, h) a 5′ group I intron fragment, i) a 3′ external spacer, and j) a 3′ external duplex forming region. In some embodiments, the circular RNA polynucleotide is made via circularization of a RNA polynucleotide comprising, in the following order: a) a first polyA sequence, b) a 5′ external duplex forming region, c) a 5′ external spacer, d) a 3′ group I intron fragment, e) a 5′ internal spacer comprising a 5′ internal duplex forming region, f) an IRES, g) an expression sequence, h) a 3′ internal spacer comprising a 3′ internal duplex forming region, i) a 5′ group I intron fragment, j) a 3′ external spacer, k) a 3′ external duplex forming region, and l. a second polyA sequence. In some embodiments, the circular RNA polynucleotide is made via circularization of a RNA polynucleotide comprising, in the following order: a) a first polyA sequence, b) a 5′ external spacer, c) a 3′ group I intron fragment, d) a 5′ internal spacer comprising a 5′ internal duplex forming region, e) an IRES, f) an expression sequence, g) a 3′ internal spacer comprising a 3′ internal duplex forming region, h) a 5′ group I intron fragment, i) a 3′ external spacer, and j) a second poly A sequence.

In some embodiments, the circular RNA polynucleotide is made via circularization of a RNA polynucleotide comprising, in the following order: a) a first polyA sequence, b) a 5′ external spacer, c) a 3′ group I intron fragment, d) a 5′ internal spacer comprising a 5′ internal duplex forming region, e) an IRES, f) an expression sequence, g) a stop codon cassette, h) a 3′ internal spacer comprising a 3′ internal duplex forming region, i) a 5′ group I intron fragment, j) a 3′ external spacer, and k) a second polyA sequence.

In some embodiments, at least one of the 3′ or 5′ internal or external spacers has a length of about 8 to about 60 nucleotides. In some embodiments, the 3′ and 5′ external duplex forming regions each has a length of about 10 to about-50 nucleotides. In some embodiments, the 3′ and 5′ internal duplex forming regions each has a length of about 6 to about 30 nucleotides.

In some embodiments, the modified nucleic acid is a capped RNA, whereby the 5′ and/or 3′ ends are capped by a chemical alteration.

In more preferred embodiments, the synthetic scaffold domain comprises at least two desired modification sites for multiplexing. For instance, a second glycan is paired with a Y RNA to modify a second target region of the nucleic acid sequence. Accordingly, a plurality of glycans paired with the respective nucleic acid sequence, e.g., RNA, is used to modify a number of target regions of the nucleic acid sequence.

The present invention also provides nucleic acid molecules that hybridize under stringent conditions to the above-described nucleic acid molecules. As defined above, and as is well known in the art, stringent hybridizations are performed at about 25° C. below the thermal melting point (Tₘ) for the specific DNA hybrid under a particular set of conditions, where the Tₘ is the temperature at which 50% of the target sequence hybridizes to a perfectly matched probe. Stringent washing is performed at temperatures about 5° C. lower than the Tₘ for the specific DNA hybrid under a particular set of conditions.

Nucleic acid molecules comprising a fragment of any one of the above-described nucleic acid sequences are also provided. These fragments preferably contain at least 20 contiguous nucleotides. More preferably the fragments of the nucleic acid sequences contain at least about 25, at least about 30, at least about 35, at least about 40, at least about 45, at least about 50, at least about 60, at least about 70, at least about 80, at least about 90, at least about 100 or even more contiguous nucleotides.

The nucleic acid sequence fragments of the present invention display utility in a variety of systems and methods. For example, the fragments may be used as probes in various hybridization techniques. Depending on the method, the target nucleic acid sequences may be either DNA or RNA. The target nucleic acid sequences may be fractionated (e.g., by gel electrophoresis) prior to the hybridization, or the hybridization may be performed on samples in situ. One of skill in the art will appreciate that nucleic acid probes of known sequence find utility in determining chromosomal structure (e.g., by Southern blotting) and in measuring gene expression (e.g., by Northern blotting). In such experiments, the sequence fragments are preferably detectably labeled, so that their specific hybridization to target sequences can be detected and optionally quantified. One of skill in the art will appreciate that the nucleic acid fragments of the present invention may be used in a wide variety of blotting techniques not specifically described herein.

It should also be appreciated that the nucleic acid sequence fragments optionally conjugated to glycans disclosed herein also find utility as probes when immobilized on microarrays. Methods for creating microarrays by deposition and fixation of nucleic acids onto support substrates are well known in the art. Reviewed in DNA Microarrays: A Practical Approach (Practical Approach Series), Schena (ed.), Oxford University Press (1999) (ISBN: 0199637768); Nature Genet. 21(1)(suppl): 1-60 (1999); Microarray Biochip: Tools and Technology, Schena (ed.), Eaton Publishing Company/BioTechniques Books Division (2000) (ISBN: 1881299376), the disclosures of which are incorporated herein by reference in their entireties. Analysis of, for example, gene expression using microarrays comprising nucleic acid sequence fragments, such as the nucleic acid sequence fragments disclosed herein, is a well-established utility for sequence fragments in the field of cell and molecular biology. Other uses for sequence fragments immobilized on microarrays are described in Gerhold et al., Trends Biochem. Sci. 24:168-173 (1999) and Zweiger, Trends Biotechnol. 17:429-436 (1999); DNA Microarrays: A Practical Approach (Practical Approach Series), Schena (ed.), Oxford University Press (1999) (ISBN: 0199637768); Nature Genet. 21(1)(suppl): 1-60 (1999); Microarray Biochip: Tools and Technology, Schena (ed.), Eaton Publishing Company/BioTechniques Books Division (2000) (ISBN: 1881299376), the disclosure of each of which is incorporated herein by reference in its entirety.

As is well known in the art, enzyme activities can be measured in various ways. For example, the pyrophosphorolysis of OMP may be followed spectroscopically (Grubmeyer et al., (1993) J. Biol. Chem. 268:20299-20304). The activity of the enzyme can be followed using chromatographic techniques, such as by high performance liquid chromatography (Chung and 47 Sloan, (1986) J. Chromatogr. 371:71-81). As another alternative, the activity can be indirectly measured by determining the levels of product made from the enzyme activity. These levels can be measured with techniques including aqueous chloroform/methanol extraction as known and described in the art (Cf. M. Kates (1986) Techniques of Lipidology; Isolation, analysis and identification of Lipids. Elsevier Science Publishers, New York (ISBN: 0444807322)). More modern techniques include using gas chromatography linked to mass spectrometry (Niessen, W. M. A. (2001). Current practice of gas chromatography—mass spectrometry. New York, N.Y: Marcel Dekker. (ISBN: 0824704738)). Additional modern techniques for identification of recombinant protein activity and products including liquid chromatography-mass spectrometry (LCMS), high performance liquid chromatography (HPLC), capillary electrophoresis, Matrix-Assisted Laser Desorption Ionization time of flight-mass spectrometry (MALDI-TOF MS), nuclear magnetic resonance (NMR), near-infrared (NIR) spectroscopy, viscometry (Knothe, G (1997) Am. Chem. Soc. Symp. Series, 666: 172-208), titration for determining free fatty acids (Komers (1997) Fett/Lipid, 99(2): 52-54), enzymatic methods (Bailer (1991) Fresenius J. Anal. Chem. 340(3): 186), physical property-based methods, wet chemical methods, etc. can be used to analyze the levels and the identity of the product produced by the organisms of the present invention. Other methods and techniques may also be suitable for the measurement of enzyme activity, as would be known by one of skill in the art.

### Isolated Polypeptides

According to another aspect of the present invention, isolated polypeptides (including muteins, allelic variants, fragments, derivatives, and analogs) encoded by the nucleic acid molecules of the present invention are provided. In an alternative embodiment of the present invention, the isolated polypeptide comprises a polypeptide sequence at least 85% identical to identical to one or more encoded polypeptide sequences. Preferably the isolated polypeptide of the present invention has at least about 50%, at least about 60%, at least about 70%, at least about 80%, at least about 85%, at least about 90%, at least about 95%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.9% or even higher identity to one or more encoded polypeptide sequences.

According to other embodiments of the present invention, isolated polypeptides comprising a fragment of the above-described polypeptide sequences are provided. These fragments can include at least about 5, at least about 6, at least about 7, at least about 8, at least about 9, at least about 10, at least about 11, at least about 12, at least about 13, at least about 14, at least about 15, at least about 16, at least about 17, at least 18 about, at least 19 about, at least about 20, or more contiguous amino acids. These fragments preferably include at least about 20 contiguous amino acids, more preferably at least about 25, at least about 30, at least about 35, at least about 40, at least about 45, at least about 50, at least about 60, at least about 70, about 80, at least about 90, at least about 100 or even more contiguous amino acids.

The polypeptides of the present invention also include fusions between the above-described polypeptide sequences and heterologous polypeptides. The heterologous sequences can, for example, include sequences designed to facilitate purification, e.g., histidine tags, and/or visualization of recombinantly-expressed proteins. Other non-limiting examples of protein fusions include those that permit display of the encoded protein on the surface of a phage or a cell, fusions to intrinsically fluorescent proteins, such as green fluorescent protein (GFP), and fusions to the IgG Fc region.

### Glycan Synthesis and Selection

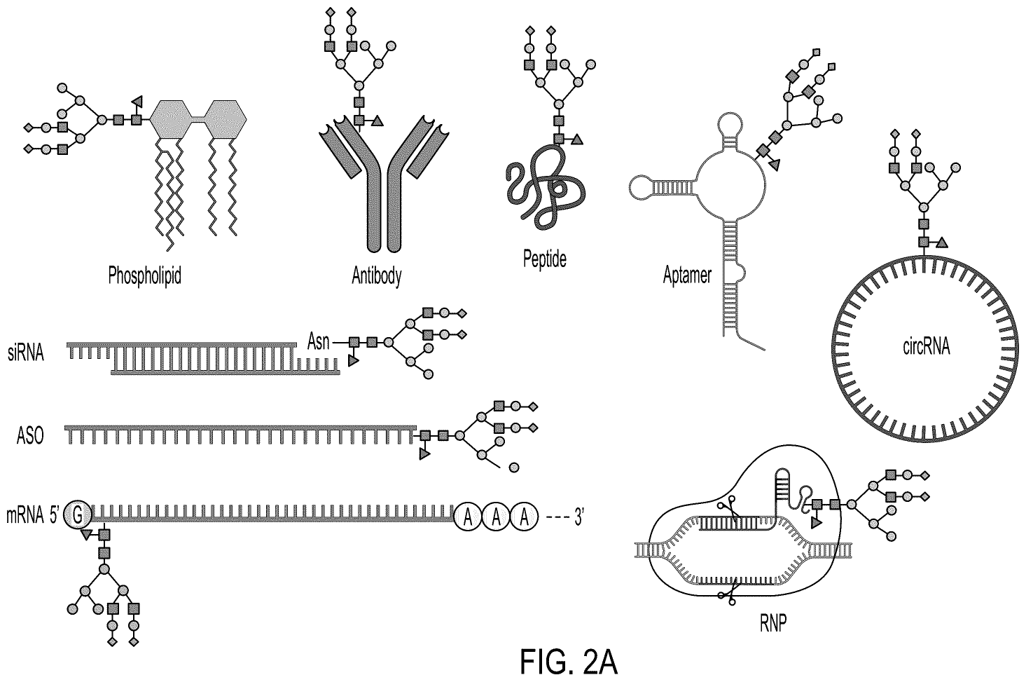

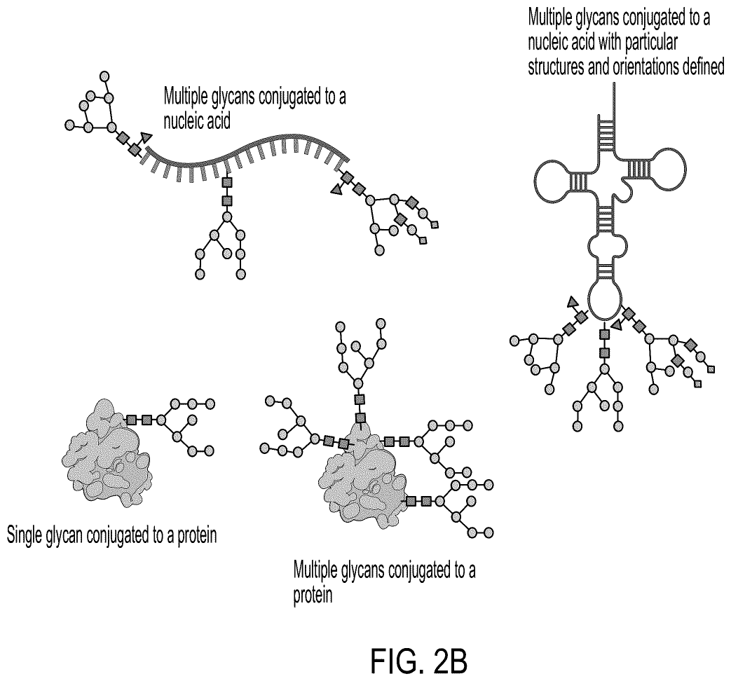

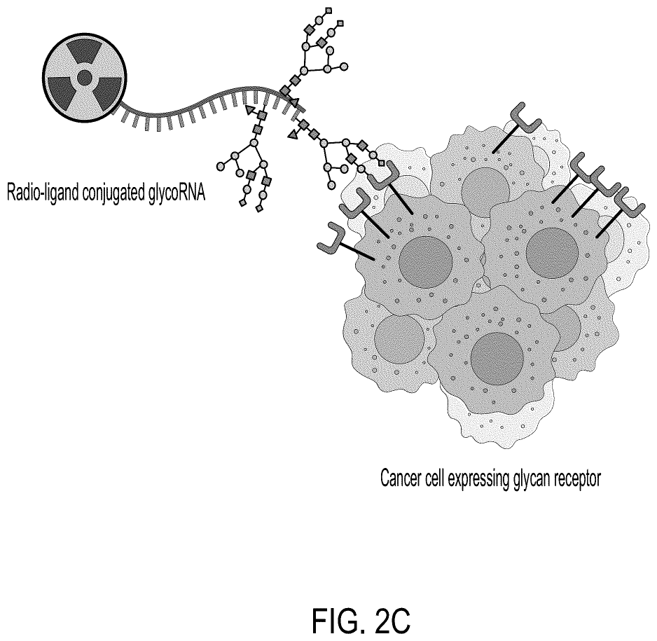

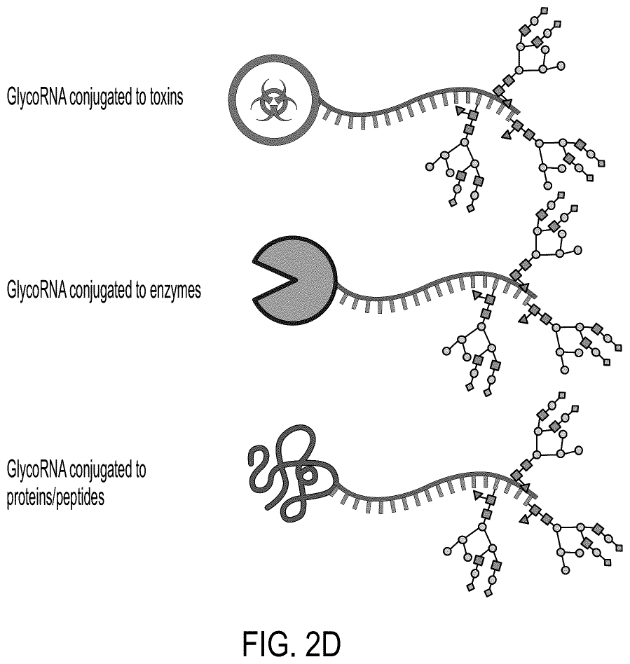

Provided herein are methods and compositions for selecting or synthesizing one or more glycan components of the glyco-ligand compositions. Preferably, a glyco-ligand composition is produced by synthesizing or selecting a desired glycan based on its purported association with cell signaling and conjugating the glycan onto a synthetic scaffold domain (e.g., FIG. 2A). One or more glycans are selected or synthesized as cell signaling molecules to contact one or more cell surface proteins of target cells to modulate a desired biological effect.

A library of naturally occurring N-glycans can be produced for example through chemoenzymatic synthesis or other suitable methods. See, e.g., Gao et al., 2019 Cell Chem Biology, Volume 26, Issue 4, 2019. Based on Gao et al., UDP-sugar substrates are transferred to glycan acceptor substrates using known glycosyltransferases chemoenzymatically. See, for instance, Example 1.

As an alternative to chemoenzymatic synthesis of glycans, glycans can be synthesized, purified and/or isolated using any suitable method to generate a desired glycan type.

Other existing methods to generate glycans include recombinant or heterologous glycan biosynthesis, e.g., through overexpression or heterologous expression of one or more glycosyltransferases, glycosidases, sugar nucleotide donors, epimerases (UDP-GlcNAc and UDP-Gal), UDP-N-acetylglucosamine transporter, GDP-Fucose Transporter, UDP-Galactose Transporter, CMP-N-Acetylneuraminic Acid (CMP-Sialic Acid) Transporter (e.g., UDP-N-acetylglucosamine, UDP-N-acetylgalactosamine, CMP-N-acetylneuraminic acid, UDP-galactose, GDP-fucose, etc.), wherein glycans are synthesized in the cytosol and transported into the Golgi, where they are attached to the core oligosaccharide by glycosyltransferases. See, for example, (Sommers and Hirschberg, 1981 J. Cell Biol. 91 (2): A406-A406; Sommers and Hirschberg 1982 J. Biol. Chem. 257(18): 811-817; Perez and Hirschberg 1987 Methods in Enzymology 138:709-715). Analogous recombinant or heterologous glycan biosynthesis methods can be used to catalyze the assembly of desired glycans and if desired, followed by isolation from host cells including CHO cells, yeast cells, insect cells and plant cells.

In various aspects of the invention, the carbohydrate moiety, e.g., glycans conjugated on glyco-ligands, comprises one or more or a combination of sugar residues including but not limited to D-glucose (“Glc”), galactose (“Gal”), mannose (“Man”), fucose (“Fuc”), N-acetylgalactosamine (“GalNAc”), N-acetylglucosamine (“GlcNAc”), N-acetyllactosamine (“LacNAc”), sialic acid (e.g., N-acetylneuraminic acid (“NANA” or “NeuAc”, where “Neu” is neuraminic acid and “Ac” refers to “acetyl”)), D-glucosamine (“GlcN”), D-Glucuronic Acid (“GlcA”), β-muramic acid (“Mur”), Mannuronic Acid (“ManA”), N-Acetyl-Muramic Acid (“MurNAc”), Legionaminic acid (“Leg”), Acinetaminic acid (“Aci”), D-Xylose (“Xyl”), N-Acetyl-L-Fucosamine (“FucNAc”), Pseudaminic acid (“Pse”) and L-Iduronic Acid (“IdoA”).

As used herein, chemical modification “9-N-biphenyl Carboxamide” or “BPC” refers to a moiety of structure:

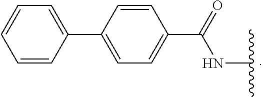 [SMILES: *NC(=O)c1ccc(-c2ccccc2)cc1]

The oligosaccharide structure attached to a nucleic acid molecule, e.g., found in naturally occurring RNA, while not yet fully characterized can be divided into two classes (as is done for glycoproteins), “N-linked glycans” or N-linked oligosaccharides” and “O-linked glycans” or “O-linked oligosaccharides.” Glycans can comprise mono-, di- and oligosaccharides. Without being bound by theory, the processing of the carbohydrate moiety on non-amino acid molecules, e.g., RNA, can occur co-translationally in the lumen of the ER and continues in the Golgi apparatus similar to N-linked glycoproteins.

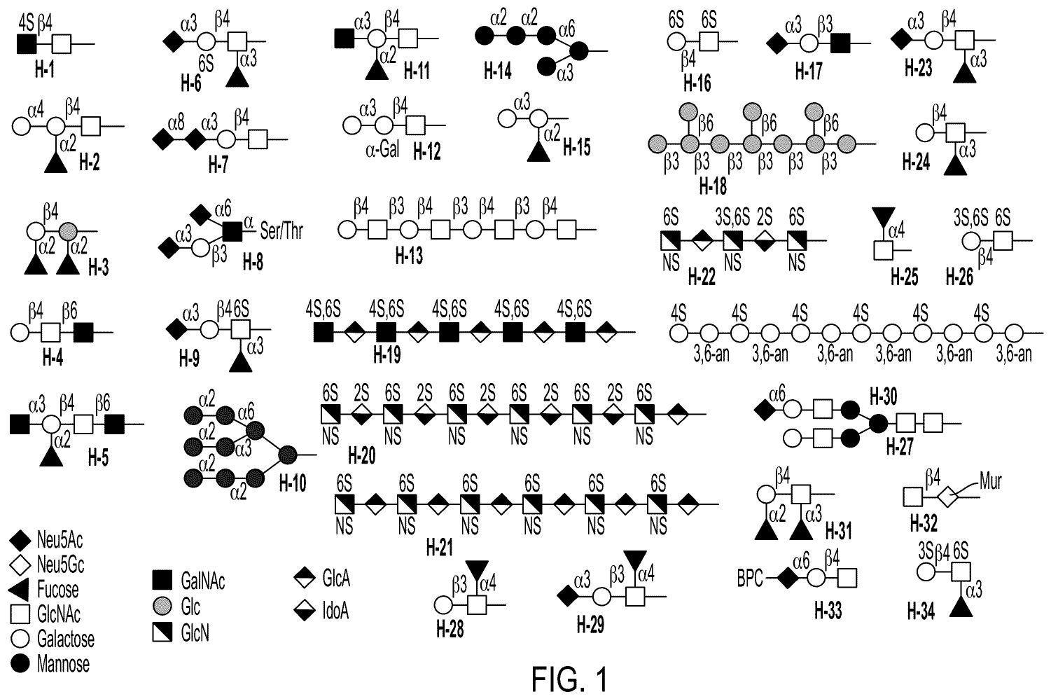

A wide variety of glycans can be selected for conjugation to a desired scaffold (e.g., synthetic scaffold domain) including N-linked type glycans, such as hybrid or complex, branched, oligomannose glycans or O-linked type glycans. In some embodiments, a glyco-ligand of the invention comprises a glycan depicted in FIG. 1. In certain embodiments, the glycan is a complex type N-glycan. In certain embodiments, the glycan is a multiple antennary complex type N-glycan. In certain embodiments, the glycan is a hybrid type N-glycan. In certain embodiments, the glycan is an oligomannose glycan. In certain embodiments, the glycan is an O-linked type glycan.

In some embodiments, a glyco-ligand of the invention comprises a glycan selected from those listed in Tables 1A-1D below. In some embodiments, a glyco-ligand of the invention comprises a glycan selected from those listed in Tables 1A-1F. For example, the glyco-ligand can comprise a glycan selected from those depicted in Tables 1E or 1F.

In some embodiments, a glycan targeting moiety of the present disclosure comprises a glycan selected from those listed in Table 1A below:

TABLE 1A
Exemplary Glycans

| Glycan # | Structure | IUPAC Name |
|---|---|---|
| G-1 | 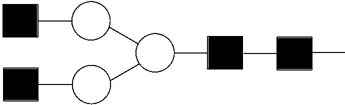 | GlcNAc(b1-2)Man(a1- 3)[GlcNAc(b1-2)Man(a1- 6)]Man(b1-4)GlcNAc(b1- 4)GlcNAc |
| G-2 | 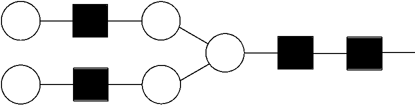 | Gal(b1-4)GlcNAc(b1-2)Man(al- 3)[Gal(b1-4)GlcNAc(b1- 2)Man(a1-6)]Man(b1- 4)GlcNAc(b1-4)GlcNAc |
| G-3 | 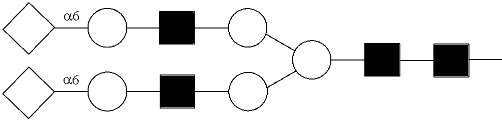 | Neu5Ac(a2-6)Gal(b1- 4)GlcNAc(b1-2)Man(a1- 3)[Neu5Ac(a2-6)Gal(b1- 4)GlcNAc(b1-2)Man(a1- 6)]Man(b1-4)GlcNAc(b1- 4)GlcNAc |
| G-4 | 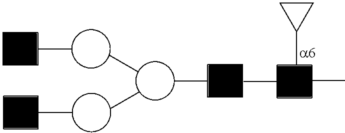 | GlcNAc(b1-2)Man(a1- 3)[GlcNAc(b1-2)Man(a1- 6)]Man(b1-4)GlcNAc(b1- 4)[Fuc(a1-6)]GlcNAc |
| G-5 | 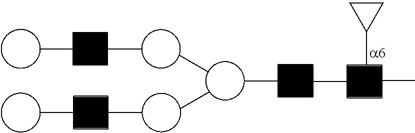 | Gal(b1-4)GlcNAc(b1-2)Man(a1- 3)[Gal(b1-4)GlcNAc(b1- 2)Man(a1-6)]Man(b1- 4)GlcNAc(b1-4)[Fuc(al- 6)]GlcNAc |
| G-6 | 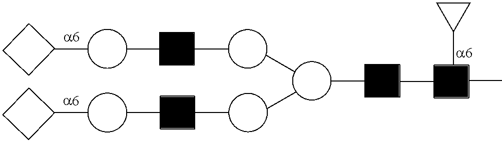 | Neu5Ac(a2-6)Gal(b1- 4)GlcNAc(b1-2)Man(a1- 3)[Neu5Ac(a2-6)Gal(b1- 4)GlcNAc(b1-2)Man(al- 6)]Man(b1-4)GlcNAc(b1- 4)[Fuc(a1-6)]GlcNAc |
| G-7 | 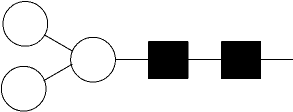 | Man(a1-3)[Man(a1-6)]Man(b1- 4)GlcNAc(b1-4)GlcNAc |
| G-8 | 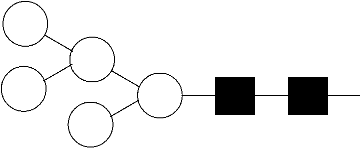 | Man(a1-6)[Man(a1-3)]Man(a1- 6)[Man(a1-3)]Man(b1- 4)GlcNAc(b1-4)GlcNAc |
| G-9 | 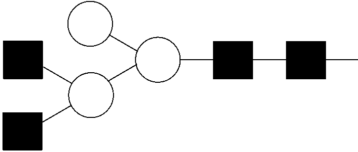 | Man(a1-6)[GlcNAc(b1- 4)[GlcNAc(b1-2)]Man(a1- 3)]Man(b1-4)GlcNAc(b1- 4)GlcNAc |
| G-10 | 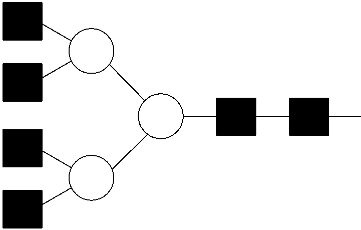 | GlcNAc(b1-6)[GlcNAc(b1- 2)]Man(a1-6)[GlcNAc(b1- 4)[GlcNAc(b1-2)]Man(a1- 3)]Man(b1-4)GlcNAc(b1- 4)GlcNAc |
| G-11 | 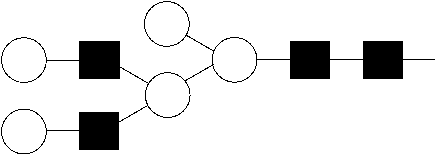 | Man(a1-6)[Gal(b1-4)GlcNAc(b1- 4)[Gal(b1-4)GlcNAc(b1- 2)]Man(a1-3)]Man(b1- 4)GlcNAc(b1-4)GlcNAc |
| G-12 | 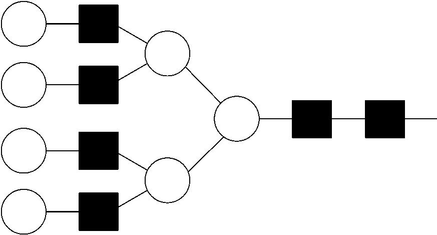 | Gal(b1-4)GlcNAc(b1-6)[Gal(b1- 4)GlcNAc(b1-2)]Man(a1- 6)[Gal(b1-4)GlcNAc(b1- 4)[Gal(b1-4)GlcNAc(b1- 2)]Man(a1-3)]Man(b1- 4)GlcNAc(b1-4)GlcNAc |
| G-13 | 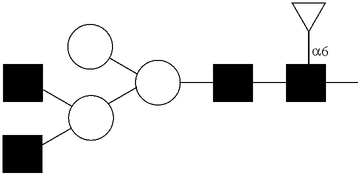 | Man(a1-6)[GlcNAc(b1- 4)[GlcNAc(b1-2)]Man(a1- 3)]Man(b1-4)GlcNAc(b1- 4)[Fuca1-6]GlcNAc |
| G-14 | 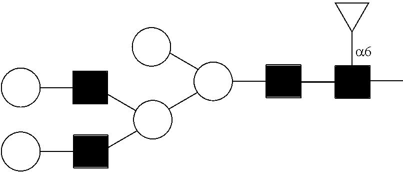 | Man(a1-6)[Gal(b1-4)GlcNAc(b1- 4)[Gal(b1-4)GlcNAc(b1- 2)]Man(a1-3)]Man(b1- 4)GlcNAc(b1-4)[Fuc(a1- 6)]GlcNAc |
| G-15 | 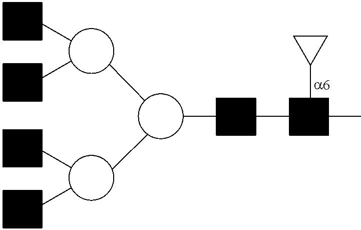 | GlcNAc(b1-6)[GlcNAc(b1- 2)]Man(a1-6)[GlcNAc(b1- 4)[GlcNAc(b1-2)]Man(a1- 3)]Man(b1-4)GlcNAc(b1- 4)[Fuc(a1-6)]GlcNAc |
| G-16 | 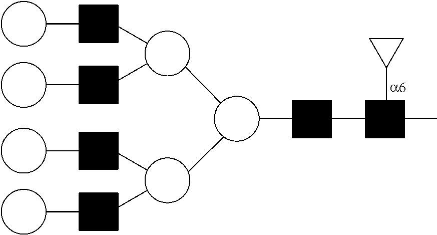 | Gal(b1-4)GlcNAc(b1-6)[Gal(b1- 4)GlcNAc(b1-2)]Man(a1- 6)[Gal(b1-4)GlcNAc(b1- 4)[Galb1-4GlcNAcb1-2]Mana1- 3]Manb1-4GlcNAcb1-4[Fuca1- 6]GlcNA |
| G-17 | 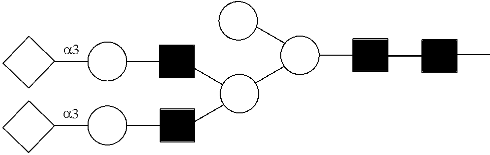 | Man(a1-6)[Neu5Ac(a2-3)Gal(b1- 4)GlcNAc(b1-4)[Neu5Ac(a2- 3)Gal(b1-4)GlcNAc(b1- 2)]Man(a1-3)]Man(b1- 4)GlcNAc(b1-4)GlcNAc |
| G-18 | 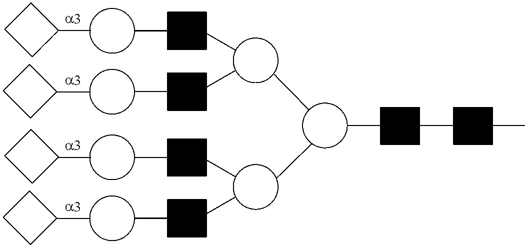 | Neu5Ac(a2-3)Gal(b1- 4)GlcNAc(b1-6)[Neu5Ac(a2- 3)Gal(b1-4)GlcNAc(b1- 2)]Man(a1-6)[Neu5Ac(a2- 3)Gal(b1-4)GlcNAc(b1- 4)[Neu5Ac(a2-3)Gal(b1- 4)GlcNAc(b1-2)]Man(a1- 3)]Man(b1-4)GlcNAc(b1- 4)GlcNAc |
| G-19 | 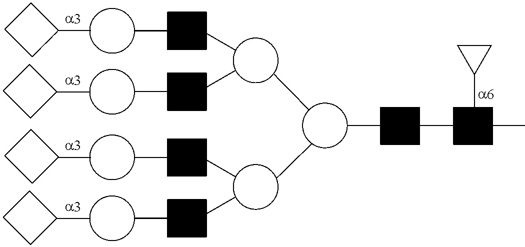 | Neu5Ac(a2-3)Gal(b1- 4)GlcNAc(b1-6)[Neu5Ac(a2- 3)Gal(b1-4)GlcNAc(b1- 2)]Man(a1-6)[Neu5Ac(a2- 3)Gal(b1-4)GlcNAc(b1- 4)[Neu5Ac(a2-3)Gal(b1- 4)GlcNAc(b1-2)]Man(a1- 3)]Man(b1-4)GlcNAc(b1- 4)[Fuc(a1-6)]GlcNAc |
| G-20 | 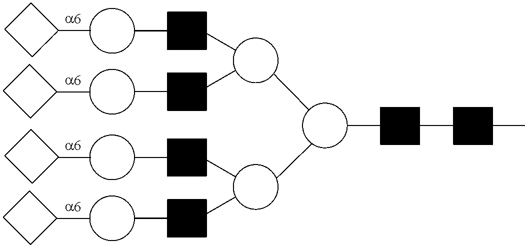 | Neu5Ac(a2-6)Gal(b1- 4)GlcNAc(b1-6)[Neu5Ac(a2- 6)Gal(b1-4)GlcNAc(b1- 2)]Man(a1-6)[Neu5Ac(a2- 6)Gal(b1-4)GlcNAc(b1- 4)[Neu5Ac(a2-6)Gal(b1- 4)GlcNAc(b1-2)]Man(a1- 3)]Man(b1-4)GlcNAc(b1- 4)GlcNAc |
| G-21 | 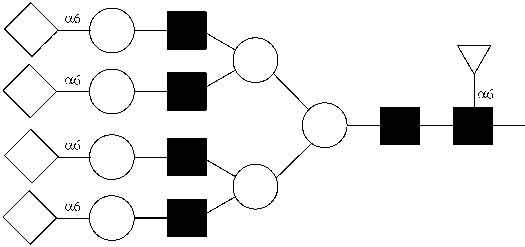 | Neu5Ac(a2-6)Gal(b1- 4)GlcNAc(b1-6)[Neu5Ac(a2- 6)Gal(b1-4)GlcNAc(b1- 2)]Man(a1-6)[Neu5Ac(a2- 6)Gal(b1-4)GlcNAc(b1- 4)[Neu5Ac(a2-6)Gal(b1- 4)GlcNAc(b1-2)]Man(a1- 3)]Man(b1-4)GlcNAc(b1- 4)[Fuc(a1-6)]GlcNAc |
| G-22 | 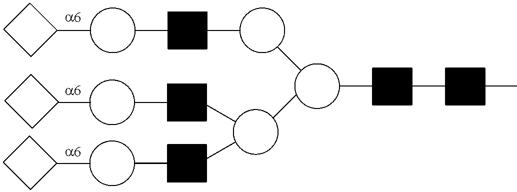 | Neu5Ac(a2-6)Galb1- 4GlcNAc(b1-2)Manal- 6[Neu5Ac(a2-6)Galb1- 4GlcNAc(b1-4)[Neu5Ac(a2- 6)Gal(b1-4)GlcNAc(b1- 2)]Man(a1-3)]Man(b1- 4)GlcNAc(b1-4)GlcNAc |
| G-23 | 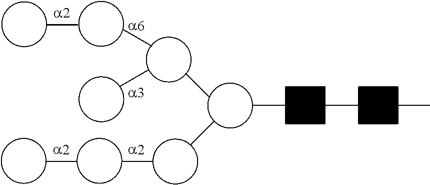 | Man(a1-2)Man(a1-6)[Man(a1- 3)]Man(a1-6)[Man(a1-2)Man(a1- 2)Man(a1-3)]Man(b1- 4)GlcNAc(b1-4)GlcNAc |
| G-24 | 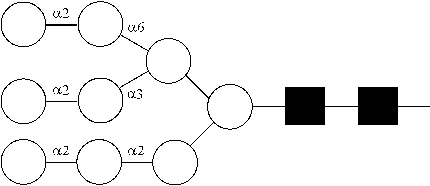 | Man(a1-2)Man(a1-6)[Man(a1- 2)Man(a1-3)]Man(a1-6)[Man(a1- 2)Man(a1-2)Man(a1-3)]Man(b1- 4)GlcNAc(b1-4)GlcNAc |
| G-25 | 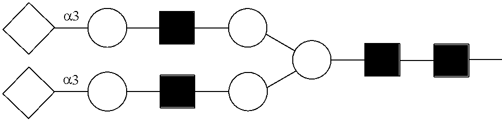 | Neu5Ac(a2-3)Gal(b1- 4)GlcNAc(b1-2)Man(a1- 6)[Neu5Ac(a2-3)Gal(b1- 4)GlcNAc(b1-2)Man(a1- 3)]Man(b1-4)GlcNAc(b1- 4)GlcNAc |
| G-26 | 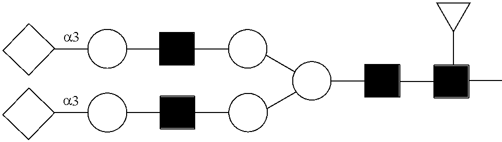 | Neu5Ac(a2-3)Gal(b1- 4)GlcNAc(b1-2)Man(a1- 6)[Neu5Ac(a2-3)Gal(b1- 4)GlcNAc(b1-2)Man(a1- 3)]Man(b1-4)GlcNAc(b1- 4)[Fuc(a1-6)]GlcNAc |
| G-27 | 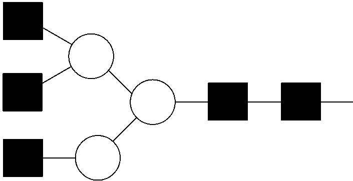 | GlcNAc(b1-6)[GlcNAc(b1- 2)]Man(a1-6)[GlcNAc(b1- 2)Man(a1-3)]Man(b1- 4)GlcNAc(b1-4)GlcNAc |
| G-28 | 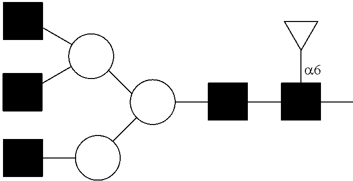 | GlcNAc(b1-6)[GlcNAc(b1- 2)]Man(a1-6)[GlcNAc(b1- 2)Man(a1-3)]Man(b1- 4)GlcNAc(b1-4)[Fuc(a1- 6)]GlcNAc |
| G-29 | 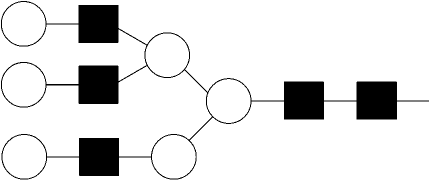 | Gal(a1-4)GlcNAc(b1-6)[Gal(a1- 4)GlcNAc(b1-2)]Man(a1- 6)[Gal(a1-4)GlcNAc(b1- 2)Man(a1-3)]Man(b1- 4)GlcNAc(b1-4)GlcNAc |
| G-30 | 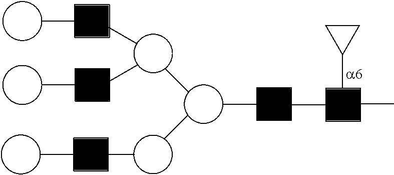 | Gal(b1-4)GlcNAc(b1-6)[Gal(b1- 4)GlcNAc(b1-2)]Man(a1- 6)[Gal(b1-4)GlcNAc(b1- 2)Man(a1-3)]Man(b1- 4)GlcNAc(b1-4)[Fuc(a1- 6)]GlcNAc |
| G-31 | 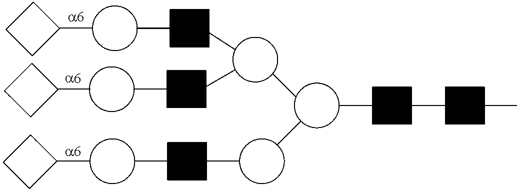 | Neu5Ac(a2-6)Gal(b1- 4)GlcNAc(b1-6)[Neu5Ac(a2- 6)Gal(b1-4)GlcNAc(b1- 2)]Man(a1-6)[Neu5Ac(a2- 6)Gal(b1-4)GlcNAc(b1- 2)Man(a1-3)]Man(b1- 4)GlcNAc(b1-4)GlcNAc |
| G-32 | 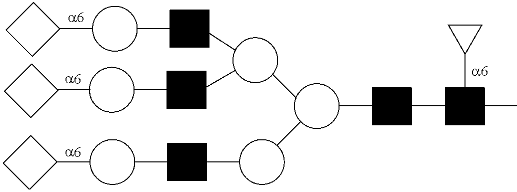 | Neu5Ac(a2-6)Gal(b1- 4)GlcNAc(b1-6)[Neu5Ac(a2- 6)Gal(b1-4)GlcNAc(b1- 2)]Man(a1-6)[Neu5Ac(a2- 6)Gal(b1-4)GlcNAc(b1- 2)Man(a1-3)]Man(b1- 4)GlcNAc(b1-4)[Fuc(a1- 6)]GlcNAc |
| G-33 | 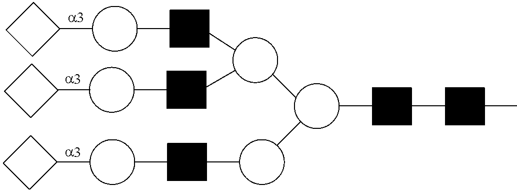 | Neu5Ac(a2-3)Gal(b1- 4)GlcNAc(b1-6)[Neu5Ac(a2- 3)Gal(b1-4)GlcNAc(b1- 2)]Man(a1-6)[Neu5Ac(a2- 3)Gal(b1-4)GlcNAc(b1- 2)Man(a1-3)]Man(b1- 4)GlcNAc(b1-4)GlcNAc |
| G-34 | 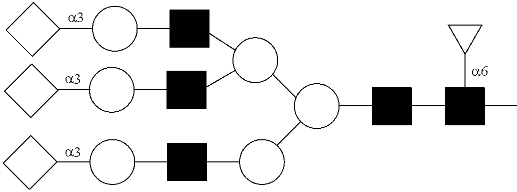 | Neu5Ac(a2-3)Gal(b1- 4)GlcNAc(b1-6)[Neu5Ac(a2- 3)Gal(b1-4)GlcNAc(b1- 2)]Man(a1-6)[Neu5Ac(a2- 3)Gal(b1-4)GlcNAc(b1- 2)Man(a1-3)]Man(b1- 4)GlcNAc(b1-4)[Fuc(a1- 6)]GlcNAc |
| G-35 | 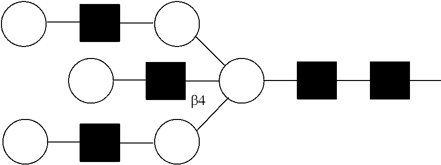 | Gal(b1-4)GlcNAc(b1-2)Man(a1- 6)[Gal(b1-4)GlcNAc(b1- 4)][Gal(b1-4)GlcNAc(b1- 2)Man(a1-3)]Man(b1- 4)GlcNAc(b1-4)GlcNAc |
| G-36 | 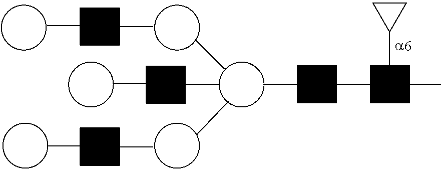 | Gal(b1-4)GlcNAc(b1-2)Man(a1- 6)[Gal(b1-4)GlcNAc(b1- 4)][Gal(b1-4)GlcNAc(b1- 2)Man(a1-3)]Man(b1- 4)GlcNAc(b1-4)[Fuc(a1- 6)]GlcNAc |
| G-37 | 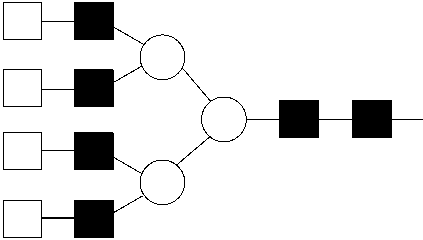 | GalNAc(b1-4)GlcNAc(b1- 6)[GalNAc(b1-4)GlcNAc(b1- 2)]Man(a1-6)[GalNAc(b1- 4)GlcNAc(b1-4)[GalNAc(b1- 4)GlcNAc(b1-2)]Man(a1- 3)]Man(b1-4)GlcNAc(b1- 4)GlcNAc |
| G-38 | 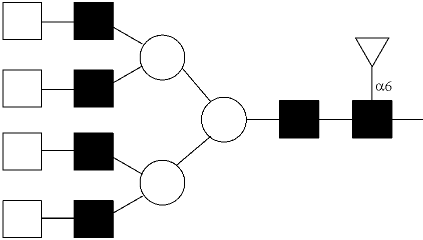 | GalNAc(b1-4)GlcNAc(b1- 6)[GalNAc(b1-4)GlcNAc(b1- 2)]Man(a1-6)[GalNAc(b1- 4)GlcNAc(b1-4)[GalNAc(b1- 4)GlcNAc(b1-2)]Man(a1- 3)]Man(b1-4)GlcNAc(b1-4) [(Fuca1-6)]GlcNAc |
| G-39 | 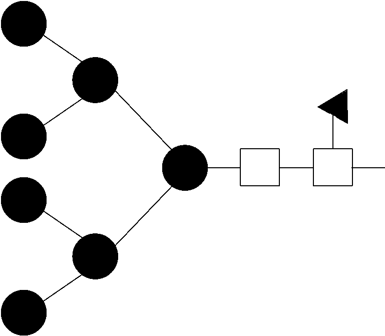 | Man(a1-6)[Man(a1-3)]Man(a1- 6)[Man(a1-6)[Man(a1-3)]Man(a1- 3)]Man(b1-4)GlcNAc(b1- 4)[(Fuca1-6)]GlcNAc |

In some embodiments, a glycan targeting moiety of the present disclosure comprises a glycan selected from those listed in Table 1B below:

TABLE 1B
Exemplary Glycans

| Glycan # | Structure | IUPAC Name |
|---|---|---|
| H-1 | 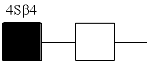 | [4S]GalNAc(b1- 4)GlcNAc |
| H-2 | 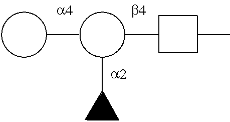 | Gal(a1-4)[Fuc(a1- 2)]Gal(b1-4)GlcNAc |
| H-3 | 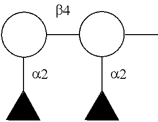 | Fuc(a1-2)Gal(b1- 4)[Fuc(a1-2)]Glc |
| H-4 | 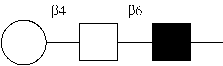 | Gal(b1-4)GlcNAc(b1- 6)GalNAc |
| H-5 | 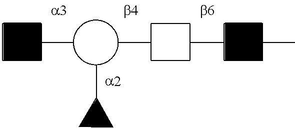 | GalNAc(a1-3)[Fuc(a1- 2)]Gal(b1-4)GlcNAc(b1- 6)GalNAc |
| H-6 | 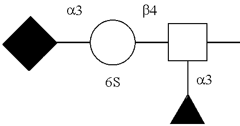 | Neu5Ac(a2-3)[6S]Gal(b1- 4)[Fuc(a1-3)]GlcNAc |
| H-7 | 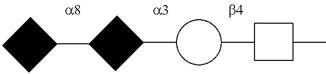 | Neu5Ac(a2- 8)Neu5Ac(a2-3)Gal(b1- 4)GlcNAc |
| H-8 | 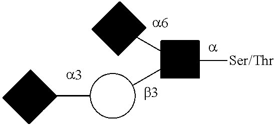 | Neu5Ac(a2-3)Gal(b1-3) [Neu5Ac(a2- 6)]GalNAc(a)Ser/Ther |
| H-9 | 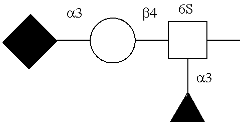 | Neu5Ac(a2-3)Gal(b1- 4)[Fuc(a1-3)][6S]GlcNAc |
| H-10 | 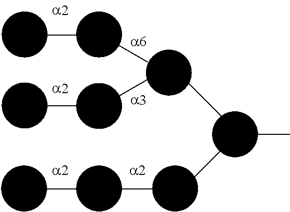 | Man(a1-2)Man(a1- 6)[Man(a1-2)Man(a1- 3)]Man(a1-6)[Man(a1- 2)Man(a1-2)Man(a1- 3)]Man |
| H-11 | 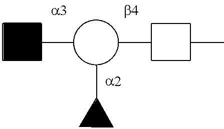 | GalNAc(a1-3)[Fuc(a1- 2)]Gal(b1-4)GlcNAc |
| H-12 | 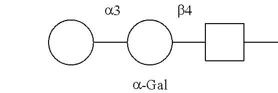 | Gal(a1-3)Gal(b1- 4)GlcNAc |
| H-13 | 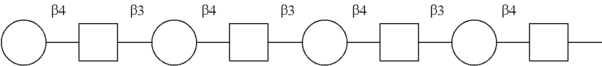 | Gal(b1-4)GlcNAc(b1- 3)Gal(b1-4)GlcNAc(b1- 3)Gal(b1-4)GlcNAc(b1- 3)Gal(b1-4)GlcNAc |
| H-14 | 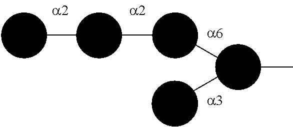 | Man(a1-2)Man(a1- 2)Man(a1-6)[Man(a1- 3)]Man |
| H-15 | 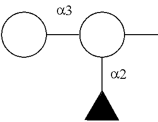 | Gal(a1-3)[Fuc(a1-2)]Gal |
| H-16 | 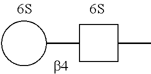 | (6S)Gal(b1- 4)(6S)GlcNAc |
| H-17 | 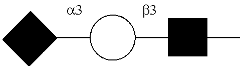 | Neu5Ac(a2-3)Gal(b1- 3)GalNAc |
| H-18 | 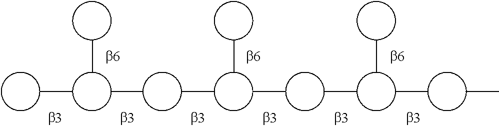 | Glc(b1-3)[Glc(b1- 6)]Glc(b1-3)Glc(b1-3) [Glc(b1-6)]Glc(b1- 3)Glc(b1-3)[Glc(b1- 6)]Glc(b1-3)Glc |
| H-19 | 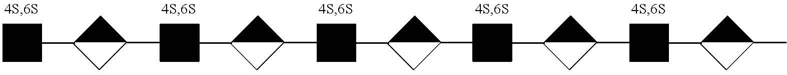 | [6S][4S]GalNAc(b1- 4)GlcA(b1- 3)[6S][4S]GalNAc(b1- 4)GlcA(b1- 3)[4S][6S]GalNAc(b1- |
| | | 4)GlcA(b1- |
| | | 3)[6S][4S]GalNAc(b1- |
| | | 4)GlcA(b1- |
| | | 3)[4S][6S]GalNAc(b1- |
| | | 4)GlcA |
| H-20 | 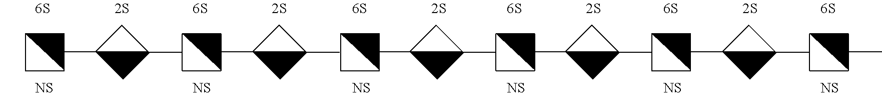 | [2S][6S]GlcN(a1- 4)[2S]IdoA(b1-4) [2S][6S]GlcN(a1- 4)[2S]IdoA(b1-4) [2S][6S]GlcN(a1- 4)[2S]IdoA(b1-4) |
| | | [25][6S]GlcN(a1- |
| | | 4)[2S]IdoA(b1-4) |
| | | [2S][6S]GlcN(a1- |
| | | 4)[2S]IdoA(b1- |
| | | 4)[2S][6S]GlcN(a1- |
| | | 4)GlcA |
| H-21 | 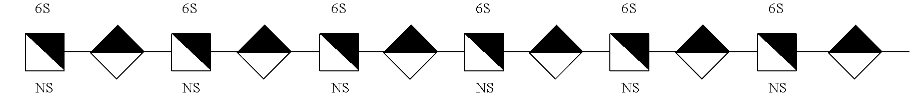 | [25][6S]GlcN(a1- 4)GlcA(b1-4) [2S][6S]GlcN(a1- 4)GlcA(b1-4) [2S][6S]GlcN(a1- 4)GlcA(b1-4) |
| | | [25][6S]GlcN(al- |
| | | 4)GlcA(b1-4) |
| | | [2S][6S]GlcN(al- |
| | | 4)GlcA(b1-4) |
| | | [25][6S]GlcN(a1-4)GlcA |
| H-22 | 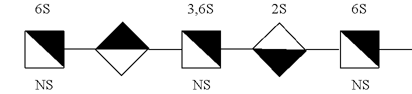 | [25][6S]GlcN(a1- 4)GlcA(b1-4) [3S][6S]GlcN(a1- 4)[2S]IdoA(b1- 4)[2S][6S]GlcN |
| H-23 | 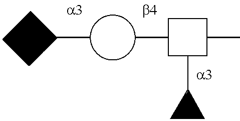 | Neu5Ac(a2-3)Gal(b1- 4)[Fuc(a1-3)]GlcNAc |
| H-24 | 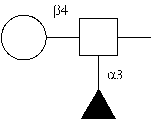 | Gal(b1-4)[Fuc(a1- 3)]GlcNAc |
| H-25 | 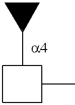 | Fuc(a1-4)GlcNAc |
| H-26 | 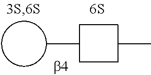 | [6S][3S]Gal(b1- 4)[6S]GlcNAc |
| H-27 | 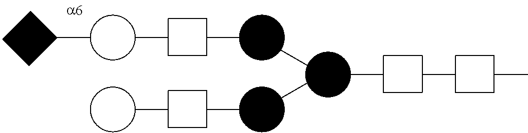 | Neu5Ac(a2-6)Gal(b1- 4)GlcNAc(b1-2)Man(a1- 3)[Gal(b1-4)GlcNAc(b1- 2)Man(a1-6)]Man(b1- 4)GlcNAc(b1- 4)GlcNAc(b1 |
| H-28 | 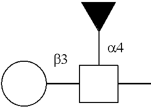 | Gal(b1-3)[Fuc(a1- 4)]GlcNAc |
| H-29 | 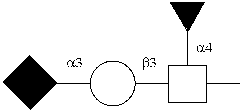 | Neu5Ac(a2-3)Gal(b1- 3)[Fuc(a1-4)]GlcNAc |
| H-30 | 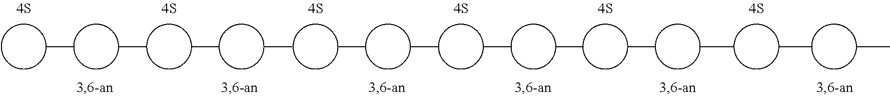 | [4S]Gal(b1-4)[3,6- anhydro]Gal(b1-4) [4S]Gal(b1-4)[3,6- anhydro]Gal(b1-4) [4S]Gal(b1-4)[3,6- anhydro]Gal(b1-4) |
| | | [4S]Gal(b1-4)[3,6- |
| | | anhydro]Gal(b1-4) |
| | | [4S]Gal(b1-4)[3,6- |
| | | anhydro]Gal(b1-4) |
| | | [4S]Gal(b1-4)[3,6- |
| | | anhydro]Gal |
| H-31 | 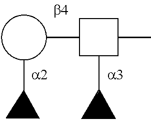 | Fuc(a1-2)Gal(b1- 4)[Fuc(a1-3)]GlcNAc |
| H-32 | 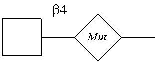 | GlcNAc(b1-4)Mur |
| H-33 | 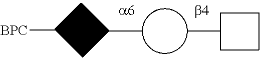 | [9-N-biphenyl Carboxamide] Neu5Ac(a2-6)Gal(b1- 4)GlcNAc |
| H-34 | 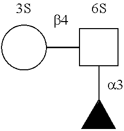 | [3S]Gal(b1-4)[Fuc(a1- 3)][6S]GlcNAc |
| H-35 | 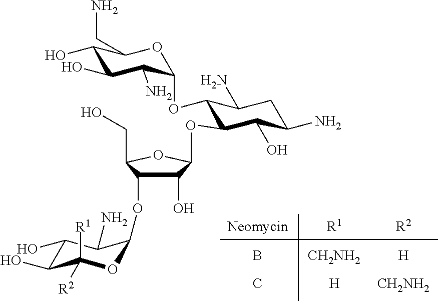 | “Neomycin” (2R,3S,4R,5R,6R)-5- amino-2-(aminomethyl)- 6-[(1R,2R,3S,4R,6S)-4,6- diamino-2- [(2S,3R,4S,5R)-4- [(2R,3R,4R,5S,6S)-3- amino-6-(aminomethyl)- 4,5-dihydroxyoxan-2- yl]oxy-3-hydroxy-5- (hydroxymethyl)oxolan-2- yl]oxy-3- hydroxycyclohexyl]oxy- oxane-3,4-diol |
| H-36 | 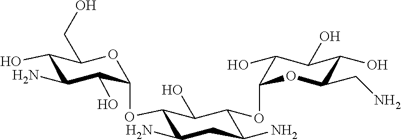 | “Kanamycin” (2R,3S,4S,5R,6R)-2- (aminomethyl)-6- [(1R,2R,3S,4R,6S)-4,6- diamino-3- [(2S,3R,4S,5S,6R)-4- amino-3,5-dihydroxy-6- (hydroxymethyl)oxan-2- yl]oxy-2- |
| | | hydroxycyclohexyl]oxyox |
| | | ane-3,4,5-triol |
| H-37 | 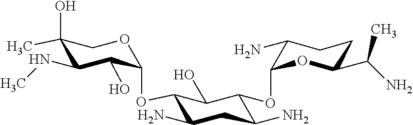 | “Gentamicin” (3R,4R,5R)-2- {[(1S,2S,3R,4S,6R)-4,6- diamino-3-{[(2R,3R,6S)- 3-amino-6-[(1R)- 1- (methylamino)ethyl]oxan- 2-yl]oxy }- |
| | | 2- |
| | | hydroxycyclohexyl]oxy}- |
| | | 5-methyl- |
| | | 4-(methylamino)oxane- |
| | | 3,5-diol |
| H-38 | 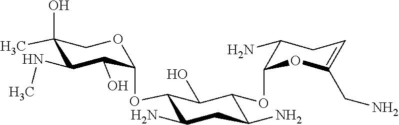 | “Sisomicin” (2R,3R,4R,5R)-2- {[(1S,2S,3R,4S,6R)-4,6- diamino-3-{[(2S,3R)-3- amino-6-(aminomethyl)- 3,4-dihydro-2H-pyran-2- yl]oxy}-2- hydroxycyclohexyl]oxy}- |
| | | 5-methyl-4- |
| | | (methylamino)oxane-3,5- |
| | | diol |
| H-39 | 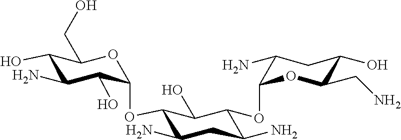 | “Tobramycin” (2S,3R,4S,5S,6R)-4- amino-2- {[(1S,2S,3R,4S,6R)-4,6- diamino-3- {[(2R,3R,5S,6R)-3- amino-6-(aminomethyl)- 5-hydroxyoxan-2- yl]oxy}-2- |
| | | hydroxycyclohexyl]oxy }- |
| | | 6-(hydroxymethyl)oxane- |
| | | 3,5-diol |
| H-40 | 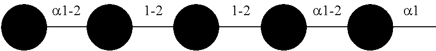 | Mana1-2Man1-2Man1- 2Mana1-2Mana1- |
| H-41 | 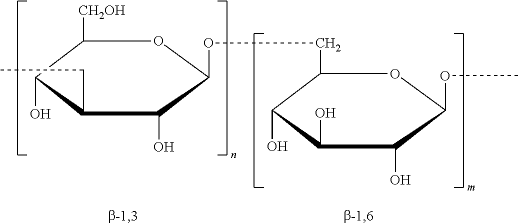 | Laminarin |
| H-42 | 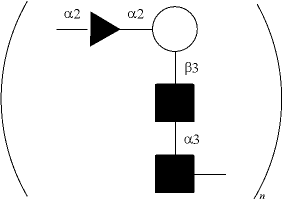 | (a2-Fuca2-Galb3- GalNAca3-GalNAc-)ₙ |
| H-43 | 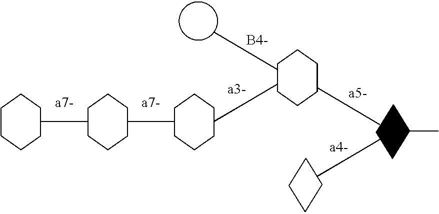 [SMILES: C1CCCCC1 ; C1CCCCC1 ; C1CCCCC1 ; "B4-" ; C1CCCCC1] | GlcB4-(MurNAca7- MurNAca7-MurNAca3-) MurNAca5-(Lega4-) Acia6- |
| H-44 | 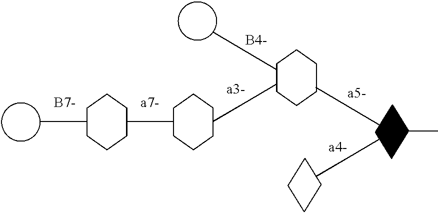 [SMILES: "B7-" ; C1CCCCC1 ; C1CCCCC1 ; "B4-" ; C1CCCCC1] | GlcB4-(GalB7- MurNAca7-MurNAca3-) MurNAca5-(Lega4-) Acia6- |
| H-45 | 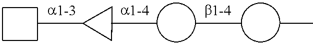 | GlcNAca1-3Rhaa1- 4Glcb1-4Glc |
| H-46 | 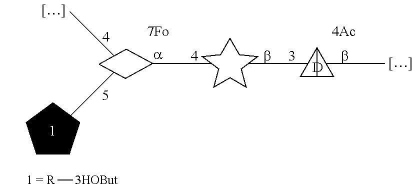 | -4)[Fo(1-7),IR3HOBut(1- 5)]aXPsep(2-4)bDXylp(1- 3)[Ac(1-4), Ac(1- 2)]bDFucpN(1- |
| H-47 | 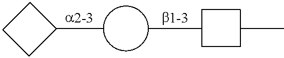 | Neu5Gca2-3Galb1- 3GlcNAc |
| H-48 | 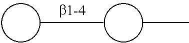 | Glcb1-4Glc(D-cellulose) |
| H-49 | 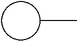 | Gal |
| H-50 | 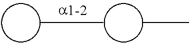 | Gala1-2Gal |
| H-51 | 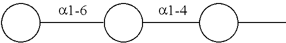 | Glca1-6Glca1-4Glc |
| H-52 | 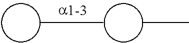 | Gala1-3Gal |
| H-53 | 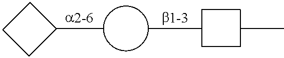 | Neu5Gca2-6Galb1- 3GlcNAc |
| H-54 | 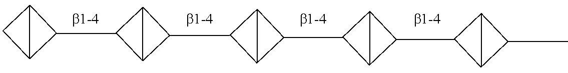 | ManAb1-4ManAb1- 4ManAb1-4ManAb1- 4ManA(D- pentamannuronic acid) |
| H-55 | 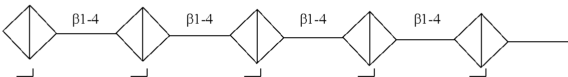 | [L]ManAb1- 4[L]ManAb1- 4[L]ManAb1- 4[L]ManAb1-4[L]ManA (L-pentaguluronicacidb- |
| | | Sp1) |
| H-56 | 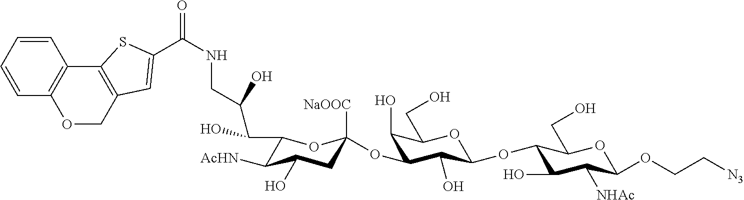 | 9-N-4H-thieno[3,2- c]chromene-2-carbamoyl- Neu5Aca(2-6)Galb(1- 4)GlcNAcb-O-ethyl azide |
| H-57 | 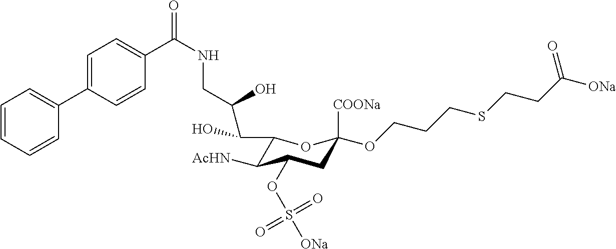 | 9-N-biphenylcarbamoyl- [4S]Neu5Aca(2-6)Galb(1- 4)GlcNAcb-O-ethyl azide |
| H-58 | 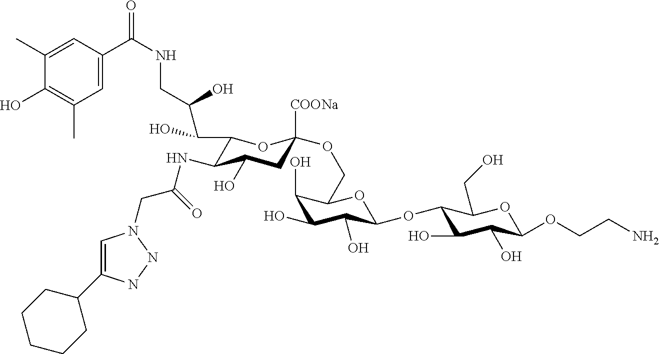 | 9-N-4-hydroxy-3,5- dimethylbenzyl- carbamoyl-[5-N-2-(4- cyclohexyl-1H-1,2,3- triazol-1- yl)acetylcarbamoyl]Neua (2-6)Galb(1-4)GlcNAcb- O-ethyl azide |
| H-59 | 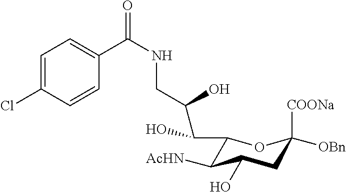 | 9-N-4-chlorobenzyl carbamoyl-Neu5Aca-O- ethyl azide |
| H-60 | 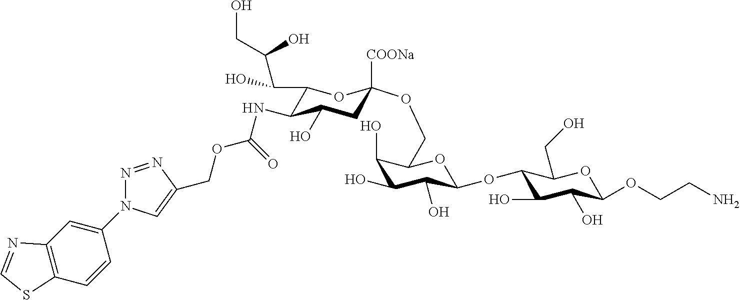 | 5-N-(1-(benzo[d]thiazol- 5-yl)-1H-1,2,3-triazol-4- yl)methoxylcarbamoyl- Neua(2-6)Galb(1- 4)GlcNAcb-O-ethyl azide |
| H-61 | 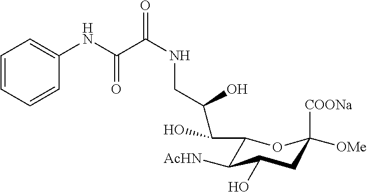 | 9-N-2-oxo-2- (phenylamino)acetamido- Neu5Aca-O-ethyl azide |
| H-62 | 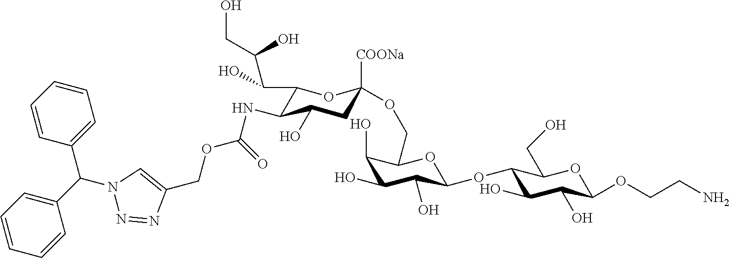 | 5-N-(1-benzhydryl-1H- 1,2,3-triazol-4-yl) methoxylcarbamoyl- Neua(2-6)Galb(1- 4)GlcNAcb-O-ethyl azide |
| H-63 | 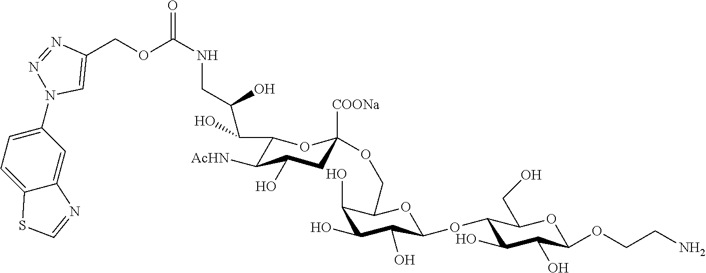 | 9-N-(1-(benzo[d]thiazol- 5-yl)-1H-1,2,3-triazol-4- yl)methoxylcarbamoyl- Neu5Aca(2-6)Galb(1- 4)GlcNAcb-O-ethyl azide |
| H-64 | 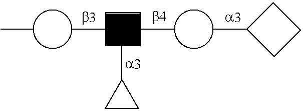 | Neu5Ac(a2-3)Gal(b1- 4)[Fuc(a1-3)]GlcNAc(b1- 3)Gal |
| H-65 | 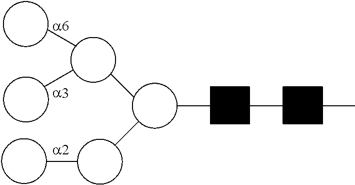 | Man(a1-6)[Man(a1- 3)]Man(a1-6)[Man(a1- 2)Man(a1-3)]Man(b1- 4)GlcNAc(b1-4)GlcNAc |

In some embodiments, a glycan targeting moiety of the present disclosure comprises a glycan selected from those listed in Table 1C below:

TABLE 1C
Exemplary Glycans

| Glycan | | |
|---|---|---|
| # | Structure | IUPAC Name |
| J-1 | 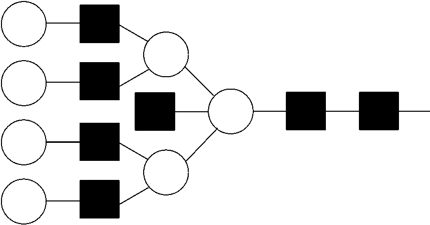 | Gal(b1-4)GlcNAc(b1- 6)[Gal(b1-4)GlcNAc(b1- 2)]Man(a1-6)[Gal(b1- 4)GlcNAc(b1-4)[Gal(b1- 4)GlcNAc(b1-2)]Man(al- 3)][GlcNAc(b1- 4)]Man(b1-4)GlcNAc(b1- 4)GlcNAc |
| J-2 | 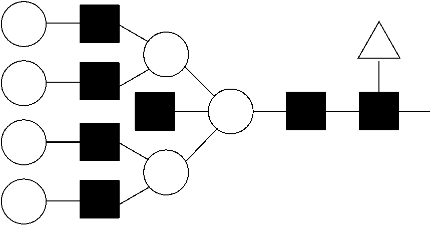 | Gal(b1-4)GlcNAc(b1- 6)[Gal(b1-4)GlcNAc(b1- 2)]Man(a1-6)[Gal(b1- 4)GlcNAc(b1-4)[Gal(b1- 4)GlcNAc(b1-2)]Man(al- 3)][GlcNAc(b1- 4)]Man(b1-4)GlcNAc(b1- 4)[Fuc(a1-6)]GlcNAc |
| J-3 | 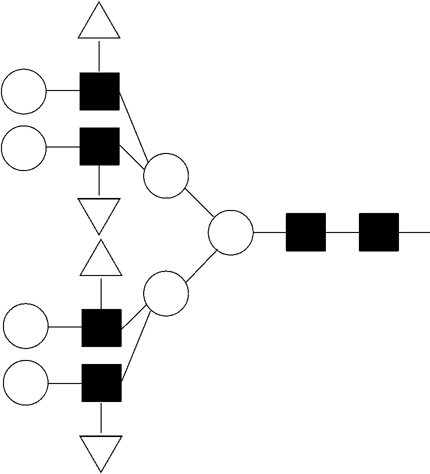 | Gal(b1-4)[Fuc(a1- 3)]GlcNAc(b1-6)[Gal(b1- 4)[Fuc(a1-3)]GlcNAc(b1- 2)]Man(a1-6)[Gal(b1- 4)[Fuc(a1-3)]GlcNAc(b1- 4)[Gal(b1-4)[Fuc(al- 3)]GlcNAc(b1- 2)]Man(a1-3)]Man(b1- 4)GlcNAc(b1-4)GlcNAc |
| J-4 | 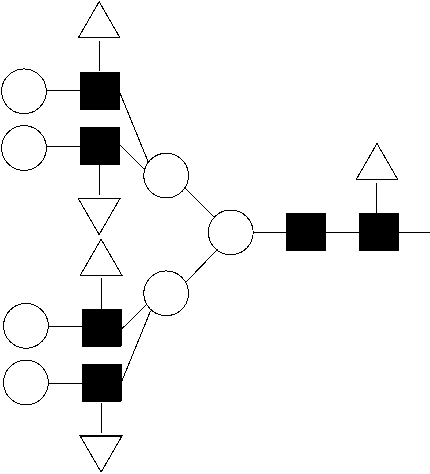 | Gal(b1-4)[Fuc(a1- 3)]GlcNAc(b1-6)[Gal(b1- 4)[Fuc(a1-3)]GlcNAc(b1- 2)]Man(a1-6)[Gal(b1- 4)[Fuc(a1-3)]GlcNAc(b1- 4)[Gal(b1-4)[Fuc(al- 3)]GlcNAc(b1- 2)]Man(a1-3)]Man(b1- 4)GlcNAc(b1-4)[Fuc(al- 6)]GlcNAc |
| J-5 | 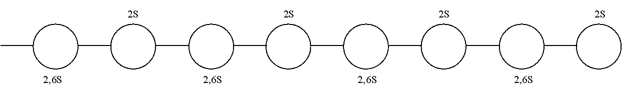 | [2,6S]Gal(b1- 4)[2S]Gal[2,6S]Gal(b1- 4)[2S]Gal[2,6S]Gal(b1- 4)[2S]Gal[2,6S]Gal(b1- 4)[2S]Gal |
| J-6 | 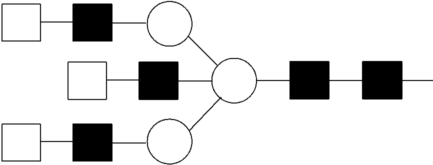 | GalNAc(b1- 4)GlcNAc(b1-2)Man(a1- 6)[GalNAc(b1- 4)GlcNAc(b1- 4)][GalNAc(b1- 4)GlcNAc(b1-2)Man(al- 3)]Man(b1-4)GlcNAc[b1- 4)GlcNAc |
| J-7 | 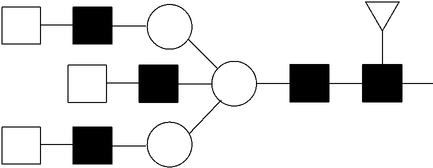 | GalNAc(b1- 4)GlcNAc(b1-2)Man(a1- 6)[GalNAc(b1- 4)GlcNAc(b1- 4)][GalNAc(b1- 4)GlcNAc(b1-2)Man(a1- 3)]Man(b1-4)GlcNAc[b1- 4)[Fuc(a1-6)]GlcNAc |
| J-8 | 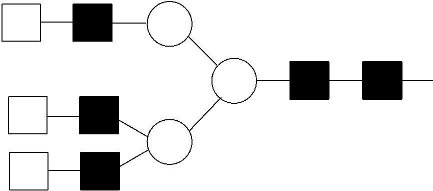 | GalNAc(b1- 4)GlcNAc(b1-2)Man(a1- 6)[GalNAc(b1- 4)GlcNAc(b1- 4)[GalNAc(b1- 4)GlcNAc(b1-2)]Man(a1- 3)]Man(b1-4)GlcNAc(b1- 4)GlcNAc |
| J-9 | 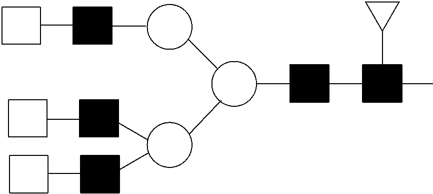 | GalNAc(b1- 4)GlcNAc(b1-2)Man(a1- 6)[GalNAc(b1- 4)GlcNAc(b1- 4)[GalNAc(b1- 4)GlcNAc(b1-2)]Man(a1- 3)]Man(b1-4)GlcNAc(b1- 4)[Fuc(a1-6)]GlcNAc |
| J-10 | 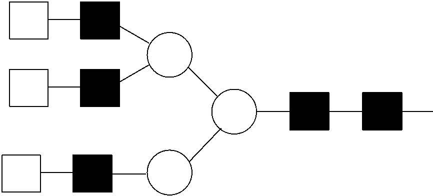 | GalNAc(b1- 4)GlcNAc(b1- 6)[GalNAc(b1- 4)GlcNAc(b1-2)]Man(a1- 6)[GalNAc(b1- 4)GlcNAc(b1-2)Man(a1- 3)]Man(b1-4)GlcNAc(b1- 4)GlcNAc |
| J-11 | 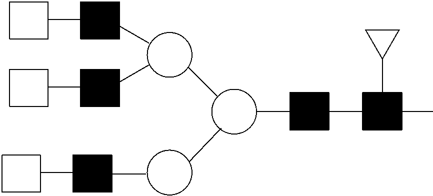 | GalNAc(b1- 4)GlcNAc(b1- 6)[GalNAc(b1- 4)GlcNAc(b1-2)]Man(a1- 6)[GalNAc(b1- 4)GlcNAc(b1-2)Man(a1- 3)]Man(b1-4)GlcNAc(b1- 4)[Fuc(al-6)]GlcNAc |
| J-12 | 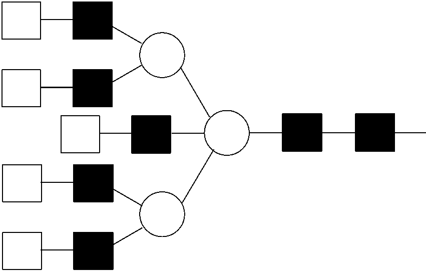 | GalNAcb1-4GlcNAcb1- 6(GalNAcb1-4GlcNAcb1- 2)Mana1-6(GalNAcb1- 4GlcNAcb1- 4)(GalNAcb1- 4GlcNAcb1-4(GalNAcb1- 4GlcNAcb1-2)Mana1- 3)Manb1-4GlcNAcb1- 4GlcNAc |
| J-13 | 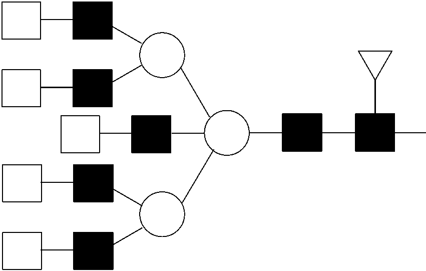 | GalNAcb1-4GlcNAcb1- 6(GalNAcb1-4GlcNAcb1- 2)Mana1-6(GalNAcb1- 4GlcNAcb1- 4)(GalNAcb1- 4GlcNAcb1-4(GalNAcb1- 4GlcNAcb1-2)Mana1- 3)Manb1-4GlcNAcb1- 4(Fuca1-6)GlcNAc |
| J-14 | 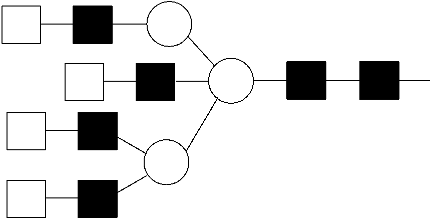 | GalNAcb1-4GlcNAcb1- 2Mana1-6(GalNAcb1- 4GlcNAcb1- 4)(GalNAcb1- 4GlcNAcb1-4(GalNAcb1- 4GlcNAcb1-2)Mana1- 3)Manb1-4GlcNAcb1- 4GlcNAc |
| J-15 | 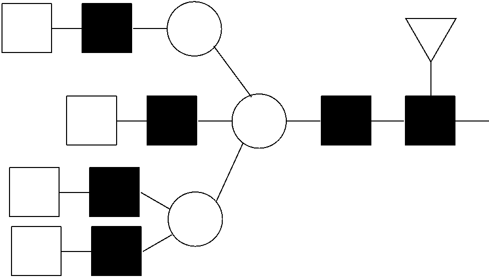 | GalNAcb1-4GlcNAcb1- 2Mana1-6(GalNAcb1- 4GlcNAcb1- 4)(GalNAcb1- 4GlcNAcb1-4(GalNAcb1- 4GlcNAcb1-2)Mana1- 3)Manb1-4GlcNAcb1- 4(Fuca1-6)GlcNAc |
| J-16 | 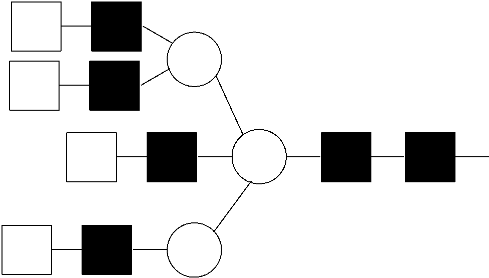 | GalNAcb1-4GlcNAcb1- 6(GalNAcb1-4GlcNAcb1- 2)Mana1-6(GalNAcb1- 4GlcNAcb1- 4)(GalNAcb1- 4GlcNAcb1-2Mana1- 3)Manb1-4GlcNAcb1- 4GlcNAc |
| J-17 | 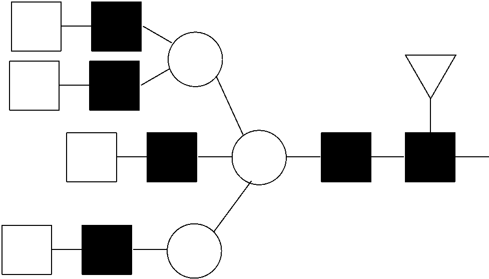 | GalNAcb1-4GlcNAcb1- 6(GalNAcb1-4GlcNAcb1- 2)Mana1-6(GalNAcb1- 4GlcNAcb1- 4)(GalNAcb1- 4GlcNAcb1-2Mana1- 3)Manb1-4GlcNAcb1- 4(Fuca1-6)GlcNAc |
| J-18 | 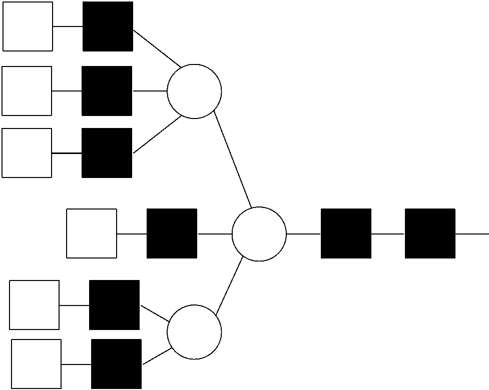 | GalNAcb1-4GlcNAcb1- 6(GalNAcb1-4GlcNAcb1- 4)(GalNAcb1- 4GlcNAcb1-2)Mana1- 6(GalNAcb1-4GlcNAcb1- 4)(GalNAcb1- 4GlcNAcb1-4(GalNAcb1- 4GlcNAcb1-2)Mana1- 3)Manb1-4GlcNAcb1- 4GlcNAc |
| J-19 | 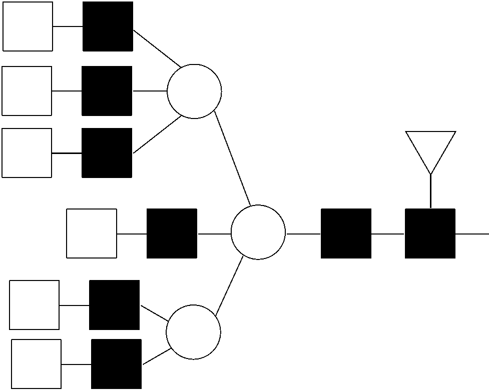 | GalNacb1-4GlcNAcb1- 6(GalNAcb1-4GlcNAcb1- 4)(GalNAcb1- 4GlcNAcb1-2)Mana1- 6(GalNAcb1-4GlcNAcb1- 4)(GalNAcb1- 4GlcNAcb1-4(GalNAcb1- 4GlcNAcb1-2)Mana1- 3)Manb1-4GlcNAcb1- 4(Fuca1-6)GlcNAc |

In some embodiments, a glycan targeting moiety of the present disclosure comprises a glycan selected from those listed in Table 1D below:

TABLE 1D
Exemplary Glycans

| Glycan # | Structure | IUPAC Name |
|---|---|---|
| K-1 | 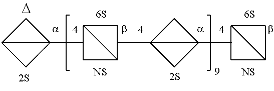 | ΔUA,2S-GlcNS,6S- [IdoUA,2S-GlcNS,6S]₉ |
| K-2 | 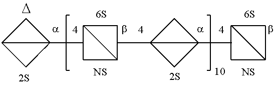 | ΔUA,2S-GlcNS,6S- [IdoUA,2S-GlcNS,6S]₁₀ |
| K-3 | 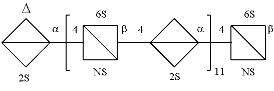 | ΔUA,2S-GlcNS,6S- [IdoUA,2S-GlcNS,6S]₁₁ |
| K-4 | 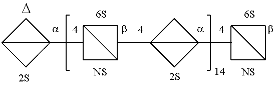 | ΔUA,2S-GlcNS,6S- [IdoUA,2S-GlcNS,6S]₁₄ |
| K-5 | 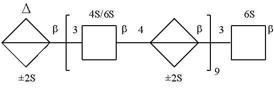 | ΔUA-[GalNAc,6S or 4S- GlcA +/− 2S]₉- GalNAc,6S, |
| K-6 | 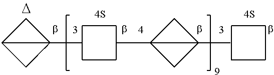 | ΔUAβ1,3-GalNAc,4S- [IdoA-GalNAc,4S]₉ |
| K-7 | 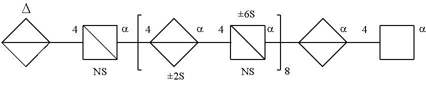 | ΔUA-GlcNS-[IdoA +/- 2S-GlcNS]₈-IdoA- GlcNAc |
| K-8 | 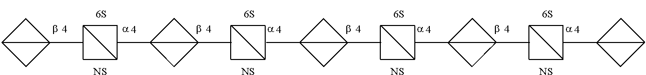 | GlcAβ1-4GlcNS6Sα1- 4GlcAβ1-4GlcNS6Sα1- 4GlcAβ1-4GlcNS6Sα1- 4GlcAβ1-4GlcNS6Sα1- 4GlcA |
| K-9 | 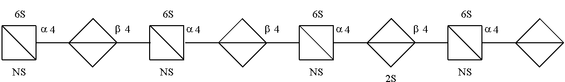 | GlcNS6Sα1-4GlcAβ1- 4GlcNS6Sα1-4GlcAβ1- 4GlcNS6Sα1-4IdoA2Sβ1- 4GlcNS6Sα1-4GlcA |
| K-10 | 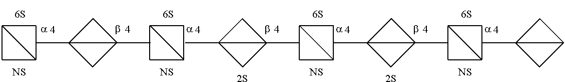 | GlcNS6Sα1-4GlcAβ1- 4GlcNS6Sα1-4IdoA2Sβ1- 4GlcNS6Sα1-4IdoA2Sβ1- 4GlcNS6Sα1-4GlcA |
| K-11 | 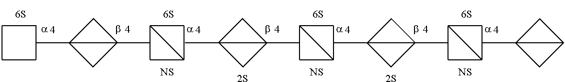 | GlcNAc6Sα1-4GlcAβ1- 4GlcNS6Sα1-4IdoA2Sβ1- 4GlcNS6Sα1-4IdoA2Sβ1- 4GlcNS6Sα1-4GlcA |
| K-12 | 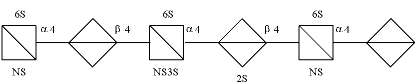 | GlcNS6Sα1-4GlcAβ1- 4GlcNS3S6Sα1- 4IdoA2Sβ1-4GlcNS6Sα1- 4GlcA |
| K-13 | 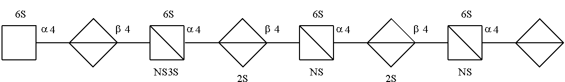 | GlcNAc6Sα1-4GlcAβ1- 4GlcNS3S6Sα1- 4IdoA2Sβ1-4GlcNS6Sα1- 4IdoA2Sβ1-4GlcNS6Sα1- 4GlcA |
| K-14 | 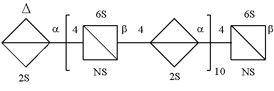 | ΔUA,2S-GlcNS,6S- [IdoUA,2S- GlcNS,6S]10 |
| K-15 | 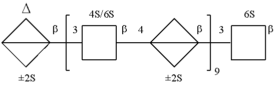 | ΔUA-[GalNAc,6S or 4S- GlcA +/− 2S]9- GalNAc,6S |
| K-16 | 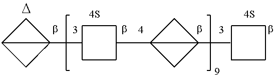 | ΔUAβ1,3-GalNAc,4S- [IdoA-GalNAc,4S]9 |
| K-17 | 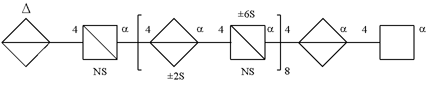 | ΔUA-GIcNS-[IdoA +/− 2S-GlcNS]8-IdoA- GlcNAc |
| K-18 | 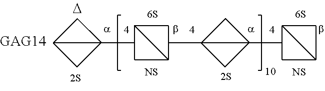 | ΔUA,2S-GlcNS,6S- [IdoUA,2S- GlcNS,6S]10 |
| K-19 | 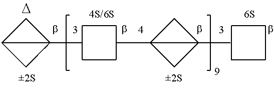 | ΔUA-[GalNAc,6S or 4S- GlcA +/− 2S]9- GalNAc,6S |
| K-20 | 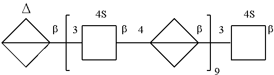 | ΔUAβ1,3-GalNAc,4S- [IdoA-GalNAc,4S]9 |
| K-21 | 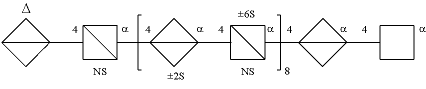 | ΔUA-GlcNS-[IdoA +/− 2S-GlcNS]8-IdoA- GlcNAc |
| K-22 | 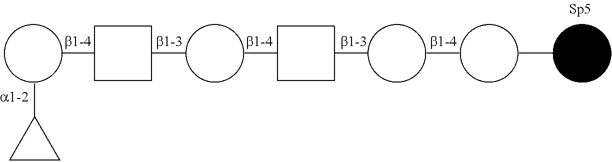 | Fucα1-2Galb1- 4GlcNAcb1-3Galb1- 4GlcNAcb1-3Galb1-4Glc |
| K-23 | 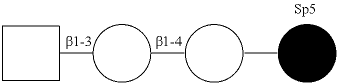 | GlcNAcb1-3Galb1-4Glc |
| K-24 | 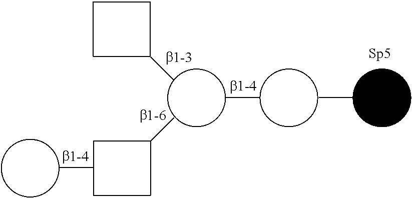 | GlcNAcb1-3(Galb1- 4GlcNAcb1-6)Galb1- 4Glc |
| K-25 | 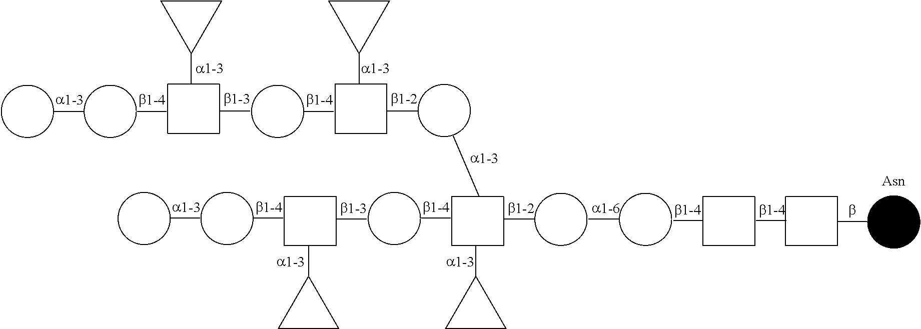 | Gala1-3Galb1-4(Fuca1- 3)GlcNAcb1-3Galb1- 4(Fuca1-3)GlcNAcb1- 3Galb1-4(Fuca1- 3)GlcNAcb1-2Mana1- 3(Gala1-3Galb1-4(Fucal- 3)GlcNAcb1-3Galb1- 4(Fuca1-3)GlcNAcb1- 3Galb1-4(Fuca1- 3)GlcNAcb1-2Mana1- 6)Manb1-4GlcNAcb1- 4GlcNAcb |
| K-26 | 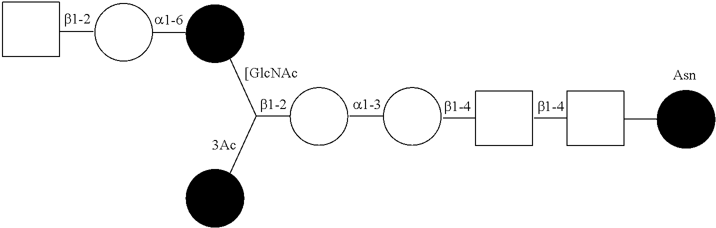 | GlcNAcb1-2Mana1- 6[GlcNAc(3Ac)b1- 2Mana1-3Manb1- 4GlcNAcb1-4GlcNAc |
| K-27 | 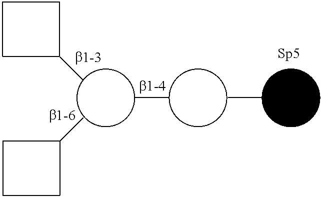 | GlcNAcb1-3(GlcNAcb1- 6)Galb1-4Glc |
| K-28 | 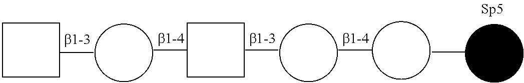 | GlcNAcb1-3Galb1- 4GlcNAcb1-3Galb1-4Glc |
| K-29 | 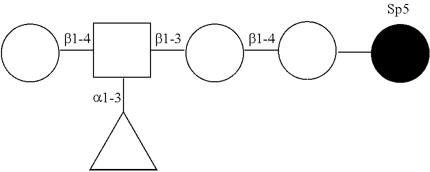 | Galb1-4(Fucα1- 3)GlcNAcb1-3Galb1- 4Glc |
| K-30 | 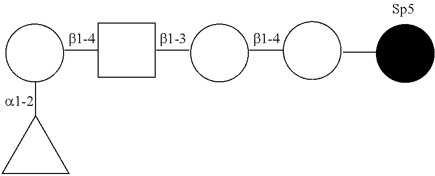 | Fuca1-2Galb1- 4GlcNAcb1-3Galb1-4Glc |
| K-31 | 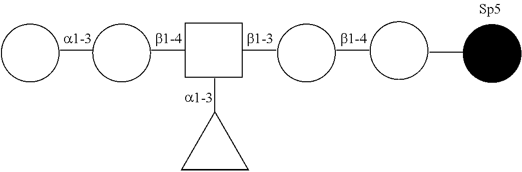 | Gala1-3Galb1-4(Fuca1- 3)GlcNAcb1-3Galb1- 4Glc |
| K-32 | 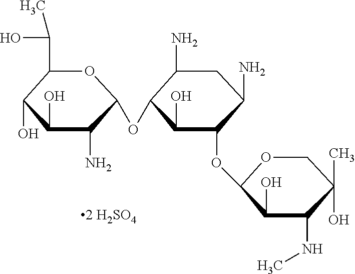 | ″Geneticin disulfate″ (2R,3S,4R,5R,6S)-5- Amino-6- {[(1R,2S,3S,4R,6S)-4,6- diamino-3- {[(2R,3R,4R,5R)-3,5- dihydroxy-5-methyl-4- (methylamino)oxan-2- yl]oxy}-2- hydroxycyclohexyl]oxy}- 2-[(1R)-1- hydroxyethyl]oxane-3,4- diol disulfate |
| K-33 | 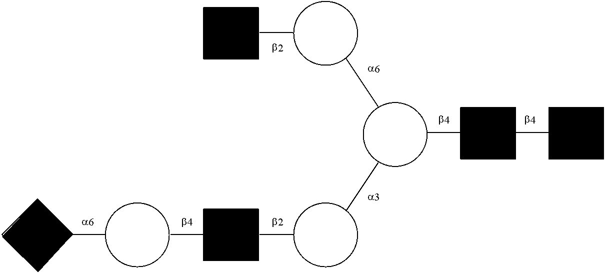 | GlcNAcβ1-2Manα1- 6(Neu5Acα2-6Galβ1- 4GlcNAcβ1-2Manα1- 3)Manβ1-4GlcNAcβ1- 4GlcNAc- |
| K-34 | 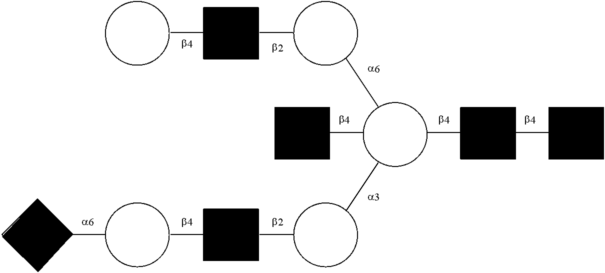 | GlcNAcβ1-2Manα1- 6(GlcNAcβ1- 4)(Neu5Acα2-6Galβ1- 4GlcNAcβ1-2Manα1- 3)Manβ1-4GlcNAcβ1- 4GlcNAc- |
| K-35 | 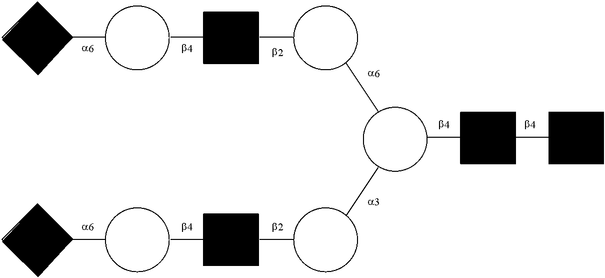 | Neu5Acα2-6Galβ1- 4GlcNAcβ1-2Manα1- 6(Neu5Acα2-6Galβ1- 4GlcNAcβ1-2Manα1- 3)Manβ1-4GlcNAcβ1- 4GlcNAc- |
| K-36 | 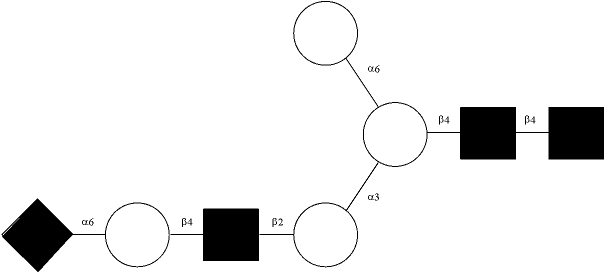 | Manα1-6(Neu5Acα2- 6Galβ1-4GlcNAcβ1- 2Manα1-3)Manβ1- 4GlcNAcβ1-4GlcNAc- |
| K-37 | 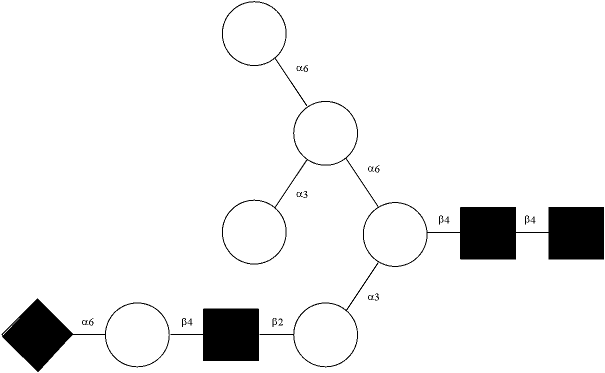 | Manα1-6(Manα1- 3)Manα1-6(Neu5Acα2- 6Galβ1-4GlcNAcβ1- 2Manα1-3)Manβ1- 4GlcNAcβ1-4GlcNAc- |
| K-38 | 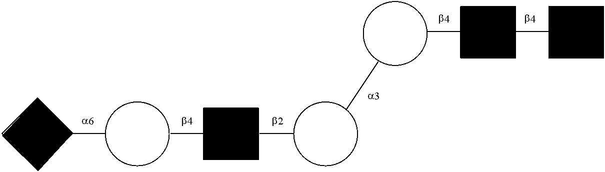 | Neu5Acα2-6Galβ1- 4GlcNAcβ1-2Manα1- 3Manβ1-4GlcNAcβ1- 4GlcNAc- |
| K-39 | 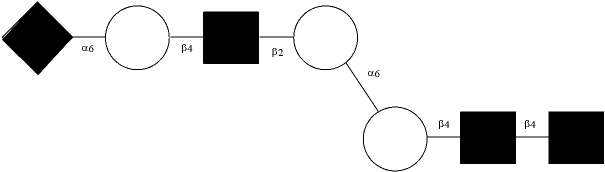 | Neu5Acα2-6Galβ1- 4GlcNAcβ1-2Manα1- 6Manβ1-4GlcNAcβ1- 4GlcNAc- |
| K-40 | 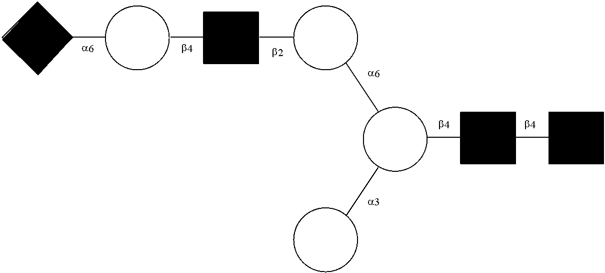 | Neu5Acα2-6Galβ1- 4GlcNAcβ1-2Manα1- 6(Manα1-3)Manβ1- 4GlcNAcβ1-4GlcNAc- |
| K-41 | 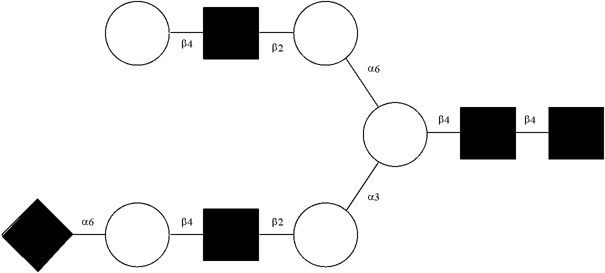 | Galβ1-4GlcNAcβ1- 2Manα1-6(Neu5Acα2- 6Galβ1-4GlcNAcβ1- 2Manα1-3)Manβ1- 4GlcNAcβ1-4GlcNAc- |
| K-42 | 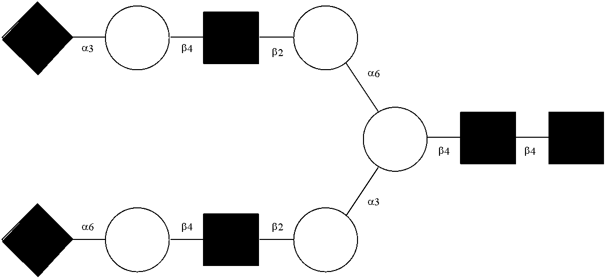 | Neu5Acα2-3Galβ1- 4GlcNAcβ1-2Manα1- 6(Neu5Acα2-6Galβ1- 4GlcNAcβ1-2Manα1- 3)Manβ1-4GlcNAcβ1- 4GlcNAc- |
| K-43 | 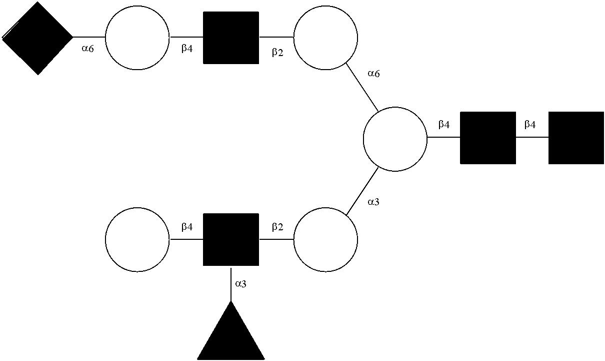 | Neu5Acα2-6Galβ1- 4GlcNAcβ1-2Manα1- 6(Galβ1-4(Fucα1- 3)GlcNAcβ1-2Manα1- 3)Manβ1-4GlcNAcβ1- 4GlcNAc- |
| K-44 | 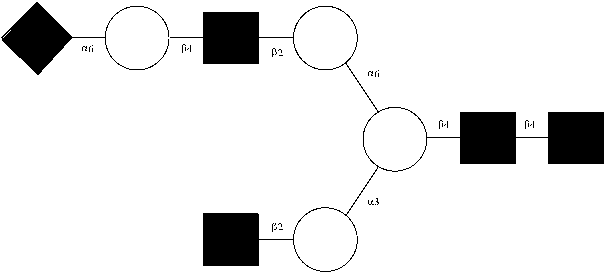 | Neu5Acα2-6Galβ1- 4GlcNAcβ1-2Manα1- 6(GlcNAcβ1-2Manα1- 3)Manβ1-4GlcNAcβ1- 4GlcNAc- |
| K-45 | 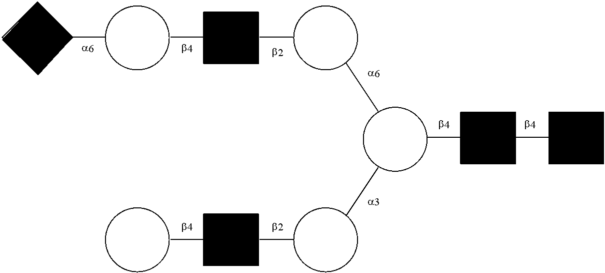 | Neu5Acα2-6Galβ1- 4GlcNAcβ1-2Manα1- 6(Galβ1-4GlcNAcβ1- 2Manα1-3)Manβ1- 4GlcNAcβ1-4GlcNAc- |
| K-46 | 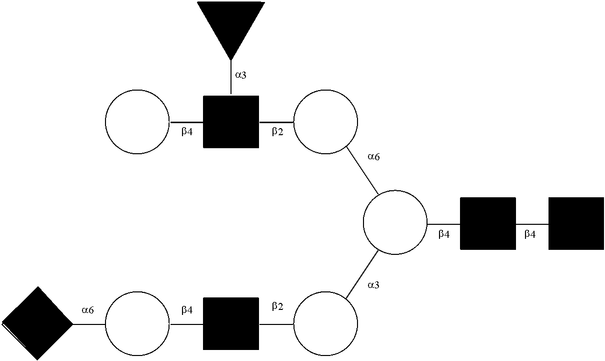 | Galβ1-4(Fucα1- 3)GlcNAcβ1-2Manα1- 6(Neu5Acα2-6Galβ1- 4GlcNAcβ1-2Manα1- 3)Manβ1-4GlcNAcβ1- 4GlcNAc- |
| K-47 | 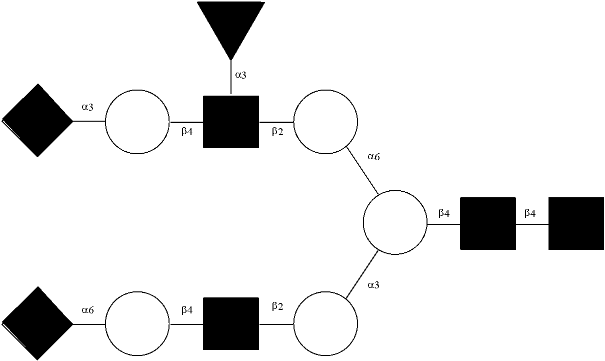 | Neu5Acα2-3Galβ1- 4(Fucα1-3)GlcNAcβ1- 2Manα1-6(Neu5Acα2- 6Galβ1-4GlcNAcβ1- 2Manα1-3)Manβ1- 4GlcNAcβ1-4GlcNAc- |
| K-48 | 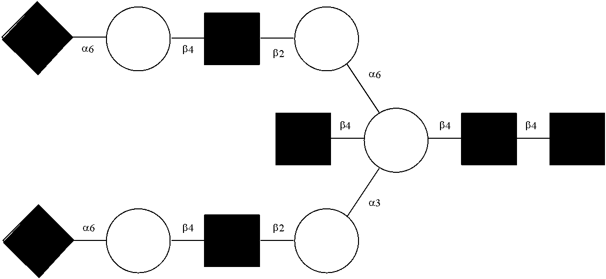 | Neu5Acα2-6Galβ1- 4GlcNAcβ1-2Manα1- 6(GlcNAcβ1- 4)(Neu5Acα2-6Galβ1- 4GlcNAcβ1-2Manα1- 3)Manβ1-4GlcNAcβ1- 4GlcNAc- |
| K-49 | 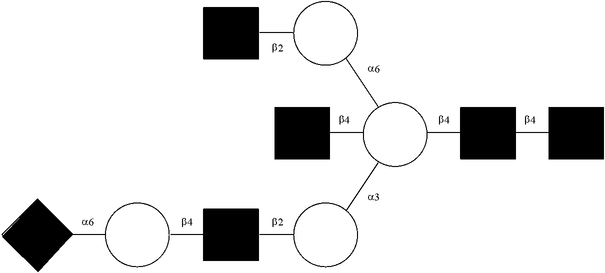 | GlcNAcβ1-2Manα1- 6(GlcNAcβ1- 4)(Neu5Acα2-6Galβ1- 4GlcNAcβ1-2Manα1- 3)Manβ1-4GlcNAcβ1- 4GlcNAc- |
| K-50 | 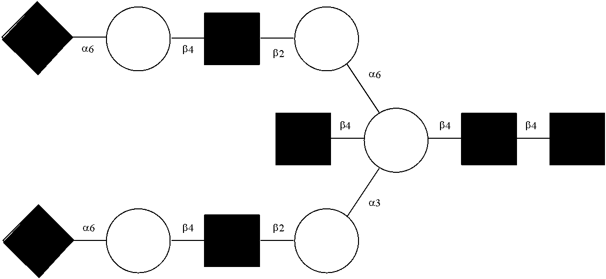 | Neu5Acα2-3Galβ1- 4GlcNAcβ1-2Manα1- 6(GlcNAcβ1- 4)(Neu5Acα2-6Galβ1- 4GlcNAcβ1-2Manα1- 3)Manβ1-4GlcNAcβ1- 4GlcNAc- |
| K-51 | 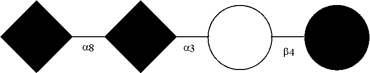 | Neu5Ac(a2- 8)Neu5Ac(a2-3)Gal(b1- 4)Glc |
| K-52 | 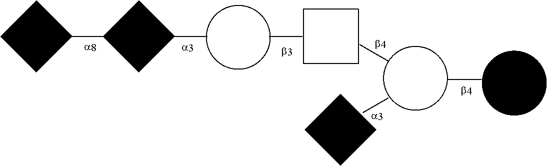 | Neu5Acα2-8Neu5Acα2- 3Galβ1-3GalNAcβ1- 4(Neu5Acα2-3)Galβ1- 4Glc |
| K-53 | 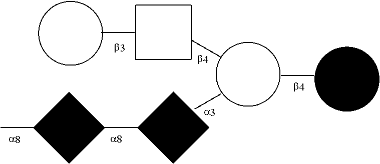 | Neu5Ac(a2- 8)Neu5Ac(a2- 8)Neu5Ac(a2-3)[Glc(b2- 3)GalNAc(b1-4)]Gal(b1- 4)Glc |

In some embodiments, a glycan targeting moiety of the present disclosure comprises a glycan selected from those listed in Table 1E below:

TABLE 1E
Exemplary Glycans

| Glycan | | |
|---|---|---|
| # | Structure | IUPAC Name |
| M-1 | 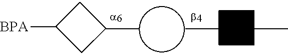 | [9-N-(bipheny1)-4- methylCarbamoyl] Neu5Gc(a2-6)Gal(b1- 4)GlcNAc |
| M-2 | 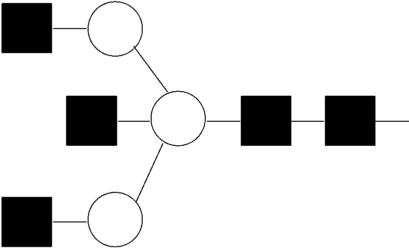 | GlcNAcb1-2Mana1- 6(GlcNAcb1- 4)(GlcNAcb1-2Mana1- 3)Manb1-4GlcNAcb1- 4GlcNAc |
| M-3 | 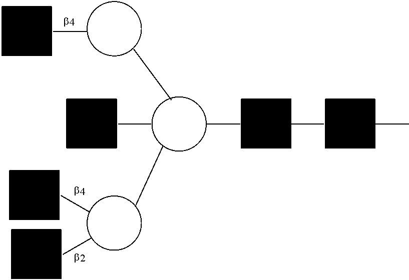 | GlcNAcb1-4Mana1- 6(GlcNAcb1- 4)(GlcNAcb1- 4(GlcNAcb1-2)Mana1- 3)Manb1-4GlcNAcb1- 4GlcNAc |
| M-4 | 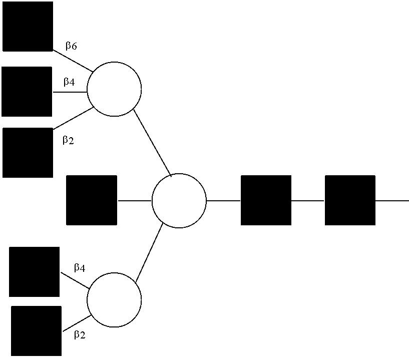 | GlcNAcb1-6(GlcNAcb1- 4)(GlcNAcb1-2)Mana1- 6(GlcNAcb1- 4)(GlcNAcb1- 4(GlcNAcb1-2)Mana1- 3)Manb1-4GlcNAcb1- 4GlcNAc |
| M-5 | 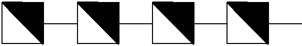 | GlcNb1-4GlcNb1- 4GlcNb1-4GlcN |
| M-6 | 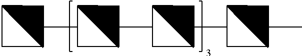 | GlcNb1-4GlcNb1- 4GlcNb1-4GlcNb1- 4GlcNb1-4GlcNb1- 4GlcNb1-4GlcN |
| M-7 | 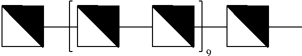 | GlcNb1-4GlcNb1- 4GlcNb1-4GlcNb1- 4GlcNb1-4GlcNb1- 4GlcNb1-4GlcNb1- |
| | | 4GlcNb1-4GlcNb1- |
| | | 4GlcNb1-4GlcNb1- |
| | | 4GlcNb1-4GlcNb1- |
| | | 4GlcNb1-4GlcNb1- |
| | | 4GlcNb1-4GlcNb1- |
| | | 4GlcNb1-4GlcN |
| M-8 | 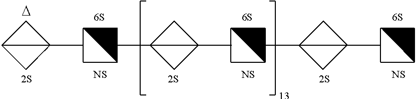 | ΔUA2Sb1-4GlcNS6Sal- 4[IdoA2Sb1- 4GlcNS6S]14 |
| M-9 | 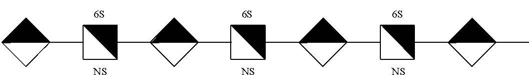 | GlcAβ1-4GlcNS6Sα1- 4GlcAβ1-4GlcNS6Sα1- 4GlcAβ1-4GlcNS6Sα1- 4GlcAβ1-4GlcNS6Sα1- 4GlcA |
| M-10 | 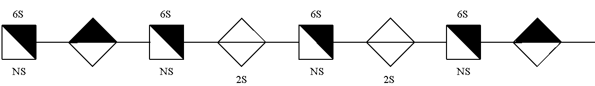 | GlcNS6Sα1-4GlcAβ1- 4GlcNS6Sα1-4IdoA2Sβ1- 4GlcNS6Sα1-4IdoA2Sβ1- 4GlcNS6Sα1-4GlcA |
| M-11 | 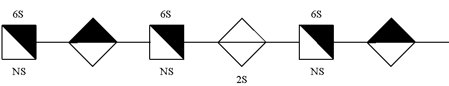 | GlcNS6Sα1-4GlcAβ1- 4GlcNS3S6Sα1- 4IdoA2Sβ1-4GlcNS6Sα1- 4GlcA |
| M-12 | 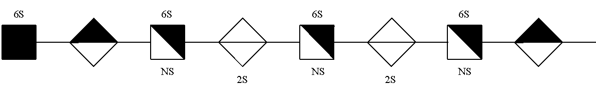 | GlcNAc6Sα1-4GlcAβ1- 4GlcNS3S6Sα1- 4IdoA2Sβ1-GlcNS6Sα- 4IdoA2Sβ1-4GlcNS6Sα1- 4GlcA |
| M-13 | 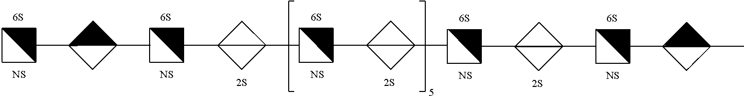 | GlcNS6Sa1-4GlcAb1- 4[GlcNS6Sal- 4IdoA2S]7b1- 4GlcNS6Sa1-4GlcA |
| M-14 | 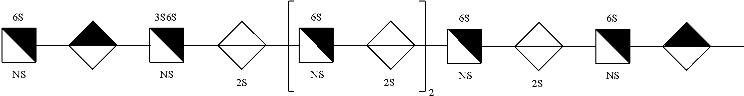 | GlcNS6Sa1-4GlcAb1-4- GlcNS3S6Sa1-4IdoA2S [GlcNS6Sal- 4IdoA2S]3b1- 4GlcNS6Sa1-4GlcA |
| M-15 | 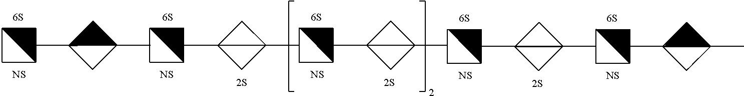 | GlcNS6Sa1-4GlcAb1- 4[GlcNS6Sal- 4IdoA2S]4b1- 4GlcNS6Sa1-4GlcA |
| M-16 | 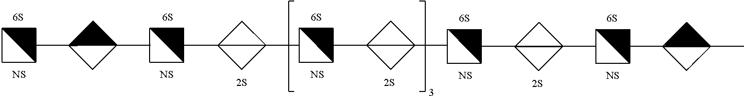 | GlcNS6Sa1-4GlcAb1- 4[GlcNS6Sal- 4IdoA2S]5b1- 4GlcNS6Sa1-4GlcA |
| M-17 | 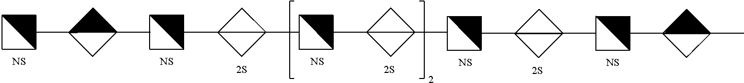 | GlcNSa1-4GlcAb1- 4[GlcNSa1-4IdoA2S]7b1- 4GlcNSa1-4GlcA |

In some embodiments, a glycan targeting moiety of the present disclosure comprises a glycan of a ganglioside compound. Gangliosides are a group of complex lipids which are naturally present in the gray matter of the brain, central nervous system and/or peripheral nervous system. Generally, gangliosides comprise one or more sialic acid moieties as part of a glyco-sphingolipid. In some embodiments, a glycan targeting moiety of the present disclosure comprises a glycan selected from those described in Table 1F.

TABLE 1F
Exemplary Ganglioside Glycans

| Common Name | Glycan moiety |
|---|---|
| GM1 / GM1A | Galb(1-3)GalNAcb(1-4)[Neu5Aca(2-3)]Galb(1-4)Glcb(1-1)Cer |
| GM1b | Neu5Aca(2-3)Galb(1-3)GalNAcb(1-4)Galb(1-4)Glcb(1-1)Cer |
| GM2-1 | Neu5Aca(2-3)Galb(1-?)bDGalNAc(1-?)bDGalNAc(1-?)Glcb(1-1)Cer |
| GM2 / GM2a | GalNAcb(1-4)[Neu5Aca(2-3)]Galb(1-4)Glcb(1-1)Cer |
| GM3 | Neu5Aca(2-3)Galb(1-4)Glcb(1-1)Cer |
| GM2b | Neu5Aca(2-8)Neu5Aca(2-3)Galb(1-4)Glcb(1-1)Cer |
| asialo-GM1, GA1 | Galb(1-3)GalNAcb(1-4)Galb(1-4)Glcb(1-1)Cer |
| asialo-GM2, GA2 | GalNAcb(1-4)Galb(1-4)Glcb(1-1)Cer |
| GD3 | Neu5Aca(2-8)Neu5Aca(2-3)Galb(1-4)Glcb(1-1)Cer |
| GD2 | GalNAcb(1-4)[Neu5Aca(2-8)Neu5Aca(2-3)]Galb(1-4)Glcb(1-1)Cer |
| GD1a | Neu5Aca(2-3)Galb(1-3)GalNAcb(1-4)[Neu5Aca(2-3)]Galb(1-4)Glcb(1-1)Cer |
| GD1alpha | Neu5Aca(2-3)Galb(1-3)[Neu5Aca(2-6)]GalNAcb(1-4)Galb(1-4)Glcb(1-1)Cer |
| GD1b | Galb(1-3)GalNAcb(1-4)[Neu5Aca(2-8)Neu5Aca(2-3)]Galb(1-4)Glcb(1-1)Cer |
| GT1a | Neu5Aca(2-8)Neu5Aca(2-3)Galb(1-3)GalNAcb(1-4)[Neu5Aca(2-3)]Galb(1- |
| | 4)Glcb(1-1)Cer |
| GT1, GT1b | Neu5Aca(2-3)Galb(1-3)GalNAcb(1-4)[Neu5Aca(2-8)Neu5Aca(2-3)]Galb(1- |
| | 4)Glcb(1-1)Cer |
| OAc-GT1b | Neu5Aca(2-3)Galb(1-3)GalNAcb(1-4)[Neu5Ac9Aca(2-8)Neu5Aca(2-3)]Galb(1- |
| | 4)Glcb(1-1)Cer |
| GT1c | Galb(1-3)GalNAcb(1-4)[Neu5Aca(2-8)Neu5Aca(2-8)Neu5Aca(2-3)]Galb(1- |
| | 4)Glcb(1-1)Cer |
| GT3 | Neu5Aca(2-8)Neu5Aca(2-8)Neu5Aca(2-3)Galb(1-4)Glcb(1-1)Cer |
| GQ1b | Neu5Aca(2-8)Neu5Aca(2-3)Galb(1-3)GalNAcb(1-4)[Neu5Aca(2-8)Neu5Aca(2- |
| | 3)]Galb(1-4)Glcb(1-1)Cer |
| GGal | Neu5Aca(2-3)Galb(1-1)Cer |
| NGc GM3 | Neu5Gca(2-3)Galb(1-4)Glcb(1-1)Cer |
| GT2 | GalNAcb(1-4)[Neu5Aca(2-8)Neu5Aca(2-8)Neu5Aca(2-3)]Galb(1-4)Glcb(1-1)Cer |
| GQ1c | Neu5Aca(2-3)Galb(1-3)GalNAcb(1-4)[Neu5Aca(2-8)Neu5Aca(2-8)Neu5Aca(2- |
| | 3)]Galb(1-4)Glcb(1-1)Cer |
| GA1 | Galb(1-3)GalNAcb(1-4)Galb(1-4)Glcb(1-1)Cer |
| GD1c | Neu5Aca(2-8)Neu5Aca(2-3)Galb(1-3)GalNAcb(1-4)Galb(1-4)Glcb(1-1)Cer |
| GP1c | Neu5Aca(2-8)Neu5Aca(2-3)Galb(1-3)GalNAcb(1-4)[Neu5Aca(2-8)Neu5Aca(2- |
| | 8)Neu5Aca(2-3)]Galb(1-4)Glcb(1-1)Cer |
| DSGb5 | Neu5Aca(2-3)Galb(1-3)[Neu5Aca(2-6)]GalNAcb(1-3)Gala(1-4)Galb(1-4)Glcb(1- |
| | 1)Cer |
| GT1aalpha | Neu5Aca(2-3)bDGalp(1-3)[Neu5Aca(2-6)]GalNAcb(1-4)[Neu5Aca(2- |
| | 3)]bDGalp(1-4)bDGlcp(1-1)Cer |
| GQ1balpha | Neu5Aca(2-3)Galb(1-3)[Neu5Aca(2-6)]GalNAcb(1-4)[Neu5Aca(2-8)Neu5Aca(2- |
| | 3)]Galb(1-4)bDGlcp(1-1)Cer |
| GP1calpha | Neu5Aca(2-8)Neu5Aca(2-3)[Neu5Aca(2-6)]Galb(1-3)GalNAcb(1-4)[Neu5Aca(2- |
| | 8)Neu5Aca(2-8)Neu5Aca(2-3)]Galb(1-4)Glcb(1-1)Cer |
| GA2 | GalNAcb(1-4)Galb(1-4)Glcb(1-1)Cer |
| GA1 | Galb(1-3)GalNAcb(1-4)Galb(1-4)Glcb(1-1)Cer |
| Gb3cer | Gala(1-4)Galb(1-4)Glcb(1-1)Cer |
| Gb4cer | GalNAcb(1-3)Gala(1-4)Galb(1-4)Glcb(1-1)Cer |
| Gb5cer | Galb(1-3)GalNAcb(1-3)Gala(1-4)Galb(1-4)Glcb(1-1)Cer |
| sialylGb5Cer | Neu5Aca(2-3)Galb(1-3)GalNAcb(1-3)Gala(1-4)Galb(1-4)Glcb(1-1)Cer |
| Lc3Cer | GlcNAcb(1-3)Galb(1-4)Glcb(1-1)Cer |
| nLc4Cer | Galb(1-4)GlcNAcb(1-3)Galb(1-4)Glcb(1-1)Cer |
| III³Fuca-nLc4Cer | Galb(1-4)[Fuca(1-3)]GlcNAcb(1-3)Galb(1-4)Glcb(1-1)Cer |

In some embodiments, the glycan moiety is or comprises a glycan that differs from a glycan of FIG. 1, or Tables 1A-1D by the replacement of a single monosaccharide. In some embodiments, the glycan moiety is or comprises a glycan that differs from a glycan of FIG. 1, or Tables 1A-1D by the replacement of two monosaccharides. As a non-limiting example, the glycan moiety can comprise a glycan of FIG. 1, or Tables 1A-1D, wherein a mannose is replaced by a galactose (or vice versa), but otherwise the rest of the glycan moiety remains the same.

In some embodiments, the glycan moiety is or comprises a glycan that differs from a glycan of FIG. 1, or Tables 1A-1F by the replacement of a single monosaccharide. In some embodiments, the glycan moiety is or comprises a glycan that differs from a glycan of FIG. 1, or Tables 1A-1F by the replacement of two monosaccharides. As a non-limiting example, the glycan moiety can comprise a glycan of FIG. 1, or Tables 1A-1F, wherein a mannose is replaced by a galactose (or vice versa), but otherwise the rest of the glycan moiety remains the same.

The glycan moiety can include one or more stereocenters and can have any desired configuration at the one or more stereocenters. For example, the glycan moiety can comprise a glycan that differs from a glycan of FIG. 1, Tables 1A-1D, or Tables 1A-1F by the configuration at one or more stereocenters (e.g., one or more stereocenters, two or more stereocenters, three or more stereocenters, four or more stereocenters, five or more stereocenters, six or more stereocenters). The number of stereocenters a glycan can differ from the glycan of FIG. 1, Tables 1A-1D, or Tables 1A-1F can be 1, 2, 3, 4, 5, 6, 7, 8, 9, or 10. The glycan moiety can differ by the configuration at an anomeric position (e.g., an alpha anomeric configuration of a monosaccharide, a beta anomeric configuration of a monosaccharide, a racemic anomeric configuration of a monosaccharide) of the glycan. The glycan moiety can differ by the configuration at a non-anomeric position of the glycan (e.g., at the 2, 3, 4, 5, 6, 7, or 8 position).

The glycan moiety can comprise a glycan that differs from a glycan of FIG. 1, Tables 1A-1D, Tables 1A-1F, or Tables 1E and 1F by the linkage between one or more monosaccharides of the glycan (e.g., two or more, three or more, four or more, five or more linkage sites). For example, a glycan can differ from GalNAcb(1-3)Gala(1-4)Galb(1-4)Glcb(1-1)Cer (e.g., Gb4cer, Table 1F) by one linkage site (e.g., GalNAcb(1-3)Gala(1-4)Galb(1-3)Glcb(1-1)Cer). The number of glycan linkage sites differing from a glycan of FIG. 1, Tables 1A-1D, or Tables 1A-1F can be 1, 2, 3, 4, 5, 6, 7, 8, 9, or 10. The linkage site can be any linkage position including, but not limited to, a (1-1), (1-2), (1-3), (1-4), (1-5), (1-6), (1-7), (1-8), (1-9), (2-2), (2-3), (2-4), (2-5), (2-6), (2-7), (2-8), or (2-9) linkage.

In some embodiments, the glycan moiety comprises glucose, N-acetylglucosamine, mannose, galactose, N-acetylgalactosamine, sialic acid, glucuronic acid, iduronic acid, glucosamine, galactosamine, xylose and fucose. In some embodiments, the glycan moiety comprises glucose, GlcNAc, mannose, galactose, sialic acid, N-Acetylneuraminic acid (NANA) and fucose, or a subset or combination thereof. In some embodiments, the glycan moiety comprises sialic acid and fucose, or a combination thereof. In some embodiments, the glycan moiety comprises sialic acid. In some embodiments, the glycan moiety comprises glucose. In some embodiments, the glycan moiety comprises fucose. In some embodiments, the glycan moiety comprises mannose. In some embodiments, the glycan moiety comprises GlcNAc (N-Acetylglucosamine). In some embodiments, the glycan moiety comprises galactose. In some embodiments, the glycan moiety comprises a fucose linked to a GlcNAc residue. In some embodiments, the glycan moiety comprises a fucose linked to a galactose residue. In some embodiments, the glycan moiety comprises a fucose linked to a glucose residue. In some embodiments, the glycan moiety comprises GalNAc. In some embodiments, the glycan moiety does not comprise GalNAc.

In some embodiments, the glycan moiety comprises one or more hexuronate sugar. In some embodiments, the glycan moiety comprises IdoA. In some embodiments, the glycan moiety comprises GlcA.

In some embodiments, the glycan moiety comprises glucose. In some embodiments, the glycan moiety consists of a multi-antennary glycan formed only of glucose monosaccharides. In some embodiments, the glycan moiety consists of a multi-antennary glycan formed only of galactose monosaccharides.

In some embodiments, the glycan moiety comprises β-muramic acid.

In some embodiments, the glycan moiety comprises one or more non-saccharide components or modifications. For example, exemplary glycan H-33 comprises a 9-N-biphenyl carboxamide (BPC) modification. In some embodiments, the glycan moiety comprises a BPC modification. In some embodiments, the glycan moiety comprises one or more non-saccharide components or modifications selected from:

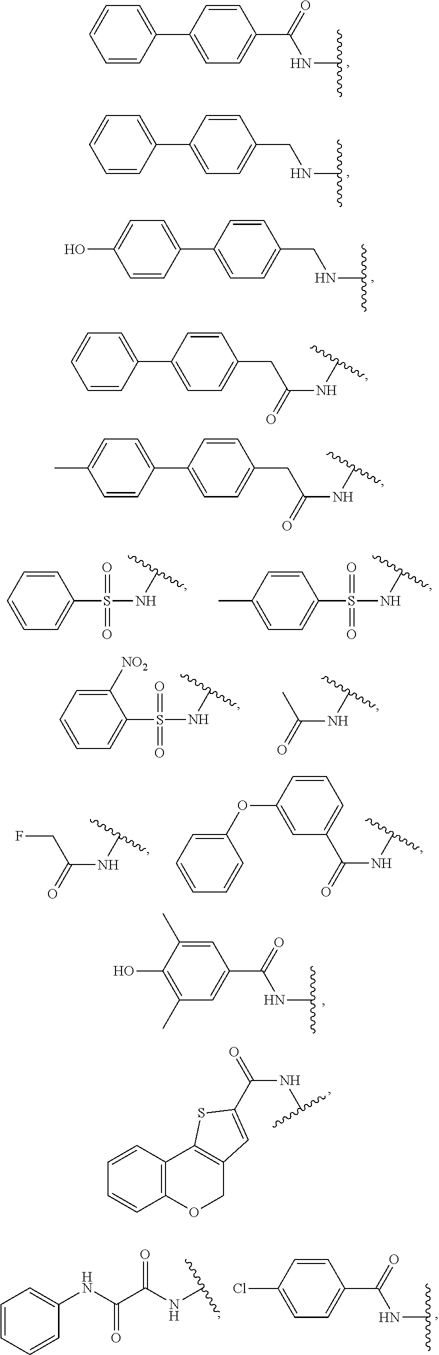

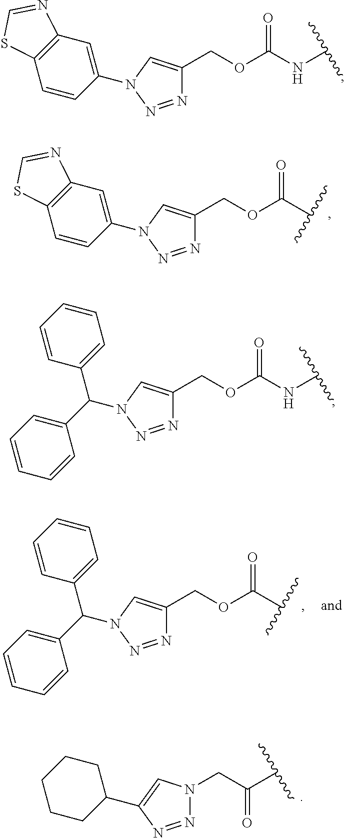

In some embodiments, the glycan moiety comprises a compound disclosed by Büll et al. (Trends in Biochemical Sciences; 41:6, P519-531, 2016), which is incorporated by reference herein in its entirety. In certain embodiments, the glycan moiety comprises one or more sialic acid mimetic chemical modifications or substituents disclosed by Büll, et al.

In some embodiments, the glycan moiety comprises a chain of two or more repeating HexNAc-Hexuronate-units. In some embodiments, the glycan moiety comprises a chain of two or more repeating GlcN-GlcA-units. In some embodiments, the glycan moiety comprises a chain of two or more repeating GlcN-IdoA-units. In some embodiments, the glycan moiety comprises a chain of two or more repeating GalNAc-GlcA-units.

In some embodiments, the glycan moiety comprises a chain of repeating Galactose-GlcNAc-units.

In some embodiments, the glycan moiety comprises a mono-antennary glycan comprising at least 2 monosaccharides. In some embodiments, the glycan moiety comprises a mono-antennary glycan comprising at least 3 monosaccharides. In some embodiments, the glycan moiety comprises a mono-antennary glycan comprising at least 4 monosaccharides. In some embodiments, the glycan moiety comprises a mono-antennary glycan comprising at least 5 monosaccharides. In some embodiments, the glycan moiety comprises a mono-antennary glycan comprising at least 6 monosaccharides. In some embodiments, the glycan moiety comprises a mono-antennary glycan comprising at least 7 monosaccharides. In some embodiments, the glycan moiety comprises a mono-antennary glycan comprising at least 8 monosaccharides. In some embodiments, the glycan moiety comprises a mono-antennary glycan comprising at least 9 monosaccharides. In some embodiments, the glycan moiety comprises a mono-antennary glycan comprising at least 10 monosaccharides. In some embodiments, the glycan moiety comprises a mono-antennary glycan comprising at least 11 monosaccharides. In some embodiments, the glycan moiety comprises a mono-antennary glycan comprising at least 12 monosaccharides.

In some embodiments, the glycan moiety comprises a multi-antennary glycan comprising one or more mannose at the position(s) where the glycan branches. In certain embodiments, the multi-antennary glycan comprises at least three mannose moieties, wherein one mannose is positioned where the glycan branches, and is bonded to two mannose moieties, one in each branch of the multi-antennary glycan. In certain embodiments, the glycan moiety comprises a branching oligosaccharide consisting only of mannose.

In some embodiments, the glycan moiety comprises a multi-antennary glycan comprising one or more mannose at the position(s) where the glycan branches. In certain 92 embodiments, the multi-antennary glycan comprises at least three mannose moieties, wherein one mannose is positioned where the glycan branches, and is bonded to two mannose moieties, one in each branch of the multi-antennary glycan.

In some embodiments, the glycan moiety comprises a bi-antennary glycan, wherein the bi-antennary glycan comprises a first terminal residue and a second terminal residue. In some embodiments, at least one of the first terminal residue or second terminal residue of the bi-antennary glycan comprises sialic acid. In some embodiments, at least one of the first terminal residue or second terminal residue of the bi-antennary glycan comprises mannose. In some embodiments, at least one of the first terminal residue or second terminal residue of the bi-antennary glycan comprises GlcNAc. In some embodiments, at least one of the first terminal residue or second terminal residue of the bi-antennary glycan comprises NANA. In some embodiments, at least one of the first terminal residue or second terminal residue of the bi-antennary glycan comprises GalNAc. In some embodiments, at least one of the first terminal residue or second terminal residue of the bi-antennary glycan comprises a sialic acid residue comprising one or more poly-sialic acid terminal modifications. In some embodiments, at least one of the first terminal residue or second terminal residue of the bi-antennary glycan comprises fucose. In some embodiments, one of the first terminal residue or second terminal residue of the bi-antennary glycan comprises fucose and the other comprises sialic acid. In some embodiments, both the first terminal residue and second terminal residue of the bi-antennary glycan comprises sialic acid. In some embodiments, both the first terminal residue and second terminal residue of the bi-antennary glycan comprises mannose. In some embodiments, both the first terminal residue and second terminal residue of the bi-antennary glycan comprises GlcNAc. In some embodiments, both the first terminal residue and second terminal residue of the bi-antennary glycan comprises NANA. In some embodiments, both the first terminal residue and second terminal residue of the bi-antennary glycan comprises GalNAc.

In some embodiments, the glycan moiety comprises a tri-antennary glycan, wherein the tri-antennary glycan comprises a first terminal residue, a second terminal residue, and a third terminal residue. In some embodiments, at least one of the first terminal residue, the second terminal residue or the third terminal residue of the tri-antennary glycan comprises sialic acid. In some embodiments, at least one of the first terminal residue, the second terminal residue or the third terminal residue of the tri-antennary glycan comprises a sialic acid residue comprising one or more poly-sialic acid terminal modifications. In some embodiments, at least one of the first terminal residue, or the second terminal residue of the tri-antennary glycan comprises fucose. In some embodiments, at least one of the first terminal residue, the second terminal residue or the third terminal residue of the tri-antennary glycan comprises sialic acid, and at least one of the remaining terminal residues comprises fucose. In some embodiments, at least one of the first terminal residue, the second terminal residue and the third terminal residue of the tri-antennary glycan comprises sialic acid. In some embodiments, at least one of the first terminal residue, the second terminal residue and the third terminal residue of the tri-antennary glycan comprises mannose. In some embodiments, at least one of the first terminal residue, the second terminal residue and the third terminal residue of the tri-antennary glycan comprises GlcNAc. In some embodiments, at least one of the first terminal residue, the second terminal residue and the third terminal residue of the tri-antennary glycan comprises NANA. In some embodiments, at least one of the first terminal residue, the second terminal residue and the third terminal residue of the tri-antennary glycan comprises GalNAc. In some embodiments, all of the first terminal residue, the second terminal residue and the third terminal residue of the tri-antennary glycan comprises sialic acid. In some embodiments, all of the first terminal residue, the second terminal residue and the third terminal residue of the tri-antennary glycan comprises mannose. In some embodiments, all of the first terminal residue, the second terminal residue and the third terminal residue of the tri-antennary glycan comprises GlcNAc. In some embodiments, all of the first terminal residue, the second terminal residue and the third terminal residue of the tri-antennary glycan comprises NANA. In some embodiments, all of the first terminal residue, the second terminal residue and the third terminal residue of the tri-antennary glycan comprises GalNAc.

In some embodiments, the glycan moiety comprises a tetra-antennary glycan, wherein the tetra-antennary glycan comprises a first terminal residue, a second terminal residue, a third terminal residue and a fourth terminal residue. In some embodiments, at least one of the first terminal residue, the second terminal residue, the third terminal residue or the fourth terminal residue of the tetra-antennary glycan comprises sialic acid. In some embodiments, at least one of the first terminal residue, the second terminal residue, the third terminal residue or the fourth terminal residue of the tetra-antennary glycan comprises a sialic acid residue comprising one or more poly-sialic acid terminal modifications. In some embodiments, at least one of the first terminal residue, the second terminal residue, the third terminal residue or the fourth terminal residue of the tetra-antennary glycan comprises fucose. In some embodiments, at least one of the first terminal residue, the second terminal residue, the third terminal residue or the fourth terminal residue of the tetra-antennary glycan comprises sialic acid, and at least one of the remaining terminal residues comprises fucose. In some embodiments, at least one of the first terminal residue, the second terminal residue, the third terminal residue and the fourth terminal residue of the tetra-antennary glycan comprises sialic acid. In some embodiments, at least one of the first terminal residue, the second terminal residue, the third terminal residue and the fourth terminal residue of the tetra-antennary glycan comprises mannose. In some embodiments, at least one of the first terminal residue, the second terminal residue, the third terminal residue and the fourth terminal residue of the tetra-antennary glycan comprises GlcNAc. In some embodiments, at least one of the first terminal residue, the second terminal residue, the third terminal residue and the fourth terminal residue of the tetra-antennary glycan comprises NANA. In some embodiments, at least one of the first terminal residue, the second terminal residue, the third terminal residue and the fourth terminal residue of the tetra-antennary glycan comprises GalNAc. In some embodiments, all of the first terminal residue, the second terminal residue, the third terminal residue and the fourth terminal residue of the tetra-antennary glycan comprises sialic acid. In some embodiments, all of the first terminal residue, the second terminal residue, the third terminal residue and the fourth terminal residue of the tetra-antennary glycan comprises mannose. In some embodiments, all of the first terminal residue, the second terminal residue, the third terminal residue and the fourth terminal residue of the tetra-antennary glycan comprises GlcNAc. In some embodiments, all of the first terminal residue, the second terminal residue, the third terminal residue and the fourth terminal residue of the tetra-antennary glycan comprises NANA. In some embodiments, all of the first terminal residue, the second terminal residue, the third terminal residue and the fourth terminal residue of the tetra-antennary glycan comprises GalNAc.

In some embodiments, the glycan moiety comprises a fucose linked to a core or base region of the glycan. In some embodiments, the glycan moiety comprises a fucose linked to a non-terminal region of the glycan. In some embodiments wherein the glycan moiety comprises a bi-antennary glycan, a tri-antennary glycan, or a tetra-antennary glycan, the glycan comprises a fucose linked to a GlcNAc residue in a core or a base region of the glycan. In some embodiments wherein the glycan moiety comprises a bi-antennary glycan, a tri-antennary glycan, or a tetra-antennary glycan, the glycan comprises a fucose linked to a GlcNAc residue in a tree, branch or arm region of the glycan.

In some embodiments, the glycan moiety comprises a bisecting glycan. In some embodiments, the glycan moiety comprises a bi-antennary glycan comprising a GlcNAc moiety bound to the monosaccharide that links the two branches of the bi-antennary glycan, thereby forming a bisecting glycan. In some embodiments, the glycan moiety comprises a tri-antennary glycan, wherein one of the three branches of the tri-antennary glycan is formed by a bisecting linkage between two other branches. In some embodiments, the glycan moiety comprises a tetra-antennary glycan, wherein at least one of the branches of the tetra-antennary glycan is formed by a bisecting linkage between two other branches.

In some embodiments, the glycan moiety comprises a bi-antennary, tri-antennary, or tetra-antennary glycan, having at least two different terminal residue monosaccharides. For example, in some embodiments, the glycan moiety is a bi-antennary glycan wherein the first terminal residue and the second terminal residue do not comprise the same monosaccharide. In some embodiments, the glycan moiety is a tri-antennary glycan wherein a first and second terminal residue comprise the same monosaccharide and a third terminal residue comprises a different monosaccharide. In some embodiments, the glycan moiety is a tri-antennary glycan wherein the first, second, and third terminal residues comprise different monosaccharides. In some embodiments, the glycan moiety is a tetra-antennary glycan wherein a first and second terminal residue comprise the same monosaccharide and the third and fourth terminal residues comprise a different monosaccharide from the first and second terminal residues, wherein the third and fourth terminal residues optionally comprise the same monosaccharide as each other. In some embodiments, the glycan moiety is a tetra-antennary glycan wherein a first, second and third terminal residue comprise the same monosaccharide and the fourth terminal residue comprises a different monosaccharide from the first, second and third terminal residues. In some embodiments, the glycan moiety is a tetra-antennary glycan wherein the first, second, third and fourth terminal residues comprise different monosaccharides.

In some embodiments, the glycan moiety is an N-linked glycan, such that the glycan is conjugated to the modified nucleic acid through a nitrogen atom.

In some embodiments, the glycan moiety comprises a glycan comprising a monosaccharide at the non-reducing terminus, further comprising a conjugation handle covalently bonded to the non-reducing end terminal monosaccharide. In some embodiments, the glycan moiety comprises a glycan comprising a N-acetylglucosamine (GlcNAc) at the non-reducing terminus, further comprising a conjugation handle covalently bonded to the non-reducing end terminal GlcNAc. As used herein, the terms “non-reducing end terminal monosaccharide” and “monosaccharide at the non-reducing terminus” refer to a monosaccharide residue that is a part of a glycan moiety and forms a terminus of said glycan at the non-reducing end. As an illustrative example, in Exemplary Glycan H-7, the “GlcNAc” at the end of the IUPAC name is the non-reducing end terminal GlcNAc:
Neu5Ac(a2-8)Neu5Ac(a2-3)Gal(b1-4)GlcNAc

In some embodiments, the glycan moiety comprises a glycan, further comprising a conjugation handle covalently bonded to the non-reducing end terminal GlcNAc. In some embodiments, the glycan moiety comprises a glycan, further comprising a conjugation handle covalently bonded to the non-reducing end terminal GalNAc. In some embodiments, the glycan moiety comprises a glycan, further comprising a conjugation handle covalently bonded to the non-reducing end terminal mannose. In some embodiments, the glycan moiety comprises a glycan, further comprising a conjugation handle covalently bonded to the non-reducing end terminal GlcA. In some embodiments, the glycan moiety comprises a glycan, further comprising a conjugation handle covalently bonded to the non-reducing end terminal IdoA. In some embodiments, the glycan moiety comprises a glycan, further comprising a conjugation handle covalently bonded to the non-reducing end terminal Glucose. In some embodiments, the glycan moiety comprises a glycan, further comprising a conjugation handle covalently bonded to the non-reducing end terminal GlcN.

In some embodiments, the glyco-ligand comprises a glycan, the glycan comprising an asparagine residue covalently bound to the glycan (e.g., at the non-reducing end terminal monosaccharide of the glycan). In some embodiments, the glyco-ligand comprises a glycan illustrated in any one of glycan of FIG. 1, or Tables 1A-1F, further comprising an asparagine residue covalently bound to the glycan (e.g., at the non-reducing end terminal monosaccharide of the glycan) as shown:

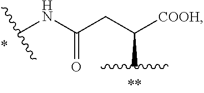 [SMILES: *NC(=O)C[C@H](*)C(=O)O]

wherein * indicates the point of attachment to the glycan (e.g., the non-reducing end terminal monosaccharide of the glycan) and ** indicates the point of attachment to the ligand (e.g., modified RNA), or a linker group attached to the ligand (e.g., modified RNA).

In some embodiments, the glyco-ligand comprises a glycan that is bonded to the ligand (e.g., a nucleic acid) through a click-chemistry reaction between an asparagine residue covalently bound to the glycan (e.g., at the non-reducing end terminal monosaccharide of the glycan) and an alkyne moiety or other suitable moiety attached to the ligand. In some embodiments the glyco-ligand comprises a glycan, and is the product of, and has the structure of the product of, a click-chemistry reaction of a glycan that further comprises asparagine azide covalently bound to the glycan (e.g., at the non-reducing end terminal monosaccharide of the glycan) as shown:

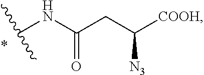 [SMILES: *NC(=O)C[C@H](N=[N+]=[N-])C(=O)O]

wherein * indicates the point of attachment to the glycan (e.g., at the non-reducing end terminal monosaccharide of the glycan), and an alkyne moiety (or other suitable moiety) attached to the ligand (e.g., a nucleic acid). Exemplary glycans of this type are shown in Table 4A.

In some embodiments, the glyco-ligand comprises a glycan, the glycan further comprising an arginine residue covalently bound to the non-reducing end terminal monosaccharide. In some embodiments, the glyco-ligand comprises a glycan, the glycan further comprising an azide click chemistry handle covalently bound to the non-reducing end terminal monosaccharide, either directly or through a linker group. In some embodiments, the linker group bridging the non-reducing end terminal monosaccharide and the azide comprises one or more peptide residues. In some embodiments, the linker group bridging the non-reducing end terminal monosaccharide and the azide comprises one or more polyethylene glycol (PEG) units. In some embodiments, the linker group bridging the non-reducing end terminal monosaccharide and the azide comprises 1-10 PEG units. In some embodiments, the linker group bridging the non-reducing end terminal monosaccharide and the azide comprises one PEG unit. In some embodiments, the linker group bridging the non-reducing end terminal monosaccharide and the azide comprises two PEG units. In some embodiments, the linker group bridging the non-reducing end terminal monosaccharide and the azide comprises three PEG units. In some embodiments, the linker group bridging the non-reducing end terminal monosaccharide and the azide comprises four PEG units. In some embodiments, the linker group bridging the non-reducing end terminal monosaccharide and the azide comprises five PEG units. In some embodiments, the linker group bridging the non-reducing end terminal monosaccharide and the azide comprises an optionally substituted aliphatic chain. In some embodiments, the optionally substituted aliphatic chain is a C₁-C₁₂ alkylene chain. In some embodiments, the optionally substituted aliphatic chain is a C₂-C₁₂ alkenylene chain.

In some embodiments, the glyco-ligand comprises a glycan that is bonded to the ligand (e.g., a nucleic acid) through a click-chemistry reaction between a conjugation handle covalently bonded to the glycan (e.g., to the non-reducing end of a terminal monosaccharide on the glycan) and an alkyne moiety or other suitable moiety attached to the ligand. In some embodiments, the conjugation handle comprises aminooxy-PEG3-azide:

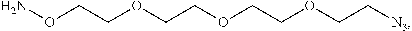 [SMILES: [N-]=[N+]=NCCOCCOCCOCCON]

that is bonded to the glycan through the amino group. In such embodiments, the glyco-ligand as a whole is the product of, and has the structure of the product of, a click-chemistry reaction between aminooxy-PEG3-azide and an alkyne moiety (or other suitable moiety) attached to the ligand (e.g., a nucleic acid) portion of the glyco-ligand.

In some embodiments, the glyco-ligand comprises a glycan, and is the product of, and has the structure of the product of, a click-chemistry reaction of a glycan that comprises aminooxy-PEG3-azide covalently bound to the glycan (e.g., at the non-reducing end terminal monosaccharide of the glycan) as shown:

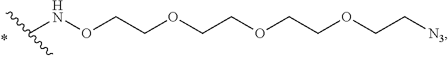 [SMILES: *NOCCOCCOCCOCCN=[N+]=[N-]]

wherein * indicates the point of attachment to the glycan (e.g., at the non-reducing end terminal monosaccharide of the glycan), and an alkyne moiety (or other suitable moiety) attached to the ligand (e.g., a nucleic acid). Exemplary glycans of this type are shown in Table 4B. In such embodiments, the glyco-ligand comprises a linker covalently bound to the glycan (e.g., at the non-reducing end terminal monosaccharide of the glycan) as shown:

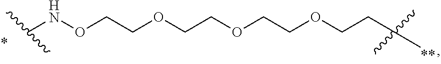 [SMILES: *CCOCCOCCOCCON*]

wherein * indicates the point of attachment to the glycan (e.g., at the non-reducing end terminal monosaccharide of the glycan) and ** indicates the point of attachment to the ligand component of the glyco-ligand (e.g., an RNA, or a modified RNA, or a linker group attached to a modified RNA).

In some embodiments, the conjugation handle comprises O-(3-azidopropyl)-N-methylhydroxylamine:

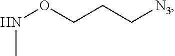

that is bonded to the glycan through the amino group. In such embodiments, the glyco-ligand as a whole is the product of, and has the structure of the product of, a click-chemistry reaction between O-(3-azidopropyl)-N-methylhydroxylamine and an alkyne moiety (or other suitable moiety) attached to the ligand (e.g., a nucleic acid) portion of the glyco-ligand.

In some embodiments, the glyco-ligand comprises a glycan, and is the product of, and has the structure of the product of, a click-chemistry reaction of a glycan that comprises O-(3-azidopropyl)-N-methylhydroxylamine covalently bound to glycan (e.g., at the non-reducing end terminal monosaccharide of the glycan) as shown:

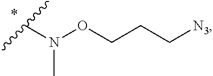 [SMILES: *N(C)OCCCN=[N+]=[N-]]

wherein * indicates the point of attachment to the glycan (e.g., at the non-reducing end terminal monosaccharide of the glycan), and an alkyne moiety (or other suitable moiety) attached to the ligand (e.g., a nucleic acid). In such embodiments, the glyco-ligand comprises a linker covalently bound to the glycan (e.g., at the non-reducing end terminal monosaccharide of the glycan) as shown:

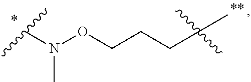 [SMILES: *CCCON(*)C]

wherein * indicates the point of attachment to the glycan (e.g., at the non-reducing end terminal monosaccharide of the glycan) and ** indicates the point of attachment to the ligand component of the glyco-ligand (e.g., an RNA, or a modified RNA, or a linker group attached to a modified RNA).

In some embodiments, the conjugation handle comprises O-(2-azidoethyl)-N-methylhydroxylamine:

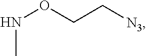

that is through bonded to the glycan the amino group. In such embodiments, the glyco-ligand as a whole is the product of, and has the structure of the product of, a click-chemistry reaction between O-(2-azidoethyl)-N-methylhydroxylamine and an alkyne moiety (or other suitable moiety) attached to the ligand (e.g., a nucleic acid) portion of the glyco-ligand.

In some embodiments, the glyco-ligand comprises a glycan, and is the product of, and has the structure of the product of, a click-chemistry reaction of a glycan that comprises O-(2-azidoethyl)-N-methylhydroxylamine covalently bound to the glycan (e.g., at the non-reducing end terminal monosaccharide of the glycan) as shown:

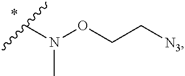 [SMILES: *N(C)OCCN=[N+]=[N-]]

wherein * indicates the point of attachment to the glycan (e.g., at the non-reducing end terminal monosaccharide of the glycan), and an alkyne moiety (or other suitable moiety) attached to the ligand (e.g., a nucleic acid). Exemplary glycans of this type are shown in Table 4C. In such embodiments, the glyco-ligand comprises a linker covalently bound to the glycan (e.g., at the non-reducing end terminal monosaccharide of the glycan) as shown:

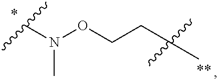 [SMILES: *CCON(*)C]

wherein * indicates the point of attachment to the glycan (e.g., at the non-reducing end terminal monosaccharide of the glycan) and ** indicates the point of attachment to the ligand component of the glyco-ligand (e.g., an RNA, or a modified RNA, or a linker group attached to a modified RNA).

In some embodiments, the conjugation handle comprises 2-azidoethanol:

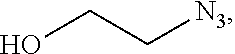

that is bonded to the glycan through the hydroxyl group (e.g., through an ether or ester that includes the hydroxyl oxygen). In such embodiments, the glyco-ligand as a whole is the product of, and has the structure of the product of, a click-chemistry reaction between 2-azidoethanol and an alkyne moiety (or other suitable moiety) attached to the ligand (e.g., a nucleic acid) portion of the glyco-ligand.

In some embodiments, the glyco-ligand comprises a glycan, and is the product of, and has the structure of the product of, a click-chemistry reaction of a glycan that comprises 2-azidoethanol covalently bound to the glycan (e.g., at the non-reducing end terminal monosaccharide of the glycan) as shown:

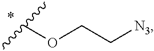 [SMILES: *OCCN=[N+]=[N-]]

wherein * indicates the point of attachment to the glycan (e.g., at the non-reducing end terminal monosaccharide of the glycan), and an alkyne moiety (or other suitable moiety) attached to the ligand (e.g., a nucleic acid). Exemplary glycans of this type are shown in Table 4D. In such embodiments, the glyco-ligand comprises a linker covalently bound to the glycan (e.g., at the non-reducing end terminal monosaccharide of the glycan) as shown:

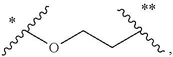 [SMILES: *CCO*]

wherein * indicates the point of attachment to the glycan (e.g., at the non-reducing end terminal monosaccharide of the glycan) and ** indicates the point of attachment to the ligand component of the glyco-ligand (e.g., an RNA, or a modified RNA, or a linker group attached to a modified RNA).

In some embodiments, the conjugation handle comprises O-(2-azidoethoxy)-PEG3-N-methylhydroxylamine:

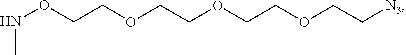 [SMILES: CNOCCOCCOCCOCCN=[N+]=[N-]]

that is bonded to the glycan through the amino group. In such embodiments, the glyco-ligand as a whole is the product of, and has the structure of the product of, a click-chemistry reaction between O-(2-azidoethoxy)-PEG3-N-methylhydroxylamine and an alkyne moiety (or other suitable moiety) attached to the ligand (e.g., a nucleic acid) portion of the glyco-ligand.

In some embodiments, the glyco-ligand comprises a glycan, and is the product of, and has the structure of the product of, a click-chemistry reaction of a glycan that comprises O-(2-azidoethoxy)-PEG3-N-methylhydroxylamine covalently bound to the glycan (e.g., at the non-reducing end terminal monosaccharide of the glycan) as shown:

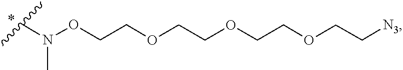 [SMILES: *N(C)OCCOCCOCCOCCN=[N+]=[N-]]

wherein * indicates the point of attachment to the glycan (e.g., at the non-reducing end terminal monosaccharide of the glycan), and an alkyne moiety (or other suitable moiety) attached to the ligand (e.g., a nucleic acid). Exemplary glycans of this type are shown in Table 4E. In such embodiments, the glyco-ligand comprises a linker covalently bound to the glycan (e.g., at the non-reducing end terminal monosaccharide of the glycan) as shown:

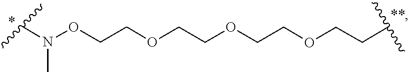 [SMILES: *CCOCCOCCOCCON(*)C]

wherein * indicates the point of attachment to the glycan (e.g., at the non-reducing end terminal monosaccharide of the glycan) and ** indicates the point of attachment to the ligand component of the glyco-ligand (e.g., an RNA, or a modified RNA, or a linker group attached to a modified RNA).

In some embodiments, the conjugation handle comprises p-azidophenol:

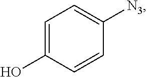

that is bonded to the glycan through the phenol group (e.g., through an ether or ester that includes the phenol oxygen). In such embodiments, the glyco-ligand as a whole is the product of, and has the structure of the product of, a click-chemistry reaction between p-azidophenol and an alkyne moiety (or other suitable moiety) attached to the ligand (e.g., a nucleic acid) portion of the glyco-ligand.

In some embodiments, the glyco-ligand comprises a glycan, and is the product of, and has the structure of the product of, a click-chemistry reaction of a glycan that comprises p-azidophenol covalently bound to the glycan (e.g., at the non-reducing end terminal monosaccharide of the glycan) as shown:

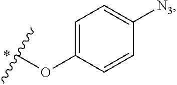 [SMILES: *Oc1ccc(N=[N+]=[N-])cc1]

wherein * indicates the point of attachment to the glycan (e.g., at the non-reducing end terminal monosaccharide of the glycan), and an alkyne moiety (or other suitable moiety) attached to the ligand (e.g., a nucleic acid). Exemplary glycans of this type are shown in Table 4F. In such embodiments, the glyco-ligand comprises a linker covalently bound to the glycan (e.g., at the non-reducing end terminal monosaccharide of the glycan) as shown:

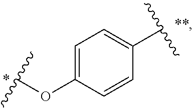 [SMILES: *Oc1ccc(*)cc1]

wherein * indicates the point of attachment to the glycan (e.g., at the non-reducing end terminal monosaccharide of the glycan) and ** indicates the point of attachment to the ligand component of the glyco-ligand (e.g., an RNA, or a modified RNA, or a linker group attached to a modified RNA).

The conjugation handle of the glyco-ligand can be a carboxylic acid or any carboxylic acid derivative including, but not limited to, an ester, an activated ester (e.g., pentafluorophenol ester, succinimidyl ester, p-nitrophenol ester), an anhydride, a carboxylic acid, thioester, and the like. Carboxylic acids and carboxylic acid derivatives can be conjugated by chemical reaction to form a covalent bond between the carboxylic acid (or carboxylic acid derivative) and another molecule (e.g., the ligand, a nucleic acid). For example, carboxylic acids can be conjugated to alcohols or amines to form esters or amides, respectively.

In some embodiments, the conjugation handle comprises 4-((methylamino)oxy)butanoic acid:

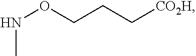

that is bonded to the glycan through the amino group. In such embodiments, the glyco-ligand as a whole is the product of, and has the structure of the product of, a conjugation reaction (e.g., amidation, Staudinger ligation, esterification) between 4-((methylamino)oxy)butanoic acid and a suitable chemical moiety (e.g., alcohol, amine) attached to the ligand (e.g., a nucleic acid) portion of the glyco-ligand.

In some embodiments, the glyco-ligand comprises a glycan, and is the product of, and has the structure of the product of, a conjugation reaction (e.g., esterification, amidation) of a glycan that comprises 4-((methylamino)oxy)butanoic acid covalently bound to the glycan (e.g., at the non-reducing end terminal monosaccharide of the glycan) as shown:

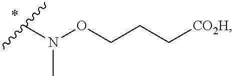 [SMILES: *N(C)OCCCC(=O)O]

wherein* indicates the point of attachment to the glycan (e.g., at the non-reducing end terminal monosaccharide of the glycan), and a suitable chemical moiety (e.g., alcohol, amine) attached to the ligand (e.g., a nucleic acid). Exemplary glycans of this type are shown in Table 4G.

In such embodiments, the glyco-ligand comprises a linker covalently bound to the glycan (e.g., at the non-reducing end terminal monosaccharide of the glycan) as shown:

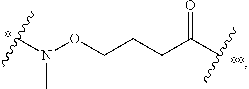 [SMILES: *C(=O)CCCON(*)C]

wherein * indicates the point of attachment to the glycan (e.g., at the non-reducing end terminal monosaccharide of the glycan) and ** indicates the point of attachment to the ligand component of the glyco-ligand (e.g., an RNA, or a modified RNA, or a linker group attached to a modified RNA).

### Glycan-RNA Conjugation and Glyco-Ligand Compositions

Additional preferred embodiments include methods for site-specific modification of a synthetic scaffold domain amenable for glycan conjugation. In certain embodiments, the modification comprises a target region of nucleic acids. The method comprises contacting one or more target nucleic acid molecule using azide/alkyne click chemistry to conjugate one or more glycans onto one or more donor nucleic acid sequences (Meng, G., Guo, T., Ma, T. et al. Modular click chemistry libraries for functional screens using a diazotizing reagent. Nature 574, 86-89 (2019)). In various embodiments, the donor nucleic acid sequence comprises: one or more modified sequence modified at a specific site for operably conjugating one or more glycans (Meng, G., Guo, T., Ma, T. et al., (2019)).

As described in Gao et al., using an integrated chemoenzymatic approach to efficiently generate a library of complex multiantennary Asn-linked N-glycan isomers in sub-milligram quantities, a sialylated glycopeptide (SGP) (Seko A., Koketsu M., Nishizono M., Enoki Y., Ibrahim H. R., Juneja L. R., Kim M., Yamamoto T. Occurrence of a sialylglycopeptide and free sialylglycans in hen's egg yolk. Biochim. Biophys. Acta. 1997; 1335:23-32) can be purified from chicken egg yolk powder in large quantities, and a set of recombinant human glycosyltransferases to yield a library of 32 N-glycosylasparagine isomers, all of which occur naturally in human and other mammals (see the database of UniCarbKB [Campbell et al., 2014], and CFG for information). Gao et al., also identified a method to convert the Asn-linked glycans to free reducing oligosaccharides using sodium hypochlorite (NaClO; bleach). These compounds can be conveniently converted to free reducing glycans.

Accordingly, using a highly efficient chemoenzymatic approach, a library of naturally occurring isomeric asparagine-linked glycans is generated and the resulting free reducing glycans are conjugated onto a synthetic scaffold domain.

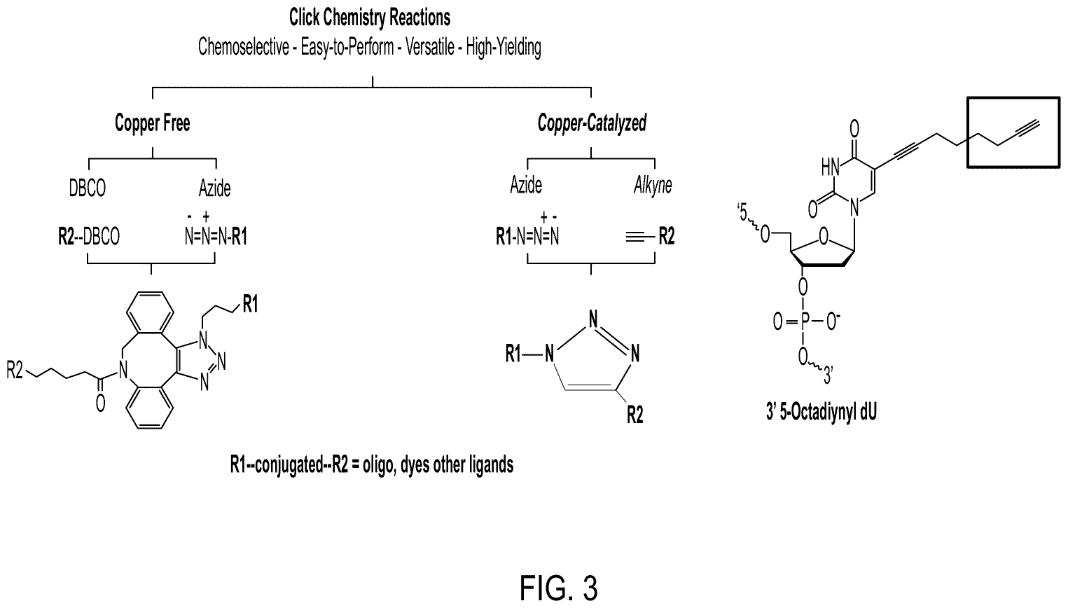

Chang et al., employed azido-sugars that were incorporated into glycans where such azido-sugars were then be labeled by various alkyne-containing probes. (Chang P V, Prescher J A, Sletten E M, Baskin J M, Miller I A, Agard N J, Lo A, Bertozzi C R. Copper-free click chemistry in living animals. Proc Natl Acad Sci USA. 2010 Feb. 2; 107(5):1821-6.). Tornøe et al., converted a terminal amine to an azide so that the glycan could be used in click chemistry. (Tornøe, C. W., Christensen, C. & Meldal, M. Peptidotriazoles on solid phase: [1,2,3]-triazoles by regiospecific copper(I)-catalyzed 1,3-dipolar cycloadditions of terminal alkynes to azides. J. Org. Chem. 67, 3057-3064 (2002)). By employing click chemistry reactions using an alkyne on the nucleic acid and an azide on the glycan, a covalent conjugation of glycans to nucleic acids can be accomplished. See FIG. 3.

In some embodiments, the glyco-ligand comprises a glycan conjugated to a nucleic acid ligand via linker formed from a click chemistry reaction. In some embodiments, the click chemistry reaction is selected from copper-catalyzed azide-alkyne cyclization (CuAAC), strain-promoted azide-alkyne cycloaddition (SPAAC), transcyclooctyne (TCO)-tetrazine ligation, transcyclooctene-tetrazine ligation, alkene-tetrazine ligation, cross-linking between a primary amine and a N-hydroxysuccinimide ester (NHS ester), a transcyclooctyne-azide coupling, or a cyclopropane-azide coupling, azide-Staudinger ligation.

In some embodiments, the glycan and conjugated nucleic acid are linked through a chemical reaction between two click chemistry handles.

A click chemistry handle or click-chemistry handle can be a reactant, or a reactive group, that can partake in a click chemistry reaction. For example, a strained alkyne, e.g., a cyclooctyne, is a click chemistry handle, since it can partake in a strain-promoted cycloaddition. In general, click chemistry reactions require at least two molecules comprising click chemistry handles that can react with each other. Such click chemistry handle pairs that are reactive with each other are sometimes referred to herein as partner click chemistry handles. For example, an azide is a partner click chemistry handle to a cyclooctyne or any other alkyne. Exemplary click chemistry handles (click-chemistry handle 1 and click-chemistry handle 2) suitable for use according to some aspects of this invention are described herein, for example, in Tables 2A and 2B. Other suitable click chemistry handles are known to those of skill in the art. For two molecules to be conjugated via click chemistry, the click chemistry handles of the molecules are reactive with each other, for example, in that the reactive moiety of one of the click chemistry handles can react with the reactive moiety of the second click chemistry handle to form a covalent bond. Such reactive pairs of click chemistry handles are well known to those of skill in the art and include, but are not limited to, those described in Table 2A:

TABLE 2A
Exemplary Click Chemistry Handles and Reactions

| Scheme | Reaction name |
|---|---|
| 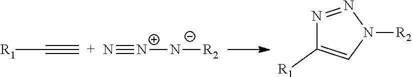 | 1,3-dipolar cycloaddition |
| terminal azide Alkyne | |

| | |
|---|---|
| 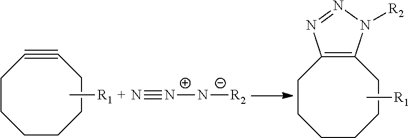 | Strain-promoted cycloaddition |
| strained azide Alkyne | |
| 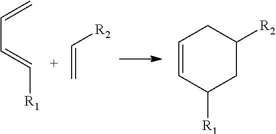 | Diels-Alder reaction |
| diene dienophile | |
| 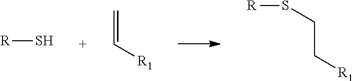 | Thiol-ene reaction |
| thiol alkene | |

In some embodiments, click chemistry handles are used that can react to form covalent bonds in the absence of a metal catalyst. Such click chemistry handles are well known to those of skill in the art and include the click chemistry handles described in Becer, Hoogenboom, and Schubert, Click Chemistry beyond Metal-Catalyzed Cycloaddition, Angewandte Chemie International Edition (2009) 48:4900-4908. See Table 2B below.

TABLE 2B
Exemplary Click Chemistry Handles and Reactions

| | Reagent A | Reagent B | Mechanism | Notes on reaction |
|---|---|---|---|---|
| 0 | Azide | Alkyne | Cu-catalyzed [3 + 2] azide- | 2 hours at 60° C. in H₂O |
| | | | alkyne cycloaddition | |
| | | | (CuAAC) | |
| 1 | Azide | Cyclooctyne | Strain-promoted [3 + 2] azide- | 1 hour at room |
| | | | alkyne cycloaddition | temperature |
| | | | (SPAAC) | |
| 2 | Azide | Activated | [3 + 2] Huisgen cycloaddition | 4 hours at 50° C. |
| | | alkyne | | |
| 3 | Azide | Electron- | [3 + 2] cycloaddition | 12 hours at room |
| | | deficient | | temperature in H₂O |
| | | alkyne | | |
| 4 | Azide | Aryne | [3 + 2] cycloaddition | 4 hours at room |
| | | | | temperature in THF |
| | | | | with crown ether or 24 |
| | | | | hours at room |
| | | | | temperature in CH₃CN |
| 5 | Tetrazine | Alkene | Diels-Alder retro-[4 + 2] | 40 minutes at 25° C. |
| | | | cycloaddition | (100% yield); N₂ is the |
| | | | | only by-product |
| 6 | Tetrazole | Alkene | 1,3-dipolar cycloaddition | Few minutes UV |
| | | | (photoclick) | irradiation and then |
| | | | | overnight at 4° C. |
| 7 | Dithioester | Diene | Hetero-Diels-Alder | 10 minutes at room |
| | | | cycloaddition | temperature |
| 8 | Anthracene | Maleimide | [4 + 2] Diels-Alder reaction | 2 days at reflux in |
| | | | | toluene |
| 9 | Thiol | Alkene | Radical addition (thio click) | 30 minutes UV |
| | | | | (Quantitative conv.) or |
| | | | | 24 hours UV |
| | | | | irradiation (>96%) |
| 10 | Thiol | Enone | Michael addition | 24 hours at room |
| | | | | temperature in CH₃CN |
| 11 | Thiol | Maleimide | Michael addition | 1 hour at 40° C. in THF |
| | | | | or 16 hours at room |
| | | | | temperature in dioxane |
| 12 | Thiol | Para-fluoro | Nucleophilic substitution | Overnight at room |
| | | | | temperature in DMF or |
| | | | | 60 minutes at 40° C. in |
| | | | | DMF |
| 13 | Amine | Para-fluoro | Nucleophilic substitution | 20 minutes MW at 95° |
| | | | | C. in NMP as solvent |

RT = room temperature,
DMF = N,N-dimethylformamide,
NMP = N-methylpyrolidone,
THF = tetrahydrofuran,
CH₃CN = acetonitrile

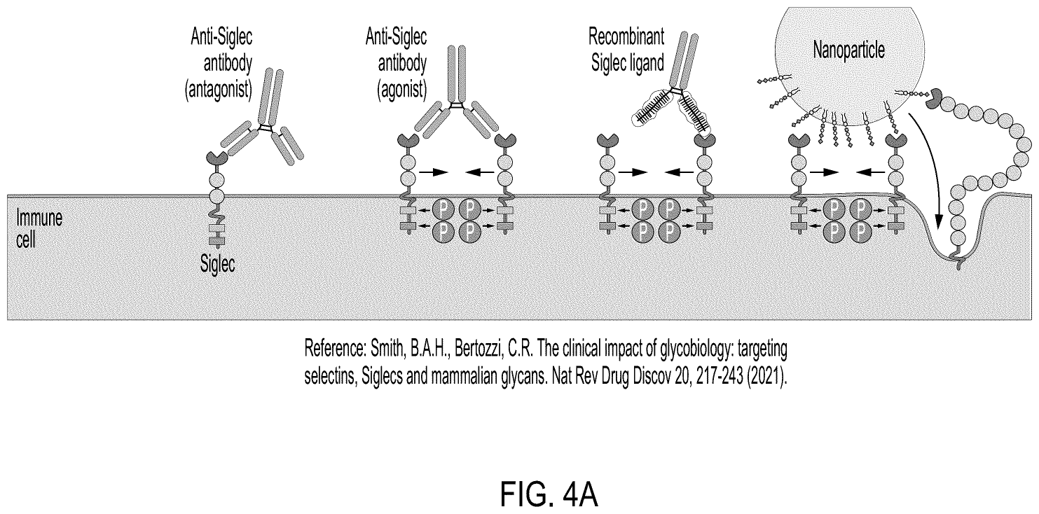

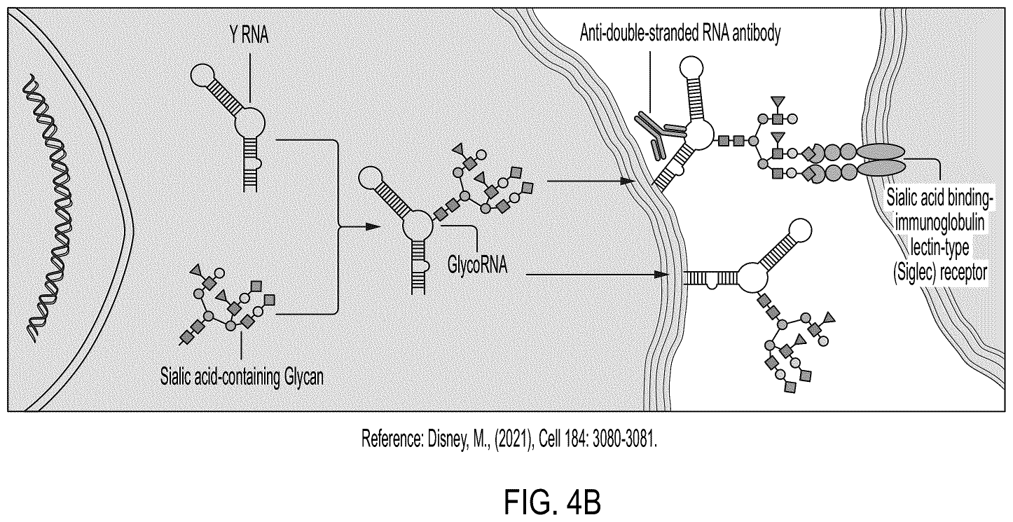

Methods to produce glyco-ligands can include modified nucleosides in transcription reactions or ligation to long RNAs; and through chemical synthesis. The glyco-ligands are preferably <120 nts and can be configured as a defined structure and can be in one or more desired orientations for glycans to engage receptors. See FIG. 4A.

Another example of glycan conjugation is found in Sampaolesi et al., (2019), Future Med. Chem. 11(1): 43-60. Sampaolesi et a. show three reaction schemes for collagen neoglycosylation. The first is an insertion of a thiol and subsequent thiol-ene reaction with (a) α-allyl-glucoside and (b) β-allyl-galactoside). The second shows collagen glycosylated by reductive amination with maltose, Neu5acα2-6-Galβ1-4Glc- and Neu5acα2-3-Galβ1-4Glc to expose (a) glucose, (b) Neu5acα2-6-Gal and (c) Neu5acα2-3-Gal. The third is (a) one-step aminolysis with glucosamine; (b) two-step aminolysis with diamino linkers, followed by reductive amination. [Sampaolesi et al., (2019), Future Med. Chem. 11(1): 43-60.]

Preferably, the site of glycosylation includes one or more sites on a nucleotide base where the glycans are displayed to, for instance, a circRNA, 5′ or 3′ end of a linear RNA such as siRNA, ASO or mRNA, one or more hairpin loops, multiloop, internal loop, external loop, stem, bulge, pseudoknot in tRNA, 5′ or 3′ end of a guideRNA, crRNA or tracrRNA in a RNP complex, glycans displayed on an aptamer. See FIG. 2A.

In alternative embodiments, the method comprises covalent conjugation of one or more glycan moieties, N-acetylgalactosamine (GalNAc), to a synthetic scaffold domain, e.g., RNA.

Double-stranded RNA (dsRNA) is a signal for gene-specific silencing of expression in a number of organisms. Phillip A. Sharp, Genes & Dev. 1999. 13:139-141 Cold Spring Harbor Laboratory Press. GalNAc conjugates have become a breakthrough approach in the therapeutic oligonucleotide field with enormous potential. See Sehgal, A. et al. Nat. Med. 21, 492-497 (2015). The ligands derived from GalNAc are compatible with solid-phase oligonucleotide synthesis and deprotection conditions, with synthesis yields comparable to those of standard oligonucleotides. See Nair, J. K. et al. J. Am. Chem. Soc. 136, 16958-16961 (2014). A complete GalNAc-siRNA can be synthesized on a solid-state oligonucleotide synthesizer and chemically defined by mass spectrometry. Additionally, conjugation methods on the 5′ end of ASOs have been reported in the literature. Østergaard, M. E. et al. Bioconj. Chem. 26, 1451-1455 (2015). Similar to siRNAs, conjugation of ASOs to GalNAc ligands has been shown to improve potency of ASOs in hepatocytes. (2) Prakash, T. P. et al. Nucleic Acids Res. 42, 8796-8807 (2014).

Accordingly, in preferred embodiments, to conjugate glycans to siRNAs, conjugation of the glycans to the passenger strand is generally preferred so as not to hinder the on-target silencing activity of the guide strand and, conversely, to diminish the off-target gene silencing potential of the passenger strand. GalNAc can be placed either at the 3′ or 5′ ends of the siRNA sense strand. Janas, M. M. et al. Nat. Commun. 9, 723 (2018); Nair, J. K. et al. J. Am. Chem. Soc. 136, 16958-16961 (2014). To enhance pharmacokinetics properties, siRNAs are made up of patterns of alternating of 2′-O-methyl and 2′-O-fluoro nucleotides with insertion of phosphorothioate bonds (PS) at the extremities of the strands. The modification of the 5′ end of the antisense strand of siRNA using a stable phosphate analog, vinyl phosphonate, brought even more stability and potency for siRNA GalNAc conjugates. This protects the end of the siRNA from degradation and impeding the cell to phosphorylate the double strand prior its insertion into the RISC (RNA induced silencing complex). The latter effect can increase the potency of the siRNA up to 10-fold. [Elkayam E, Parmar R, Brown C R, Willoughby J L, Theile C S, Manoharan M, Joshua-Tor L. siRNA carrying an (E)-vinylphosphonate moiety at the 5′ end of the guide strand augments gene silencing by enhanced binding to human Argonaute-2. Nucleic Acids Res. 2017 Apr. 7; 45(6):3528-3536]. In preferred embodiments, the 5′ end of RNA is modified. [Elkayam E, Parmar R, Brown C R, Willoughby J L, Theile C S, Manoharan M, Joshua-Tor L. siRNA carrying an (E)-vinylphosphonate moiety at the 5′ end of the guide strand augments gene silencing by enhanced binding to human Argonaute-2. Nucleic Acids Res. 2017 Apr. 7; 45(6):3528-3536].

In other embodiments, to conjugate glycan moieties to antisense oligonucleotides (ASO), the glycans are conjugated on both the 3′ and 5′-end of the oligonucleotide. Østergaard, M. E. et al. Bioconj. Chem. 26, 1451-1455 (2015). Conjugation of a GalNAc ligand to the 5′ end of an ASO increases the potency by 10-fold for hepatocyte targets in rodents. Prakash, T. P. et al. Nucleic Acids Res. 42, 8796-8807 (2014).

In alternative embodiments, the present invention provides enriched glyco-ligand comprising enriched GalNAc residues on the scaffold domain (e.g., synthetic scaffold domain).

The biosynthesis of all eukaryotic N-glycans begins on the cytoplasmic face of the ER membrane with the transfer of GlcNAc-P from UDP-GlcNAc to the lipid-like precursor dolichol phosphate (Dol-P) to generate dolichol pyrophosphate N-acetylglucosamine (Dol-P-P-GlcNAc). Fourteen sugars (Glc₃Man₉GlcNAc₂) are sequentially added to Dol-P before en bloc transfer of the entire glycan to an Asn-X-Ser/Thr sequence in a protein that is being synthesized and translocated through the ER membrane. The protein-bound N-glycan is subsequently remodeled in the ER and Golgi by a complex series of reactions catalyzed by membrane-bound glycosidases and glycosyltransferases. Many of these enzymes are exquisitely sensitive to the physiological and biochemical state of the cell in which the glycoprotein is expressed. Thus, the populations of sugars attached to each glycosylated asparagine in a mature glycoprotein will depend on the cell type in which the glycoprotein is expressed and on the physiological status of the cell, a status that may be regulated during development and differentiation and altered in disease.

All oligosaccharyl transferases (OST) subunits are trans-membrane proteins with between one and eight transmembrane domains [See Chapter 8, Varki A, Cummings R D, Esko J D, et al., editors. Essentials of Glycobiology. 2nd edition. Cold Spring Harbor (NY): Cold Spring Harbor Laboratory Press; 2009]. Three OST complexes have been identified in mammals. All contain ribophorins I and II, OST48, and DAD1 (defender against apoptotic cell death), which encode proteins related to Ost1p, Swp1p, Wbp1p, and Ost2p, respectively. In addition, mammalian OST contains other associated proteins and one of two Stt3p proteins (A or B), two distinct Stt3p isoforms that are differentially expressed in different cell types. Mammalian OST-I, OST-II, and OST-III differ in their kinetic properties and in their abilities to transfer Dol-P-P-glycans that have fewer than 14 sugars. Such immature N-glycan species are generated in Alg yeast mutants and in patients with congenital disorders of glycosylation (FIG. 8.3; see Chapter 42 of Varki A, Cummings R D, Esko J D, et al., editors. Essentials of Glycobiology. 2nd edition. Cold Spring Harbor (NY): Cold Spring Harbor Laboratory Press; 2009].

Accordingly, in some aspects, conjugation of glycans onto ligands can be achieved chemoenzymatically or recombinantly using enzymes such as oligosaccharyl transferases (OSTs) where one or more glycans are transferred from dolichol-linked donor substrate (lipid-linked) to N-linked residues on a ligand.

In other embodiments, the glycans are synthesized prior to en bloc OST transfer of the entire glycan of interest. In other embodiments, additional modifications are made post en bloc OST transfer of the glycan assembly.

Circulating RNA, such as messenger RNA, long noncoding RNA, and small noncoding RNA, including microRNA and Y RNA, is contained in exosomes and microvesicles. Nachtergaele, S. and Krishnan, Y. New Vistas for Cell-Surface GlycoRNAs. N. Engl. J. Med. 385; 7 (2021). In some embodiments, glycans are attached to RNA for packaging into exosomes and microvesicles.

In other preferred embodiments, the method provides an efficient site-specific attachment of one or more glycans to a nucleic acid or a nucleobase. Preferably, the efficiency is greater than 20%, at least 30%, at least 40%, at least 50%, at least 60%, at least 70%, at least 80%, at least 90%, at least 95%, at least 97%, at least 98%, at least 99%, at least 99.6%, at least 99.8%, at least 99.9%, or greater.

More preferably, the conjugation efficiency is greater than 70%, at least 70.5%, at least 71%, at least 72%, at least 73%, at least 74%, at least 75%, at least 76%, at least 77%, at least 78%, at least 79%, at least 80%, at least 85%, at least 90%, at least 95%, or at least 99%.

In some embodiments, a glyco-ligand of the present disclosure has a molar ratio of a nucleic acid unit to the glycan moiety selected from 1:1, 1:2, 1:3, 1:4, 1:4, 1:5, 1:6, 1:7, 1:8, 1:9, 1:10, or 1:10 to 1:100.

In various aspects, the pharmaceutical composition comprising the glyconucleic acid nanostructure is characterized by glycan site occupancy, multivalency, or heteromultivalency on a nucleobase greater than 10%, greater than 20%, greater than 30%, greater than 40%, greater than 50%, greater than 60%, greater than 70%, greater than 80%, greater than 90% or higher. Preferably, described herein are glyco-ligands comprising: one or more glycans; and a nucleic acid operably linked via covalent bond (directly or through a linker) to the one or more glycans. More preferably, glyco-ligands comprising more than one glycan are characterized as having a glycan site occupancy on the nucleic acid greater than 10%, greater than 20%, greater than 30%, greater than 40%, greater than 50%, greater than 60%, greater than 70%, greater than 80%, greater than 90% or higher.

Additional embodiments include high-throughput glycan conjugation of the target region of a RNA molecule, which can be accomplished via 96-well plate, tube or flask in liquid culture. More preferred embodiments include plating and/or culturing in liquid and subculturing in liquid in serial passaging.

Provided also are methods and compositions for desired modifications to a preferred RNA sequence. Preferably, the one or more modified sequence is selected from but is not limited to one or more sequences associated with the following phenotypes: receptor binding, cell penetrating, low proteolytic degradation, high conformational stability, high and/or low pH sensitivity, high and/or low temperature tolerance, UV resistance, low or no immunogenicity, improved or increased sequence stability, pk/pD and glycan conjugation efficiency.

Provided herein are also methods and compositions for multiplexing. In certain embodiments, for modification of one or more target region of a synthetic scaffold domain, the method comprises contacting one or more glycan to one or more target RNA molecule with one or more reactive functional groups to a nucleic acid sequence.

In certain aspects, the glyco-ligand composition comprises one or more of the same or different glycans on the synthetic scaffold domain.

Also described herein are methods and compositions for producing homogenous glyco-ligand wherein the synthetic scaffold domain comprises one predominant glycan. In some aspects, the scaffold comprises at least, 50%, at least 60%, at least 70%, at least 70.5%, at least 71%, at least 72%, at least 73%, at least 74%, at least 75%, at least 76%, at least 77%, at least 78%, at least 79%, at least 80%, at least 85%, at least 90%, at least 95%, or at least 99% one predominant glycan. In other embodiments, the synthetic scaffold domain comprises one or more predominant glycans.

In alternative embodiments, the scaffold (e.g., synthetic scaffold domain) comprises a heterogenous mixture of glycans. Such mixtures may include incompletely processed or under-processed glycans, fully processed glycans, complex glycans, hybrid glycans, N-glycans or O-glycans.

As noted in Colgrave et al., (Site occupancy and glycan compositional analysis of two soluble recombinant forms of the attachment glycoprotein of Hendra virus, Glycobiology, Volume 22, Issue 4, April 2012, Pages 572-584) glycosylated proteins often exhibit both macroheterogeneity (variable occupancy of glycosylation sites) and microheterogeneity (variable degree of type, trimming and elongation of the glycan attached to one glycosylation site), adding to their complexity.

Accordingly, described herein are methods and compositions for increasing site occupancy and/or homogeneity of the glyco-ligand. In some aspects, the glycan occupancy is greater than 20%, at least 30%, at least 40%, at least 50%, at least 60%, at least 70%, at least 75%, at least 76%, at least 77%, at least 78%, at least 79%, at least 80%, at least 85%, at least 90%, at least 95%, or at least 99%.

The glyco-ligand is preferably produced chemoenzymatically by conjugating one or more glycans to an RNA base.

In embodiments, site occupancy may render glycans to be sterically hindered, partially masked or hidden in the RNA conformation.

In contrast, the exposed glycan may be prominently displayed based on the RNA conformation enabling extensive glycan display matrix and diversity for biological function, e.g., cell-cell interaction and/or cell-cell communication.

The invention contemplates site specific glycosylation in a number of sites and in preferred site occupancy based on the desired targets.

Preferably, the glyco-ligand composition comprises a single predominant glycoform. In certain embodiments, the predominant glycoform comprise at least 20%, at least 30%, at least 40%, at least 50%, at least 60%, at least 70%, at least 80%, at least 90%, 9 at least 5% or greater amount of N-linked glycans of the total glycoforms. In preferred embodiments, the predominant glycoform comprise at least 20%, at least 30%, at least 40%, at least 50%, at least 60%, at least 70%, at least 80%, at least 90%, at least 95% or greater amount of paucimannose glycans of the total glycoforms. In yet other embodiments, the predominant glycoform comprise at least 20%, at least 30%, at least 40%, at least 50%, at least 60%, at least 70%, at least 80%, at least 90%, at least 95% or greater amount of complex and/or hybrid glycans of the total glycoforms.

In other embodiments, the predominant glycoform comprise at least 20%, at least 30%, at least 40%, at least 50%, at least 60%, at least 70%, at least 80%, at least 90%, at least 95% or greater amount of O-linked glycans of the total glycoforms. In other embodiments, the predominant glycoform comprise at least 20%, at least 30%, at least 40%, at least 50%, at least 60%, at least 70%, at least 80%, at least 90%, at least 95% or greater amount of Core 1, Core 2, Core 3, Core 4, Core 5, Core 6, Core 7, Core 8 or N-acetyllactosamine glycans of the total glycoforms.

In embodiments, the glyco-ligand composition comprises heterogenous glycoforms. In some embodiments, the glyco-ligand compositions comprise a mixture of one or more glycoforms selected from N-linked glycans and O-linked glycans. In additional embodiments, the heterogenous mixture of glycans comprise at least 20%, at least 30%, at least 40%, at least 50%, at least 60%, at least 70%, at least 80%, at least 90%, at least 95% or greater amount of N-linked glycans of N-glycans of the total glycoforms. Preferably, the total glycoform comprises at least 20%, at least 30%, at least 40%, at least 50%, at least 60%, at least 70%, at least 80%, at least 90%, at least 95% or greater amount of complex N-glycans. In yet other embodiments, the heterogenous mixture of glycans comprise at least 20%, at least 30%, at least 40%, at least 50%, at least 60%, at least 70%, at least 80%, at least 90%, at least 95% or greater amount of O-linked glycans of the total glycoforms. In further embodiments, the heterogenous mixture of glycans comprise at least 20%, at least 30%, at least 40%, at least 50%, at least 60%, at least 70%, at least 80%, at least 90%, at least 95% or greater amount of sialylated glycans of the total glycoforms. In other embodiments, the heterogenous mixture of glycans comprise at least 20%, at least 30%, at least 40%, at least 50%, at least 60%, at least 70%, at least 80%, at least 90%, at least 95% or greater amount of fucosylated glycans of the total glycoforms. In other embodiments, the heterogenous mixture of glycans comprise at least 20%, at least 30%, at least 40%, at least 50%, at least 60%, at least 70%, at least 80%, at least 90%, at least 95% or greater amount of terminal galactose residues of the total glycoforms. In other embodiments, the heterogenous mixture of glycans comprise at least 20%, at least 30%, at least 40%, at least 50%, at least 60%, at least 70%, at least 80%, at least 90%, at least 95% or greater amount of terminal mannose residues of the total glycoforms. In other embodiments, the heterogenous mixture of glycans comprise at least 20%, at least 30%, at least 40%, at least 50%, at least 60%, at least 70%, at least 80%, at least 90%, at least 95% or greater amount of terminal GalNAc residues of the total glycoforms.

In other aspects, the glyco-ligand composition comprises glycans further comprising one or more bisecting, branched, or multiple antennary structures. Preferred glyco-ligand compositions include typical mammalian N-glycans antennary glycan structures including bi-antennary, bisecting GlcNAc, tri-antennary, and tetra-antennary structures. Such multiple antennary structures are catalyzed by one or more N-acetylglucosaminyltransferase enzymes (e.g., GnT I, GnT II, GnT III, GnT IV, and GnT V). Additional sugar residues such as galactose or sialic acid are attached on GlcNAc residues to form glycans with terminal galactose or sialic acid residues.

In various aspects, the pharmaceutical compositions comprising the glyco-ligand comprise a predominantly uniform product. For instance, the glycan component of the pharmaceutical composition comprises a predominant glycoform of at least 50%, at least 60%, at least 70%, at least 80%, at least 90%, at least 100% on the scaffold (e.g., synthetic scaffold domain).

Additional methods are employed to analyze the glycan site occupancy including MS-based labeling and label-free technologies for quantification of N-glycosylation site occupancy (Zhang et al., 2017). Accordingly, in preferred embodiments, the rate of site occupancy of glycans on the glyco-ligand compositions is least 50%, at least 60%, at least 70%, at least 80%, at least 90%, or at least 100%.

In some preferred embodiments, glycans may comprise at least two structurally different structures on the scaffold (e.g., synthetic scaffold domain). For example, the scaffold (e.g., synthetic scaffold domain) comprises a polypeptide, a first glycan is attached to the N-terminus of the scaffold (e.g., synthetic scaffold domain) and a second glycan is attached to the C-terminus. In certain embodiments wherein the scaffold (e.g., synthetic scaffold domain) comprises a polynucleotide, a first glycan is attached to the 3′ terminus of the scaffold (e.g., synthetic scaffold domain) and a second glycan is attached to the 5′ terminus of the scaffold (e.g., synthetic scaffold domain).

### Exemplary Glycan—Nucleic Acid Conjugates

In one aspect, the present disclosure provides compounds of Formula (I):

A-L-B (I),
or a salt, co-crystal, tautomer, stereoisomer, solvate, hydrate, polymorph, or an isotopically enriched derivative thereof, wherein:
A is a nucleic acid of deoxyribonucleic acid (DNA) or ribonucleic acid (RNA) comprising a first click-chemistry handle;
B is an asparagine-linked glycan (N-glycan) comprising a second click-chemistry handle; and
L comprises a linker formed by a bioorthogonal click chemistry reaction between the first click-chemistry handle and the second click-chemistry handle.

In certain embodiments of Formula (I), A is a nucleic acid of deoxyribonucleic acid (DNA) or ribonucleic acid (RNA); B is an asparagine-linked glycan (N-glycan); and L comprises a linker. In certain embodiments, L is any linker as defined herein.

In certain embodiments of Formula (I), A is a nucleic acid of deoxyribonucleic acid (DNA) or ribonucleic acid (RNA); B is an asparagine-linked glycan (N-glycan); and L comprises a linker formed by a bioorthogonal click chemistry reaction between a first click-chemistry handle and a second click-chemistry handle.

In certain embodiments of Formula (I), A is a nucleic acid of deoxyribonucleic acid (DNA) or ribonucleic acid (RNA); B is an asparagine-linked glycan (N-glycan); and L comprises a linker formed by a bioorthogonal click chemistry reaction between a first click-chemistry handle and a second click-chemistry handle, wherein the first click-chemistry handle was attached to A prior to the click chemistry reaction and the second click-chemistry handle was attached to B prior to the click chemistry reaction.

In certain embodiments, in Formula (I), A is DNA (e.g., comprising a first click-chemistry handle). In certain embodiments, in Formula (I), A is an antisense oligonucleotide (ASO). In certain embodiments, in Formula (I), A is an antisense oligonucleotide (ASO) (e.g., comprising a first click-chemistry handle). In certain embodiments, in Formula (I), A is single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, or viral DNA. In certain embodiments, in Formula (I), A is single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, or viral DNA; comprising a first click-chemistry handle.

In certain embodiments, in Formula (I), A is RNA, comprising a first click-chemistry handle. In certain embodiments, in Formula (I), A is small interfering RNA (siRNA). In certain embodiments, in Formula (I), A is small interfering RNA (siRNA), comprising a first click-chemistry handle. In certain embodiments, in Formula (I), A is siRNA comprising a modification (e.g., at the 2′ position). In certain embodiments, in Formula (I), A is siRNA comprising a modification selected from the group consisting of a 2′OMe modification, a fluorine modification (e.g., at the 2′ position), a phosphorothioate modification. In certain embodiments, in Formula (I), A is siRNA comprising a modification selected from the group consisting of a 2′OMe modification, a fluorine modification, a phosphorothioate modification, which also comprises a first click-chemistry handle. In certain embodiments, in Formula (I), A is mRNA. In certain embodiments, in Formula (I), A is mRNA, comprising a first click-chemistry handle. In certain embodiments, in Formula (I), A is guideRNA. In certain embodiments, in Formula (I), A is guideRNA, comprising a first click-chemistry handle. In certain embodiments, in Formula (I), A is circular RNA (circRNA). In certain embodiments, in Formula (I), A is circular RNA (circRNA), comprising a first click-chemistry handle. In certain embodiments, in Formula (I), A is aptamer RNA. In certain embodiments, in Formula (I), A is aptamer RNA, comprising a first click-chemistry handle. In certain embodiments, in Formula (I), A is single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, or viral satellite RNA. In certain embodiments, in Formula (I), A is single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, or viral satellite RNA, comprising a first click-chemistry handle.

In certain embodiments, A comprises the first click-chemistry handle that is an alkyne. In certain embodiments, A comprises the first click-chemistry handle that is an alkyne, for example, wherein the alkyne comprises structure:

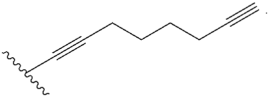 [SMILES: *C#CCCCCC#C]

In certain embodiments, A comprises the first click-chemistry handle comprising DBCO (also known as Azadibenzocyclooctyne-amine, and 3-Amino-1-[(5-aza-3,4:7,8-dibenzocyclooct-1-yne)-5-yl]-1-propanone). In certain embodiments, A comprises the first click-chemistry handle comprising the structure below, or a portion thereof:

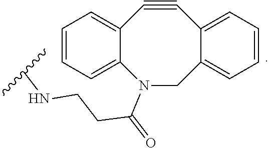 [SMILES: *NCCC(=O)N1Cc2ccccc2C#Cc2ccccc21]

In certain embodiments, the nucleic acid A comprises the first click-chemistry handle that is an alkyne attached to a base of the nucleic acid. In certain embodiments, A comprises the structure:

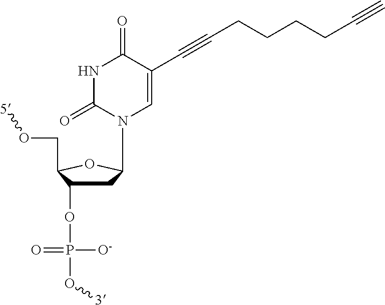

(5-Octadiynyl dU, aka i5OctdU), and A is RNA or DNA. In certain embodiments, A comprises the first click-chemistry handle that is an alkene (vinyl) and B comprises a second click-chemistry handle that is a tetrazine. In certain embodiments, A comprises the first click-chemistry handle that is an alkene (vinyl) (e.g., in FIGS. 2B and/or 2C in Kubota et al.) in Kubota et al., “Expanding the Scope of RNA Metabolic Labeling with Vinyl Nucleosides and Inverse Electron-Demand Diels-Alder Chemistry.” ACS Chemical Biology vol. 14, 8(2019): 1698-1707, incorporated herein by reference. In certain embodiments, A comprises the first click-chemistry handle that is an alkene (vinyl) (e.g., in FIGS. 2B and/or 2C in Kubota et al.) and a second click-chemistry handle that is a tetrazine (e.g., in FIG. 3A Kubota et al.) from Kubota et al., “Expanding the Scope of RNA Metabolic Labeling with Vinyl Nucleosides and Inverse Electron-Demand Diels-Alder Chemistry.” ACS Chemical Biology vol. 14, 8 (2019): 1698-1707, incorporated herein by reference. In certain embodiments, A comprises the first click-chemistry handle that is an alkene, wherein A comprises

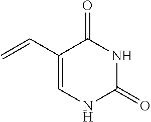

### (5-VU, 1),

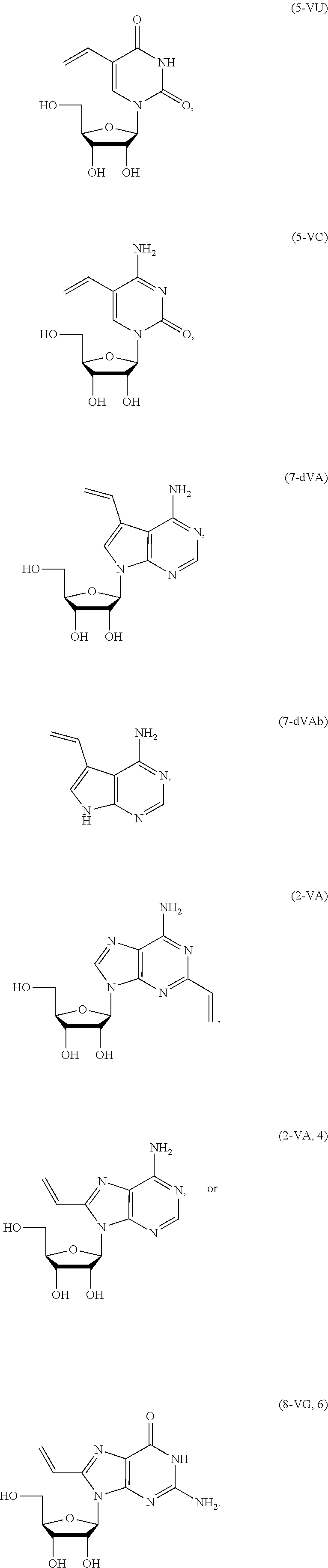

In certain embodiments, L is or comprises substituted or unsubstituted alkylene, alkenylene, substituted or unsubstituted alkenylene, substituted or unsubstituted heteroalkylene, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof; and each R^{A} is independently hydrogen or substituted or unsubstituted alkyl.

In certain embodiments, L is or comprises a substituted or unsubstituted alkylene, alkenylene, substituted or unsubstituted heteroalkylene, substituted or unsubstituted carbocyclylene, substituted or unsubstituted heterocyclylene, substituted or unsubstituted arylene, substituted or unsubstituted heteroarylene, —O—, —N(R^{A})—, —S—, or a combination thereof; and each R^{A} is independently hydrogen or substituted or unsubstituted alkyl.

In certain embodiments, L is or comprises a substituted or unsubstituted alkylene, alkenylene, substituted or unsubstituted heteroalkylene, substituted or unsubstituted carbocyclylene, substituted or unsubstituted heterocyclylene, substituted or unsubstituted arylene, substituted or unsubstituted heteroarylene, —O—, or a combination thereof.

In certain embodiments, L is or comprises a substituted or unsubstituted alkylene, alkenylene, substituted or unsubstituted carbocyclylene, substituted or unsubstituted heterocyclylene, substituted or unsubstituted arylene, substituted or unsubstituted heteroarylene, —O—, or a combination thereof.

In certain embodiments, L is or comprises a combination of alkenylene, substituted or unsubstituted alkylene, and substituted or unsubstituted heteroarylene. In certain embodiments, L is or comprises a combination of alkenylene, unsubstituted alkylene, and unsubstituted heteroarylene,

In certain embodiments, L is or comprises a substituted or unsubstituted heteroarylene. In certain embodiments, L is or comprises a substituted or unsubstituted 5-6 membered heteroarylene. In certain embodiments, L is or comprises a substituted or unsubstituted 5-6 membered heteroarylene having 2-3 nitrogen atoms in the heteroaryl ring. In certain embodiments, L is or comprises substituted or unsubstituted 5-membered heteroarylene having 2-3 nitrogen atoms in the heteroaryl ring. In certain embodiments, L is or comprises a substituted or unsubstituted triazole.

In certain embodiments, L comprises a substituted or unsubstituted heterocyclylene. In certain embodiments, L comprises a substituted or unsubstituted heterocyclylene fused to a substituted or unsubstituted carbocyclylene. In certain embodiments, L comprises a substituted or unsubstituted heterocyclylene fused to a substituted or unsubstituted cyclooctylene. In certain embodiments, L comprises a substituted or unsubstituted 6-membered heterocyclylene fused to a substituted or unsubstituted cyclooctylene. In certain embodiments, L comprises a substituted or unsubstituted dihydropyridazine fused to a substituted or unsubstituted cyclooctylene. In certain embodiments, L comprises a substituted dihydropyridazine fused to an unsubstituted cyclooctylene. In certain embodiments, L comprises an octahydrocycloocta[d]pyridazine.

In certain embodiments, L comprises a substituted or unsubstituted heteroarylene fused to a substituted or unsubstituted carbocyclylene. In certain embodiments, L comprises a substituted or unsubstituted heteroarylene fused to a substituted or unsubstituted cyclooctylene. In certain embodiments, L comprises a substituted or unsubstituted 5-membered heteroarylene fused to a substituted or unsubstituted cyclooctylene. In certain embodiments, L comprises a substituted or unsubstituted triazole fused to a substituted or unsubstituted cyclooctylene.

In certain embodiments, in Formula (I), L is of formula:

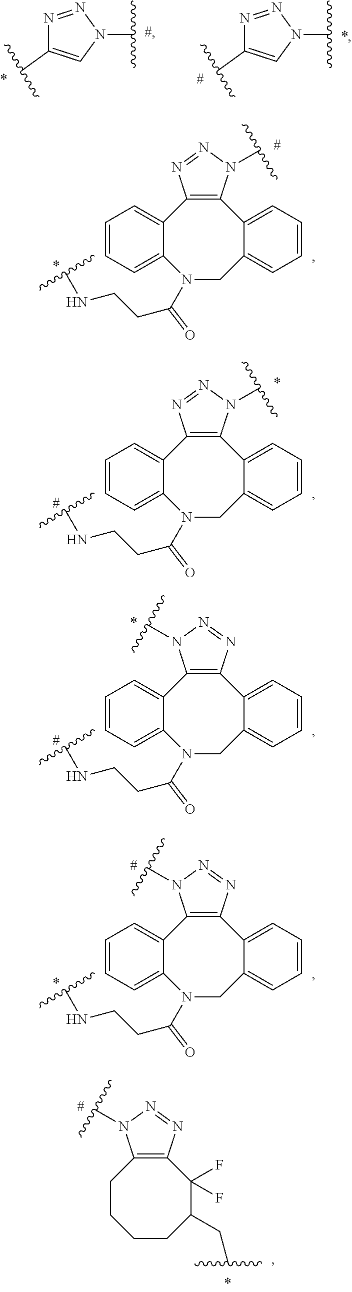

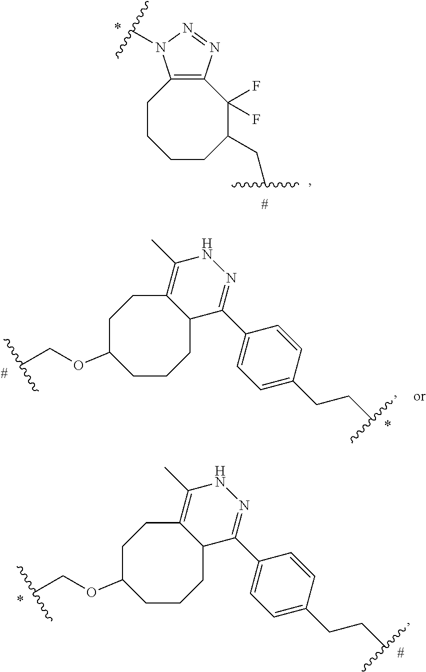

wherein * indicates the point of attachment to A, and #indicates the point of attachment to B. In certain embodiments, in Formula (I), L is of formula:

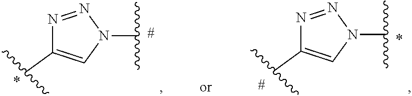 [SMILES: *c1cn(*)nn1 ; *c1cn(*)nn1]

wherein * indicates the point of attachment to A, and #indicates the point of attachment to B. In certain embodiments, L is of formula:

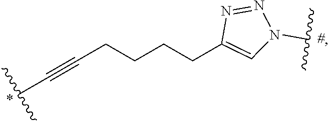 [SMILES: *C#CCCCCc1cn(*)nn1]

wherein * indicates the point of attachment to A, and #indicates the point of attachment to B.

In certain embodiments, in Formula (I), L is attached to a base of the nucleic acid A. In certain embodiments, in Formula (I), L is attached to the 2′OH position of a ribose, 3′OH position of a ribose or deoxyribose, or 5′OH position of a ribose or deoxyribose of the nucleic acid A. In certain embodiments, in Formula (I), L is attached to the 2′OH position of a ribose of the nucleic acid A. In certain embodiments, in Formula (I), L is attached to the 3′OH position of a ribose or deoxyribose of the nucleic acid A. In certain embodiments, in Formula (I), L is attached to an internal portion of the nucleic acid A, the 3′ end of the nucleic acid A, or the 5′ end of the nucleic acid A. In certain embodiments, in Formula (I), L is attached to an internal portion of the nucleic acid A. In certain embodiments, in Formula (I), A is circular RNA (circRNA), and L is attached to an internal portion of A. In certain embodiments, in Formula (I), L is attached to the 5′OH position of a ribose or deoxyribose of the nucleic acid A. In certain embodiments, in Formula (I), L is attached to the non-reducing end of N-glycan B. In certain embodiments, B is an N-glycan that is a mono-antennary N-glycan, a bi-antennary N-glycan, a tri-antennary N-glycan, a tetra-antennary N-glycan or a penta-antennary N-glycan. In certain embodiments, B is an N-glycan that is a mono-antennary N-glycan. In certain embodiments, B is an N-glycan that is a bi-antennary N-glycan. In certain embodiments, B is an N-glycan that is a tri-antennary N-glycan. In certain embodiments, B is an N-glycan that is a tetra-antennary N-glycan. In certain embodiments, B is an N-glycan that is a penta-antennary N-glycan.

In certain embodiments, B comprises a glycan selected from those depicted in FIG. 1, or Tables 1A-1D. In embodiments, B comprises a glycan selected from those depicted in FIG. 1, or Tables 1A-1F, for example, B can comprise a glycan depicted in Tables 1E or 1F. In some embodiments, B comprises a glycan of FIG. 1, or Tables 1A-1D, further comprising an asparagine residue, or a modified asparagine residue, covalently bound to the non-reducing end terminal monosaccharide. In embodiments, B comprises a glycan of FIG. 1 or Tables 1A-1F, further comprising an asparagine residue, or a modified asparagine residue, covalently bound to the non-reducing end terminal monosaccharide, for example, in such embodiments B can comprise a glycan depicted in Tables 1E or 1F. In some embodiments, B comprises a glycan of FIG. 1, or Tables 1A-1D, further comprising aminooxy-PEG3-azide residue covalently bound to the non-reducing end terminal monosaccharide. In embodiments, B comprises a glycan of FIG. 1 or Tables 1A-1F, further comprising aminooxy-PEG3-azide residue covalently bound to the non-reducing end terminal monosaccharide, for example, in such embodiments B can comprise a glycan depicted in Tables 1E or 1F.

### Modulation of Surface Proteins on Target Cells and Receptor Mediated Signaling

Provided herein are methods and compositions for contacting a glyco-ligand on a cell surface protein of target cells. A variety of cell surface proteins of target cells can be contacted with a glyco-ligand to modulate a biological effect.

Provided herein are methods and compositions for modulating cell surface proteins on target cells. Various embodiments are provided for modulating a target cell by contacting a glyco-ligand composition to a cell surface protein wherein the glyco-ligand composition comprises one or more glycans operably linked to one or more sites on a synthetic scaffold domain. Additional targets are modulated by one or more glyco-ligand composition of the invention. Certain embodiments provide agonizing a cell surface protein or protein complex on the surface of a target cell or cell population by contacting one or more glycans on a glyco-ligand. Other embodiments provide antagonizing a cell surface protein or protein complex on the surface of a target cell or cell population. Accordingly, glyco-ligand composition of the invention induces signal transduction or a signaling cascade in a target cell or a cell population.

In various embodiments, methods and compositions for modulating cell surface proteins on target cells include synthesizing or selecting one or more desired glycans, conjugating the glycans onto a synthetic scaffold domain wherein the scaffold (e.g., synthetic scaffold domain) is modified to accept the glycan, contacting one or more cell surface protein comprising a receptor, receptor complex or glycan binding proteins.

Delivery of one or more glyco-ligand composition of the invention can address a number of drawbacks in protein therapeutics such as changes in protein folding, solubility, proteolytic degradation, trafficking, transport, compartmentalization, secretion, recognition by other proteins or factors, antigenicity, or allergenicity or even RNA therapeutics such as targeted delivery, specificity, stability, immunogenicity and off-target toxicity.

A variety of cell surface proteins of target cells can be modulated with a glyco-ligand to induce a biological effect. Cell surface proteins include receptors, glycan binding proteins, lectins, or other proteins containing carbohydrate recognition domains. The glyco-ligand can engage a cell surface protein to produce a desired biological effect. In certain preferred aspects of the invention, one or more lectins targeted by the glyco-ligand are selected from Table 3 below [Raposo C D, Canelas A B, Barros M T. Human Lectins, Their Carbohydrate Affinities and Where to Find Them. Biomolecules. 2021 Jan. 29; 11(2): 188]. In some embodiments, the glycan component of the glyco-ligand is a glycan that binds to a lectin selected from those disclosed in Table 3. In some embodiments, the glycan component of the glyco-ligand is a glycan that selectively binds to a lectin selected from those disclosed in Table 3.

TABLE 3
List of Select Lectins

| | | | |
|---|---|---|---|
| | | | Protein |
| Common Name (HUGO Name | | Carbohydrate Preferential | Expression in |
| if Different) | Gene Symbol | Affinity | the Organs |

C-type superfamily
Proteoglycans or lecticans

| | | | |
|---|---|---|---|
| Aggrecan | ACAN | Hyaluronic acid | Cartilage, soft |
| | | | tissue |
| Brevican | BCAN | Hyaluronic acid | Brain |
| Neurocan | NCAN | Hyaluronic acid | Brain |
| Versican | VCAN | Hyaluronic acid | Brain |
| FRAS1 related extracellular | FREM1 | Unknown | Adrenal gland, |
| matrix 1 | | | appendix, |
| | | | colon, |
| | | | duodenum, |
| | | | epididymis, |
| | | | kidney, lung, |
| | | | pancreas, |
| | | | placenta, |
| | | | rectum, |
| | | | salivary gland, |
| | | | small intestine, |
| | | | stomach, testis, |
| | | | tonsil, thyroid |
| | | | gland |

Type II transmembrane receptors

| | | | |
|---|---|---|---|
| Blood Dendritic Cell Antigen 2 | CLEC4C | Gal-β-(1-3 or 1-4)- | Adipose and |
| (C-type lectin domain family 4 | | GlcNAc-β-(1-2)-Man | soft tissue, |
| member C) | | trisaccharides | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| DC-SIGN (CD209 molecule) | CD209 | High N-linked D- | Bone marrow, |
| | | Mannose- | lung |
| | | oligosaccharides, and | |
| | | branched L-fucose, both | |
| | | with free OH-3 and OH-4. | |
| | | (N-linked glycans, N- | |
| | | acetyl-D-glucosamine, | |
| | | Lewis a, b, x and y) | |
| DC-SIGN2 | CLEC4M | High N-linked D- | Brain, |
| | | Mannose- | gastrointestinal |
| | | oligosaccharides, | tract, lung |
| | | branched L-fucose, N- | |
| | | linked glycans, N-acetyl- | |
| | | D-glucosamine, Lewis a, | |
| | | b and y | |
| Dectin-2 (C-type lectin domain | CLEC6A | α-(1-2) or α-(1-4) | Blood |
| containing 6A) | | mannans and other high- | |
| | | α-D-mannose | |
| | | carbohydrates | |
| Dendritic cell immunoreceptor | CLEC4A | Mannose, fucose and | Bone marrow, |
| (DCIR) (C-type lectin domain | | weakly interacts with N- | spleen, lung |
| family 4 member A) | | acetylglucosamine | |
| Fc fragment of IgE receptor II | FCER2 | Mannose, | Lymph node, |
| | | immunoglobulin E, | bone marrow, |
| | | CD21, galactose | spleen, |
| | | | appendix, |
| | | | tonsil, skin |
| Hepatic Asialoglycoprotein | ASGR1 | Terminal β-D-galactose | Stomach, liver, |
| Receptor 1 | | and N- | gallbladder |
| | | acetylgalactosamine units | |
| Hepatic Asialoglycoprotein | ASGR2 | Terminal β-D-galactose | Liver |
| Receptor 2 | | and N- | |
| | | acetylgalactosamine units | |
| Kupffer Cell receptor (C-type | CLEC4F | Galactose, fucose, and N- | Liver |
| lectin domain family 4 member | | acetylgalactosamine [39] | |
| F) | | High-mannose | |
| | | oligosaccharides, | |
| | | mannose, N- | |
| | | acetylglucosamine, | |
| | | fucose. Note that OH-3 | |
| | | and OH-4 should be free | |
| | | for recognition, and | |
| | | preferentially equatorial. | |
| Langerin (CD207 molecule) | CD207 | N-acetylmannosamine | Lymph node, |
| | | showed less affinity; | tonsil, skin, |
| | | thereby axial derivatives | spleen |
| | | should be avoided. | |
| | | Sulfated mannosylated | |
| | | glycans, keratan sulfate | |
| | | and β-glucans | |
| Liver sinusoidal epithelial cell | CLEC4G | Mannose, N- | Lymph node, |
| lectin (LSECtin) (C-type lectin | | acetylglucosamine and | brain, colon, |
| domain family 4 member G) | | fucose | kidney, liver, |
| | | | testis |
| Macrophage | CLEC10A | Terminal galactose and N- | Bone marrow, |
| Asialoglycoprotein Receptor | | acetylgalactosamine | brain, lymph |
| | | residues | node, oral |
| | | | mucosa, skin, |
| | | | spleen, tonsil |
| Macrophage C-type Lectin | CLEC4D | Trehalose 6,6′- | Bone marrow, |
| (MCL) | | dimycolate, α-D- | lung, lymph |
| | | mannans18 (however it | node, spleen, |
| | | was suggested that MCL | tonsil |
| | | is not a carbohydrate- | |
| | | binding lectin) | |
| MINCLE (C-type lectin | CLEC4E | α-mannose, trehalose-6′6- | Unknown |
| domain family 4 member E) | | dimycolate, glucose | |

Collectins

| | | | |
|---|---|---|---|
| Collectin-K1 (collectin | COLEC11 | High mannose | Unknown |
| subfamily member 11) | | oligosaccharides with at | |
| | | least a mannose-α-(1-2)- | |
| | | mannose residue | |
| Collectin-L1 (collectin | COLEC10 | Galactose, mannose, | Unknown |
| subfamily member 10) | | fucose, N- | |
| | | acetylglucosamine, N- | |
| | | acetylgalactosamine | |
| Mannose-binding lectin 2 | MBL2 | Mannose, fucose, N- | Liver |
| | | acetylglucosamine | |
| Pulmonary surfactant protein 1 | SFTPA1 | N-acetylmannosamine, L- | Lung |
| (surfactant protein A1) | | fucose, mannose, glucose, | |
| | | poorly to galactose. | |
| | | Preferentially | |
| | | oligosaccharides | |
| Pulmonary surfactant protein 2 | SFTPA2 | N-acetylmannosamine, L- | Lung |
| (surfactant protein A2) | | fucose, mannose, glucose, | |
| | | poorly to galactose. | |
| | | Preferentially | |
| | | oligosaccharides | |
| Pulmonary surfactant protein B | SFTPB | Unknown | Lung |
| (surfactant protein B) | | | |
| Pulmonary surfactant protein C | SFTPC | Lipopolysaccharides | Lung |
| (surfactant protein C) | | | |
| Pulmonary surfactant protein D | SFTPD | Maltose, glucose, | Lung |
| (surfactant protein D) | | mannose, poorly to | |
| | | galactose. Preferentially | |
| | | oligosaccharides | |
| Scavenger receptor with CTLD | COLEC12 | D-galactose, L- and D- | Brain, lung, |
| (SRCL) (collectin subfamily | | fucose, N- | placenta |
| member 12) | | acetylgalactosamine | |
| | | (internalizes specifically | |
| | | in nurse-like cells), sialyl | |
| | | Lewis X, or a | |
| | | trisaccharide and asialo- | |
| | | orosomucoid (ASOR). | |
| | | May also play a role in the | |
| | | clearance of amyloid-beta | |
| | | in Alzheimer disease | |

Selectins

| | | | |
|---|---|---|---|
| Selectin E | SELE | Sialyl Lewis x, a | Bone marrow, |
| | | | colon, |
| | | | nasopharynx |
| Selectin L | SELL | Sialyl Lewis x | Appendix, |
| | | | bone marrow, |
| | | | lymph node, |
| | | | spleen, tonsil |
| Selectin P | SELP | Sialyl Lewis x | Bone marrow, |
| | | | colon |

Natural Killer (NK)

| | | | |
|---|---|---|---|
| C-type lectin domain family 2 | CLEC2L | Unknown | Brain, skeletal |
| member L | | | muscle |
| C-type lectin domain | CLEC5A | Fucose, mannose, N- | Blood |
| containing 5A | | acetylglucosamine, N- | |
| | | acetylmuramic acid-β(1- | |
| | | 4)-N-acetylglucosamine | |
| CD72 molecule | CD72 | Unknown | Appendix, |
| | | | bone marrow, |
| | | | lymph node, |
| | | | spleen, tonsil |
| Killer cell lectin-like receptor | KLRG1 | Mannose | Appendix, |
| G1 | | | cervix |
| | | | (uterine), |
| | | | colon, |
| | | | duodenum, |
| | | | small intestine, |
| | | | stomach, tonsil |
| Killer cell lectin-like receptor | KLRG2 | Unknown | Adipose and |
| G2 | | | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| CD69 molecule | CD69 | Fucoidan (weak). N- | Appendix, |
| | | acetylamine was reported | bone marrow, |
| | | but not supported by a | lymph node, |
| | | second report. Does not | spleen, tonsil |
| | | bind glucose, galactose, | |
| | | mannose, fucose or N- | |
| | | acetylglucosamine | |
| Killer cell lectin-like receptor | KLRF1 | Predicted to not bind | Blood |
| F1 | | carbohydrates | |
| C-type lectin domain family 2 | CLEC2B | Unknown carbohydrate | Adipose and |
| member B | | binding; Known to bind to | soft tissue, |
| | | KLRF1 | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | proximal |
| | | | digestive tract, |
| | | | skin |
| Oxidized low-density | OLR1 | Predicted to not bind to | Unknown |
| lipoprotein receptor 1 | | carbohydrates | |
| Killer cell lectin-like receptor | KLRD1 | α-(2-3)-linked NeuAc on | Unknown |
| D1 | | multi-antennary N-glycan, | |
| | | heparin, sulfate- | |
| | | containing | |
| | | polysaccharides | |
| C-type lectin domain family 1 | CLEC1A | Unknown | Unknown |
| member A | | | |
| C-type lectin domain family 1 | CLEC1B | Predicted to not bind to | Unknown |
| member B | | carbohydrates | |
| C-type lectin domain family 12 | CLEC12B | Unknown | Unknown |
| member B | | | |
| C-type lectin-like 1 | CLECL1 | Predicted to not bind to | Unknown |
| | | carbohydrates | |
| C-type lectin domain family 12 | CLEC12A | Unknown | Bone marrow, |
| member A | | | lung, spleen |
| DNGR (C-type lectin domain | CLEC9A | Specific interactions were | Unknown |
| containing 9A) | | not discovered yet, | |
| | | although it is known that | |
| | | this lectin binds to α-actin | |
| | | filaments and β-spectrin | |
| C-type lectin domain family 2 | CLEC2A | Unknown | Skin |
| member A | | | |
| Dectin-1 (C-type lectin domain | CLEC7A | β-(1-3)- and β-(1-6)-D- | Blood, bone |
| containing 7A) | | Glycans (neither mono- or | marrow |
| | | short | |
| | | oligosaccharides/polymers | |
| | | are recognized) | |
| C-type lectin domain family 2 | CLEC2D | High molecular weight | Adipose and |
| member D | | sulfated | soft tissue, |
| | | glycosaminoglycans | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Killer cell lectin-like receptor | KLRB1 | Terminal Gal-α-(1-3)-Gal, | Adipose and |
| B1 | | N-acetyllactosamine, | soft tissue, |
| | | Sucrose octasulphate | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Killer cell lectin-like receptor | KLRC1 | Unknown | Unknown |
| C1 | | | |
| Killer cell lectin-like receptor | KLRC2 | Unknown | Unknown |
| C2 | | | |
| Killer cell lectin-like receptor | KLRC3 | Unknown | Colon, |
| C3 | | | duodenum, |
| | | | small intestine, |
| | | | stomach, tonsil |
| Killer cell lectin-like receptor | KLRC4 | Unknown | Unknown |
| C4 | | | |
| Killer cell lectin-like receptor | KLRK1 | α-(2-3)-NeuAc-containing | Appendix, |
| K1 | | N- glycans, heparin, | lymph node, |
| | | heparan sulfate | spleen, tonsil |

Macrophage Mannose Receptor (MMR)

| | | | |
|---|---|---|---|
| Endo180 (Mannose receptor C | MRC2 | Mannose, fucose, N- | Adipose and |
| type 2) | | acetylglucosamine | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Lymphocyte antigen 75 | LY75 | Predicted to not bind | Appendix, |
| | | carbohydrates | breast, |
| | | | bronchus, |
| | | | cervix |
| | | | (uterine), |
| | | | duodenum, |
| | | | endometrium, |
| | | | fallopian tube, |
| | | | gallbladder, |
| | | | liver, lung, |
| | | | lymph node, |
| | | | nasopharynx, |
| | | | pancreas, |
| | | | placenta, |
| | | | rectum, spleen, |
| | | | stomach, |
| | | | thyroid gland, |
| | | | tonsil, urinary |
| | | | bladder |
| Mannose receptor C-type 1 | MRC1 | Mannose, fucose, glucose, | Colon, |
| | | N-acetylglucosamine (C- | endometrium, |
| | | type), 4-O-sulphated | kidney, lung, |
| | | GalNAc (R-type) | rectum, skin, |
| | | | soft tissue, |
| | | | testis |
| Phospholipase A2 receptor | PLA2R1 | Predicted to not bind | Kidney |
| | | carbohydrates but known | |
| | | to bind collagen | |

Free C-type Lectin Domains (CTLDs)

| | | | |
|---|---|---|---|
| C-type lectin domain | CLEC19A | Unknown | Unknown |
| containing 19A | | | |
| Lithostathine-alpha | REG1A | Unknown | Duodenum, |
| (Regenerating family member | | | pancreas, small |
| 1 alpha) | | | intestine, |
| | | | stomach |
| Lithostathine-beta | REG1B | Unknown | Duodenum, |
| (Regenerating family member | | | pancreas, small |
| 1 beta) | | | intestine, |
| | | | stomach |
| Regenerating family member 3 | REG3A | Peptidoglycan (binding | Appendix, |
| alpha | | affinity increases with the | duodenum, |
| | | length of the carbohydrate | skin, small |
| | | moiety) | intestine, |
| | | | stomach |
| Regenerating family member 3 | REG3G | Peptidoglycan | Unknown |
| gamma | | | |
| Regenerating family member 4 | REG4 | Mannans, heparin | Appendix, |
| | | | colon, |
| | | | duodenum, |
| | | | rectum, small |
| | | | intestine |

Type I receptors

| | | | |
|---|---|---|---|
| Chondrolectin | CHODL | Unknown | Appendix, |
| | | | colon, |
| | | | duodenum, |
| | | | rectum, small |
| | | | intestine, testis |
| Layilin | LAYN | Hyaluronan | Adipose and |
| | | | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |

Tetranectin

| | | | |
|---|---|---|---|
| Cartilage-derived C-type lectin | CLEC3A | Expected to bind sulfated | Unknown |
| (C-type lectin domain family 3 | | polysaccharides such as | |
| member A) | | heparin | |
| Stem cell growth factor | CLEC11A | Unknown | Bone marrow, |
| (SCGF) (C-type lectin domain | | | soft tissue |
| containing 11A) | | | |

Polycystin

| | | | |
|---|---|---|---|
| Polycystin 1 like 3, transient | PKD1L3 | Predicted to not bind | Unknown |
| receptor potential channel | | carbohydrates | |
| interacting | | | |
| Polycystin 1, transient receptor | PKD1 | Predicted to bind | Adipose and |
| potential channel interacting | | galactosyl and glucosyl | soft tissue, |
| | | residues. Might bind | bone marrow |
| | | oligosaccharides with | and lymphoid |
| | | mannosyl moieties | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | pancreas, |
| | | | proximal |
| | | | digestive tract, |
| | | | skin |

Attractin

| | | | |
|---|---|---|---|
| Attractin | ATRN | Unknown | Adipose and |
| | | | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | pancreas, |
| | | | proximal |
| | | | digestive tract, |
| | | | skin |
| Attractin-like 1 | ATRNL1 | Unknown | Unknown |

CTLD/acidic neck

| | | | |
|---|---|---|---|
| CD302 molecule | CD302 | Unknown | Unknown |
| Proteoglycan 2, pro eosinophil | PRG2 | Heparin | Bone marrow, |
| major basic protein | | | placenta |
| Proteoglycan 3, pro eosinophil | PRG3 | Unknown | Bone marrow |
| major basic protein 2 | | | |

Endosialin

| | | | |
|---|---|---|---|
| CD93 molecule | CD93 | Unknown | Bone marrow, |
| | | | brain, colon, |
| | | | kidney, lung, |
| | | | spleen |
| C-type lectin domain | CLEC14A | Unknown | Appendix, |
| containing 14A | | | brain, cervix |
| | | | (uterine), |
| | | | colon, |
| | | | duodenum, |
| | | | esophagus, |
| | | | gallbladder, |
| | | | heart muscle, |
| | | | kidney, lung, |
| | | | pancreas, |
| | | | prostate, |
| | | | rectum, skin, |
| | | | small intestine, |
| | | | stomach, testis |
| Endosialin (CD248 molecule) | CD248 | Unknown | Adipose and |
| | | | soft tissue, |
| | | | bone marrow |
| | | | and lymphoid |
| | | | tissues, brain, |
| | | | female tissues, |
| | | | gastrointestinal |
| | | | tract, kidney |
| | | | and urinary |
| | | | bladder, |
| | | | muscle tissues, |
| | | | pancreas, skin |
| Thrombomodulin | THBD | Unknown | Cervix |
| | | | (uterine), |
| | | | colon, |
| | | | esophagus, |
| | | | lymph node, |
| | | | oral mucosa, |
| | | | placenta, skin, |
| | | | tonsil, urinary |
| | | | bladder, vagina |

Others

| | | | |
|---|---|---|---|
| C-type lectin domain family 18 | CLEC18A | Fucoidan, β-glucans, β- | Unknown |
| member A | | galactans | |
| Prolectin (C-type lectin domain | CLEC17A | Terminal α-D-mannose | Appendix, |
| containing 17A) | | and fucose residues | lymph node, |
| | | | spleen, |
| | | | stomach, tonsil |
| DiGeorge syndrome critical | DGCR2 | Unknown | Pancreas |
| region gene 2 | | | |
| FRAS1 related extracellular | FREM1 | Unknown | Adrenal gland, |
| matrix 1 | | | appendix, |
| | | | colon, |
| | | | duodenum, |
| | | | epididymis, |
| | | | kidney, lung, |
| | | | pancreas, |
| | | | placenta, |
| | | | rectum, |
| | | | salivary gland, |
| | | | small intestine, |
| | | | stomach, testis, |
| | | | tonsil, thyroid |
| | | | gland |

Chitolectins

| | | | |
|---|---|---|---|
| Chitinase 3 like 1 | CHI3L1 | Chitin | Unknown |
| Chitinase 3 like 2 | CHI3L2 | Chitooligosaccharides | Adipose and |
| | | ((GlcNAc)5 and | soft tissue, |
| | | (GlcNAc)6 showed the | bone marrow |
| | | highest affinities) | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | proximal |
| | | | digestive tract |
| Oviductin (Oviductal | OVGP1 | Chitin | Fallopian tube |
| glycoprotein 1) | | | |
| Stabilin-1 interacting chitinase- | SI-CLP | GalNAc, GlcNAc, ribose, | Unknown |
| like protein | | mannose. Prefers to bind | |
| | | oligosaccharides with a | |
| | | four-sugar ring core | |

F-Type Lectins

| | | | |
|---|---|---|---|
| Coagulation factor V | F5 | Fucose | Unknown |
| APC, WNT signaling pathway | APC | Unknown | Adipose and |
| regulator | | | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | proximal |
| | | | digestive tract |
| | | | skin |

F-Box Lectins

| | | | |
|---|---|---|---|
| Cyclin F | CCNF | Unknown | Appendix, |
| | | | bone marrow, |
| | | | lung, lymph |
| | | | node, skin |
| | | | spleen, tonsil |
| F-box protein 2 | FBXO2 | N-acetylglucosamine | Breast, ovary, |
| | | disaccharide chitobiose | pancreas |
| F-box protein 3 | FBXO3 | Unknown | Unknown |
| F-box protein 4 | FBXO4 | Unknown | Unknown |
| F-box protein 5 | FBXO5 | Unknown | Adipose and |
| | | | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| F-box protein 6 | FBXO6 | High-mannose | Adipose and |
| | | glycoproteins | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| F-box protein 7 | FBXO7 | Unknown | Adipose and |
| | | | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| F-box protein 8 | FBXO8 | Unknown | Bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | pancreas, |
| | | | proximal |
| | | | digestive tract, |
| | | | skin |
| F-box protein 9 | FBXO9 | Unknown | Unknown |
| F-box protein 10 | FBXO10 | Unknown | Cervix |
| | | | (uterine), |
| | | | colon, |
| | | | duodenum, |
| | | | endometrium, |
| | | | fallopian tube, |
| | | | lung, prostate, |
| | | | rectum, |
| | | | seminal |
| | | | vesicle, small |
| | | | intestine, testis |
| F-box protein 11 | FBXO11 | Unknown | Unknown |
| F-box protein 15 | FBXO15 | Unknown | Unknown |
| F-box protein 16 | FBXO16 | Unknown | Adipose and |
| | | | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| F-box protein 17 | FBXO17 | Sulfated and galactose- | Unknown |
| | | terminated glycoproteins | |
| F-box protein, helicase, 18 | FBXO18 | Unknown | Adipose and |
| | | | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| LIM domain 7 | LMO7 | Unknown | Unknown |
| F-box protein 21 | FBXO21 | Unknown | Adipose and |
| | | | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | proximal |
| | | | digestive tract, |
| | | | skin |
| F-box protein 22 | FBXO22 | Unknown | Unknown |
| Tetraspanin 17 | TSPAN17 | Unknown | Unknown |
| F-box protein 24 | FBXO24 | Unknown | Unknown |
| F-box protein 25 | FBXO25 | Unknown | Unknown |
| F-box protein 27 | FBXO27 | Unknown | Adipose and |
| | | | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | proximal |
| | | | digestive tract, |
| | | | skin |
| F-box protein 28 | FBXO28 | Unknown | Unknown |
| F-box protein 30 | FBXO30 | Unknown | Unknown |
| F-box protein 31 | FBXO31 | Unknown | Adipose and |
| | | | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | proximal |
| | | | digestive tract, |
| | | | skin |
| F-box protein 32 | FBXO32 | Unknown | Unknown |
| F-box protein 33 | FBXO33 | Unknown | Adipose and |
| | | | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| F-box protein 34 | FBXO34 | Unknown | Adrenal gland, |
| | | | bronchus, |
| | | | colon, |
| | | | epididymis, |
| | | | endometrium, |
| | | | gallbladder, |
| | | | placenta, |
| | | | seminal |
| | | | vesicle, |
| | | | skeletal |
| | | | muscle, skin, |
| | | | stomach, testis, |
| | | | thyroid gland |
| F-box protein 36 | FBXO36 | Unknown | Unknown |
| F-box protein 38 | FBXO38 | Unknown | Unknown |
| F-box protein 39 | FBXO39 | Unknown | Unknown |
| F-box protein 40 | FBXO40 | Unknown | Unknown |
| F-box protein 41 | FBXO41 | Unknown | Unknown |
| F-box protein 42 | FBXO42 | Unknown | Bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | pancreas |
| F-box protein 43 | FBXO43 | Unknown | Unknown |
| F-box protein 44 | FBXO44 | Unknown | Adipose and |
| | | | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| F-box protein 45 | FBXO45 | Unknown | Unknown |
| F-box protein 46 | FBXO46 | Unknown | Unknown |
| F-box protein 47 | FBXO47 | Unknown | Unknown |
| F-box protein 48 | FBXO48 | Unknown | Esophagus, |
| | | | kidney, oral |
| | | | mucosa, |
| | | | parathyroid |
| | | | gland, skin, |
| | | | stomach |

Ficolins

| | | | |
|---|---|---|---|
| Ficolin 1 | FCN1 | GlcNAc, GalNAc; sialic | Unknown |
| | | acid | |
| Ficolin 2 | FCN2 | GlcNAc (acetyl group); β- | Unknown |
| | | (1-3)-D-glucan | |
| Ficolin 3 | FCN3 | N-acetylglucose; N- | Unknown |
| | | acetylgalactose, fucose, | |
| | | lipopolysaccharides | |

I-Type Lectins

| | | | |
|---|---|---|---|
| Siglec1 (Sialic acid binding Ig | SIGLEC1 | α-(2-3)-Sialic acid, α-(2- | Bone marrow, |
| like lectin 1) | | 6)-Sialic acid, α-(2-8)- | lung |
| | | Sialic acid | |
| Siglec2 (CD22 molecule) | CD22 | α-(2-6)-Sialic acid | Appendix, |
| | | | lymph node, |
| | | | spleen, tonsil |
| Siglec3 (CD33 molecule) | CD33 | α-(2-6)-Sialic acid, α-(2- | Appendix, |
| | | 3)-Sialic | bone marrow, |
| | | acid | lung, lymph |
| | | | node, skin, |
| | | | spleen, tonsil |
| Siglec4a, MAG (Myelin | MAG | α-(2-3)-Sialic acid | Brain |
| associated glycoprotein) | | | |
| Siglec5 (Sialic acid binding Ig | SIGLEC5 | α-(2-3)-Sialic acid, α-(2- | Bone marrow, |
| like lectin 5) | | 6)-Sialic acid, α-(2-8)- | lymph node, |
| | | Sialic acid | placenta, |
| | | | spleen, tonsil |
| Siglec6 (Sialic acid binding Ig | SIGLEC6 | Sialic acid-α-(2-6)-N- | Placenta |
| like lectin 6) | | acetylgalactosamine | |
| | | (Sialyl-Tn) | |
| Siglec7 | SIGLEC7 | α-(2-6)-Sialic acid, α-(2- | Unknown |
| | | 8)-Sialic acid, α-(2-3)- | |
| | | Sialic acid and | |
| | | disialogangliosides | |
| Siglec8 | SIGLEC8 | α-(2-3)-Sialic acid, α-(2- | Adipose and |
| | | 6)-Sialic acid | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Siglec9 (Sialic acid binding Ig | SIGLEC9 | α-(2-3)-Sialic acid, Sialyl | Adipose and |
| like lectin 9) | | Lewis x, α-(2-6)-Sialic | soft tissue, |
| | | acid, α-(2-8)-Sialic acid | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Siglec10 (Sialic acid binding Ig | SIGLEC10 | α-(2-3)-Sialic acid, α-(2- | Appendix, |
| like lectin 10) | | 6)-Sialic acid | bone marrow, |
| | | | lymph node, |
| | | | soft tissue, |
| | | | spleen, tonsil |
| Siglec11 (Sialic acid binding Ig | SIGLEC11 | α-(2-8)-Sialic acid | Unknown |
| like lectin 11) | | | |
| Siglec14 (Sialic acid binding Ig | SIGLEC14 | Sialic acid-α-(2-6)-N- | Adipose and |
| like lectin 14) | | acetylgalactosamine | soft tissue, |
| | | (Sialyl-Tn), N- | bone marrow |
| | | acetylneuraminic acid | and lymphoid |
| | | | tissues, brain, |
| | | | endocrine |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Siglec15 (Sialic acid binding Ig | SIGLEC15 | Sialyl-Tn | Unknown |
| like lectin 15) | | | |
| CD2 molecule | CD2 | N-glycans with fucose | Appendix, |
| | | | lymph node, |
| | | | spleen, tonsil |
| CD83 molecule | CD83 | Sialic acid | Appendix, |
| | | | bone marrow, |
| | | | lung, lymph |
| | | | node, spleen, |
| | | | tonsil |
| Intercellular adhesion molecule | ICAM1 | Hyaluronan | Appendix, |
| 1 | | | bone marrow, |
| | | | brain, |
| | | | endometrium, |
| | | | fallopian tube, |
| | | | kidney, lung, |
| | | | lymph node, |
| | | | spleen, testis, |
| | | | tonsil |
| L1 cell adhesion molecule | L1CAM | α-(2-3)-Sialic acid | Adipose and |
| | | | soft tissue, |
| | | | bone marrow |
| | | | and lymphoid |
| | | | tissues, brain, |
| | | | female tissues, |
| | | | gastrointestinal |
| | | | tract, kidney |
| | | | and urinary |
| | | | bladder, lung, |
| | | | male tissues, |
| | | | muscle tissues, |
| | | | proximal |
| | | | digestive tract, |
| | | | skin |
| Myelin protein zero | MPZ | SO₄-−3GlucA-β-(1-3)- | Bronchus, |
| | | Gal-β-(1-4)- GlcNAc | esophagus, |
| | | (HNK-1 | fallopian tube, |
| | | antigen) | small intestine, |
| | | | soft tissue, |
| | | | stomach, testis |
| Neural cell adhesion molecule | NCAM1 | High N-linked D-mannose | Brain, colon, |
| 1 | | | hearth muscle, |
| | | | pancreas, |
| | | | smooth |
| | | | muscle, soft |
| | | | tissue, thyroid |
| | | | gland |
| Neural cell adhesion molecule | NCAM2 | Unknown | Brain, |
| 2 | | | bronchus, |
| | | | colon, |
| | | | duodenum, |
| | | | gallbladder, |
| | | | ovary, rectum, |
| | | | small intestine, |
| | | | soft tissue, |
| | | | testis |

L-Type Lectins

| | | | |
|---|---|---|---|
| Calnexin | CANX | Non-reducing glucose | Adipose and |
| | | residues in an | soft tissue, |
| | | oligosaccharide | bone marrow |
| | | (Glc(Man)9(GlcNAc)2) | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Calreticulin | CALR | Non-reducing glucose | Bone marrow |
| | | residues in an | and lymphoid |
| | | oligosaccharide | tissues, brain, |
| | | (Glc(Man)9(GlcNAc)2) | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | pancreas, skin |
| Calreticulin 3 | CALR3 | Unknown | Testis |
| Lectin, mannose-binding 1 | LMAN1 | α-(1-2) mannans with free | Adipose and |
| | | OH-3, OH-4 and OH-6 | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Lectin, mannose-binding 1 like | LMAN1L | Unknown | Unknown |
| Lectin, mannose-binding 2 | LMAN2 | High α-(1-2) mannans, | Bone marrow |
| | | Low affinity for D- | and lymphoid |
| | | glucose and N- | tissues, brain, |
| | | acetylglucosamine | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | pancreas |
| Lectin, mannose-binding 2 like | LMAN2L | α-(1-2) trimannose | Adipose and |
| | | | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Adhesion G protein-coupled | ADGRD1 | Unknown | Adipose and |
| receptor D1 | | | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Adhesion G protein-coupled | ADGRD2 | Unknown | Unknown |
| receptor D2 | | | |
| Amyloid P component, serum | APCS | Heparin, dextran sulfate | Unknown |
| | | proteoglycans | |
| C-reactive protein | CRP | Galactose 6-phosphate, | Liver, |
| | | Gal-β-(1-3)-GalNAc, Gal- | gallbladder, |
| | | β-(1-4)-GalNAc, Gal-β- | soft tissue |
| | | (1-4)-Gal-β-(1-4)- | |
| | | GlcNAc, other phosphate- | |
| | | containing ligands | |
| Neuronal pentraxin 1 | NPTX1 | Unknown | Brain, testis |
| Neuronal pentraxin 2 | NPTX2 | Unknown | Adrenal gland, |
| | | | brain, |
| | | | pancreas, |
| | | | pituitary gland, |
| | | | testis |
| Neuronal pentraxin receptor | NPTXR | Unknown | Brain |
| Pentraxin 3 | PTX3 | Heparin | Unknown |
| Sushi, von Willebrand factor | SVEP1 | Unknown | Adipose and |
| type A, EGF and pentraxin | | | soft tissue, |
| domain containing 1 | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas |

M-Type Lectins

| | | | |
|---|---|---|---|
| Mannosidase alpha class 1A | MAN1A1 | α-(1-2)-mannans | Adipose and |
| member 1 | | | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Mannosidase alpha class 1A | MAN1A2 | α-(1-2)-mannans | Bone marrow |
| member 2 | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Mannosidase alpha class 1B | MAN1B1 | α-(1-2)-mannans | Adipose and |
| member 1 | | | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Mannosidase alpha class 1C | MAN1C1 | α-(1-2)-mannans | Bone marrow |
| member 1 | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas |

P-Type Lectins

| | | | |
|---|---|---|---|
| Mannose-6-phosphate receptor, | M6PR | Mannose-6-phosphate | Adipose and |
| cation dependent | | residues | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Insulin-like growth factor 2 | IGF2R | Mannose-6-phosphate | Unknown |
| receptor | | residues (either α or β). | |
| | | Mannose-6-phosphate | |
| | | analogues with | |
| | | carboxylate or malonate | |
| | | groups | |

R-Type Lectins

| | | | |
|---|---|---|---|
| Polypeptide N- | GALNT1 | GalNAc | Adipose and |
| acetylgalactosaminyltransferase | | | soft tissue, |
| 1 | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Polypeptide N- | GALNT2 | GalNAc | Bone marrow |
| acetylgalactosaminyltransferase | | | and lymphoid |
| 2 | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Polypeptide N- | GALNT3 | GalNAc | Adipose and |
| acetylgalactosaminyltransferase | | | soft tissue, |
| 3 | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Polypeptide N- | GALNT4 | GalNAc, GalNAc- | Unknown |
| acetylgalactosaminyltransferase | | glycosylated substrates | |
| 4 | | | |
| Polypeptide N- | GALNT5 | GalNAc | Appendix, |
| acetylgalactosaminyltransferase | | | bronchus, |
| 5 | | | cervix |
| | | | (uterine), |
| | | | colon, |
| | | | duodenum, |
| | | | esophagus, |
| | | | gallbladder, |
| | | | lung, oral |
| | | | mucosa, |
| | | | rectum, |
| | | | salivary gland, |
| | | | small intestine, |
| | | | stomach, |
| | | | tonsil, vagina |
| Polypeptide N- | GALNT6 | GalNAc | Bone marrow |
| acetylgalactosaminyltransferase | | | and lymphoid |
| 6 | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Polypeptide N- | GALNT7 | GalNAc, GalNAc- | Bone marrow |
| acetylgalactosaminyltransferase | | glycosylated substrates | and lymphoid |
| 7 | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract |
| Polypeptide N- | GALNT8 | GalNAc | Bone marrow |
| acetylgalactosaminyltransferase | | | and lymphoid |
| 8 | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | skin |
| Polypeptide N- | GALNT9 | GalNAc | Unknown |
| acetylgalactosaminyltransferase | | | |
| 9 | | | |
| Polypeptide N- | GALNT10 | GalNAc | Adipose and |
| acetylgalactosaminyltransferase | | | soft tissue, |
| 10 | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Polypeptide N- | GALNT11 | GalNAc | Adipose and |
| acetylgalactosaminyltransferase | | | soft tissue, |
| 11 | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Polypeptide N- | GALNT12 | GalNAc | Appendix, |
| acetylgalactosaminyltransferase | | | bone marrow, |
| 12 | | | brain, breast, |
| | | | cervix |
| | | | (uterine), |
| | | | endometrium, |
| | | | fallopian tube, |
| | | | prostate, soft |
| | | | tissue, thyroid |
| | | | gland, tonsil, |
| | | | skin |
| Polypeptide N- | GALNT13 | GalNAc | Adrenal gland, |
| acetylgalactosaminyltransferase | | | lung, salivary |
| 13 | | | gland |
| Polypeptide N- | GALNT14 | GalNAc | Unknown |
| acetylgalactosaminyltransferase | | | |
| 14 | | | |
| Polypeptide N- | GALNT15 | GalNAc | Unknown |
| acetylgalactosaminyltransferase | | | |
| 15 | | | |
| Polypeptide N- | GALNT16 | GalNAc | Bone marrow |
| acetylgalactosaminyltransferase | | | and lymphoid |
| 16 | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Polypeptide N- | GALNT17 | GalNAc | Brain |
| acetylgalactosaminyltransferase | | | |
| 17 | | | |
| Polypeptide N- | GALNT18 | GalNAc | Adipose and |
| acetylgalactosaminyltransferase | | | soft tissue, |
| 18 | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Polypeptide N- | GALNTL5 | Unknown | Testis |
| acetylgalactosaminyltransferase | | | |
| like 5 | | | |

S-Type Lectins
Galectin 1

| | | | |
|---|---|---|---|
| Galectin 1 | LGALS1 | β-D-galactosides, poly-N- | Bone marrow, |
| | | acetyllactosamine- | brain, cervix |
| | | enriched glycoconjugates | (uterine), |
| | | | endometrium, |
| | | | lymph node, |
| | | | ovary, |
| | | | parathyroid |
| | | | gland, |
| | | | placenta, |
| | | | smooth |
| | | | muscle, skin, |
| | | | spleen, testis, |
| | | | tonsil, vagina |
| Galectin 2 | LGALS2 | β-D-galactosides, lactose | Appendix, |
| | | | colon, |
| | | | duodenum, |
| | | | gallbladder, |
| | | | kidney, liver, |
| | | | lymph node, |
| | | | pancreas, |
| | | | rectum, small |
| | | | intestine, |
| | | | spleen, tonsil |

Galectin 3

| | | | |
|---|---|---|---|
| Galectin 3 | LGALS3 | β-D-galactosides, | Adipose and |
| | | LacNAc | soft tissue, |
| | | | bone marrow |
| | | | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Galectin 3 binding protein | LGALS3BP | β-D-galactosides, lactose | Adipose and |
| | | | soft tissue, |
| | | | bone marrow |
| | | | and lymphoid |
| | | | tissues, brain, |
| | | | female tissues, |
| | | | gastrointestinal |
| | | | tract, kidney |
| | | | and urinary |
| | | | bladder, lung, |
| | | | male tissues, |
| | | | muscle tissues, |
| | | | proximal |
| | | | digestive tract, |
| | | | skin |
| Galectin 4 | LGALS4 | β-D-galactosides, lactose | Appendix, |
| | | | colon, |
| | | | duodenum, |
| | | | gallbladder, |
| | | | pancreas, |
| | | | rectum, small |
| | | | intestine, |
| | | | stomach |
| Galectin 7 | LGALS7 | Gal, GalNAc, Lac, | Cervix |
| | | LacNAc | (uterine), |
| | | | esophagus, |
| | | | oral mucosa, |
| | | | salivary gland, |
| | | | skin, tonsil, |
| | | | vagina |
| Galectin 8 | LGALS8 | β-D-galactosides. | Adipose and |
| | | Preferentially binds to 3′- | soft tissue, |
| | | O-sialylated and 3′-O- | bone marrow |
| | | sulfated glycans | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Galectin 9 | LGALS9 | β-D-galactosides. | Adipose and |
| | | Forssman | soft tissue, |
| | | pentasaccharide, lactose, | bone marrow |
| | | N-acetyllactosamine | and lymphoid |
| | | | tissues, brain, |
| | | | endocrine |
| | | | tissues, female |
| | | | tissues, |
| | | | gastrointestinal |
| | | | tract, kidney |
| | | | and urinary |
| | | | bladder, lung, |
| | | | male tissues, |
| | | | muscle tissues, |
| | | | pancreas, |
| | | | proximal |
| | | | digestive tract, |
| | | | skin |
| Galectin 9B | LGALS9B | β-D-galactosides | Appendix, |
| | | | bone marrow, |
| | | | breast, lymph |
| | | | node, spleen, |
| | | | tonsil |
| Galectin 9C | LGALS9C | β-D-galactosides | Appendix, |
| | | | bronchus, |
| | | | colon, |
| | | | duodenum, |
| | | | gallbladder, |
| | | | lung, pancreas, |
| | | | spleen, |
| | | | stomach, tonsil |
| Galectin 10 (Charcot-Leyden | LGALS10 | Binds weakly to lactose, | Lymph node, |
| crystal galectin, CLC) | | N-acetyl-D-glucosamine | spleen, tonsil |
| | | and D-mannose | |
| Galectin 12 | LGALS12 | β-D-galactose and lactose, | Unknown |
| | | N-acetyl-lactosamine, | |
| | | mannose and N-acetyl- | |
| | | galactosamine | |
| Galectin 13 | LGALS13 | Contrary to other | Kidney, |
| | | galectins, Galectin 13 | placenta, |
| | | does not bind β-D- | spleen, urinary |
| | | galactosides | bladder |
| Placental Protein 13 (Galectin | LGALS14 | N-acetyl-lactosamine | Adrenal gland, |
| 14) | | | colon, kidney |
| Galectin 16 | LGALS16 | N-acetyl-lactosamine, β- | Placenta |
| | | D-galactose, and lactose | |

X-Type Lectins

| | | | |
|---|---|---|---|
| Intelectin 1 | ITLN1 | Terminal acyclic 1,2-diol- | Appendix, |
| | | containing structures, | colon, |
| | | including β-D- | duodenum, |
| | | galactofuranose, D- | rectum, small |
| | | phosphoglycerol-modified | intestine |
| | | glycans, D-glycero-D- | |
| | | talo-oct-2-ulosonic acid, | |
| | | 3-deoxy-D-manno-oct-2- | |
| | | ulosonic acid | |
| Intelectin 2 | ITLN2 | Unknown | Appendix, |
| | | | colon, |
| | | | duodenum, |
| | | | rectum, small |
| | | | intestine |

In some embodiments, the glycan component of the glyco-ligand is a glycan that binds a plasma membrane lectin. In some embodiments, the glycan component of the glyco-ligand is a glycan that selectively binds a plasma membrane lectin.

In some embodiments, the glycan component of the glyco-ligand is a glycan that binds to MRC1 (macrophage Man-type receptor). In some embodiments, the glycan component of the glyco-ligand is a glycan that selectively binds to MRC1 (macrophage Man-type receptor). In some embodiments, the glycan that binds MRC1 is H-1 or H-2.

In some embodiments, the glycan component of the glyco-ligand is a glycan that binds to DC-SIGN. In some embodiments, the glycan component of the glyco-ligand is a glycan that selectively binds to DC-SIGN. In some embodiments, the glycan that binds DC-SIGN is H-3.

In some embodiments, the glycan component of the glyco-ligand is a glycan that binds to MGL (macrophage galactose-type lectin). In some embodiments, the glycan component of the glyco-ligand is a glycan that selectively binds to MGL (macrophage galactose-type lectin). In some embodiments, the glycan that binds MGL is H-4 or H-5.

In some embodiments, the glycan component of the glyco-ligand is a glycan that binds to Siglec 3. In some embodiments, the glycan component of the glyco-ligand is a glycan that selectively binds to Siglec 3.

In some embodiments, the glycan component of the glyco-ligand is a glycan that binds to Siglec 8. In some embodiments, the glycan component of the glyco-ligand is a glycan that selectively binds to Siglec 8.

In some embodiments, the glycan component of the glyco-ligand is a glycan that binds to Siglec 3 and Siglec 8. In some embodiments, the glycan that binds Siglec 3 and Siglec 8 is H-6.

In some embodiments, the glycan component of the glyco-ligand is a glycan that binds to Siglec 9. In some embodiments, the glycan component of the glyco-ligand is a glycan that selectively binds to Siglec 9. In some embodiments, the glycan that binds Siglec 9 is H-9.

In some embodiments, the glycan component of the glyco-ligand is a glycan that binds to Siglec 2. In some embodiments, the glycan component of the glyco-ligand is a glycan that selectively binds to Siglec 2. In some embodiments, the glycan that binds Siglec 2 is H-33.

In some embodiments, the glycan component of the glyco-ligand is a glycan that binds to Siglec 4a. In some embodiments, the glycan component of the glyco-ligand is a glycan that selectively binds to Siglec 4a. In some embodiments, the glycan that binds Siglec 4a is H-17.

In some embodiments, the glycan component of the glyco-ligand is a glycan that binds to langerin. In some embodiments, the glycan component of the glyco-ligand is a glycan that selectively binds to langerin. In some embodiments, the glycan that binds langerin is H-14, H-15 or H-16.

In some embodiments, the glycan component of the glyco-ligand is a glycan that binds to Dectin-1. In some embodiments, the glycan component of the glyco-ligand is a glycan that selectively binds to Dectin-1. In some embodiments, the glycan that binds Dectin-1 is H-18.

In some embodiments, the glycan component of the glyco-ligand is a glycan that binds to Dectin-2. In some embodiments, the glycan component of the glyco-ligand is a glycan that selectively binds to Dectin-2. In some embodiments, the glycan that binds Dectin-2 is H-10.

In some embodiments, the glycan component of the glyco-ligand is a glycan that binds to CLEC4E. In some embodiments, the glycan component of the glyco-ligand is a glycan that selectively binds to CLEC4E. In some embodiments, the glycan that binds CLEC4E is H-47, H-48, H-49, H-50, or H-51.

In some embodiments, the glycan component of the glyco-ligand is a glycan that binds to CLEC12A. In some embodiments, the glycan component of the glyco-ligand is a glycan that selectively binds to CLEC12A. In some embodiments, the glycan that binds CLEC12A is H-45 or H-46.

In some embodiments, the glycan component of the glyco-ligand is a glycan that binds to CLEC14A. In some embodiments, the glycan component of the glyco-ligand is a glycan that selectively binds to CLEC14A. In some embodiments, the glycan that binds CLEC14A is H-19 or H-20.

In some embodiments, the glycan component of the glyco-ligand is a glycan that binds to CLEC4A. In some embodiments, the glycan component of the glyco-ligand is a glycan that selectively binds to CLEC4A. In some embodiments, the glycan that binds CLEC4A is H-26.

In some embodiments, the glycan component of the glyco-ligand is a glycan that binds to CLEC4C. In some embodiments, the glycan component of the glyco-ligand is a glycan that selectively binds to CLEC4C. In some embodiments, the glycan that binds CLEC4C is H-27.

In some embodiments, the glycan component of the glyco-ligand is a glycan that binds to CLEC5A. In some embodiments, the glycan component of the glyco-ligand is a glycan that selectively binds to CLEC5A. In some embodiments, the glycan that binds CLEC5A is H-25 or H-32.

In some embodiments, the glycan component of the glyco-ligand is a glycan that binds to CLEC2D. In some embodiments, the glycan component of the glyco-ligand is a glycan that selectively binds to CLEC2D. In some embodiments, the glycan that binds CLEC2D is H-30.

In some embodiments, the glycan component of the glyco-ligand is a glycan that binds to CD2. In some embodiments, the glycan component of the glyco-ligand is a glycan that selectively binds to CD2. In some embodiments, the glycan that binds CD2 is H-24. In some embodiments, the glycan that binds CD2 is one of K-4, K-10, K-12 and K-13. In some embodiments, the glycan that binds CD2 enables delivery of the glyco-ligand to T-cells and NK-cells in a subject. In some embodiments, the glycan that binds CD2 comprises one or more glycosaminoglycans (GAGs).

In some embodiments, the glycan component of the glyco-ligand is a glycan that binds to E selectin. In some embodiments, the glycan component of the glyco-ligand is a glycan that selectively binds to E selectin. In some embodiments, the glycan that binds E Selectin is H-23. In some embodiments, the glycan that binds E selectin is H-64. In some embodiments, the glycan that binds E selectin enables delivery of the glyco-ligand to endothelial cells in a subject. In some embodiments, the glycan that binds E selectin comprises a Lewis glycan.

In some embodiments, the glycan component of the glyco-ligand is a glycan that binds to P Selectin. In some embodiments, the glycan component of the glyco-ligand is a glycan that selectively binds to P Selectin. In some embodiments, the glycan that binds P Selectin is H-29.

In some embodiments, the glycan component of the glyco-ligand is a glycan that binds to L Selectin. In some embodiments, the glycan component of the glyco-ligand is a glycan that selectively binds to L Selectin. In some embodiments, the glycan that binds L Selectin is H-9 or H-34.

In some embodiments, the glycan component of the glyco-ligand is a glycan that binds to Thrombomodulin. In some embodiments, the glycan component of the glyco-ligand is a glycan that selectively binds to Thrombomodulin. In some embodiments, the glycan that binds thrombomodulin is H-22 or H-31.

In some embodiments, the glycan component of the glyco-ligand is a glycan that binds to SRCL. In some embodiments, the glycan component of the glyco-ligand is a glycan that selectively binds to SRCL. In some embodiments, the glycan that binds SRCL is H-24.

In some embodiments, the glycan component of the glyco-ligand is a glycan that binds a serum lectin. In some embodiments, the glycan component of the glyco-ligand is a glycan that selectively binds a serum lectin.

In some embodiments, the glycan component of the glyco-ligand is a glycan that binds to MBL (Man-binding lectin). In some embodiments, the glycan component of the glyco-ligand is a glycan that selectively binds to MBL (Man-binding lectin). In some embodiments, the glycan that binds MBL is H-10.

In some embodiments, the glycan component of the glyco-ligand is a glycan that binds to Galectin-2 (LGACS2). In some embodiments, the glycan component of the glyco-ligand is a glycan that selectively binds to Galectin-2 (LGACS2). In some embodiments, the glycan that binds Galectin-2 is H-11.

In some embodiments, the glycan component of the glyco-ligand is a glycan that binds to Anti-α-Gal antibody. In some embodiments, the glycan component of the glyco-ligand is a glycan that selectively binds to Anti-α-Gal antibody. In some embodiments, the glycan that binds Anti-α-Gal antibody is H-12.

In some embodiments, the glycan component of the glyco-ligand is a glycan that binds to Galectin-3. In some embodiments, the glycan component of the glyco-ligand is a glycan that selectively binds to Galectin-3.

In some embodiments, the glycan component of the glyco-ligand is a glycan that binds to Galectin-8. In some embodiments, the glycan component of the glyco-ligand is a glycan that selectively binds to Galectin-8.

In some embodiments, the glycan component of the glyco-ligand is a glycan that binds to Galectin-3 and Galectin-8. In some embodiments, the glycan that binds Galectin-3 and Galectin-8 is H-13.

In some embodiments, the glycan component of the glyco-ligand is a glycan that binds Siglec-11. In some embodiments, the glycan component of the glyco-ligand is a glycan that selectively binds to Siglec-11. In some embodiments, the glycan that binds Siglec-11 is selected from H-35, H-36, H-37, H-38, H-39, H-40, H-41, H-42, H-43, and H-44.

In some embodiments, the glycan component of the glyco-ligand is a glycan that binds to CD161. In some embodiments, the glycan component of the glyco-ligand is a glycan that selectively binds to CD161. In some embodiments, the glycan that binds CD161 is H-17, H-48, H-52, H-53, H-54, and H-55.

In some embodiments, the glycan component of the glyco-ligand is a glycan that binds Siglec-1. In some embodiments, the glycan component of the glyco-ligand is a glycan that selectively binds to Siglec-1. In some embodiments, the glycan that binds Siglec-1 is H-56.

In some embodiments, the glycan component of the glyco-ligand is a glycan that binds Siglec-2. In some embodiments, the glycan component of the glyco-ligand is a glycan that selectively binds to Siglec-2. In some embodiments, the glycan that binds Siglec-2 is H-57.

In some embodiments, the glycan component of the glyco-ligand is a glycan that binds Siglec-3. In some embodiments, the glycan component of the glyco-ligand is a glycan that 165 selectively binds to Siglec-3. In some embodiments, the glycan that binds Siglec-3 is H-57. In some embodiments, the glycan that binds Siglec-3 is H-6. In some embodiments, the glycan that binds Siglec-3 enables delivery of the glyco-ligand to one or more tissues or cells selected from bone marrow, lymphoid progenitors, myeloid progenitors, macrophages, monocytes, microglia and granulocytes, in a subject. In some embodiments, the glycan that binds Siglec-3 comprises a sulfo-Lewis glycan.

In some embodiments, the glycan component of the glyco-ligand is a glycan that binds Siglec-4. In some embodiments, the glycan component of the glyco-ligand is a glycan that selectively binds to Siglec-4. In some embodiments, the glycan that binds Siglec-4 is H-58.

In some embodiments, the glycan component of the glyco-ligand is a glycan that binds Siglec-5. In some embodiments, the glycan component of the glyco-ligand is a glycan that selectively binds to Siglec-5. In some embodiments, the glycan that binds Siglec-5 is H-59.

In some embodiments, the glycan component of the glyco-ligand is a glycan that binds Siglec-7. In some embodiments, the glycan component of the glyco-ligand is a glycan that selectively binds to Siglec-7. In some embodiments, the glycan that binds Siglec-7 is H-60. In some embodiments, the glycan that binds Siglec-7 is one of K-51, K-52, K-53, H-7, and H-8. In some embodiments, the glycan that binds Siglec-7 enables delivery of the glyco-ligand to NK-cells in a subject. In some embodiments, the glycan that binds Siglec-7 comprises ganglioside glycans. In some embodiments, the glycan that binds Siglec-7 comprises an O-glycan.

In some embodiments, the glycan component of the glyco-ligand is a glycan that binds Siglec-9. In some embodiments, the glycan component of the glyco-ligand is a glycan that selectively binds to Siglec-9. In some embodiments, the glycan that binds Siglec-9 is H-61. In some embodiments, the glycan that binds Siglec-9 is H-9. In some embodiments, the glycan that binds Siglec-9 enables delivery of the glyco-ligand to immune cells, including but not limited to B-cells, T-cells and NK-cells, in a subject. In some embodiments, the glycan that binds Siglec-9 comprises a sulfo-Lewis glycan.

In some embodiments, the glycan component of the glyco-ligand is a glycan that binds Siglec-10. In some embodiments, the glycan component of the glyco-ligand is a glycan that selectively binds to Siglec-10. In some embodiments, the glycan that binds Siglec-10 is H-62.

In some embodiments, the glycan component of the glyco-ligand is a glycan that binds CD28. In some embodiments, the glycan component of the glyco-ligand is a glycan that selectively binds to CD28. In some embodiments, the glycan that binds CD28 is one of K-1, K-2, K-3, K-4, K-5, K-6, K-7, K-8, K-9, K-10, K-11, K-12, and K-13. In some embodiments, the glycan that binds CD28 is one of K-4, K-10, K-12 and K-13. In some embodiments, the glycan that binds CD28 enables delivery of the glyco-ligand to T-cells and NK-cells in a subject. In some embodiments, the glycan that binds CD28 comprises one or more glycosaminoglycans (GAGs).

In some embodiments, the glycan component of the glyco-ligand is a glycan that binds CD22. In some embodiments, the glycan component of the glyco-ligand is a glycan that selectively binds to CD22. In some embodiments, the glycan that binds CD22 is one of K-33, K-34, K-35, K-36, K-37, K-38, K-39, K-40, K-41, K-42, K-43, K-44, K-45, K-46, K-47, K-48, K-49, and K-50. In some embodiments, the glycan that binds CD22 is one of G-3, G-6, G-20, G-21, H-33, and M-1. In some embodiments, the glycan that binds CD22 enables delivery of the glyco-ligand to B-cells in a subject. In some embodiments, the glycan that binds CD22 comprises a sialylated glycan. In some embodiments, the glycan that binds CD22 comprises a sialylated N-glycan. In some embodiments, the glycan that binds CD22 comprises one or more chemically modified sialo-oligosaccharides, or in other words a sialo-oligosaccharide comprising one or more non-saccharide chemical modifications.

In some embodiments, the glycan component of the glyco-ligand is a glycan that binds CD83. In some embodiments, the glycan component of the glyco-ligand is a glycan that selectively binds to CD83. In some embodiments, the glycan that binds CD83 is one of K-14, K-15, K-16, and K-17. In some embodiments, the glycan that binds CD83 is one of K-4, K-10, K-12 and K-13. In some embodiments, the glycan that binds CD83 enables delivery of the glyco-ligand to T-cells and NK-cells in a subject. In some embodiments, the glycan that binds CD83 comprises one or more glycosaminoglycans (GAGs).

In some embodiments, the glycan component of the glyco-ligand is a glycan that binds KLRF1. In some embodiments, the glycan component of the glyco-ligand is a glycan that selectively binds to KLRF1. In some embodiments, the glycan that binds KLRF1 is one of K-18, K-19, K-20, and K-21. In some embodiments, the glycan that binds KLRF1 is one of K-4, K-10, K-12 and K-13. In some embodiments, the glycan that binds KLRF1 enables delivery of the glyco-ligand to T-cells and NK-cells in a subject. In some embodiments, the glycan that binds KLRF1 comprises one or more glycosaminoglycans (GAGs).

In some embodiments, the glycan component of the glyco-ligand is a glycan that binds CD93. In some embodiments, the glycan component of the glyco-ligand is a glycan that selectively binds to CD93. In some embodiments, the glycan that binds CD93 is one of K-31, H-37, H-35, and H-39.

In some embodiments, the glycan component of the glyco-ligand is a glycan that binds DC-SignR. In some embodiments, the glycan component of the glyco-ligand is a glycan that selectively binds to DC-SignR. In some embodiments, the glycan that binds DC-SignR is one of K-22, K-23, K-24, K-25, K-26, K-27, K-28, K-29, K-30, and K-31. In some embodiments, the glycan that binds DC-SignR is H-3. In some embodiments, the glycan that binds DC-SignR is one of K-22, K-23, K-24, K-27, K-28, K-29, K-30, K-31, and H-3. In some embodiments, the glycan that binds DC-SignR enables delivery of the glyco-ligand to liver sinusoidal endothelial cells (LSECs) in a subject.

In some embodiments, the glycan component of the glyco-ligand is a glycan that binds ASGR1. In some embodiments, the glycan component of the glyco-ligand is a glycan that selectively binds to ASGR1.

In some embodiments, the glycan component of the glyco-ligand is a glycan that binds ASGR2. In some embodiments, the glycan component of the glyco-ligand is a glycan that selectively binds to ASGR2.

In some embodiments, the glycan component of the glyco-ligand is a glycan that binds ASGPR. In some embodiments, the glycan component of the glyco-ligand is a glycan that selectively binds to ASGPR. In some embodiments, the glycan that binds ASGPR comprises an N-glycan comprising one or more terminal Gal or GalNAc. In some embodiments, the glycan that binds ASGPR comprises a glycan comprising one or more terminal Gal or GalNAc. In some embodiments, the glycan that binds ASGPR is one of G-2, G-5, G-29, G-30, G-12, G-16, G-37, and G-38.

In some embodiments, the glycan component of the glyco-ligand is a glycan that binds megalin. In some embodiments, the glycan component of the glyco-ligand is a glycan that selectively binds to megalin. In some embodiments, the glycan that binds megalin comprises one or more chitosan units. In some embodiments, the glycan that binds megalin is one of M-5, M-6, and M-7. In some embodiments, the glycan that binds megalin enables delivery of the glyco-ligand to kidney of a subject. In some embodiments, the megalin targeting glyco-ligand is preferentially taken up by proximal tubule epithelial cells (PTECs).

In some embodiments, the glycan component of the glyco-ligand is a glycan that localizes the glyco-ligand in the lymph nodes of a subject. In some embodiments, the glycan that localizes the glyco-ligand in the lymph nodes comprises one or more terminal GlcNAcs. In some embodiments, the glycan that localizes the glyco-ligand in the lymph nodes comprises an N-glycan comprising one or more terminal GlcNAcs. In some embodiments, the glycan that localizes the glyco-ligand in the lymph nodes is one of M-2, M-3, M-4, G-1, G-4, G-9, G-10, G-13, G-15, G-27, and G-28.

In some embodiments, the glycan component of the glyco-ligand is a glycan that binds CD206. In some embodiments, the glycan component of the glyco-ligand is a glycan that selectively binds to CD206. In some embodiments, the glycan that binds CD206 comprises a high mannose or oligomannose glycan. In some embodiments, the glycan that binds CD206 comprises a high mannose or oligomannose N-glycan. In some embodiments, the glycan that binds CD206 is one of G-23, G-24, G-8, H-65, H-14, H-10, and H-40.

In certain embodiments, the glycan component comprises one or more sialic acid moieties and facilitates penetration of the blood-brain barrier. It has been reported that upregulation of sialyltransferases and the resultant hypersialylation of tumor cell surfaces are established hallmarks of several cancers, including lung, breast, ovarian, pancreatic and prostate cancer (Dobie, et al. British Journal of Cancer volume 124, pages 76-90 (2021)). Hypersialylation promotes tumor metastasis by several routes, including enhancing immune evasion and tumor cell survival and stimulating tumor invasion and migration. Bos, et al., Nature volume 459, pages 1005-1009 (2009) reports that epidermal growth factor receptor (EGFR) ligand HBEGF, and the α2,6-sialyltransferase ST6GALNAC5 are mediators of cancer cell passage through the blood-brain barrier. Sialylated glycans, including but not limited to the sialyl-Lewisx tetrasaccharide H-23, can enable transport of payloads through the blood brain barrier, thereby enabling treatment of diseases and disorders of the brain. In certain embodiments, the glyco-ligands of the present disclosure are useful for treating diseases and disorders of the brain.

About 100 glycan-binding receptors exist that are known in humans indicating the types of glycan-receptor binding and their selectivity. [Taylor M E, Drickamer K, Schnaar R L, Etzler M E & Varki A (2015) Discovery and classification of glycan-binding proteins. In Essentials of Glycobiology (A Varki, R D Cummings, J D Esko, P Stanley, G W Hart, M Aebi, A G Darvill, T Kinoshita, N H Packer, J H Prestegard, R L Schnaar & P H Seeberger, eds), pp. 361-372. Cold Spring Harbor Laboratory Press, Cold Spring Harbor, NY.]

The four largest groups of glycan-binding receptors contain distinct types of carbohydrate recognition domains (CRDs). These are the Siglecs, in which the CRDs are based on the immunoglobulin fold; the galectins, which have CRDs formed from a different β sandwich fold; the C-type lectins, in which sugars are ligated directly to a calcium ion bound to the CRD; lectins containing R-type CRDs, related in structure to the plant toxin ricin, and at least 10 additional structural categories of CRDs found in one or more types of mammalian glycan-binding receptors. [Taylor, M. E. and Drickamer, K. (2019) Mammalian sugar-binding receptors: known functions and unexplored roles. FEBS J, 286:1800-1814].

The selectins represent by far the best characterized paradigm for glycan-binding receptors that play this role, mediating initial transient interaction between leukocytes and endothelial cells, which results in rolling of the leukocytes along the endothelial surface [Taylor, M. E. and Drickamer, K. (2019) Mammalian sugar-binding receptors: known functions and unexplored roles. FEBS J, 286:1800-1814].

The sialyl-Lewis tetrasaccharide on endothelial cells at sites of inflammation serves as an attachment point for the C-type CRD of the selectin, mediating an initial weak adhesion that results in leukocytes rolling along the endothelium [Taylor, M. E. and Drickamer, K. (2019) Mammalian sugar-binding receptors: known functions and unexplored roles. FEBS J, 286:1800-1814].

The molecular mechanisms of interaction of the C-type CRD in the extracellular portion of each selectin with the glycan ligand involve direct ligation of the fucose residue in the sialyl-Lewis^{x} tetrasaccharide to the conserved calcium ion that is characteristic of the C-type CRDs, along with additional secondary interactions with other sugar residues in the tetrasaccharide [Taylor, M. E. and Drickamer, K. (2019) Mammalian sugar-binding receptors: known functions and unexplored roles. FEBS J, 286:1800-1814].

The same phenotype is seen in mice lacking expression of two GlcNAc-6-O-sulfotransferases, GlcNAc6ST-1 and GlcNAc6ST-2, that are required to generate the sialyl 6-sulfo Lewis glycan ligand for L-selectin on glycoproteins of high endothelial venules [Taylor, M. E. and Drickamer, K. (2019) Mammalian sugar-binding receptors: known functions and unexplored roles. FEBS J, 286:1800-1814].

Glycoprotein transport toward the cell surface is facilitated by glycan-binding receptors in the endoplasmic reticulum-Golgi intermediate compartment and trafficking of hydrolytic enzymes to lysosomes is directed by mannose 6-phosphate receptors [Taylor, M. E. and Drickamer, K. (2019) Mammalian sugar-binding receptors: known functions and unexplored roles. FEBS J, 286:1800-1814].

Mannose receptors bind to oligomannose or high mannose glycans. Patients with Gaucher disease, a lysosomal storage disease, are now routinely treated successfully by enzyme replacement therapy, in which missing lysosomal hydrolases bearing appropriate mannose-containing glycans are injected into the circulation for uptake into macrophages via the mannose [Taylor, M. E. and Drickamer, K. (2019) Mammalian sugar-binding receptors: known functions and unexplored roles. FEBS J, 286:1800-1814].

ASGR for removal of terminal sialic acid, e.g., galactose or GalNAc. Lewis trisaccharide for scavenger receptor C-type lectin which is found on glycoproteins released from secondary granules of neutrophils [Taylor, M. E. and Drickamer, K. (2019) Mammalian sugar-binding receptors: known functions and unexplored roles. FEBS J, 286:1800-1814].

Glycoproteins bound by scavenger receptor C-type lectin (SRCL) are rapidly internalized into cells and degraded. Thus, it appears likely that SRCL has a role similar to the mannose receptor in clearing potentially dangerous glycoproteins released at sites of inflammation. [Taylor, M. E. and Drickamer, K. (2019) Mammalian sugar-binding receptors: known functions and unexplored roles. FEBS J, 286:1800-1814].

Therapies based on targeting the asialoglycoprotein receptor are also in development, taking advantage of the ability to control expression of proteins in hepatocytes by delivering interfering RNA molecules [Foster D J, Brown C R, Shaikh S, Trapp C, Schlegel M K, Qian K, Sehgal A, Rajeev K G, Jadhav V, Manoharan M et al. (2018) Advanced siRNA designs further improve in vivo performance of GalNAc-siRNA conjugates. Mol Ther 26, 708-717.]. Knowledge of the asialoglycoprotein receptor glycoprotein turnover mechanism also informs development of appropriately glycosylated therapeutic glycoproteins such as erythropoietin to ensure that they have suitable serum half-life [Taylor, M. E. and Drickamer, K. (2019) Mammalian sugar-binding receptors: known functions and unexplored roles. FEBS J, 286:1800-1814]. In addition to the C-type CRDs that bind to mannose-containing oligosaccharides, the mannose receptor contains an R-type CRD that binds selectively to terminal 4-SO4-GalNAc [Taylor, M. E. and Drickamer, K. (2019) Mammalian sugar-binding receptors: known functions and unexplored roles. FEBS J, 286:1800-1814].

Specific aspects of the glycan structures attached to glycoproteins can have a significant effect on their interaction with the receptor. Glycoproteins in which sialic acid is in 2-6 linkage to galactose or GalNAc residues, rather than in 2-3 linkage, can bind to the receptor without removal of the sialic acid and are thus cleared constitutively [Taylor, M. E. and Drickamer, K. (2019) Mammalian sugar-binding receptors: known functions and unexplored roles. FEBS J, 286:1800-1814].

The levels of these glycoproteins increase in mice lacking the receptor. More highly branched tri- and tetra-antennary glycans bind with higher affinity to the receptor, which may create a hierarchy of clearance rates [Taylor, M. E. and Drickamer, K. (2019) Mammalian sugar-binding receptors: known functions and unexplored roles. FEBS J, 286:1800-1814].

The glyco-ligand of the present invention can interact with cells containing glycan binding proteins, which include T cells, B cells, NK cells, RBCs, macrophages, monocytes, platelets, granulocytes, gamma delta T cells, other immune cells, and immune-modulatory intracellular signaling domains.

### Glycan Binding Receptors

Glycan binding receptors include immunotyrosine inhibitory motifs (ITIMs) in the cytoplasmic domains of many of the Siglecs such as CD22 on B lymphocytes [Taylor, M. E. and Drickamer, K. (2019) Mammalian sugar-binding receptors: known functions and unexplored roles. FEBS J, 286:1800-1814]. Following interaction with sialylated glycans, such as those on host cells, the ITIMs interact with SHP-1 phosphatase, which leads to inhibition of B-cell activation by modulating Ca2+-dependent signaling [Taylor, M. E. and Drickamer, K. (2019) Mammalian sugar-binding receptors: known functions and unexplored roles. FEBS J, 286:1800-1814]. This pathway may prevent targeting of self-antigens that are extensively sialylated. The dendritic cell inhibitory receptor (DCIR) functions in a somewhat similar way and contains an ITIM in the cytoplasmic domain, although in this case the extracellular sugar-binding domain is a C-type CRD and the ligands bound contain mannose [Taylor, M. E. and Drickamer, K. (2019) Mammalian sugar-binding receptors: known functions and unexplored roles. FEBS J, 286:1800-1814].

C-type lectins mincle and dectin-2 on macrophages as well as blood dendritic cell antigen 2 (BDCA-2) on plasmacytoid dendritic cells lack signaling motifs but interact with the common Fc receptor γ chain [Taylor, M. E. and Drickamer, K. (2019) Mammalian sugar-binding receptors: known functions and unexplored roles. FEBS J, 286:1800-1814].

The CRDs are generally rigid and the binding sites do not change upon ligand binding [Taylor, M. E. and Drickamer, K. (2019) Mammalian sugar-binding receptors: known functions and unexplored roles. FEBS J, 286:1800-1814]. In addition, the CRDs are often spaced away from the cell surface by stalk regions. It is more likely that activation involves induced interactions between multiple receptor polypeptides, either as dimers or as larger clusters. One way that dimerization could initiate signaling has been suggested for dectin-1, because engagement with β glucan brings together two receptor polypeptides to create a fully functional ITAM from the hemi-ITAMs present in the cytoplasmic domain of each polypeptide [Taylor, M. E. and Drickamer, K. (2019) Mammalian sugar-binding receptors: known functions and unexplored roles. FEBS J, 286:1800-1814].

Galectins interacting with glycosylated membrane receptors provide an alternative model for how glycan-binding proteins can modulate signaling [Taylor, M. E. and Drickamer, K. (2019) Mammalian sugar-binding receptors: known functions and unexplored roles. FEBS J, 286:1800-1814]. Galectins are typically at least bivalent, either because of the presence of tandem CRDs in a single polypeptide or because noncovalent oligomers are formed from single CRDs [Taylor, M. E. and Drickamer, K. (2019) Mammalian sugar-binding receptors: known functions and unexplored roles. FEBS J, 286:1800-1814]. At the cell surface, multivalent galectins can bring together glycoproteins to form a lattice, which can either stimulate or inhibit signals. For example, galectin-1 crosslinking of CD45 results in activation of the phosphatase domains in the cytoplasmic domain of the receptor, which can modulate T-cell responses such as apoptosis [Taylor, M. E. and Drickamer, K. (2019) Mammalian sugar-binding receptors: known functions and unexplored roles. FEBS J, 286:1800-1814]. In contrast, lattice formation between multivalent galectins and T-cell receptors bearing multiple glycans prevents close clustering of the cytoplasmic domains of the receptor polypeptides, increasing the threshold for activation of the receptor by antigen [Taylor, M. E. and Drickamer, K. (2019) Mammalian sugar-binding receptors: known functions and unexplored roles. FEBS J, 286:1800-1814].

The CRDs in pathogen-binding receptors often have extended binding sites which bind common disaccharide motifs such as Manα1-2Man, which is a common terminal structure on mannans of yeast and other fungi, or GlcNAcβ1-2Man, which is exposed on under-processed viral glycans [Taylor, M. E. and Drickamer, K. (2019) Mammalian sugar-binding receptors: known functions and unexplored roles. FEBS J, 286:1800-1814]. Some of the CRDs have even more extended sugar-binding sites, such as the cleft in the CRD of DC-SIGN that binds several mannose residues in high mannose oligosaccharides that are present on the surface of HIV [Taylor, M. E. and Drickamer, K. (2019) Mammalian sugar-binding receptors: known functions and unexplored roles. FEBS J, 286:1800-1814].

In preferred aspects, the present invention provides glyco-ligand compositions that act as direct ligands for Siglec receptors and modulate immune regulation in a subject. In some embodiments, negatively charged or enriched glycans comprising one or more sialic acid residues mediate glycan-receptor binding to Siglec active sites containing a conserved arginine residue.

In further aspects, the glyco-ligands of the invention may be characterized as either positive or negative regulators of target receptors. Ablation of N-glycosylation of CD28 expressed on T-cells, binding of CD28 to CD80 significantly increased and amplification of downstream signal activation, which indicates the negative regulation of CD28 function by N-linked glycosylation. Ma, Bruce Y et al. “CD28 T cell costimulatory receptor function is negatively regulated by N-linked carbohydrates.” Biochemical and biophysical research communications vol. 317, 1 (2004): 60-7. In additional aspects, the glyco-ligands of the invention are characterized as exhibiting bidirectional regulation. Nitschke L, Carsetti R, Ocker B, Köhler G, Lamers M C (February 1997). “CD22 is a negative regulator of B-cell receptor signalling.”

Binding of the glyco-ligand can be mediated by or trigger Siglec intracellular signaling upon contact by desired glycans that are presented in an orientation that leads to clustering of signaling proteins. Glyco-ligands can bind in a specific orientation or conformation or bind to multiple receptors to mediate a biological effect. See FIG. 4A. Accordingly, preferred embodiments of the invention provide glyco-ligands that engage target receptors via glycan-glycan interactions. For instance, a glycan on the glyco-ligand interacts with a glycan on the lectin or the receptor.

Siglec receptors are expressed on different cell types including but not limited to macrophage, monocyte, B cell, Schwann cell, ODC, DC, osteoclasts, MyPro, monocyte, granulocyte, microglia, mast cell, neutrophil, trophoblast, NK cell, T cell, eosinophil, basophil, and platelet. Preferably one or more Siglec receptors selected from Siglec-2, Siglec-3, Siglec-4A, Siglec-5, Siglec-6 Siglec-7, Siglec-8, Siglec-9, Siglec-10, Siglec-11, Siglec-14, or Siglec-16 are modulated by the glyco-ligand of the invention. Similarly, CD33 and conserved Siglecs including sialoadhesin, MAG, CD22, and Siglec-15 are also modulated by the glyco-ligand of the invention.

The glyco-ligands of the present invention can also include targeting groups, e.g., a cell or tissue targeting agent or group, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer.

Targeting groups can be proteins, e.g., glycoproteins, or peptides, e.g., molecules having a specific affinity for a co-ligand, or antibodies, e.g., an antibody, that binds to a specified cell type such as a cancer cell, endothelial cell, or bone cell. Targeting groups may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, multivalent fucose, or aptamers.

The targeting group can be any ligand that is capable of targeting a specific receptor. Examples include, without limitation, folate, GalNAc, galactose, mannose, mannose-6P, aptamers, integrin receptor ligands, chemokine receptor ligands, transferrin, biotin, serotonin receptor ligands, prostate-specific membrane antigen (PSMA), endothelin, glutamate carboxypeptidase II (GCPII), somatostatin, LDL, and HDL ligands. In particular embodiments, the targeting group is an aptamer. The aptamer can be unmodified or have any combination of modifications disclosed herein.

In still other embodiments, glyco-ligands are covalently conjugated to a cell penetrating polypeptide. The cell-penetrating peptide may also include a signal sequence. The conjugates of the invention can be designed to have increased stability; increased cell transfection; and/or altered biodistribution (e.g., targeted to specific tissues or cell types) in comparison to unconjugated molecules (e.g., unconjugated RNA, unconjugated DNA).

Conjugating moieties may be added to glycan-interacting antibodies such that they allow labeling or flagging targets for clearance. Such tagging/flagging molecules include, but are not limited to ubiquitin, fluorescent molecules, human influenza hemagglutinin (HA), c-myc [a 10 amino acid segment of the human protooncogene myc with sequence EQKLISEEDL (SEQ ID NO: 10)], histidine (His), flag [a short peptide of sequence DYKDDDDK (SEQ ID NO: 11)], glutathione S-transferase (GST), V5 (a paramyxovirus of simian virus 5 epitope), biotin, avidin, streptavidin, horse radish peroxidase (HRP) and digoxigenin.

In some embodiments, glycan-interacting antibodies may be combined with one another or other molecules in the treatment of a disease or condition.

In some embodiments, the glyco-ligand composition binds to a chimeric antigen receptor (CARs) or ligand binding domain of T-cell receptors (TCRs), alpha and/or beta subunits. The CARs and TCRs can comprise an antigen-binding domain, a transmembrane domain, and an intracellular domain. In some embodiments, the glyco-ligand composition binds to one or more antigen-binding protein comprising an antigen-binding domain, a transmembrane domain, and an intracellular signaling domain. In some embodiments, the antigen binding domain is linked to the transmembrane domain, which is linked to the intracellular signaling domain to produce a chimeric antigen receptor. In some embodiments, the antigen-binding domain binds to a tumor antigen, a tolerogen, or a pathogen antigen, or the antigen is a tumor antigen, or a pathogen antigen. In some embodiments, the antigen-binding domain is an antibody or antibody fragment thereof (e.g., scFv, Fv, Fab, dAb). In some embodiments, the antigen binding domain is a bispecific antibody.

In some embodiments, the bispecific antibody has first immunoglobulin variable domain that binds a first epitope and a second immunoglobulin variable domain that binds a second epitope. In some embodiments, the first epitope and the second epitope are the same. In some embodiments, the first epitope and the second epitope are different. In some embodiments, the transmembrane domain links the binding domain and the intracellular signaling domain. In some embodiments, the transmembrane domain is a hinge protein (e.g., immunoglobulin hinge), a polypeptide linker (e.g., GS linker), a KIR2DS2 hinge, a CD8a hinge, or a spacer.

In some embodiments, the intracellular signaling domain comprises at least a portion of a T-cell signaling molecule. In some embodiments, the intracellular signaling domain comprises an immunoreceptor tyrosine-based activation motif. In some embodiments, the intracellular signaling domain comprises at least a portion of CD3zeta, common FcRgamma (FCER1G), Fc gamma Rlla, FcRbeta (Fc Epsilon Rib), CD3 gamma, CD3delta, CD3epsilon, CD79a, CD79b, DAP10, DAP12, or any combination thereof. In some embodiments, the intracellular signaling domain further comprises a costimulatory intracellular signaling domain.

In some embodiments, the costimulatory intracellular signaling domain comprises at least one or more of a TNF receptor protein, immunoglobulin-like protein, a cytokine receptor, an integrin, a signaling lymphocytic activation molecule, or an activating NK cell receptor protein. In some embodiments, the costimulatory intracellular signaling domain comprises at least one or more of CD27, CD28, 4-1BB, 0X40, GITR, CD30, CD40, PD-1, ICOS, BAFFR, HVEM, ICAM-1, LFA-1, CD2, CDS, CD7, CD287, LIGHT, NKG2C, NKG2D, SLAMF7, NKp80, NKp30, NKp44, NKp46, CD160, CD19, CD4, CD8alpha, CD8beta, IL2R beta, IL2R gamma, IL7R alpha, ITGA4, VLA1, CD49a, IA4, CD49D, ITGA6, VLA6, CD49f, ITGAD, CD103, ITGAL, ITGAM, ITGAX, ITGB1, CD29, ITGB2, CD18, ITGB7, TNFR2, TRAN CE/TRANKL, CD226, SLAMF4, CD84, CD96, CEACAM1, CRTAM, CD229, CD 160, PSGL1, CD100, CD69, SLAMF6, SLAMF1, SLAMF8, CD162, LTBR, LAT, GADS, SLP-76, PAG/Cbp, CD19a, B7-H3, or a ligand that binds to CD83.

### Specific Cell-Targeting Ligands to Bring Other Bioactive Molecules to Particular Target Cells

In other aspects, the pharmaceutical composition further comprises targeting or effector molecules (e.g. a bioactive molecules) associated with or operably linked to the glycan-conjugated nucleic acid molecule. For instance, radio-ligands, toxins, enzymes, protein or peptide can be conjugated to the glyco-ligand (FIGS. 2A, 2B, 2C and 2D).

In some embodiments, the glyco-ligand compositions are conjugated to one or more proteins enzymatically by using stop codon suppression for 1-2 modifications or incorporation of a non-natural amino acid to lead to either a single conjugation position or every amino acid to be a conjugation position.

In other embodiments, glyco-ligand compositions are conjugated to one or more proteins by chemical synthesis. These glyco-ligand compositions are programmable when conjugated to peptides with <12 amino acids, but it is challenging to make long peptides that fold correctly. Preferably the conjugated peptides are folded correctly.

In other embodiments, glyco-ligand compositions are configured on to nucleic acids enzymatically, which can include modified nucleosides in transcription reactions or ligation to long RNAs. In yet other embodiments, glyco-ligand compositions are conjugated via chemical syntheses that are programmable (<120 nts) and can define particular structures and orientations for glycans to engage receptors. See FIG. 2B, which shows that a glyco-ligand in a specific orientation or conformation facilitates binding to a receptor or multiple receptors. In one embodiment, the glyco-ligand is in a specific orientation or conformation to facilitate binding to a receptor or multiple receptors.

In preferred embodiments, the glyco-ligands are operably linked to one or more bioactive molecules to bind to target cells. In some embodiments, the bioactive molecules comprise toxins such as azaribine, anastrozole, azacytidine, bleomycin, bortezomib, bryostatin-1, busulfan, camptothecin, 10-hydroxycamptothecin, carmustine, celebrex, chlorambucil, cisplatin, irinotecan, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin, ethinyl estradiol, estramustine, etoposide, etoposide glucuronide, floxuridine, fludarabine, flutamide, fluorouracil, fluoxymesterone, gemcitabine, hydroxyprogesterone caproate, hydroxyurea, idarubicine, ifosfamide, leucovorin, lomustine, mechlorethamine, medroxyprogesterone acetate, megestrol acetate, melphalan, mercaptopurine, methotrexate, mitoxantrone, mithramycin, mitomycin, mitotane, phenylbutyrate, prednisone, procarbazine, paclitaxel, pentostatin, semustine, streptozocin, tamoxifen, taxanes, testosterone propionate, thalidomide, thioguanine, thiotepa, teniposide, topotecan, uracil mustard, vinblastine, vinorelbine and vincristine. In other embodiments, the bioactive molecules comprise enzymes such as glycosidases, e.g., sialidase, galactosidase, hexosamindiase, fucosidase, mannosidase, PNGase, etc. In some embodiments, the bioactive molecules comprise proteins and peptides.

### Glyco-Ligand Analysis

Analysis of glyco-ligands can be performed using MALDI-TOF-MS, NMR spectroscopy, glycosidase degradation and other known methods in glycobiology.

Glycans are purified from the medium typically by chromatography and then released using glycosidases such as peptide-N-glycosidase F (PNGaseF). The glycans are detected by MALDI-TOF-MS as described in Example 4. Typically, the mass of a particular glycan correlates to the structure of the glycan+/−ionization.

Since the measurement of glycans through MS only provide mass of ionized glycans, structures of specific hexose glycans cannot be discerned without glycosidic analysis. Accordingly, NMR is used to detect glycosidic linkages, the specific glycan linkages (alpha, beta) between the glycan structures. NMR protocol and analysis are adapted from Gao et al.

¹H and ¹³C NMR spectra are recorded on a Bruker Avance II 600 MHz and an Agilent 700 MHz NMR Magnet System. The compounds are deuterium oxide exchanged three times before reconstitution in deuterium oxide for analysis. Characterization of the generated glycans/glycan conjugates are as follows: Chemical shift (in parts per million (ppm) relative to water as the internal standard), multiplicity (s=singlet, d=doublet, t=triplet, dd=doublet of doublet, m=multiplet and/or multiple resonances), coupling constant in Hertz (Hz), integration. All NMR signals are assigned on the basis of ¹H NMR, ¹H-¹H COSY, ¹H-¹H TCOSY, and ¹H-¹³C HSQC experiments.

### Cell-Based Assays

Also provided herein are methods to detect glyco-ligand bioactivity and interaction of the glyco-ligand on a cell surface protein of target cells.

In some embodiments, glyco-ligands as provided herein are characterized through enzyme-linked lectin assay, fluorescence based solid-phase assay or cell-based assays. Cell-based assays can be carried out in vitro with cells in culture or in vivo. For instance, cells used in cell-based assays may express one or more target receptors recognized by one or more glyco-ligands of the invention. The target receptors may be naturally expressed by such cells or cells may be induced to express one or more desired target receptors. Induced expression may be through one or more treatments that upregulate gene expression of the protein that regulate the receptor. In some embodiments, induced expression may include transfection, transduction, or other form of introduction of one or more genes or transcripts for the endogenous expression overexpression of one or cell surface proteins involved in regulation of the receptor.

In certain embodiments, cell-based assays may include the use of cancer cells, macrophages, microglia, neutrophils, monocytes, B cells, T cells, NK cells and eosinophils.

In certain embodiments, cell-based assays may include the use of cancer cells, which express the target receptor or may be induced to express target receptor. Additionally, cancer cell lines may be used to test the glyco-ligand of the invention, where the cancer cell lines are representative of cancer stem cells (CSC).

In some embodiments, ovarian cancer cell lines may be used. Such cell lines may include, but are not limited to SKOV3, OVCAR3, OV90 and A2870 cell lines. In some cases, CSC cells may be isolated from these cell lines by isolating cells expressing CD44 and/or CD133 cell markers.

OVCAR3 cells were first established using malignant ascites obtained from a patient suffering from progressive ovarian adenocarcinoma (Hamilton, T. C. et al., 1983. Cancer Res. 43:5379-89). Cancer stem cell populations may be isolated from OVCAR3 cell cultures through selection based on specific cell surface markers such as CD44 (involved in cell adhesion and migration), CD133 and CD117 (Liang, D. et al., 2012. BMC Cancer. 12:201, the contents of which are herein incorporated by reference in their entirety). OV90 cells are epithelial ovarian cancer cells that were similarly derived from human ascites (see U.S. Pat. No. 5,710,038). OV-90 cells may also express CD44 when activated (Meunier, L. et al., 2010. Transl Oncol. 3(4): 230-8).

In some embodiments, cell lines derived from gastric cancers may be used. Such cell lines may include, but are not limited to SNU-16 cells (see description in Park J. G. et al., 1990. Cancer Res. 50:2773-80, the contents of which are herein incorporated by reference in their entirety). SNU-16 cells express STn naturally, but at low levels.

### Methods of Treatment

Also provided are methods of treating a disease or condition comprising administering to a subject in need thereof a therapeutically effective amount of a pharmaceutical composition of the present disclosure comprising a glyco-ligand described herein. In certain embodiments, wherein the synthetic scaffold domain is or comprises a therapeutic polynucleotide, such as an mRNA or siRNA, the present disclosure contemplates administering to a subject a therapeutically effective amount of a glyco-RNA, such that the one or more glycan moieties enable and promote delivery of the therapeutic polynucleotide to an organ or cell of interest. In some embodiments, the one or more glycan moieties result in increased delivery efficiency of the therapeutic polynucleotide (and therefore a greater therapeutic effect), as compared to a nonfunctionalized analog. In some embodiments, the disease or condition is any disease or condition that can be treated by the therapeutic polynucleotide. Exemplary diseases and conditions that can be treated by the methods of the present disclosure include, but are not limited to, cancers, metabolic diseases, clotting diseases, anti-clotting diseases, autoimmune diseases, and infections (e.g., viral infections, bacterial infections).

Also provided are glyco-ligands of the present disclosure for the manufacture of a medicament for the treatment of a disease or a condition. Further provided are methods of using a pharmaceutical composition disclosed herein for the treatment of a disease or a condition in a subject in need thereof.

### Vectors & Delivery Vehicles

Also provided are vectors, including expression vectors, which comprise the nucleic acid molecules of the present invention, as described further herein. In a first embodiment, the vectors include the isolated nucleic acid molecules described above. In an alternative embodiment, the vectors of the present invention include the above-described nucleic acid molecules operably linked to one or more expression control sequences. The vectors of the instant invention may thus be used to express a polypeptide. Vectors useful for expression of nucleic acids are well known in the art.

In another aspect of the present invention, delivery of the glyco-ligands includes non-viral compositions. In certain embodiments delivery vehicles include nanoparticles, lipids, lipid-based nanoparticles and polymers comprising the nucleic acid molecules of the present invention wherein one or more of the vehicles carry the glycan conjugated nucleic acid sequences of the present invention.

Delivery vehicles are selected based on lower toxicity and immunogenicity, improved half-life, increased stability, and efficiency.

Treatment of Cancer and Combinations with Other Drugs

In one embodiment, the invention is directed to a method of killing cancer cells in a subject by administering to the subject a therapeutically effective amount of glyconucleic acids, such as glycoRNAs and glycoDNAs. In one aspect of this embodiment, glyconucleic acids, such as glycoRNAs and glycoDNAs, are administered intravenously to the subject. In another aspect of this embodiment, glyconucleic acids, such as glycoRNAs and glycoDNAs, are administered into a tumor in the subject. In still another aspect of this embodiment, glyconucleic acids, such as glycoRNAs and glycoDNAs, are administered in proximity to the tumor or administered systemically in a vehicle that allows delivery to the tumor.

In another embodiment, the invention is directed to a method of treating a cancer in a subject by administering to the subject a therapeutically effective amount of a glyconucleic acid, such as glycoRNA and glycoDNA. In one aspect of this embodiment, glycoRNA is administered intravenously to the subject. In another aspect of this embodiment, glycoRNA is administered into a tumor in the subject. In still another aspect of this embodiment, glycoRNA is administered in proximity to the tumor or administered systemically in a vehicle that allows delivery to the tumor.

The cancer (and the cancer cells) is any cancer that afflicts a subject. Such cancers include liver, colon, pancreatic, lung, and bladder cancer. The liver cancer can be a primary liver cancer or a cancer that has metastasized to the liver from another tissue. Primary liver cancers include hepatocellular carcinoma and hepatoblastoma. Metastasized cancers include colon and pancreatic cancer.

In one embodiment, the invention is directed to a method of killing cancer cells in a subject by administering to the subject a therapeutically effective amount of an immune checkpoint inhibitor with the therapeutically effective amount of glyconucleic acid, such as glycoRNA and glycoDNA. In one aspect of this embodiment, the administration of the immune checkpoint inhibitor with the glyconucleic acid (e.g., glycoRNA) increases the efficacy of the glyconucleic acid (e.g., glycoRNA). Administration of the immune checkpoint inhibitor with the glyconucleic acid (e.g., glycoRNA) can increase the efficacy of the checkpoint inhibitor.

In another embodiment, the invention is directed to a method of treating a cancer in a subject by administering to the subject a therapeutically effective amount of an immune checkpoint inhibitor with the therapeutically effective amount of glyconucleic acid, such as glycoRNA and glycoDNA. In one aspect of this embodiment, the administration of the immune checkpoint inhibitor with the glyconucleic acid (e.g., glycoRNA) increases the efficacy of the glyconucleic acid (e.g., glycoRNA).

As stated above, the immune checkpoint inhibitor and the glyconucleic acid, such as glycoRNA and glycoDNA, are administered intravenously to the subject, into a tumor in the subject in proximity to the tumor, or systemically in a vehicle that allows delivery to the tumor. In one aspect of this embodiment, the immune checkpoint inhibitor is a monoclonal antibody that blocks the interaction between receptors, such as PD-1, CTLA4, Lag3, and Tim3, and ligands (e.g., PD-L1, PD-L2, B7-1, B7-2, MHC class II, fibrinogen-link 1 protein (FGL1), Galectin-3, lymph node sinusoidal endothelial cell C-type lectin (LSECtin), alpha-synuclein fibrils, Galectin-9, high-mobility group protein B1 (HMGB1), Ceacam-1, phosphatidylserine) for those receptors on mammalian cells, such as human cells. In a particular aspect, the monoclonal antibody is a monoclonal antibody to PD-1 or PDL1. Examples of monoclonal antibodies include Atezolizumab, Durvalumab, Nivolumab, Pembrolizumab, and Ipilimumab.

In still another aspect of this embodiment, the immune checkpoint inhibitor is a small molecule that blocks the interaction between receptors, such as PD-1, CTLA4, Lag3, and Tim3, and ligands (e.g., PD-L1, PD-L2, B7-1, B7-2, MHC class II, fibrinogen-link 1 protein (FGL1), Galectin-3, lymph node sinusoidal endothelial cell C-type lectin (LSECtin), alpha-synuclein fibrils, Galectin-9, high-mobility group protein B1 (HMGB1), Ceacam-1, phosphatidylserine) for those receptors on mammalian cells, such as human cells. In a particular aspect, the small molecule blocks binding between PD-1 and PD-L1. BMS202 and similar ligands are examples of such small molecules. The immune checkpoint inhibitor administered with the glyconucleic acid, such as glycoRNA and glycoDNA, molecules is a monoclonal antibody or a small molecule as described above. It can be administered before, after, or concurrently with the combination of the glyconucleic molecules.

In another embodiment, a pharmaceutical composition described herein is used in connection with an immune checkpoint inhibitor as described herein. Thus, this embodiment of the invention is directed to a combination of therapeutic drugs comprising an immune checkpoint inhibitor and a pharmaceutical composition comprising a glyconucleic acid, such as glycoRNA and glycoDNA, in a pharmaceutically acceptable carrier as described herein.

In another embodiment, the pharmaceutical composition comprising a glyconucleic acid, such as glycoRNA and glycoDNA, is used in connection with a chemotherapeutic agent. Illustrative examples of chemotherapeutic agents which may be administered with the pharmaceutical composition and have a cytotoxic effect include: azaribine, anastrozole, azacytidine, bleomycin, bortezomib, bryostatin-1, busulfan, camptothecin, 10-hydroxycamptothecin, carmustine, celebrex, chlorambucil, cisplatin, irinotecan, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin, ethinyl estradiol, estramustine, etoposide, etoposide glucuronide, floxuridine, fludarabine, flutamide, fluorouracil, fluoxymesterone, gemcitabine, hydroxyprogesterone caproate, hydroxyurea, idarubicine, ifosfamide, leucovorin, lomustine, mechlorethamine, medroxyprogesterone acetate, megestrol acetate, melphalan, mercaptopurine, methotrexate, mitoxantrone, mithramycin, mitomycin, mitotane, phenylbutyrate, prednisone, procarbazine, paclitaxel, pentostatin, semustine, streptozocin, tamoxifen, taxanes, testosterone propionate, thalidomide, thioguanine, thiotepa, teniposide, topotecan, uracil mustard, vinblastine, vinorelbine and vincristine.

In some embodiments, the chemotherapeutic agent is selected from the group consisting of panobinostat, actinomycin, all-trans retinoic acid, azacitidine, azathioprine, bleomycin, bortezomib, carboplatin, capecitabine, cisplatin, chlorambucil, cyclophosphamide, cytosine arabinoside, daunorubicin, docetaxel, 5-fluorouracil, deoxyfluorouridine, doxorubicin, epirubicin, epothilone, etoposide, gemcitabine, hydroxyurea, idarubicin, imatinib, irinotecan, nitrogen mustard, Mercaptopurine, methotrexate, mitoxantrone, oxaliplatin, paclitaxel, pemetrexed, teniposide, thioguanine, topotecan, valrubicin, vemurafenib, vinblastine, vincristine, vindesine, vinorelbine and hydroxycamptothecin.

In some embodiments, the chemotherapeutic agent is selected from the group consisting of docetaxel, panobinostat, 5-fluorouracil, paclitaxel, cisplatin, irinotecan, topotecan, and etoposide.

If desired, a therapeutic moiety, such as a radioisotope, a chemotherapeutic agent or any of the therapeutic agents disclosed herein can be conjugated to the glyconucleic acid, such as glycoRNA and glycoDNA. If desired the glyconucleic acid, such as glycoRNA and glycoDNA, can be conjugated to a targeting antibody or antibody fragment. This can provide for enhanced targeting of the glyconucleic acid to a desired cell or organ, and can further stabilize (e.g., increase the serum half-life of) the glyconucleic acid.

The term “chemotherapeutic agent” is a biological (macromolecule) or chemical (small molecule) compound that can be used to treat cancer. The types of chemotherapeutic 184 drugs include, but are not limited to, histone deacetylase inhibitor (HDACI), alkylating agents, antimetabolites, alkaloids, cytotoxic/anti-cancer antibiotics, topoisomerase inhibitors, tubulin inhibitors, proteins, antibodies, kinase inhibitors, and the like.

Chemotherapeutic drugs include compounds for targeted therapy and non-targeted compounds of conventional chemotherapy. Non-limiting examples of chemotherapeutic agents include: erlotinib, afatinib, docetaxel, 5-FU (5-fluorouracil), panobinostat, gemcitabine, cisplatin, carboplatin, paclitaxel, bevacizumab, trastuzumab, pertuzumab, metformin, temozolomide, tamoxifen, doxorubicin, rapamycin, lapatinib, hydroxycamptothecin, trimetinib. Further examples of chemotherapeutic drugs include: oxaliplatin, bortezomib, sunitinib, letrozole, imatinib, PI3K inhibitor, fulvestrant, leucovorin, lonafarnib, sorafenib, gefitinib, crizotinib, irinotecan, topotecan, valrubicin, vemurafenib, telbivinib, capecitabine, vandetanib, chloranmbucil, panitumumab, cetuximab, rituximab, tositumomab, temsirolimus, everolimus, pazopanib, canfosfamide, thiotepa, cyclophosphamide, alkyl sulfonates (e.g., busulfan, improsulfan and piposulfan), ethyleneimine, benzodopa, carboquone, meturedopa, uredopa, methylmelamine, including altretamine, triethylenemelamine, triethyl phosphamide, triethyl thiophosphamide and trimethylenemelamine, bullatacin, bullatacinone, bryostatin, callystatin, CC-1065 (including its adozelesin, carzelesin, bizelesin synthetic analogue), cryptophycin (in particular, cryptophycin 1 and cryptophycin 8), dolastatin, duocarmycin (including synthetic analogues such as KW-2189 and CB1-TM1), eleutherobin; pancratistatin, sarcodictyin, spongistatin, nitrogen mustards (e.g., chlorambucil, chlornaphazine, cyclophosphamide, estramustine, ifosfamide, bis-chloroethyl-methylamine, Mechlorethamine oxide, melphalan, novembichin, phenesterine, prednimustine, trofosfamide, uramustine), nitrosoureas (e.g., carmustine, chlorozotocin, fotemustine, lomustine, nimustine, ranimustine), antibiotics (e.g., enediyne antibiotics (e.g., calicheamicin, calicheamicin γ1I, calicheamicin ωI1, dynemicin, dynemicin A, esperamicin, and neocarzinostatin chromophore and related chromoproteins containing an enediyne antibiotic chromophore), bisphosphonate (e.g., clodronate), aclacinomycin, actinomycin (e.g., actinomycin C, actinomycin D), all-trans retinoic acid, anthramycin, azaserine, bleomycin, carabicin, carminomycin, carzinophilin, chromomycinis, daunorubicin, deoxy-fluorouridine, detorubicin, 6-dizao-5-oxo-L-norleucine, morpholinodoxorubicin, cyno-morpholinodoxorubicin, 2-pyrroline-doxorubicin, epoxy doxorubicin, epirubicin, esorubicin, idarubicin, marcellomycin, mitomycin, mycophenolic acid, nogalamycin, olivomycin, peplomycin, porfiromycin, puromycin, quelamycin, rodorubicin, streptonigrin, streptozocin, tubercidin, ubenimex, zinostatin, zorubicin, antimetabolites (e.g., methotrexate), folate analogues (e.g., dimethylfolate, methotrexate, pteropterin, trimetrexate), purine analogue (e.g., fludarabine, 6-mercaptopurine, methotrexate, thiamiprine, tioguanine), pyrimidine analogues (e.g., ancitabine, azacitidine), azathioprine, bleomycin, 6-nitrouridine, carmofur, cytarabine, dideoxyuridine, doxifluridine, enocitabine, floxuridine, androgen, calusterone, dromostanolone propionate, epitiostanol, mepitiostane, testolactone, antiadrenergic agent, e.g. aminoglutethimide, mitotane, trilostane, folate supplement (e.g., folinate), aceglatone, aldophosphamide glycoside, aminolevulinic acid, eniluracil, amsacrine, bestrabucil, bisantrene, edatraxate, defofamine, demecolcine, diaziquone, eflornithine, elliptinium acetate, epothilone, etoglucid, gallium nitrate, hydroxycarbamide, lentinan, lonidamine, maytansinoid, maytansine, ansamitocin, mitoguazone, mitoxantrone, mopidamol, nitraerine, pentostatin, phenamet, pirarubicin, losoxantrone, podophyllinic acid, 2-ethylhydrazine; procarbazine, PSK® polysaccharide complex (JHS Natural Products, Eugene, Oreg.), razoxane, rhizoxin, sizofiran, spirogermanium, tenuazonic acid, triaziquone, 2,2′,2″-trichloro-triethylamine, trichothecene (in particular, T-2 toxin, verracurin A, roridin A and anguidine); urethane, vindesine, dacarbazine, mannomustine, dibromomannitol, dibromodulcitol, pipobroman, gacytosine, arabinoside (“Ara-C”), cyclophosphamide, thiotepa, tioguanine, 6-mercaptopurine; methotrexate, Vinblastine, etoposide, ifosfamide, mitoxantrone, vincristine, vinorelbine, novantrone, emetrexed, teniposide, edatrexate, daunomycin, aminopterin, ibandronate, CPT-11, topoisomerase inhibitor RFS 2000, DMFO, retinoid (e.g., Retinoic acid), and pharmaceutically acceptable salts or derivatives thereof.

### Target Biology

In various aspects, pharmaceutical compositions produced by the methods of the invention are used as therapies to treat diseases or health conditions. Such diseases or health conditions include but are not limited to autoimmune disease, antiself-antibody-mediated diseases, complement dysregulation-associated diseases, immune complex associated diseases, amyloidoses, diseases associated with infectious agents or pathogens (e.g., bacterial, fungal, viral, parasitic infections), disease associated with toxic proteins, diseases associated with the accumulation of lipids, diseases associated with apoptotic, necrotic, aberrant or oncogenic mammalian cells, metabolic disease and rare congenital conditions.

In various embodiments of the invention, the desired target receptor is selected from an exemplary list in Table 3. In certain aspects, the glyco-ligand binds to at least one of the following receptors: lectins, galactose, DC-SIGN, GLUT transporter, Gp120, SIGN-R-1. In other aspects, the target ranges from macrophage, liver, glioma, inflammation, antitumor immune response.

Additional aspects of the invention contemplate a matrix of glycans as signal molecules to modulate one or more desired receptors in a target host cell to mediate a biological effect. Such glyco-ligands contact or bind directly to a receptor on the target cell. In some instances, the glyco-ligands are internalized in the target cell. Production of various defined matrix of specific glyco-ligand structures can be deployed to interrogate one or more targets to determine receptor binding affinity, avidity, specificity, pharmacokinetic properties (half-life) and subsequent biological effect.

Glyco-ligand structures may also be bound to a receptor and then internalized to express a payload. For instance, as previously demonstrated with a GalNAc-conjugated siRNA molecule (e.g., givosiran), targeted delivery of GalNAc-conjugated siRNA includes liver hepatocytes. In such instances, the tri-GalNAc-conjugated siRNA is bound to asialoglycoprotein receptor (ASGPR), which then proceeds to endocytosis. The GalNAc residues are released or dissociated from ASGPR wherein the glycans are degraded in the lysosome and the ASGPR is recycled to the cell surface. Similarly, in certain embodiments, methods and compositions provide targeted delivery of various glyco-ligands to one or more receptors.

In certain embodiments, once the synthetic scaffold domain, e.g., mRNA, is dissociated in the cytoplasm, mRNA is translated. See Aaron D. Springer and Steven F. Dowdy. Nucleic Acid Therapeutics. June 2018. 109-118.

In preferred embodiments, the glyco-ligands are specific for receptors demonstrating nanomolar or picomolar binding affinity constants for target antigens e.g., 10⁹ M, 10¹⁰ M, 10¹¹ M, 10¹² M, 10¹³ M or tighter). Typical conventional analytical techniques such as surface plasmon resonance (SPR) BIAcore™ instrumentation is used.

The products of the invention, therefore, can be used directly or used with minimal processing for research, diagnostic, therapeutic uses. The glyco-ligands of the invention can be used as reagents in immunoassays, radioimmunoassays (RIA), enzyme-linked immunosorbent assays (ELISA) or protein arrays.

Preferred aspects of applications include mediating cell-cell interaction and/or cell-cell communication through the glyco-ligands.

In various aspects of the invention is provided conserved small noncoding RNAs operably linked to sialylated and/or fucosylated glycans, glycans enriched in sialic acid and/or fucose residues, synthetic glycans displaying terminal sialic acid and/or fucose residues. Additional embodiments include small noncoding RNAs operably linked to at least 20%, at least 30%, at least 40%, at least 50%, at least 60%, at least 70%, at least 80%, at least 90%, at least 95% or greater amount sialylated glycans. Further embodiments include small noncoding RNAs operably linked to at least 20%, at least 30%, at least 40%, at least 50%, at least 60%, at least 70%, at least 80%, at least 90%, at least 95% or greater amount of fucosylated glycans. Yet other embodiments include small noncoding RNAs operably linked to at least at least 20%, at least 30%, at least 40%, at least 50%, at least 60%, at least 70%, at least 80%, at least 90%, at least 95% or greater amount of sialylated and fucosylated glycans.

Certain aspects of the invention provide glyco-ligands that mediate cell-cell interaction or cell-cell communication. In some preferred embodiments, the glyco-ligand of the invention mediates glycosylated RNA, glycosylated lipid and/or glycoproteins linked to a cell surface of a target cell.

### Target Cell Surface Protein

In certain aspects, the glyco-ligand compositions are administered on one or more target cells including without limitation macrophage, monocyte, B cell, Schwann cell, ODC, DC, osteoclasts, MyPro, monocyte, granulocyte, microglia, mast cell, neutrophil, trophoblast, NK cell, T cell, eosinophil, basophil and platelets.

Siglec are a class of receptor molecules expressed on different cell types and amenable for drugging via glyco-ligands. Flynn et al. 2021 show that a glycoRNA can attach to two specific sialic acid-binding immunoglobulin-type lectins (Siglecs), which are associated with a family of immune receptors implicated in several diseases, including systemic lupus erythematosus (SLE), which may suggest involvement in immune signaling. Flynn et al., (2021), Cell 184(12): 3109-3124.

Accordingly, the glyco-ligand compositions are administered to bind to one or more Siglec receptors. Certain glyco-ligand compositions are contemplated to be specific and bioactive to modulate one or more Siglec receptors. See FIG. 4B.

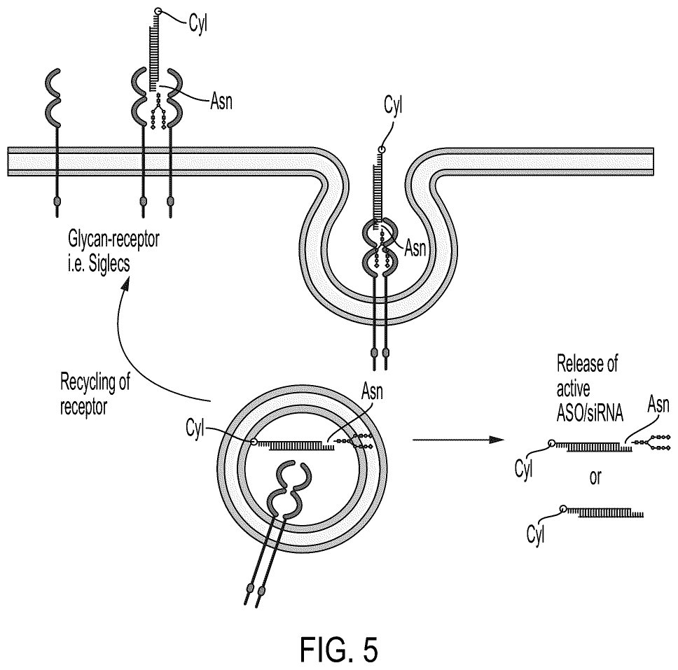

In various aspects, the glyco-ligand compositions are used as a delivery vehicle to deliver a payload. For instance, the glyco-ligand compositions bind to receptors (e.g., CD22), which lead to internalization and function of the nucleic acid. In one embodiment, the glyco-ligand composition binds to a receptor, forming a dimer complex, where the complex is endocytosed, the glyco-ligand is released and activated and the receptor is then recycled. See FIG. 5. In such embodiments, the glyco-ligands include, without limitation, mRNA, siRNAs, ASOs, circRNA. In related embodiments, the glyco-ligand compositions further comprise conjugation to a toxin or a radionucleotide and binds to a receptor on a target cell and kills the target cell. In other embodiments, the glyco-ligand compositions comprise one or more sequences encoding a peptide delivered into a target cell.

### Glyco-Ligand Formulation

The pharmaceutical compositions may be formulated based on the desired route of administration.

There are certain considerations in determining the amount of each component for formulation of the glyco-ligand of the invention. The glyco-ligand compositions may, include single stranded or double stranded RNA, which may be linear or circular. For instance, the synthetic scaffold domain comprising the RNA may be 50% of the pharmaceutical composition of the final product. In some instances, approximately 100% of the RNA is operably linked to one or more glycans. Such glycans may be a singular glycoform or a mixture of one or more glycoforms. Furthermore, the glyco-ligand compositions may include excipients, lyophilized using mannitol, preservative, stabilizer, minimize degradation and precipitation, and readily reconstituted in liquid for subcutaneous administration or intradermal administration via an injectable microneedle.

In addition to many advantages of the invention to ameliorate disease such as cancer, inflammatory conditions and autoimmune diseases, other advantages of the pharmaceutical compositions disclosed herein include improved stability, improved PK/PD, recalcitrant to protease degradation, increased half-life, manageable cold-chain storage and distribution, configurable and programmable, specific orientation leading to clustering of signaling proteins, altered ability of nucleic acids to aggregate with the result of altered biophysical properties, and the ability to functionalize DNA or RNA Origami structures. [Jiang Q, Song C, Nangreave J, Liu X, Lin L, Qiu D, Wang Z G, Zou G, Liang X, Yan H, Ding B. DNA origami as a carrier for circumvention of drug resistance. J Am Chem Soc. 2012 Aug. 15; 134(32):13396-403]. In preferred embodiments, a functionalized DNA or RNA Origami structure improves drug effectiveness.

### EXEMPLARY EMBODIMENTS

The following descriptive embodiments are intended to be illustrative of inventions contemplated herein:

1. A pharmaceutical composition comprising a glyco-ligand, wherein the glyco-ligand comprises one or more glycan moieties operably linked to one or more sites on a synthetic scaffold domain comprising a synthetic ribonucleic acid (RNA) polymer.

2. The pharmaceutical composition of embodiment 1, wherein the synthetic ribonucleic acid polymer comprises one or more RNA polymers selected from mRNA, siRNA, snRNA, snoRNA, dsRNA, miRNA, lncRNA, circular RNA, Y RNA, ribosomal RNA, and small RNA fragments.

3. The pharmaceutical composition of any one of embodiments 1 or 2, wherein the synthetic ribonucleic acid polymer comprises siRNA.

4. The pharmaceutical composition of embodiment 3, wherein the siRNA comprises one or more modifications to one or more nucleotides selected from 2-OMe modification, a fluorine modification, a phosphorothioate modification or any combinations thereof.

5. The pharmaceutical composition of any one of embodiments 1-4, wherein the synthetic scaffold domain comprises one or more soluble RNA.

6. The pharmaceutical composition of any one of embodiment 1-4, wherein the synthetic scaffold domain comprises one or more modified reactive functional groups suitable for glycan conjugation, whereby the one or more glycan moieties are operably linked to the synthetic scaffold domain.

7. The pharmaceutical composition of any one of the preceding embodiments, wherein the glycan moiety comprises a monosaccharide selected from D-Glucuronic Acid (“GlcA”), β-muramic acid (“Mur”), Mannuronic Acid (“ManA”), N-Acetyl-Muramic Acid (“MurNAc”), Legionaminic acid (“Leg”), Acinetaminic acid (“Aci”), D-Xylose (“Xyl”), N-Acetyl-L-Fucosamine (“FucNAc”), Pseudaminic acid (“Pse”) and L-Iduronic Acid (“IdoA”).

8. The pharmaceutical composition of any one of the preceding embodiments, wherein the glycan moiety comprises a hexuronate sugar.

9. The pharmaceutical composition of any one of the preceding embodiments, wherein the glycan moiety comprises a monosaccharide comprising a 9-N-biphenyl carboxamide (BPC) modification.

10. The pharmaceutical composition of any one of the preceding embodiments, wherein the glycan moiety comprises a monosaccharide comprising a non-saccharide substituent or modification selected from:

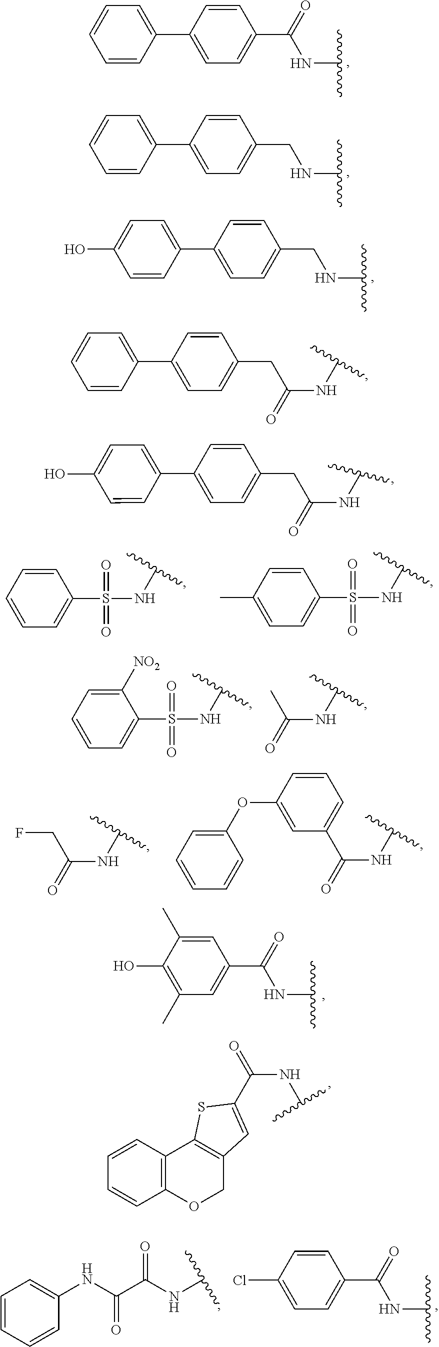

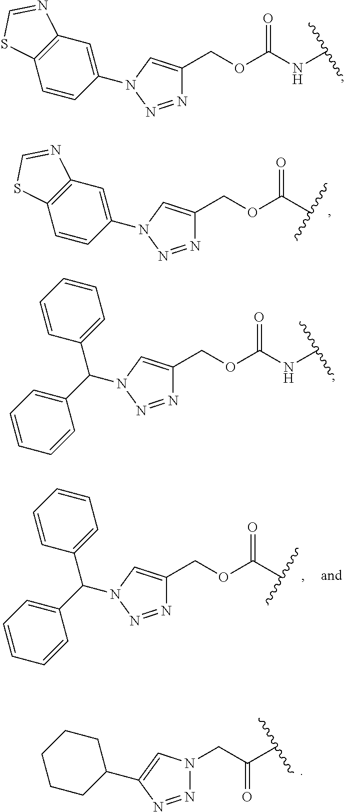

11. The pharmaceutical composition of any one of the preceding embodiments, wherein the glycan moiety comprises a chain of two or more repeating HexNAc-Hexuronate-units.

12. The pharmaceutical composition of any one of the preceding embodiments, wherein the glycan moiety comprises a chain of two or more repeating GlcN-GlcA-units.

13. The pharmaceutical composition of any one of the preceding embodiments, wherein the glycan moiety comprises a chain of two or more repeating GlcN-IdoA-units.

14. The pharmaceutical composition of any one of the preceding embodiments, wherein the glycan moiety comprises a chain of two or more repeating GalNAc-GlcA-units.

15. The pharmaceutical composition of any one of the preceding embodiments, wherein the glycan moiety comprises a chain of two or more repeating Galactose-GlcNAc-units.

16. The pharmaceutical composition of any one of the preceding embodiments, wherein the glycan moiety comprises at least one fucose bound to at least one monosaccharide selected from a GlcNAc, a galactose, and a glucose.

17. The pharmaceutical composition of any one of the preceding embodiments, wherein the glycan moiety comprises a multi-antennary glycan consisting of only mannose.

18. The pharmaceutical composition of any one of the preceding embodiments, wherein the glycan moiety comprises a glycan selected from:
a. those depicted in Table 1A (G-1 through G-39);
b. those depicted in Table 1B (H-1 through H-62);
c. those depicted in Table 1C (J-1 through J-19);
d. those depicted in Table 1D (K-1 through K-53);
e. those depicted in Table 1E (M-1 through M-14); and
f. those depicted in Table 1F.

19. The pharmaceutical composition of any one of the preceding embodiments, wherein the glycan moiety comprises a glycan selected from those depicted in Table 1B (H-1 through H-62), except for H-48 through H-52.

20. The pharmaceutical composition of any one of the preceding embodiments, wherein the glycan moiety comprises a glycan selected from those depicted in Table 1C (J-1 through J-19).

21. The pharmaceutical composition of any one of the preceding embodiments, wherein the glycan moiety comprises a glycan selected from those depicted in Table 1D (K-1 through K-53).

22. The pharmaceutical composition of any one of the preceding embodiments, wherein the glycan moiety comprises a glycan that binds to a plasma membrane lectin selected from MRC1, DC-SIGN, MGL, Siglec 3, Siglec 8, Siglec 9, Siglec 2, Siglec 4a, langerin, Dectin-1, Dectin-2, CLEC14A, CLEC4A, CLEC4C, CLEC5A, CLEC2D, CD2, E selectin, P selectin, L selectin, thrombomodulin, and SRCL.

23. The pharmaceutical composition of any one of the preceding embodiments, wherein the glycan moiety comprises a glycan that binds to a serum lectin selected from MBL, Galectin-2, Anti-α-Gal antibody, Galectin-3, and Galectin-8.

24. The pharmaceutical composition of any one of the preceding embodiments, wherein the glycan moiety comprises a glycan that binds to a protein selected from Siglec-1, Siglec-2, Siglec-3, Siglec-4, Siglec-5, Siglec-6, Siglec-7, Siglec-8, Siglec-9, Siglec-10, Siglec-11, CLEC12A, CLEC4E and CD161.

25. The pharmaceutical composition of any one of the preceding embodiments, wherein the glycan moiety comprises a glycan that binds to a protein selected from CD93, CD83, KLRF1, CD22 and CD28.

26. The pharmaceutical composition of any one of the preceding embodiments, wherein the one or more glycan moieties bind to one or more lectins selected from those disclosed in Table 3.

27. The pharmaceutical composition of any one of the preceding embodiments, wherein the glyco-ligand comprises two or more glycan moieties operably linked to two or more sites on a synthetic scaffold domain comprising a synthetic ribonucleic acid polymer.

28. The pharmaceutical composition of any one of the preceding embodiments, wherein the glyco-ligand is a heteromultivalent glyco-ligand comprising two or more distinct glycan moieties.

29. The pharmaceutical composition of embodiment 22, wherein the glyco-ligand comprises a predominant glycan, wherein the predominant glycan accounts for at least 50%, 60%, 70%, 80%, 90%, or 100% of the glycan moieties operably linked to the synthetic scaffold domain.

30. A method of treating a disease or condition comprising administering to a subject in need thereof a therapeutically effective amount of the pharmaceutical composition of any one of embodiments 1-29.

31. The use of the pharmaceutical composition of any one of embodiments 1-29 for the manufacture of a medicament for the treatment of a disease or a condition.

32. Use of the pharmaceutical composition of any one of embodiments 1-29 for the treatment of a disease or a condition in a subject in need thereof.

### EXAMPLES

The following examples are offered by way of illustration and not by way of limitation.

### Example 1: Glycan Synthesis

Chemoenzymatic glycan synthesis and purification protocol are performed as described in Gao et al., 2019. The sialoglycopeptide (SGP) is prepared from egg yolk following established protocols (Bingyang Sun, Wenzheng Bao, Xiaobo Tian, Mingjing Li, Hong Liu, Jinhua Dong, Wei Huang, A simplified procedure for gram-scale production of sialylglycopeptide (SGP) from egg yolks and subsequent semi-synthesis of Man3GlcNAc oxazoline. Carbohydrate Research, Volume 396, 2014, 62-69; Zou, Yang & Wu, Zhigang & Chen, Leilei & Liu, Xianwei & Gu, Guofeng & Xue, Mengyang & Wang, Peng & Chen, Min. (2012). An Efficient Approach for Large-Scale Production of Sialyglycopeptides from Egg Yolks. J Carbohyd. Chem. 31. 436-446) with small modifications. Briefly, egg yolk powder (Magic Flavors, purchased directly from Amazon.com, Inc.) is weighed and suspended in 3 volumes diethyl ether and washed twice. After filtration, the residue is resuspended in 3 volumes of 70% acetone and washed. The SGP is then extracted using 1.5 volumes of 40% acetone. After drying on rotary evaporator, the SGP-containing crude extract is purified using an active charcoal column (active charcoal: celite 2:1). The column is preconditioned using 3 bed volumes of acetonitrile followed by 3 bed volumes (BV) of water containing 0.1% trifluoroacetic acid (TFA). After sample loading, the column is sequentially washed with H₂O with 0.1% TFA, 5% acetonitrile with 0.1% TFA and 10% acetonitrile with 0.1% TFA, each 3 BV. The SGP is eluted by 3 BV 25% acetonitrile and the obtained fractions are combined and concentrated on rotary evaporator and lyophilized to dryness. This SGP-containing powder can be directly used in the following reactions without further purification. In embodiments, the powder can be desalted by size exclusion chromatography on Bio-Gel P2 gel (Bio-Rad Laboratories, Inc.) and the product SGP is ready for structural analysis by nuclear magnetic resonance (NMR) spectroscopy or mass spectrometry (MS).

### Preparation of the Fmoc-Labelled Asialo-Agalacto-Biantennary N-Glycan

The SGP is subjected to the following treatments to generate the substrate (G0-Fmoc) for enzymatic synthesis. The SGP powder is reconstituted in water and hydrochloric acid (HCl) is added. The final concentrations of SGP and HCl are adjusted to 20 mg/ml and 0.1 M, respectively. After incubation at 80° C. for 2 hours, the solution is neutralized by sodium hydroxide (NaOH) and the desialylated SGP (Man5-AEAB) is produced. Song et al., (2009). Novel fluorescent glycan microarray strategy reveals ligands for galectins. Chem. Biol. 16, 36-47. This solution is adjusted to pH 5.2 by addition of sodium acetate and concentrated acetic acid. Galactosidase is then added to a final concentration of 10 mg/ml and incubated at 37° C. for 4 hours. After heating at 85° C. for 10 minutes, the desialylated and degalactosylated SGP (G2-AEAB) is subjected to pronase digestion by addition of 200 mM tris base to adjust the pH to 8.0 and pronase to a final concentration of 1 mg/ml. The mixture is incubated at 55° C. and every 12 hours the same amount of pronase is added until no starting material was detected by matrix assisted laser desorption ionization mass spectrometry (MALDI-MS). After centrifugation, the supernatant is lyophilized to dryness and the residue is reconstituted in water, passed by Sep-Pak C18 solid-phase extraction (SPE) column (Waters Corporation) and purified by size exclusion chromatography on a Bio-Gel P2 gel (Bio-Rad Laboratories, Inc.) column. The Asn-linked asialo-, agalacto-biantennary N-glycan (G0) is obtained. This compound is labelled with fluorenylmethoxycarbonyl protecting group (Fmoc) by reacting with Fmoc-OSu (i.e., Fmoc-O-succinimidyl carbonate) (3 equivalents) in 1,4-dioxane: H₂O=1:2 overnight and the product (G0-N-Fmoc) is eventually purified on a preconditioned Sep-Pak C18 column (Waters Corporation).

Six glycosyltransferases, FUT8, MGAT4a, MGAT5, B4GalT1, ST3Gal4 and ST6Gal1, are expressed using suitable expression plasmids (e.g., plasmids available from Professor Kelley Moremen at the Complex Carbohydrate Research Center, University of Georgia). The constructs contain the soluble domain of the glycosyltransferase with an N-terminal poly-histidine (His) and green fluorescent protein (GFP) tags after a secretion signal (pGEn2-DEST vector). Suspension and serum free adapted HEK293 cells (Freestyle 293-F cells, Invitrogen, Thermo Fisher Scientific Inc.) are transiently transfected using polyethyleneimine. 195 Five to seven days after transfection, protein is purified from the cultural supernatant by nickel affinity chromatography with HisPur nickel nitrilotriacetic acid (Ni-NTA) resin (Thermo Fisher Scientific Inc.). After elution with imidazole containing buffer (50 mM sodium phosphate, 300 mM sodium chloride, and 400 mM Imidazole, pH 8.0), the enzymes are dialyzed against storage buffer (20 mM Tris, pH 7.5 with 300 mM sodium chloride) and flash frozen. All of the glycosyltransferases, as chimeric GFP-fusion proteins were stored at −80° C. until use.

### Glycosyltransferase Reactions

A) α1,6-Core Fucosylation Catalyzed by FUT8 (2 mg/ml)

The reaction is performed in 100 mM 2-morpholin-4-ylethanesulfonic acid (MES) buffer, pH 7.0. The final concentrations of glycans and guanosine 5′-diphospho-β-L-fucose (GDP-Fuc) are 2.5 mM and 3.75 mM, respectively. The glycosyltransferase FUT8 concentration is 1 mg/ml. The reaction is incubated at 37° C. overnight before being stopped by freezing at −80° C. The mixture is lyophilized to dryness and purified on a preconditioned Sep-Pak C18 column (Waters Corporation) by elution with an increasing amount of methanol (MeOH) from 0 to 50%. Fractions that are orcinol-positive were evaluated by MALDI-MS and those containing the predicted m z are combined and dried to harvest the targeted glycans.

B) β1,4-GlcNAc Branching Catalyzed by MGAT4a (1 mg/ml)

The reaction is performed in 500 mM 3-(morpholin-4-yl) propane-1-sulfonic acid (MOPS) buffer, pH 7.3 with 30 mM MnCl2. The final concentrations of glycans and uridine diphosphate N-acetylglucosamine (UDP-GlcNAc) are 5 mM and 10 mM, respectively. The glycosyltransferase MGAT4a is at 0.25 mg/ml. Phosphatase is also included in the mixture. The reaction is incubated at 37° C. and monitored by MALDI-MS. Typically the reaction is allowed to proceed overnight before being stopped by cooling to −80° C., after which the solution is lyophilized to dryness. The product is purified on a preconditioned Sep-Pak C18 column (Waters Corporation) by elution with an increasing amount of MeOH from 0 to 50%. Fractions that are orcinol-positive were checked by MALDI-MS and those contain the predicted m z were combined and dried to harvest the targeted glycans.

C) β1,6-GlcNAc Branching Catalyzed by MGAT5 (1 mg/ml)

The reaction is performed in 125 mM MES buffer, pH 6.25. The final concentrations of glycans and uridine diphosphate N-acetylglucosamine (UDP-GlcNAc) are 5 mM and 10 mM, respectively. The glycosyltransferase MGAT5 concentration is at 0.25 mg/ml. Phosphatase is also 196 included in the mixture to digest the product uridine diphosphate (UDP). The reaction is incubated at 37° C. overnight before being stopped by putting at −80° C. The mixture is lyophilized to dryness and purified on a preconditioned Sep-Pak C18 column (Waters Corporation) by elution with increased amount of MeOH from 0 to 50%. Fractions that are orcinol-positive were checked by MALDI-MS and those contain the predicted m z are combined and dried to harvest the targeted glycans.

D) β1,4-Galactosylation Catalyzed by B4GalT1 (2 mg/ml)

The reaction is performed in 125 mM Tris buffer, pH 7.5, with 100 mM NaCl, 50 mM MgCl2, 50 mM MnCl2. The final concentrations of glycans are at 5 mM. The concentration of uridine diphosphate galactose (UDP-Gal) varies from 15 mM for triantennary to 20 mM for tetraantennary N-glycans. B4GalT1 is added to a final concentration of 0.3 mg/ml. The products of MGAT4a and MGAT5 can also be directly elongated by B4GalT1, in which case, Tris base, NaCl, MgCl2 and MnCl2 are added to the reaction mixture to a final concentration of 125, 100, 50 and 50 mM, respectively. Hydrochloric acid is added to adjust pH to 7.5. The final concentrations of glycans, UDP-Gal and B4GalT1 were at 1.2, 7.3 mM (or 9.6 mM for tetra-antennary) and 0.47 mg/ml, respectively. In all cases, phosphatase is included. The reaction is incubated at 37° C. overnight and stopped by putting at −80° C. The mixture is lyophilized to dryness and purified on a preconditioned Sep-Pak C18 by elution with increased amount of MeOH from 0 to 50%. Fractions that are orcinol-positive are checked by MALDI-MS and those contain the predicted m z are combined and dried to harvest the targeted glycans.

E) 2,3-Sialylation Catalyzed by ST3Gal4 (1 mg/ml)

The reaction is performed in 100 mM cacodylate-Na buffer, pH 6.2, which also contained 50 mM MnCl2. The final concentration of glycans is adjusted to 2.5 mM. The cytidine monophosphate (CMP)-sialic acid is at 15, 22.5 and 30 mM for bi-, tri- and tetra-antennary N-glycans, respectively, with the ST3Gal4 at 0.3, 0.4 and 0.5 mg/ml, respectively. Phosphatase is also included in the mixture to digest the product CMP. The reaction is incubated at 37° C. overnight before being stopped by putting at −80° C. The mixture is lyophilized to dryness and the product was purified by HPLC on a Zorbax NH₂ column (250× 10 mm, Agilent Technologies, Inc.) as mentioned below. Fractions with the predicted m z are combined and dried to harvest the targeted glycans.

F) 2,6-Sialylation Catalyzed by ST6Gal1 (2 mg/ml)

The conditions for 2,6-sialylation are identical to 2,3-sialylation with the exception of the amount of the glycosyltransferase added. The ST6Gal1 is adjusted to 0.6, 0.8 and 1 mg/ml for bi-, tri- and tetra-antennary N-glycans, respectively.

### Example 2: RNA Synthesis

RNA is prepared by a standard T7 RNAP run-off transcription reaction using PCR product as a template and purified by urea-PAGE as described. The RNA yield from in vitro transcription is optimized for each individual DNA template in 25 μL trial reactions by varying the concentration of Mg2+, nucleoside 5′-triphosphates (NTPs) and incubation time. A typical large-scale 10 mL transcription reaction mixture contains 30 mM Tris (pH 8.1 at 37° C.), 15 mM Mg2+, 10 mM dithiothreitol (DTT), 2 mM spermidine, 0.01% (v/v) Triton X-100, 4 mM of each NTP, 1 mL of PCR-generated DNA template, and 0.1 mg/mL of T7 RNAP [6, 8]. After 2.5 hours of incubation at 37° C., pyrophosphate in the reaction mixture, which forms during in vitro transcription reaction, is pelleted down by centrifugation, and additional Mg2+ is added to the reaction. The reaction continues until 5 hours. The reaction mixture is concentrated using Millipore centrifugal filter units with appropriate molecular weight cut-off (MWCO). The transcription reaction screening is composed of one variable component at a time with the rest of components fixed. The tested Mg2+ concentrations include 5 mM, 15 mM, 25 mM, 35 mM, 45 mM, 55 mM, 65 mM, 75 mM, 85 mM and 95 mM while NTPs concentrations are 1 mM, 2 mM, 3 mM, 4 mM, 5 mM, 6 mM, 7 mM, 8 mM, 9 mM and 10 mM. The incubation time for transcription at 37° C. is tested at 5 hours, 6 hours, 7 hours, 8 hours, 9 hours and 10 hours. The results are evaluated through image quantification (BioRad) of the target RNA band on 12% TAE urea-PAGE. The target RNA yield peaks at 180% of stock conditions, i.e. around 45 mM Mg2+. In general, the RNA yield increases from 1 mM up to 10 mM NTP, plateauing around 8 mM each. Lastly, the reaction time is extended and a steady increase of product is observed until 9 hours. [Lu C et al., Cell Physiol. Biochem., 2018; 48: 1915-1927].

In vivo RNA synthesis is carried out in BL21 (DE3) E. coli cells using a DNA-containing plasmid with a template corresponding to the mRNA of interest. IPTG is used to induce transcription when the cell culture has a UV absorbance around 0.5 optical density (OD) at 600 nm, and the solution is shaken for 3 hours at 37° C. As described in Mao and Wang et al., after induction, 1 ml bacteria culture solution is centrifuged in a 1.5 ml centrifuge tube and the suspension is removed. The pellet is resuspended in 100 μL Buffer L, containing 10 mM Tris-HCl (pH 7.4) and 10 mM Mg(OAc)₂. The bacterial membrane is destroyed by adding 100 μL phenol solution (Sigma). The aqueous layer is removed by pipette and directly deposited into a native PAGE gel. To prepare samples for denaturing PAGE gel or gel purification, 10 μL NaOAc (3 M, pH 5.2) and ethanol (200 μL) is added to aqueous layer (100 μL), followed by an ethanol participation in dry ice to remove salts. The cell pellet is re-suspended in 15 ml Buffer L and put in an ice-water bath. Cells are then lysed by sonication (without phenol) with Branson Digital Sonifier (10% amplitude), sonicating for 5 seconds and stopping for 5 seconds; and repeating for a total of 10 minutes. lysates (1 ml) can be centrifuged at 16,000×g for 30 minutes to remove the cell debris and the upper layer can be diluted with tris-acetate-ethylenediaminetetraacetic acid (TAE)/Mg2+ buffer. [Li, M., Zheng, M., Wu, S. et al. In vivo production of RNA nanostructures via programmed folding of single-stranded RNAs. Nat Commun 9, 2196 (2018)].

Circular RNA is prepared as described in Wesselhoeft et al. Nat Commun 9, 2629 (2018). The first step is cloning and mutagenesis, in which protein coding, group I self-splicing intron, and IRES sequences are chemically synthesized (Integrated DNA Technologies) and cloned into a PCR-linearized plasmid vector containing a T7 RNA polymerase promoter by Gibson assembly using a NEBuilder HiFi DNA Assembly kit (New England Biolabs). Spacer regions, homology arms, and other minor alterations are introduced using a Q5 Site Directed Mutagenesis Kit (New England Biolabs). This is followed by circRNA design and purification. RNA structure is predicted using RNAFold18. Modified linear Gaussia luciferase (GLuc) mRNA is obtained from Trilink Biotechnologies and consists of a codon optimized Gaussia luciferase (GLuc) coding region, a proprietary synthetic 5′ untranslated region, an alpha globin 3′ untranslated region, a cap 1 structure, a 120-nucleotide poly A tail, and complete replacement of uridine and cytosine along the entire mRNA with pseudouridine and 5-methylcytosine, respectively. Modified human erythropoietin (hEpo) mRNA is also obtained from Trilink Biotechnologies and is structurally identical to the Trilink Gaussia luciferase (GLuc) mRNA described above, except that it is modified with 5-methoxyuridine and the coding region codes for human erythropoietin. Unmodified linear RNA consists of a GLuc or hEpo coding region but does not include specific untranslated regions. Unmodified linear mRNA or circRNA precursors are synthesized by in-vitro transcription from a linearized plasmid DNA template using a T7 High Yield RNA Synthesis Kit (New England Biolabs). After in vitro transcription, reactions are 199 treated with DNase I (New England Biolabs) for 20 minutes. After DNase treatment, unmodified linear mRNA is column purified using a MEGAclear Transcription Clean-up kit (Ambion®, Thermo Fisher Scientific Inc.). RNA is then heated to 70° C. for 5 minutes and immediately placed on ice for 3 minutes, after which the RNA is capped using mRNA cap-2′-O-methyltransferase (NEB) and Vaccinia capping enzyme (New England Biolabs) according to the manufacturer's instructions. Polyadenosine tails are added to capped linear transcripts using E. coli PolyA Polymerase (New England Biolabs) according to manufacturer's instructions, and fully processed mRNA is column purified. For circRNA, after DNase treatment additional guanosine triphosphate (GTP) is added to a final concentration of 2 mM, and then reactions are heated at 55° C. for 15 minutes. RNA is then column purified. In some cases, purified RNA is recircularized: RNA is heated to 70° C. for 5 minutes and then immediately placed on ice for 3 minutes, after which GTP is added to a final concentration of 2 mM along with a buffer containing magnesium (50 mM Tris-HCl, 10 mM MgCl2, 1 mM DTT, pH 7.5; New England Biolabs). RNA is then heated to 55° C. for 8 minutes, and then column purified. To enrich for circRNA, 20 μg of RNA is diluted in water (86 μL final volume) and then heated at 65° C. for 3 minutes and cooled on ice for 3 minutes. RNase R (20U) and 10 μL of 10× RNase R buffer (Epicenter) is added, and the reaction is incubated at 37° C. for 15 minutes; an additional 10U RNase R is added halfway through the reaction. RNase R-digested RNA is column purified. RNA is separated on precast 2% E-gel EX agarose gels (Invitrogen, Thermo Fisher Scientific, Inc.) on the E-gel iBase (Invitrogen, Thermo Fisher Scientific, Inc.) using the E-gel EX 1-2% program; ssRNA Ladder (New England Biolabs) is used as a standard. Bands are visualized using blue light transillumination and quantified using ImageJ software. For gel extractions, bands corresponding to the circRNA are excised from the gel and then extracted using a Zymoclean Gel RNA Extraction Kit (Zymo Research Corp.). For high-performance liquid chromatography, 30 μg of RNA is heated at 65° C. for 3 minutes and then placed on ice for 3 minutes. RNA is run through a 4.6×300 mm size-exclusion column with particle size of 5 μm and pore size of 200 Å (Sepax Technologies, Inc.; part number: 215980P-4630) on an Agilent 1100 Series HPLC (Agilent Technologies, Inc.). RNA is run in RNase-free TE buffer (10 mM Tris, 1 mM EDTA, pH: 6) at a flow rate of 0.3 mL/minute. RNA is detected by UV absorbance at 260 nm, but is collected without UV detection. Resulting RNA fractions are precipitated with 5 M ammonium acetate, resuspended in water, and then optionally treated with RNase R as described above. [Wesselhoeft, R. A., Kowalski, P. S. & Anderson, D. G. Engineering circular RNA for potent and stable translation in eukaryotic cells. Nat Commun 9, 2629 (2018)].

RNA modifications can be introduced to reduce cellular response (e.g., to decrease a cellular immune response). As described in Kariko K et al., an in vitro transcription reaction can be assembled with the replacement of one (or two) of the conventional NTPs with the corresponding triphosphate-derivative(s) of the modified nucleotide 5-methylcytidine, 5-methyluridine, 2-thiouridine, N6-methyladenosine, or pseudouridine (TriLink, San Diego, CA) to generate an RNA with modifications to reduce the cellular response. For such transcription reactions, all four nucleotides or their derivatives are present in equimolar (7.5 mM) concentration. In addition, 6 mM m7GpppG cap analog (New England BioLabs, Beverly, MA) can be included to obtain capped RNA. Kariko K et al. Immunity 23:16575 (2005). Replacement or uridine with pseudouridine, in particular, favors the suppression of RNA immunogenicity in vitro and in vivo and also enhances the translational capacity of RNA. Kariko K et al. Mol Ther 16:1833-40 (2008). The reason for the decreased immunogenicity and enhanced translational capacity of RNA modified with pseudouridine is that uridine activates RNA-dependent protein kinase R (PKR), which then phosphorylates translation initiation factor 2-alpha (eIF-2α), and inhibits translation. When pseudouridine is incorporated into the transcript, PKR is activated to a lesser degree and translation is not inhibited. Anderson B R et al. NAR (2010).

RNA can be purified by native gel purification or column purified using a MEGAclear Transcription Clean-up kit (Ambion). Mao and Wang et al., Nat Commun 9, 2196 (2018 and Wesselhoeft et al., Nat Commun 9, 2629 (2018).

RNA modifications for glycan conjugation include the use of 5-substituted pyrimidines as well as 7-substituted 7-deazapurines bearing diyne groups with terminal triple bonds, such as 3′ 5-octadiynyl dU, during RNA synthesis. Seela F, Sirivolu V R. DNA containing side chains with terminal triple bonds: Base-pair stability and functionalization of alkynylated pyrimidines and 7-deazapurines. Chem Biodivers. 2006 May; 3(5): 509-14.

### Example 3: Glyco-Ligand Conjugation

As described in Meng, G., Guo, T., Ma, T. et al., a diazotizing species, fluorosulfuryl azide (FSO₂N₃), can be used in a click chemistry reaction to generate an azide from the terminal amine of a glycan. [Meng, G., Guo, T., Ma, T. et al. Modular click chemistry libraries for functional screens using a diazotizing reagent. Nature 574, 86-89 (2019)]. A glycan with an azide group can react with an alkyne, e.g., on a nucleic acid, to produce a covalent bond. See conjugation schema in FIG. 3.

Flynn et al.'s recent work showed labeling of the precursor glycosyl with an azide group (for example, Ac4ManNAz used in this study). Once the precursor glycosyl moieties are integrated into the glycoprotein (and lipid) group in the cell, they can be combined with biotin. (Flynn et al., (2021), Cell 184(12): 3109-3124). The probes are cross-linked to be enriched and subjected to subsequent identification analysis. With the help of such a system, the author has enriched high-purity RNA samples in the labeled cells, which indicates that glycosylation may also exist on RNA.

As disclosed in U.S. Pat. No. 10,550,385 B2, a GalNAc-siRNA conjugate can be produced through a process for introducing two or more 2′-modifications into an RNA, wherein the RNA has a 2′-O substituent containing an alkyl ester functional group at the 2′-position on one or more ribose rings of a strand and a 2′-O substituent containing an alkyne functional group at the 2′-position on one or more ribose rings on the same strand, comprising: a) adding an amine compound to the RNA to form amidation reaction products with the alkyl ester functional groups; b) dissolving the modified RNA from step (a) in a solvent to form a solution; and c) adding an organic azide and a copper or ruthenium catalyst to the solution obtained in step (b) to form 2′-azide-alkyne cycloaddition reaction products with the alkyne functional groups. When the organic azide is GalNAc azide, a GalNAc-siRNA is produced.

### Example 4: Glyco-Ligand Verification

### Release of N-Linked Glycans

The glycans are released and separated from glyco-ligands or glycoproteins by a modification of a previously reported method (Papac, et al. A. J. S. (1998) Glycobiology 8, 445-454). The wells of a 96-well MultiScreen IP (Immobilon-P membrane) plate (Millipore) are wetted with 100 uL of methanol, washed with 3×150 uL of water and 50 uL of reduction and carboxymethylation (RCM) buffer (8M urea, 360 mM Tris, 3.2 mM EDTA pH 8.6), draining with gentle vacuum after each addition. The dried protein samples are dissolved in 30 uL of RCM buffer and transferred to the wells containing 10 uL of RCM buffer. The wells are drained and washed twice with RCM buffer. The proteins are reduced by addition of 60 uL of 0.1M DTT in RCM buffer for 1 hour at 37° C. The wells are washed three times with 300 uL of water and carboxymethylated by addition of 60 uL of 0.1 M iodoacetic acid for 30 minutes in the dark at room temperature. The wells are again washed three times with water and the membranes blocked by the addition of 100 uL of 1% polyvinylpyrrolidone with an average molecular weight of 360,000 Daltons (PVP 360) in water for 1 hour at room temperature. The wells are drained and washed three times with 300 uL of water and deglycosylated by the addition of 30 uL of 10 mM NH₄HCO₃ pH 8.3 containing one milliunit of N-glycanase (Glyko). After 16 hours at 37° C., the solution containing the glycans was removed by centrifugation and evaporated to dryness.

### Matrix Assisted Laser Desorption Ionization Time of Flight Mass Spectrometry

Molecular weights of the glycans are determined using a Voyager DE PRO linear MALDI-TOF (Applied Biosciences Corp.) mass spectrometer using delayed extraction. The dried glycans from each well are dissolved in 15 uL of water and 0.5 uL spotted on stainless steel sample plates and mixed with 0.5 uL of S-DHB matrix (9 mg/mL of dihydroxybenzoic acid, 1 mg/mL of 5-methoxysalicilic acid in 1:1 water/acetonitrile 0.1% TFA) and allowed to dry.

Ions are generated by irradiation with a pulsed nitrogen laser (337 nm) with a 4 nanosecond pulse time. The instrument is operated in the delayed extraction mode with a 125 nanosecond delay and an accelerating voltage of 20 kV. The grid voltage can be 93.00%, guide wire voltage can be 0.10%, the internal pressure can be less than 5×10-7 torr, and the low mass gate can be 875 Daltons. Spectra are generated from the sum of 100-200 laser pulses and acquired with a 2 GHz digitizer. Sialylated complex N-glycan NeuNAc₂Gal₂GlcNAc₂Man₃GlcNAc₂Fuc is used as an external molecular weight standard. All spectra are generated with the instrument in the positive ion mode. The estimated mass accuracy of the spectra can be about 0.5%.

The mass of the N-glycans eluted from the column is generally associated with a positive ion adduct, which increases the mass by the molecular weight of the positive ion. The most common adducts are H⁺, Na⁺ and K⁺.

### Glycan Preparation for NMR

As described in EP1910838B1, experimental procedures to conduct proton NMR analysis of glycan fractions are detailed below. Glycans are liberated from glyco-ligand by enzymatic or chemical means. Glycans are then fractionated into neutral and acidic glycan fractions by chromatography on a graphitized carbon. A useful purification step prior to NMR analysis is gel filtration high-performance liquid chromatography (HPLC). For glycans of glycoprotein or glycolipid origin, a Superdex Peptide HR10/300 column (Amersham Pharmacia) may be used. For larger glycans, chromatography on a Superdex 75 HR10/300 column may be used. Superdex columns are eluted at a flow rate of 1 ml per minute with water or with 50-200 mM ammonium bicarbonate for the neutral and acidic glycan fractions, respectively, and absorbance at 205-214 nm is recorded. Fractions are collected (typically 0.5-1 ml) and dried. Repeated dissolving in water and evaporation may be necessary to remove residual ammonium bicarbonate salts in the fractions. The fractions can be subjected to MALDI-TOF-MS and all fractions containing glycans are pooled. The pooled fractions are dissolved in deuterium oxide and evaporated. With glycan preparations containing about 100 nmol or more material, the sample is finally dissolved in 600 microliters of high-quality deuterium oxide (99.9-99.996%) and transferred to an NMR analysis tube. A roughly equimolar amount of an internal standard, e.g., acetone, is commonly added to the solution. With glycan preparations derived from small tissue specimens or from a small number of cells (5-25 million cells), the sample is preferably evaporated from very high-quality deuterium oxide (99.996%) twice or more to eliminate H₂O as efficiently as possible, and then finally dissolved in 99.996% deuterium oxide. These low-material samples are preferably analyzed by more sensitive NMR techniques. For example, NMR analysis tubes of smaller volumes can be used to obtain higher concentration of glycans. Suitable tubes of smaller volume include e.g., nanotubes (Varian Inc.) in which sample is typically dissolved in a volume of 37 microliters. In embodiments, higher sensitivity is achieved by analyzing the sample in a cryo-NMR instrument, which increases the analysis sensitivity through low electronic noise. The latter techniques allow gathering of high quality proton-NMR data from glycan samples containing about 1-5 nmol of glycan material.

### Analysis of NMR Data

It is realized that numerous studies have shown that proton-NMR data has the ability to indicate the presence of several structural features in glycan samples. In addition, by careful integration of the spectra, the relative abundancies of these structural features in the glycan sample can be obtained. For example, the proton bound to monosaccharide carbon-1, i.e., H-1, yields a distinctive signal at the lower field, well separated from the other protons of sugar residues. Most monosaccharide residues e.g., in N-glycans are identified by their H-1 signals. In addition, the H-2 signals of mannose residues are indicative of their linkages.

Sialic acids do not possess a H-1, but their H-3 signals (H-3 axial and H-3 equatorial) reside well separated from other protons of sugar residues. Moreover, differently bound sialic acids may be identified by their H-3 signals. For example, the Neu5Ac H-3 signals of Neu5Acα2-3Gal structure are found at 1.797 ppm (axial) and 2.756 ppm (equatorial). On the other hand, the Neu5Ac H-3 signals of Neu5 Acα2-6Gal structure are found at 1.719 ppm (axial) and 2.668 ppm (equatorial). By comparing the integrated areas of these signals, the molar ratio of these structural features is obtained.

Other structural reporter signals are commonly known and those familiar with the art use the extensive literature for reference in glycan NMR assignments. Fu D., Chen L. and O'Neill R. A. (1994) Carbohydr. Res. 261. 173-186. Hård K., Mekking A., Kamerling J. P., Dacremont G. A. A and Vliegenthart J. F. G (1991) Glycoconjugate J 8, 17-28. Hård K., Van Zadelhoff G., Moonen P., Kamerling J. P. and Vliegenthart J. F. G. (1992) Eur. J. Biochem. 209, 895-915. Helin J., Maaheimo H., Seppo A., Keane A. and Renkonen O. (1995) Carbohydr. Res. 266, 191-209

### Example 5: Assays to Determine how Carbohydrate Binding Receptors Interact with their Glycan Ligands

Surface plasmon resonance spectroscopy (SPR spectroscopy) can be used to determine kinetic binding parameters between the selected glyco-ligand, e.g., G2FS2 glyco-ligand, and a target receptor. To obtain the kinetic binding parameters, the receptors or proteins, e.g., Siglec 11 and Siglec 14, are immobilized on the surface of a sensor chip. The glyco-ligand is carried in a flow of buffer solution through a miniature flow cell. Binding of the glyco-ligand to an immobilized receptor or protein on the surface of the sensor chip leads to a change in refractive index at the surface layer and is monitored by a detector such as a diode array. Time-dependent changes in the refractive index are recorded as sensorgrams. The sensorgrams provide information about binding or non-binding as well as providing information about the kinetics and the strength of the interaction.

### Example 6: General Procedure for Synthesis of Azido Glycans

### Materials and Methods

Free reducing end glycans are obtained from Glycobia, Inc., Ithaca, NY, or Chemily Glycoscience, Peachtree Corners, GA and are made according to literature procedures known in the art. 205

### General Characterization Procedures

Matrix assisted laser desorption ionization (MALDI) analysis of glycan conjugates: Glycan conjugates (1 μL, 1 mM) in mini-Q water were mixed with a 2,5-Dihydroxybenzoic acid (DHB, 1 μL, 20 mg/mL) matrix in 50% (v/v) acetonitrile aqueous solution. Then the mixtures were loaded onto MTP 384 target plate. Following air drying and co-crystallization, samples were analyzed by Bruker MALDI-TOF system.

HPLC analysis of glycan conjugates: Glycan conjugates (0.2 mg/mL, 50 μL) in ethanol were analyzed by analytical reverse-phase HPLC column. Mobile phase A is methanol with 0.1% TFA and mobile phase B is water with 0.1% TFA. Flow rate is 1 mL/minute. Gradient: 75% to 100% mobile phase A.

### Asparagine Azide Functionalization

To a solution of asparagine-linked N-glycan in mini-Q water, Na₂CO₃ (20 equivalents) and FSO₂N₃ (40 equivalents) are added. The mixture is rotated at room temperature for 1 hour, and MALDI mass analysis showed complete conversion. The reaction mixture is placed under vacuum centrifugation for 30 minutes, then is lyophilized. The residue (white powder) is reconstituted in mini-Q water, then is loaded onto preconditioned Carb SPE tube. The tube is washed with distilled water (10×1.2 mL), then eluted with 50% acetonitrile with 100 mM (NH₄)₂CO₃ (4×1.2 mL). The eluent is combined and lyophilized to give the desired azido glycan.

TABLE 4A
Exemplified Asparagine Azide functionalized glycans

| Ref # | Modified Glycan | Yield | MS |
|---|---|---|---|
| A-1 | 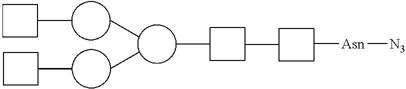 | 91% | 1495.156 [M + K]⁺ |
| | G-1 + Asparagine Azide | | |
| A-2 | 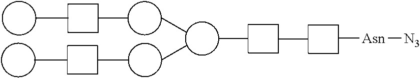 | 89% | 1819.304 [M + K]+ |
| | G-2 + Asparagine Azide | | |
| A-3 | 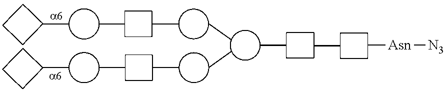 | 88% | 2515.293 [M + 4K − 3H]⁺ |
| | G-3 + Asparagine Azide | | |
| A-4 | 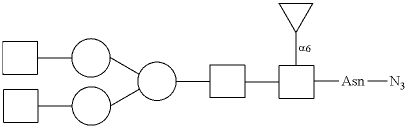 | 90% | 1641.453 [M + K]⁺ |
| | G-4 + Asparagine Azide | | |
| A-5 | 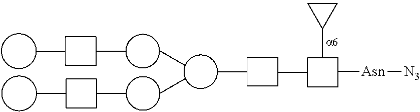 | 88% | 1965.601 [M + K]⁺ |
| | G-5 + Asparagine Azide | | |
| A-6 | 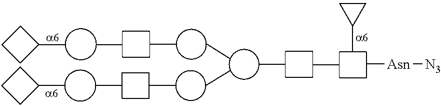 | 87% | 2661.363 [M + 4K − 3H]⁺ |
| | G-6 + Asparagine Azide | | |
| A-7 | 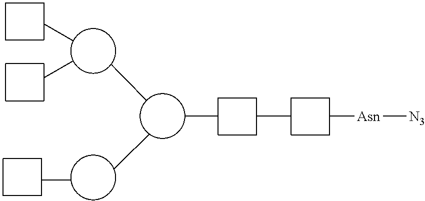 | 90% | 1736.60 [M + 2K − H]⁺ |
| | G-27 + Asparagine Azide | | |
| A-8 | 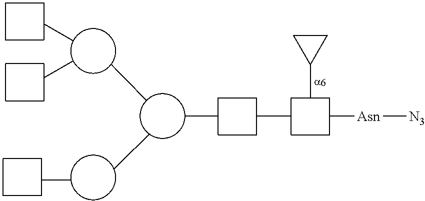 | 88% | 1882.66 [M + 2K − H]⁺ |
| | G-28 + Asparagine Azide | | |
| A-9 | 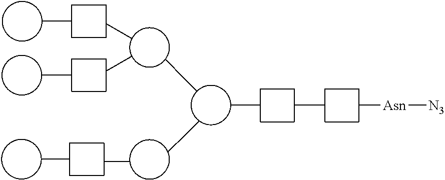 | 89% | 2222.76 [M + 2K − H]⁺ |
| | G-29 + Asparagine Azide | | |
| A-10 | 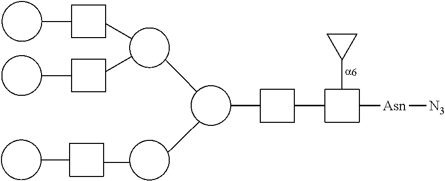 | 87% | 2368.82 [M + 2K − H]⁺ |
| | G-30 + Asparagine Azide | | |
| A-11 | 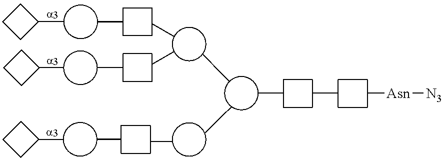 | 82% | 3018.044 [M − H]⁻ |
| | G-33 + Asparagine Azide | | |
| A-12 | 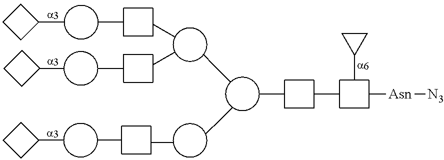 | 81% | 3164.102 [M − H]⁻ |
| | G-34 + Asparagine Azide | | |
| A-13 | 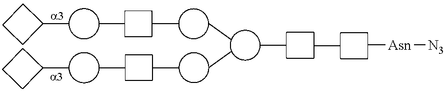 | 87% | 2515.293 [M + 4K − 3H]⁺ |
| | G-25 + Asparagine Azide | | |
| A-14 | 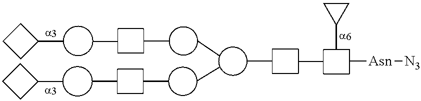 | 87% | 2661.363 [M + 4K − 3H]⁺ |
| | G-26 + Asparagine Azide | | |
| A-15 | 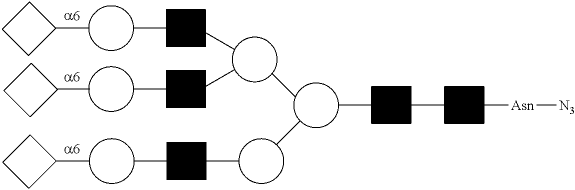 | 82% | 3018.044 [M − H]⁻ |
| | G-31 + Asparagine Azide | | |
| A-16 | 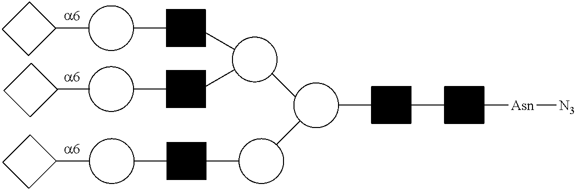 | 82% | 3164.102 [M − H]⁻ |
| | G-32 + Asparagine Azide | | |

### Aminooxy-PEG3-Azide Addition

Glycans having free reducing ends are incubated with a 10-fold molar excess of aminooxy-PEG3-azide linker O-(2-(2-(2-(2-azidoethoxy) ethoxy) ethoxy)ethyl) hydroxylamine; 234.26 g/mol molecular weight). Reactions are performed in 1× phosphate-buffered saline (PBS), pH 4.0 at 37° C. for 30 hours. Reactions are desalted using porous graphitic carbon (PGC) SPE columns (Thermo Fisher Scientific®). The column is preconditioned with 3×1 mL acetonitrile followed by 3×1 mL H₂O. Reaction mixtures are diluted up to 500 μL with water and passed through the column. After reaction mixture loading, the column is washed with 3×1 mL H₂O, then is eluted with 2×750 μL of 10 mM NH₄HCO₃ in 50/50 acetonitrile and H₂O. The acetonitrile is removed under vacuum and the product residue dried by lyophilization.

TABLE 4B
Exemplified Aminooxy-PEG3-azide functionalized glycans

| Ref # | Modified Glycan | Yield | MS |
|---|---|---|---|
| P-1 | 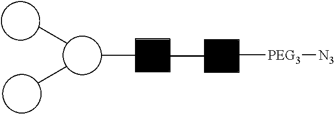 | 83% | 1149.8 [M + Na]⁺ |
| | G-7 + aminooxy-PEG3-azide | | |
| P-2 | 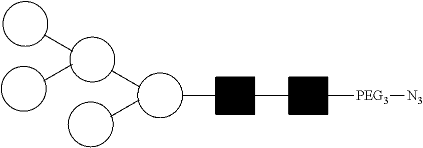 | 85% | 1473.9 [M + Na]⁺ |
| | G-8 + aminooxy-PEG3-azide | | |
| P-3 | 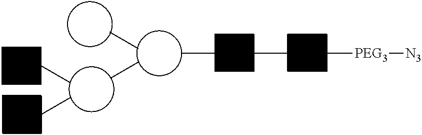 | 81% | 1555.8 [M + Na]⁺ |
| | G-9 + aminooxy-PEG3-azide | | |
| P-4 | 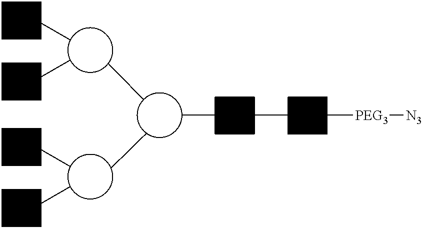 | 77% | 1961.9 [M + Na]⁺ |
| | G-10 + aminooxy-PEG3-azide | | |
| P-5 | 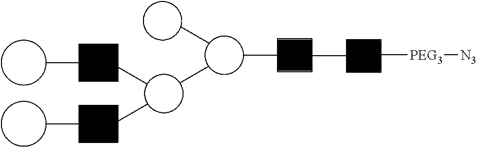 | 82% | 1879.9 [M + Na]⁺ |
| | G-11 + aminooxy-PEG3-azide | | |
| P-6 | 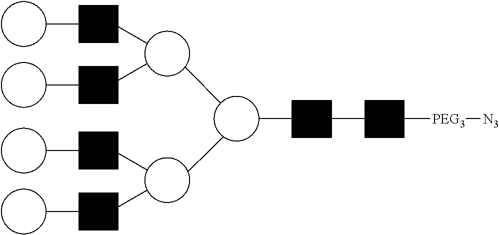 | 74% | 2610.2 [M + Na]⁺ |
| | G-12 + aminooxy-PEG3-azide | | |
| P-7 | 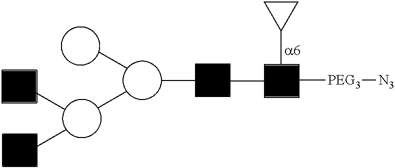 | 86% | 1701.8 [M + Na]⁺ |
| | G-13 + aminooxy-PEG3-azide | | |
| P-8 | 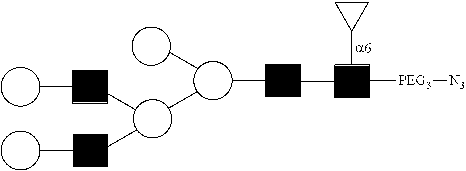 | 81% | 2026.0 [M + Na]⁺ |
| | G-14 + aminooxy-PEG3-azide | | |
| P-9 | 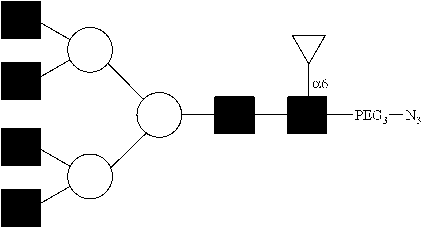 | 78% | 2108.5 [M + Na]⁺ |
| | G-15 + aminooxy-PEG3-azide | | |
| P-10 | 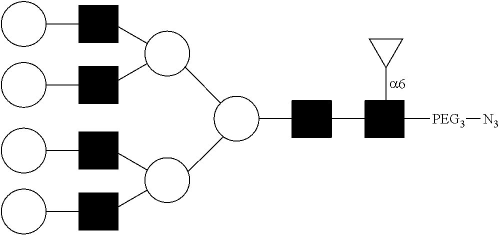 | 72% | 2757.9 [M + Na]⁺ |
| | G-16 + aminooxy-PEG3-azide | | |
| P-11 | 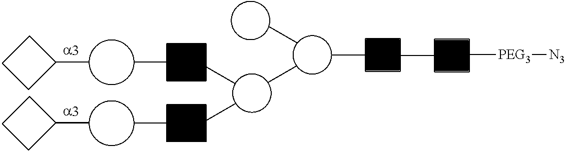 | 70% | 2438.8 [M − H]⁻ |
| | G-17 + aminooxy-PEG3-azide | | |
| P-12 | 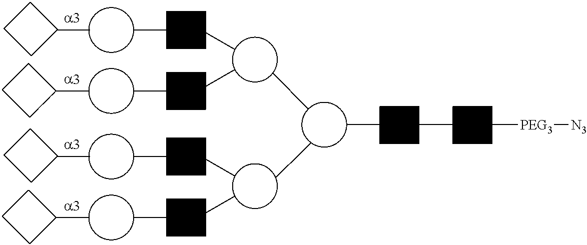 | 65% | 3750.1 [M − H]⁻ |
| | G-18 + aminooxy-PEG3-azide | | |
| P-13 | 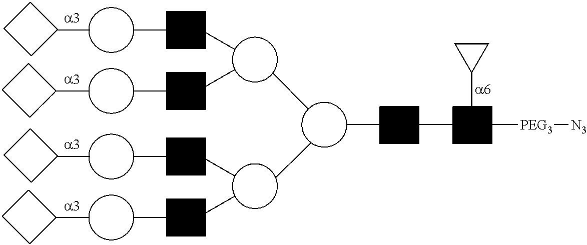 | 63% | 3897.3 [M − H]⁻ |
| | G-19 + aminooxy-PEG3-azide | | |
| P-14 | 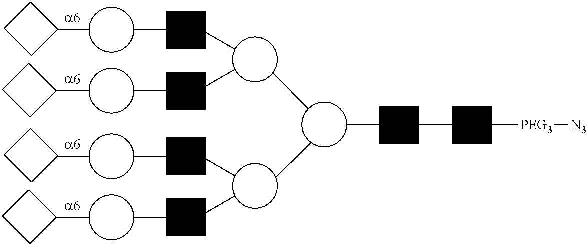 | 66% | 3752.4 [M − H]⁻ |
| | G-20 + aminooxy-PEG3-azide | | |
| P-15 | 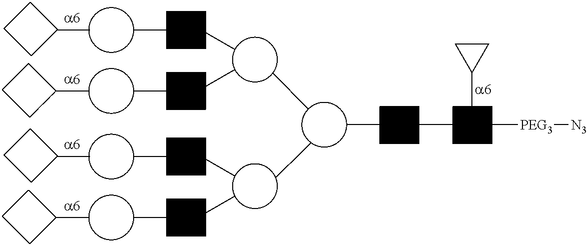 | 65% | 3897.5 [M − H]⁻ |
| | G-21 + aminooxy-PEG3-azide | | |
| P-16 | 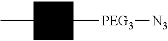 | 70% | 3095.3 [M − H]⁻ |
| | G-22 + aminooxy-PEG3-azide | | |
| P-17 | G-23 + aminooxy-PEG3-azide | 81% | 1960.2 |
| | | | [M + Na]⁺ |
| P-18 | | 80% | 2122.2 |
| | G-24 + aminooxy-PEG3-azide | | [M + Na]⁺ |
| P-19 | 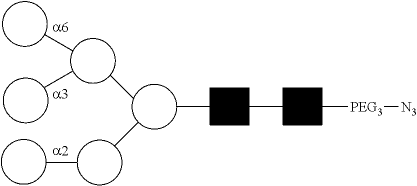 | 82% | [M + Na]⁺ |
| | H-65 + aminooxy-PEG3-azide | | |

### N-Methyloxyamine-Ethyl-Azide Linker Addition

Glycans having free reducing ends were incubated with a 100-fold molar excess of N-methyloxyamine-ethyl-amine dihydrochloride linker 2-((Methylamino)oxy)ethanamine dihydrochloride (163.05 mol wt) and 100-fold molar excess of anhydrous sodium acetate. Reactions were performed in DMSO/HOAc, (7/3, v/v) at 65° C. for 2 hours. Reactions were quenched by adding 15 volumes of acetonitrile, then centrifuged to remove supernatant and precipitates were purified by Envi-Carb SPE columns (Sigma-Aldrich®). The column was preconditioned with 3×1 mL acetonitrile followed by 3×1 mL H₂O. The precipitates were reconstituted in 300 μL water and passed through the column. After loading of precipitates, the column was washed with 5×1 mL H₂O, then was eluted with 2×750 μL of 10 mM NH₄HCO₃ in 50/50 acetonitrile and H₂O. The acetonitrile was removed under vacuum and dried by lyophilization.

To a solution of the labelled glycans described above in mini-Q water, Na₂CO₃ (20 equivalents) and FSO₂N₃ (40 equivalents) were added. The mixture was rotated at room temperature for 1 hour, and MALDI mass analysis showed complete conversion. The reaction mixture was placed under vacuum centrifugation for 30 minutes, then was lyophilized. The residue (white powder) was reconstituted in mini-Q water, then was loaded onto preconditioned Carb SPE tube. The tube was washed with distilled water (3×1 mL), then eluted with 50% acetonitrile with 10 mM NH₄HCO₃ (2×0.75 mL). The eluent was combined and lyophilized to give the desired azido glycan.

TABLE 4C
Exemplified N-methyloxyamine-ethyl-azide functionalized glycans

| Ref # | Modified Glycan | Yield | MS |
|---|---|---|---|
| N-1 | 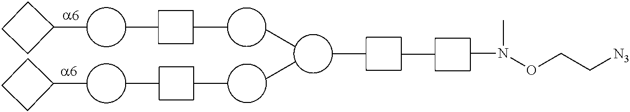 | 72% | 2319.84 [M − H]⁻ |
| | G-3 + N-methyloxyamine-ethyl-azide | | |
| N-2 | 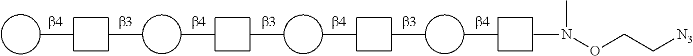 | 76% | 1599.60 [M + Na]⁺ |
| | H-13 + N-methyloxyamine-ethyl-azide | | |
| N-3 | 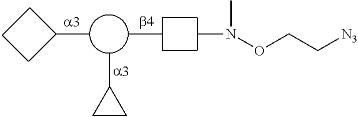 | 80% | 963.36 [M +2 Na − H]⁺ |
| | H-23 + N-methyloxyamine-ethyl-azide | | |
| N-4 | 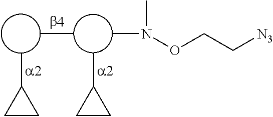 | 81% | 755.29 [M + Na]⁺ |
| | H-3 + N-methyloxyamine-ethyl-azide | | |
| N-5 | 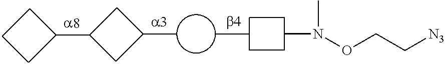 | 67% | 1062.39 [M − H]⁻ |
| | H-7 + N-methyloxyamine-ethyl-azide | | |
| N-6 | 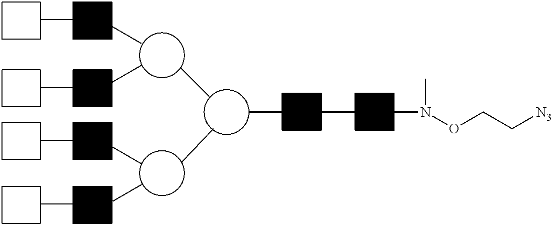 | 63% | 2656.02 [M + Na]⁺ |
| | G-37 + N-methyloxyamine-ethyl-azide | | |
| N-7 | 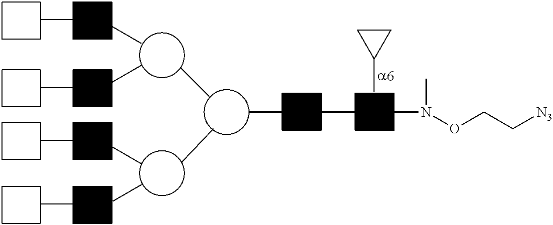 | 61% | 2802.08 [M + Na]⁺ |
| | G-38 + N-methyloxyamine-ethyl-azide | | |
| N-8 | 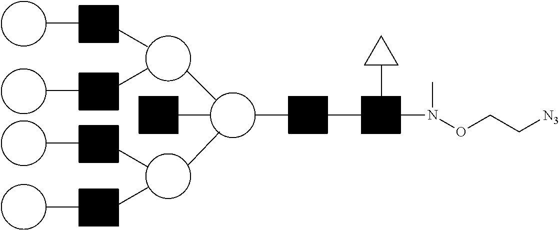 | 60% | 2841.05 [M + Na]⁺ |
| | J-2 + N-methyloxyamine-ethyl-azide | | |
| N-9 | 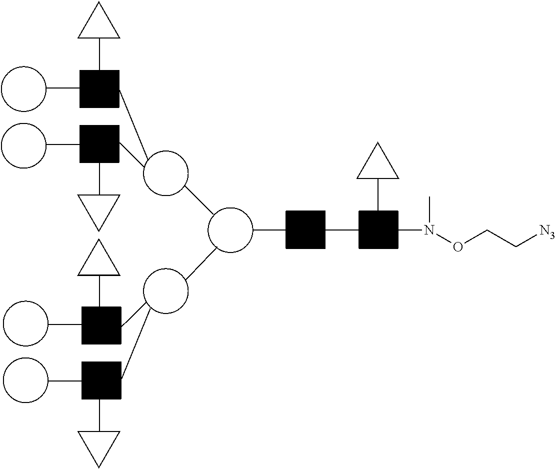 | 57% | 3222.21 [M + Na]⁺ |
| | J-4 + N-methyloxyamine-ethyl-azide | | |
| N-10 | 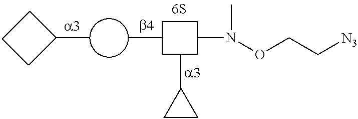 | 68% | 997.36 [M − H]⁻ |
| | H-9 + N-methyloxyamine-ethyl-azide | | |
| N-11 | 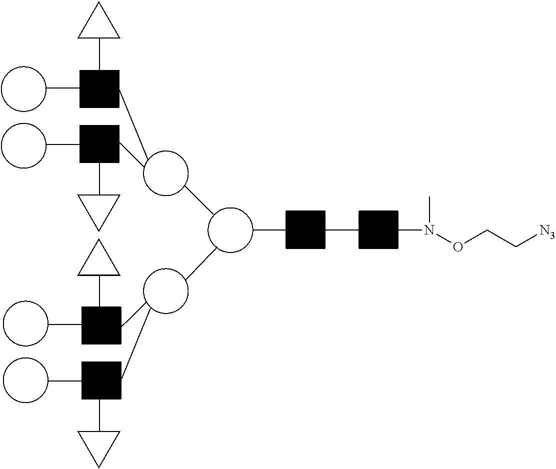 | 55% | 3076.15 [M + Na]⁺ |
| | J-3 + N-methyloxyamine-ethyl-azide | | |
| N-12 | 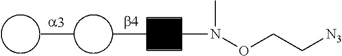 | 84% | 666.25 [M + Na]⁺ |
| | H-12 + N-methyloxyamine-ethyl-azide | | |
| N-13 | 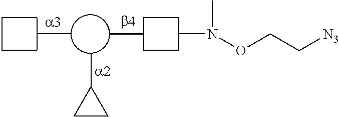 | 78% | 853.34 [M + Na]⁺ |
| | H-11 + N-methyloxyamine-ethyl-azide | | |
| N-14 | 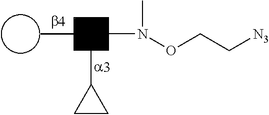 | 77% | 650.26 [M + Na]⁺ |
| | H-24 + N-methyloxyamine-ethyl-azide | | |
| N-15 | 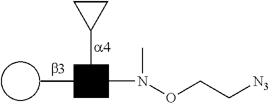 | 78% | 650.26 [M + Na]⁺ |
| | H-28 + N-methyloxyamine-ethyl-azide | | |
| N-16 | 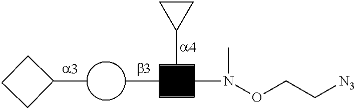 | 72% | 963.36 [M +2 Na − H]⁺ |
| | H-29 + N-methyloxyamine-ethyl-azide | | |
| N-17 | 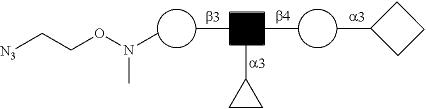 | 71% | 1125.67 [M +2 Na − H]⁺ |
| | H-64 + N-methyloxyamine-ethyl-azide | | |
| N-18 | 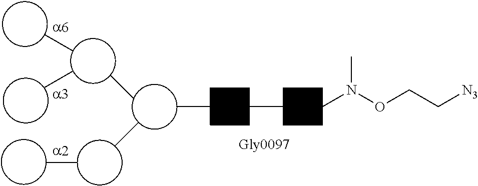 | 80% | 1535.78 [M + Na]⁺ |
| | H-65 + N-methyloxyamine-ethyl-amine | | |

### O-Ethyl-Azide Linker Addition

To a solution of O-ethyl amine labelled glycan in mini-Q water, Na₂CO₃ (20 equivalents) and FSO₂N₃ (40 equivalents) were added. The mixture was stirred at room temperature for 1 hour, and MALDI mass analysis showed complete conversion. The reaction mixture was placed under vacuum centrifugation for 30 minutes, then was lyophilized. The residue (white powder) was reconstituted in mini-Q water, then was loaded onto a preconditioned Carb SPE tube. The tube was washed with distilled water (3×1 mL), then eluted with 50% acetonitrile with 10 mM NH₄HCO₃ (2×0.75 mL). The eluent was combined and lyophilized to give the desired azido glycan.

TABLE 4D
Exemplified O-ethyl-azide functionalized glycans

| Ref # | Modified Glycan | Yield | MS |
|---|---|---|---|
| O-1 | 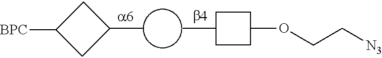 | 93% | 967.34 [M + 2 Na − H]⁺ |
| | H-33 + O-ethyl-azide | | |
| O-2 | 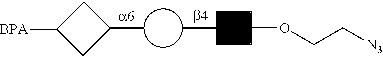 | 91% | [M + 2 Na − H]⁺ |
| | M-1 + O-ethyl-azide | | |

### N-Methyloxyamine-PEG3-Azide Linker Addition

Glycans having free reducing ends were incubated with a 100-fold molar excess of N-methyloxyamine-PEG3-azide and 100-fold molar excess of anhydrous sodium acetate. Reactions were performed in DMSO/HOAc, (7/3, v/v) at 65° C. for 2 hours. Reactions were quenched by adding 15 volumes of ethyl acetate, then centrifuged to remove supernatant and precipitates were purified by Envi-Carb SPE columns (Sigma-Aldrich®). The column was preconditioned with 3×1 mL acetonitrile followed by 3×1 mL H₂O. The precipitates were reconstituted in 300 μL water and passed through the column. After loading of precipitates, the column was washed with 5×1 mL H₂O, then was eluted with 4×750 μL of 10 mM NH₄HCO₃ in 50/50 acetonitrile and H₂O. The acetonitrile was removed under vacuum and dried by lyophilization to give product.

TABLE 4E
Exemplified N-methyloxyamine-PEG3-azide functionalized glycans

| Ref # | Modified Glycan | Yield | MS |
|---|---|---|---|
| Q-1 | 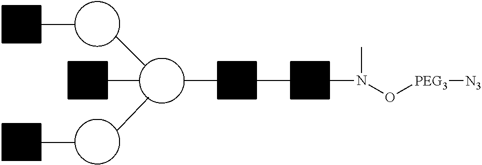 | 83% | 1728.89 [M + Na]⁺ |
| | M-2 + N-methyloxyamine-PEG3-azide | | |
| Q-2 | 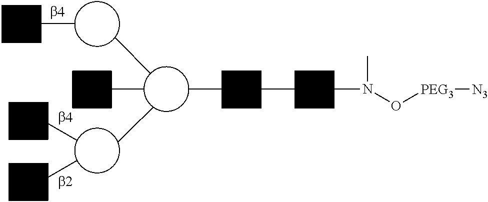 | 80% | 1932.12 [M + Na]⁺ |
| | M-3 + N-methyloxyamine-PEG3-azide | | |
| Q-3 | 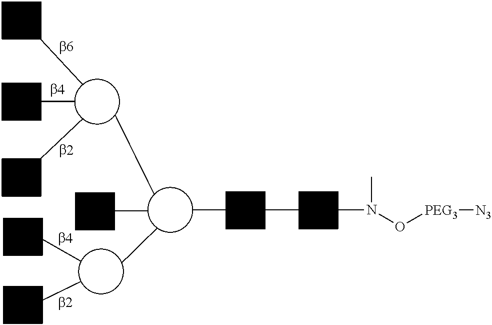 | 74% | 2338.23 [M + Na]⁺ |
| | M-4 + N-methyloxyamine-PEG3-azide | | |
| Q-4 | 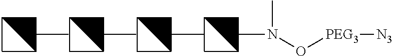 | 56% | 871.77 [M + Na]⁺ |
| | M-5 + N-methyloxyamine-PEG3-azide | | |
| Q-5 | 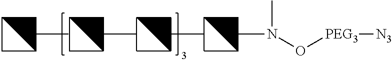 | | |
| | M-6 + N-methyloxyamine-PEG3-azide | | |
| Q-6 | 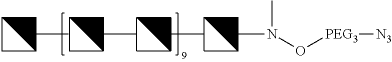 | | |
| | M-7 + N-methyloxyamine-PEG3-azide | | |
| Q-7 | 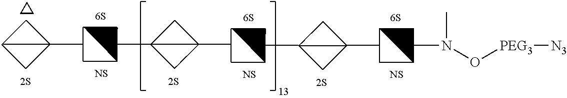 | | |
| | M-8 + N-methyloxyamine-PEG3-azide | | |
| Q-8 | 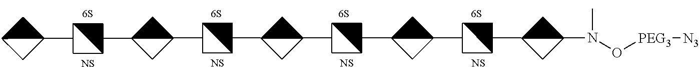 | | |
| | M-9 + N-methyloxyamine-PEG3-azide | | |
| Q-9 | 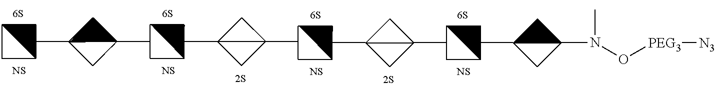 | | |
| | M-10 + N-methyloxyamine-PEG3-azide | | |
| Q-10 | 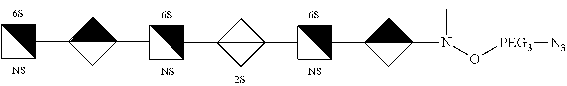 | | |
| | M-11 + N-methyloxyamine-PEG3-azide | | |
| Q-11 | 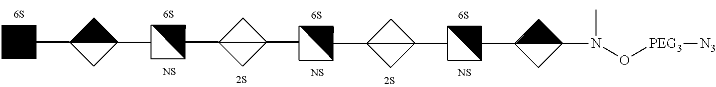 | | |
| | M-12 + N-methyloxyamine-PEG3-azide | | |

TABLE 4F
Exemplified O-phenyl-azide functionalized glycans

| Ref # | Modified Glycan | MS |
|---|---|---|
| R-1 | 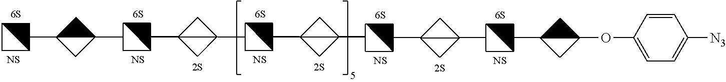 | 588.08 [M − 10H⁺ NH4]⁹⁻ |
| | M-13 + N-methyloxyamine-PEG3-azide | |
| R-2 | 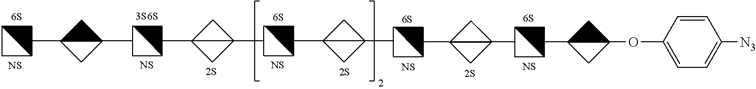 | 521.46 [M − 7H]⁷⁻ |
| | M-14 + N-methyloxyamine-PEG3-azide | |
| R-3 | 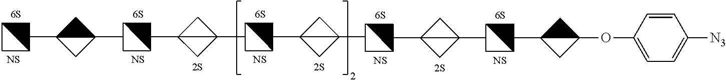 | 442.21 [M − 8H]⁸⁻ |
| | M-15 + N-methyloxyamine-PEG3-azide | |
| R-4 | 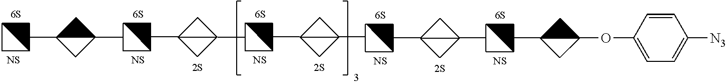 | 515.17 [M − 8H]⁸⁻ |
| | M-16 + N-methyloxyamine-PEG3-azide | |
| R-5 | 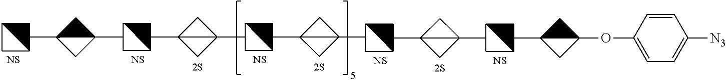 | 506.08 [M − 9H]⁹⁻ |
| | M-17 + N-methyloxyamine-PEG3-azide | |

### N-Methyloxyamine-Butanoic Acid Linker Addition

Glycans having free reducing ends were incubated with a 100-fold molar excess of N-methyloxyamine-butanoic acid and 100-fold molar excess of anhydrous sodium acetate. Reactions were performed in DMSO/HOAc, (7/3, v/v) at 65° C. for 2 hours. Reactions were quenched by adding 15 volumes of ethyl acetate, then centrifuged to remove supernatant and precipitates were purified by Envi-Carb SPE columns (Sigma-Aldrich®). The column was preconditioned with 3×1 mL acetonitrile followed by 3×1 mL H₂O. The precipitates were reconstituted in 300 μL water and passed through the column. After loading of precipitates, the column was washed with 5×1 mL H₂O, then was eluted with 2×750 μL of 10 mM NH₄HCO₃ in 50/50 acetonitrile and H₂O. The acetonitrile was removed under vacuum and dried by lyophilization to give the product N-methyloxyamine-ethyl-amine functionalized glycan.

TABLE 4G
Exemplified N-methyloxyamine-ethyl-amine functionalized glycans

| Ref # | Modified Glycan | MS |
|---|---|---|
| T-1 | 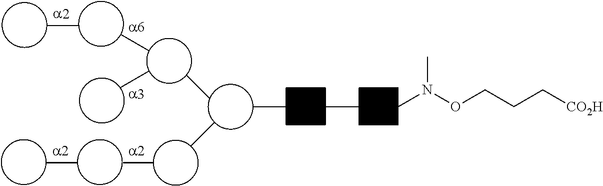 | |
| | G-23 + N-methyloxyamine-butanoic acid | |
| T-2 | 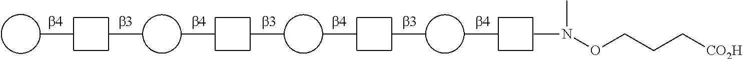 | |
| | H-13 + N-methyloxyamine-butanoic acid | |
| T-3 | 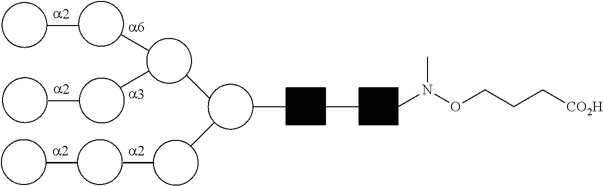 | |
| | G-24 + N-methyloxyamine-butanoic acid | |
| T-4 | 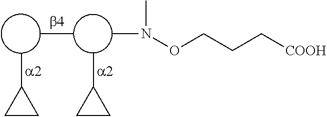 | |
| | H-3 + N-methyloxyamine-butanoic acid | |
| T-5 | 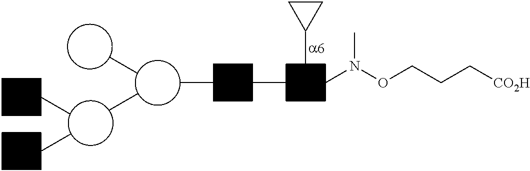 | |
| | G13 + N-methyloxyamine-butanoic acid | |
| T-6 | 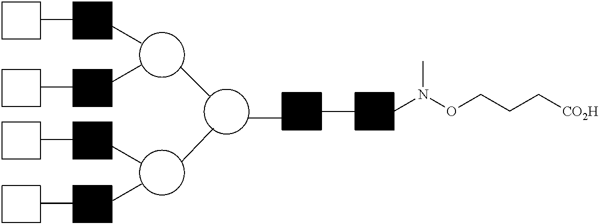 | |
| | G-37 + N-methyloxyamine-butanoic acid | |
| T-7 | 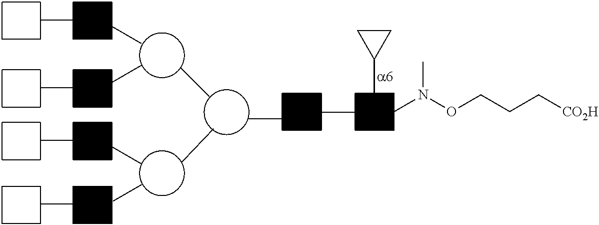 | |
| | G-38 + N-methyloxyamine-butanoic acid | |
| T-8 | 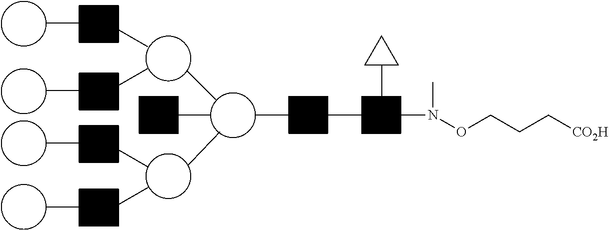 | |
| | J-2 + N-methyloxyamine-butanoic acid | |
| T-9 | 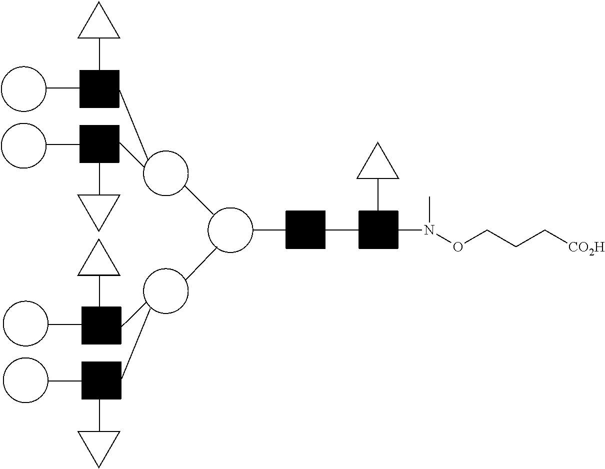 | |
| | J-4 + N-methyloxyamine-butanoic acid | |
| T-10 | 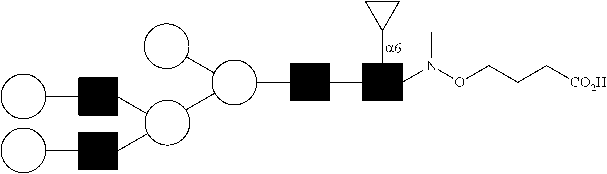 | |
| | G-14 + N-methyloxyamine-butanoic acid | |
| T-11 | 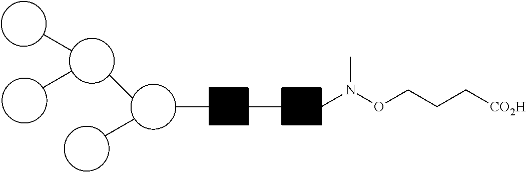 | |
| | G-8 + N-methyloxyamine-butanoic acid | |
| T-12 | 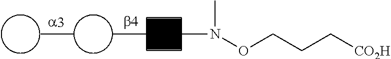 | |
| | H-12 + N-methyloxyamine-butanoic acid | |
| T-13 | 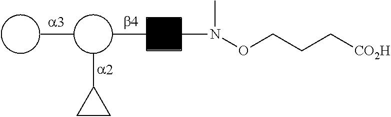 | |
| | H-11 + N-methyloxyamine-butanoic acid | |
| T-14 | 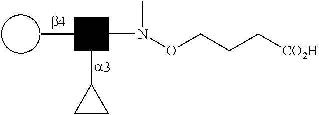 | |
| | H-24 + N-methyloxyamine-butanoic acid | |
| T-15 | 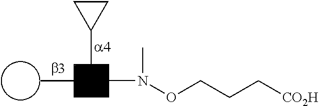 | |
| | H-28 + N-methyloxyamine-butanoic acid | |
| T-16 | 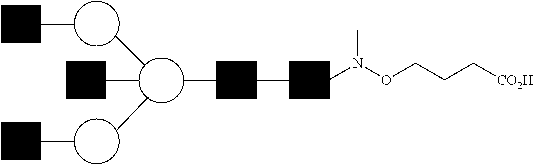 | |
| | M-2 + N-methyloxyamine-butanoic acid | |
| T-17 | 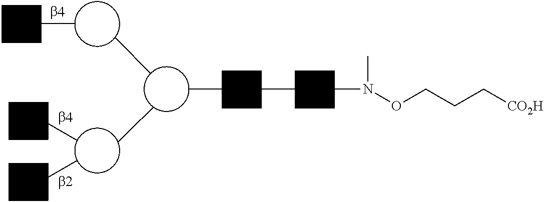 | |
| | M-3 + N-methyloxyamine-butanoic acid | |

### Example 7: General Procedure for Click-Chemistry Coupling of Azido Glycans and Modified siRNAs

siRNAs

The modified nucleic acids described in Table 5A can comprise an optional base modification, an optional sugar modification and/or an optional phosphate modification. In Table 5A, the term “pos.” refers to the nucleic acid position.

TABLE 5A
Exemplary Nucleic Acids

| | | SEQ | | Optional | |
|---|---|---|---|---|---|
| | | ID | Optional Base | Sugar | Optional Phosphate |
| Ref# | Sequence | NO | Modification | Modification | Modification |
| I-1 | UUUCGA | 1 | None | Pos. 1: 2-OMe | Pos. 1: Phosphorothioate |
| | AUCAAU | | | Ribose | linkage |
| | CCAACA | | | Pos. 2: 2- | Pos. 2: Phosphorothioate |
| | GUAGC | | | Fluororibose | linkage |
| | | | | Pos. 3: 2-OMe | Pos. 3: Phosphate (standard) |
| | | | | Ribose | Pos. 4: Phosphate (standard) |
| | | | | Pos. 4: 2- | Pos. 5: Phosphate (standard) |
| | | | | Fluororibose | Pos. 6: Phosphate (standard) |
| | | | | Pos. 5: 2-OMe | Pos. 7: Phosphate (standard) |
| | | | | Ribose | Pos. 8: Phosphate (standard) |
| | | | | Pos. 6: 2- | Pos. 9: Phosphate (standard) |
| | | | | Fluororibose | Pos. 10: Phosphate |
| | | | | Pos. 7: 2-OMe | (standard) |
| | | | | Ribose | Pos. 11: Phosphate |
| | | | | Pos. 8: 2- | (standard) |
| | | | | Fluororibose | Pos. 12: Phosphate |
| | | | | Pos. 9: 2-OMe | (standard) |
| | | | | Ribose | Pos. 13: Phosphate |
| | | | | Pos. 10: 2- | (standard) |
| | | | | Fluororibose | Pos. 14: Phosphate |
| | | | | Pos. 11: 2-OMe | (standard) |
| | | | | Ribose | Pos. 15: Phosphate |
| | | | | Pos. 12: 2-OMe | (standard) |
| | | | | Ribose | Pos. 16: Phosphate |
| | | | | Pos. 13: 2-OMe | (standard) |
| | | | | Ribose | Pos. 17: Phosphate |
| | | | | Pos. 14: 2- | (standard) |
| | | | | Fluororibose | Pos. 18: Phosphate |
| | | | | Pos. 15: 2-OMe | (standard) |
| | | | | Ribose | Pos. 19: Phosphate |
| | | | | Pos. 16: 2- | (standard) |
| | | | | Fluororibose | Pos. 20: Phosphate |
| | | | | Pos. 17: 2-OMe | (standard) |
| | | | | Ribose | Pos. 21: Phosphorothioate |
| | | | | Pos. 18: 2- | linkage |
| | | | | Fluororibose | Pos. 22: Phosphorothioate |
| | | | | Pos. 19: 2-OMe | linkage |
| | | | | Ribose | Pos. 23: Phosphate |
| | | | | Pos. 20: 2- | (standard) |
| | | | | Fluororibose | |
| | | | | Pos. 21: 2-OMe | |
| | | | | Ribose: | |
| | | | | Pos. 22: 2-OMe | |
| | | | | Ribose: | |
| | | | | Pos. 23: 2-OMe | |
| | | | | Ribose | |
| I-2 | UACUGU | 2 | 5′: Cy5 | Pos. 1: 2- | Pos. 1: Phosphorothioate |
| | UGGAUU | | 3′ DBCO | Fluororibose | linkage |
| | GAUUCG | | | Pos. 2: 2-OMe | Pos. 2: Phosphorothioate |
| | AAA | | | Ribose | linkage |
| | | | | Pos. 3: 2- | Pos. 3: Phosphate (standard) |
| | | | | Fluororibose | Pos. 4: Phosphate (standard) |
| | | | | Pos. 4: 2-OMe | Pos. 5: Phosphate (standard) |
| | | | | Ribose | Pos. 6: Phosphate (standard) |
| | | | | Pos. 5: 2- | Pos. 7: Phosphate (standard) |
| | | | | Fluororibose | Pos. 8: Phosphate (standard) |
| | | | | Pos. 6: 2-OMe | Pos. 9: Phosphate (standard) |
| | | | | Ribose | Pos. 10: Phosphate |
| | | | | Pos. 7: 2- | (standard) |
| | | | | Fluororibose | Pos. 11: Phosphate |
| | | | | Pos. 8: 2-OMe | (standard) |
| | | | | Ribose | Pos. 12: Phosphate |
| | | | | Pos. 9: 2- | (standard) |
| | | | | Fluororibose | Pos. 13: Phosphate |
| | | | | Pos. 10: 2- | (standard) |
| | | | | Fluororibose | Pos. 14: Phosphate |
| | | | | Pos. 11: 2- | (standard) |
| | | | | Fluororibose | Pos. 15: Phosphate |
| | | | | Pos. 12: 2-OMe | (standard) |
| | | | | Ribose | Pos. 16: Phosphate |
| | | | | Pos. 13: 2- | (standard) |
| | | | | Fluororibose | Pos. 17: Phosphate |
| | | | | Pos. 14: 2-OMe | (standard) |
| | | | | Ribose | Pos. 18: Phosphate |
| | | | | Pos. 15: 2- | (standard) |
| | | | | Fluororibose | Pos. 19: Phosphate |
| | | | | Pos. 16: 2-OMe | (standard) |
| | | | | Ribose | Pos. 20: Phosphate |
| | | | | Pos. 17: 2- | (standard) |
| | | | | Fluororibose | Pos. 21: Phosphate |
| | | | | Pos. 18: 2-OMe | (standard) |
| | | | | Ribose | |
| | | | | Pos. 19: 2- | |
| | | | | Fluororibose | |
| | | | | Pos. 20: 2-OMe | |
| | | | | Ribose | |
| | | | | Pos. 21: 2- | |
| | | | | Fluororibose | |
| I-3 | UACUGU | 3 | 5′ None | Pos. 1: 2- | Pos. 1: Phosphorothioate |
| | UGGAUU | | 3′ DBCO | Fluororibose: | linkage |
| | GAUUCG | | | Pos. 2: 2-OMe | Pos. 2: Phosphorothioate |
| | AAA | | | Ribose | linkage |
| | | | | Pos. 3: 2- | Pos. 3: Phosphate (standard) |
| | | | | Fluororibose | Pos. 4: Phosphate (standard) |
| | | | | Pos. 4: 2-OMe | Pos. 5: Phosphate (standard) |
| | | | | Ribose | Pos. 6: Phosphate (standard) |
| | | | | Pos. 5: 2- | Pos. 7: Phosphate (standard) |
| | | | | Fluororibose | Pos. 8: Phosphate (standard) |
| | | | | Pos. 6: 2-OMe | Pos. 9: Phosphate (standard) |
| | | | | Ribose | Pos. 10: Phosphate |
| | | | | Pos. 7: 2- | (standard) |
| | | | | Fluororibose | Pos. 11: Phosphate |
| | | | | Pos. 8: 2-OMe | (standard) |
| | | | | Ribose | Pos. 12: Phosphate |
| | | | | Pos. 9: 2- | (standard) |
| | | | | Fluororibose | Pos. 13: Phosphate |
| | | | | Pos. 10: 2- | (standard) |
| | | | | Fluororibose | Pos. 14: Phosphate |
| | | | | Pos. 11: 2- | (standard) |
| | | | | Fluororibose | Pos. 15: Phosphate |
| | | | | Pos. 12: 2-OMe | (standard) |
| | | | | Ribose | Pos. 16: Phosphate |
| | | | | Pos. 13: 2- | (standard) |
| | | | | Fluororibose | Pos. 17: Phosphate |
| | | | | Pos. 14: 2-OMe | (standard) |
| | | | | Ribose | Pos. 18: Phosphate |
| | | | | Pos. 15: 2- | (standard) |
| | | | | Fluororibose | Pos. 19: Phosphate |
| | | | | Pos. 16: 2-OMe | (standard) |
| | | | | Ribose | Pos. 20: Phosphate |
| | | | | Pos. 17: 2- | (standard) |
| | | | | Fluororibose | Pos. 21: Phosphate |
| | | | | Pos. 18: 2-OMe | (standard) |
| | | | | Ribose | |
| | | | | Pos. 19: 2- | |
| | | | | Fluororibose | |
| | | | | Pos. 20: 2-OMe | |
| | | | | Ribose | |
| | | | | Pos. 21: 2- | |
| | | | | Fluororibose | |
| I-4 | UUCGAA | 4 | None | Pos. 1: 2- | Pos. 1: Phosphorothioate |
| | UCAAUC | | | Fluororibose | linkage |
| | CAACAG | | | Pos. 2: 2-OMe | Pos. 2: Phosphate (standard) |
| | UAGC | | | Ribose | Pos. 3: Phosphate (standard) |
| | | | | Pos. 3: 2- | Pos. 4: Phosphate (standard) |
| | | | | Fluororibose | Pos. 5: Phosphate (standard) |
| | | | | Pos. 4: 2-OMe | Pos. 6: Phosphate (standard) |
| | | | | Ribose | Pos. 7: Phosphate (standard) |
| | | | | Pos. 5: 2- | Pos. 8: Phosphate (standard) |
| | | | | Fluororibose | Pos. 9: Phosphate (standard) |
| | | | | Pos. 6: 2-OMe | Pos. 10: Phosphate |
| | | | | Ribose | (standard) |
| | | | | Pos. 7: 2- | Pos. 11: Phosphate |
| | | | | Fluororibose | (standard) |
| | | | | Pos. 8: 2-OMe | Pos. 12: Phosphate |
| | | | | Ribose | (standard) |
| | | | | Pos. 9: 2- | Pos. 13: Phosphate |
| | | | | Fluororibose | (standard) |
| | | | | Pos. 10: 2-OMe | Pos. 14: Phosphate |
| | | | | Ribose | (standard) |
| | | | | Pos. 11: 2-OMe | Pos. 15: Phosphate |
| | | | | Ribose | (standard) |
| | | | | Pos. 12: 2-OMe | Pos. 16: Phosphate |
| | | | | Ribose | (standard) |
| | | | | Pos. 13: 2- | Pos. 17: Phosphate |
| | | | | Fluororibose | (standard) |
| | | | | Pos. 14: 2-OMe | Pos. 18: Phosphate |
| | | | | Ribose | (standard) |
| | | | | Pos. 15: 2- | Pos. 19: Phosphate |
| | | | | Fluororibose | (standard) |
| | | | | Pos. 16: 2-OMe | Pos. 20: Phosphorothioate |
| | | | | Ribose | linkage |
| | | | | Pos. 17: 2- | Pos. 21: Phosphorothioate |
| | | | | Fluororibose | linkage |
| | | | | Pos. 18: 2-OMe | Pos. 22: Phosphate |
| | | | | Ribose | (standard) |
| | | | | Pos. 19: 2- | |
| | | | | Fluororibose | |
| | | | | Pos. 20: 2-OMe | |
| | | | | Ribose: | |
| | | | | Pos. 21: 2-OMe | |
| | | | | Ribose: | |
| | | | | Pos. 22: 2-OMe | |
| | | | | Ribose | |
| I-5 | UACUGU | 5 | 5′: (Cy5Lumi- | Pos. 1: 2- | Pos. 1: Phosphorothioate |
| | UGGAUU | | Mal)(SHC6) | Fluororibose | linkage |
| | GAUUCG | | 3′: | Pos. 2: 2-OMe | Pos. 2: Phosphorothioate |
| | AAA | | (NHC6)(DBC | Ribose | linkage |
| | | | O-C6NHS) | Pos. 3: 2- | Pos. 3: Phosphate (standard) |
| | | | | Fluororibose | Pos. 4: Phosphate (standard) |
| | | | | Pos. 4: 2-OMe | Pos. 5: Phosphate (standard) |
| | | | | Ribose | Pos. 6: Phosphate (standard) |
| | | | | Pos. 5: 2- | Pos. 7: Phosphate (standard) |
| | | | | Fluororibose | Pos. 8: Phosphate (standard) |
| | | | | Pos. 6: 2-OMe | Pos. 9: Phosphate (standard) |
| | | | | Ribose | Pos. 10: Phosphate |
| | | | | Pos. 7: 2- | (standard) |
| | | | | Fluororibose | Pos. 11: Phosphate |
| | | | | Pos. 8: 2-OMe | (standard) |
| | | | | Ribose | Pos. 12: Phosphate |
| | | | | Pos. 9: 2- | (standard) |
| | | | | Fluororibose | Pos. 13: Phosphate |
| | | | | Pos. 10: 2- | (standard) |
| | | | | Fluororibose | Pos. 14: Phosphate |
| | | | | Pos. 11: 2- | (standard) |
| | | | | Fluororibose | Pos. 15: Phosphate |
| | | | | Pos. 12: 2-OMe | (standard) |
| | | | | Ribose | Pos. 16: Phosphate |
| | | | | Pos. 13: 2- | (standard) |
| | | | | Fluororibose | Pos. 17: Phosphate |
| | | | | Pos. 14: 2-OMe | (standard) |
| | | | | Ribose | Pos. 18: Phosphate |
| | | | | Pos. 15: 2- | (standard) |
| | | | | Fluororibose | Pos. 19: Phosphate |
| | | | | Pos. 16: 2-OMe | (standard) |
| | | | | Ribose | Pos. 20: Phosphate |
| | | | | Pos. 17: 2- | (standard) |
| | | | | Fluororibose | Pos. 21: Phosphate |
| | | | | Pos. 18: 2-OMe | (standard) |
| | | | | Ribose | |
| | | | | Pos. 19: 2- | |
| | | | | Fluororibose | |
| | | | | Pos. 20: 2-OMe | |
| | | | | Ribose | |
| | | | | Pos. 21: 2- | |
| | | | | Fluororibose | |
| I-6 | UACUGU | 6 | 5′ None | Pos. 1: 2- | Pos. 1: Phosphorothioate |
| | UGGAUU | | 3′: | Fluororibose | linkage |
| | GAUUCG | | (NHC6)(DBC | Pos. 2: 2-OMe | Pos. 2: Phosphorothioate |
| | AAA | | O-C6NHS) | Ribose | linkage |
| | | | | Pos. 3: 2- | Pos. 3: Phosphate (standard) |
| | | | | Fluororibose | Pos. 4: Phosphate (standard) |
| | | | | Pos. 4: 2-OMe | Pos. 5: Phosphate (standard) |
| | | | | Ribose | Pos. 6: Phosphate (standard) |
| | | | | Pos. 5: 2- | Pos. 7: Phosphate (standard) |
| | | | | Fluororibose | Pos. 8: Phosphate (standard) |
| | | | | Pos. 6: 2-OMe | Pos. 9: Phosphate (standard) |
| | | | | Ribose | Pos. 10: Phosphate |
| | | | | Pos. 7: 2- | (standard) |
| | | | | Fluororibose | Pos. 11: Phosphate |
| | | | | Pos. 8: 2-OMe | (standard) |
| | | | | Ribose | Pos. 12: Phosphate |
| | | | | Pos. 9: 2- | (standard) |
| | | | | Fluororibose | Pos. 13: Phosphate |
| | | | | Pos. 10: 2- | (standard) |
| | | | | Fluororibose | Pos. 14: Phosphate |
| | | | | Pos. 11: 2- | (standard) |
| | | | | Fluororibose | Pos. 15: Phosphate |
| | | | | Pos. 12: 2-OMe | (standard) |
| | | | | Ribose | Pos. 16: Phosphate |
| | | | | Pos. 13: 2- | (standard) |
| | | | | Fluororibose | Pos. 17: Phosphate |
| | | | | Pos. 14: 2-OMe | (standard) |
| | | | | Ribose | Pos. 18: Phosphate |
| | | | | Pos. 15: 2- | (standard) |
| | | | | Fluororibose | Pos. 19: Phosphate |
| | | | | Pos. 16: 2-OMe | (standard) |
| | | | | Ribose | Pos. 20: Phosphate |
| | | | | Pos. 17: 2- | (standard) |
| | | | | Fluororibose | Pos. 21: Phosphate |
| | | | | Pos. 18: 2-OMe | (standard) |
| | | | | Ribose | |
| | | | | Pos. 19: 2- | |
| | | | | Fluororibose | |
| | | | | Pos. 20: 2-OMe | |
| | | | | Ribose | |
| | | | | Pos. 21: 2- | |
| | | | | Fluororibose | |
| I-7 | UACUGU | 7 | 5′: (Cy5Lumi- | Pos. 1: 2- | Pos. 1: Phosphorothioate |
| | UGGAUU | | Mal)(SHC6) | Fluororibose | linkage |
| | GAUUCG | | 3′: None | Pos. 2: 2-OMe | Pos. 2: Phosphorothioate |
| | AAA | | | Ribose | linkage |
| | | | | Pos. 3: 2- | Pos. 3: Phosphate (standard) |
| | | | | Fluororibose | Pos. 4: Phosphate (standard) |
| | | | | Pos. 4: 2-OMe | Pos. 5: Phosphate (standard) |
| | | | | Ribose | Pos. 6: Phosphate (standard) |
| | | | | Pos. 5: 2- | Pos. 7: Phosphate (standard) |
| | | | | Fluororibose | Pos. 8: Phosphate (standard) |
| | | | | Pos. 6: 2-OMe | Pos. 9: Phosphate (standard) |
| | | | | Ribose | Pos. 10: Phosphate |
| | | | | Pos. 7: 2- | (standard) |
| | | | | Fluororibose | Pos. 11: Phosphate |
| | | | | Pos. 8: 2-OMe | (standard) |
| | | | | Ribose | Pos. 12: Phosphate |
| | | | | Pos. 9: 2- | (standard) |
| | | | | Fluororibose | Pos. 13: Phosphate |
| | | | | Pos. 10: 2- | (standard) |
| | | | | Fluororibose | Pos. 14: Phosphate |
| | | | | Pos. 11: 2- | (standard) |
| | | | | Fluororibose | Pos. 15: Phosphate |
| | | | | Pos. 12: 2-OMe | (standard) |
| | | | | Ribose | Pos. 16: Phosphate |
| | | | | Pos. 13: 2- | (standard) |
| | | | | Fluororibose | Pos. 17: Phosphate |
| | | | | Pos. 14: 2-OMe | (standard) |
| | | | | Ribose | Pos. 18: Phosphate |
| | | | | Pos. 15: 2- | (standard) |
| | | | | Fluororibose | Pos. 19: Phosphate |
| | | | | Pos. 16: 2-OMe | (standard) |
| | | | | Ribose | Pos. 20: Phosphate |
| | | | | Pos. 17: 2- | (standard) |
| | | | | Fluororibose | Pos. 21: Phosphate |
| | | | | Pos. 18: 2-OMe | (standard) |
| | | | | Ribose | |
| | | | | Pos. 19: 2- | |
| | | | | Fluororibose | |
| | | | | Pos. 20: 2-OMe | |
| | | | | Ribose | |
| | | | | Pos. 21: 2- | |
| | | | | Fluororibose | |
| I-8 | UCCUAU | 8 | 5′: None | Pos. 1: 2-OMe | Pos. 1: Phosphorothioate |
| | GACUGU | | 3′: (DBCO) | Ribose | linkage |
| | AGAUUU | | | Pos. 2: 2-OMe | Pos. 2: Phosphorothioate |
| | UAU | | | Ribose | linkage |
| | | | | Pos. 3: 2-OMe | Pos. 3: Phosphate (standard) |
| | | | | Ribose | Pos. 4: Phosphate (standard) |
| | | | | Pos. 4: 2-OMe | Pos. 5: Phosphate (standard) |
| | | | | Ribose | Pos. 6: Phosphate (standard) |
| | | | | Pos. 5: 2-OMe | Pos. 7: Phosphate (standard) |
| | | | | Ribose | Pos. 8: Phosphate (standard) |
| | | | | Pos. 6: 2-OMe | Pos. 9: Phosphate (standard) |
| | | | | Ribose | Pos. 10: Phosphate |
| | | | | Pos. 7: 2- | (standard) |
| | | | | Fluororibose | Pos. 11: Phosphate |
| | | | | Pos. 8: 2-OMe | (standard) |
| | | | | Ribose | Pos. 12: Phosphate |
| | | | | Pos. 9: 2- | (standard) |
| | | | | Fluororibose | Pos. 13: Phosphate |
| | | | | Pos. 10: 2- | (standard) |
| | | | | Fluororibose | Pos. 14: Phosphate |
| | | | | Pos. 11: 2- | (standard) |
| | | | | Fluororibose | Pos. 15: Phosphate |
| | | | | Pos. 12: 2-OMe | (standard) |
| | | | | Ribose | Pos. 16: Phosphate |
| | | | | Pos. 13: 2-OMe | (standard) |
| | | | | Ribose | Pos. 17: Phosphate |
| | | | | Pos. 14: 2-OMe | (standard) |
| | | | | Ribose | Pos. 18: Phosphate |
| | | | | Pos. 15: 2-OMe | (standard) |
| | | | | Ribose | Pos. 19: Phosphate |
| | | | | Pos. 16: 2-OMe | (standard) |
| | | | | Ribose | Pos. 20: Phosphate |
| | | | | Pos. 17: 2-OMe | (standard) |
| | | | | Ribose | Pos. 21: Phosphate |
| | | | | Pos. 18: 2-OMe | (standard) |
| | | | | Ribose | |
| | | | | Pos. 19: 2-OMe | |
| | | | | Ribose | |
| | | | | Pos. 20: 2-OMe | |
| | | | | Ribose | |
| | | | | Pos. 21: 2-OMe | |
| | | | | Ribose | |
| I-9 | AUAAAA | 9 | 5′: phosphate | Pos. 1: 2-OMe | Pos. 1: Phosphorothioate |
| | UCUACA | | 3′: None | Ribose | linkage |
| | GUCAUA | | | Pos. 2: 2- | Pos. 2: Phosphorothioate |
| | GGAAU | | | Fluororibose | linkage |
| | | | | Pos. 3: 2-OMe | Pos. 3: Phosphate (standard) |
| | | | | Ribose | Pos. 4: Phosphate (standard) |
| | | | | Pos. 4: 2-OMe | Pos. 5: Phosphate (standard) |
| | | | | Ribose | Pos. 6: Phosphate (standard) |
| | | | | Pos. 5: 2-OMe | Pos. 7: Phosphate (standard) |
| | | | | Ribose | Pos. 8: Phosphate (standard) |
| | | | | Pos. 6: 2- | Pos. 9: Phosphate (standard) |
| | | | | Fluororibose | Pos. 10: Phosphate |
| | | | | Pos. 7: 2-OMe | (standard) |
| | | | | Ribose | Pos. 11: Phosphate |
| | | | | Pos. 8: 2-OMe | (standard) |
| | | | | Ribose | Pos. 12: Phosphate |
| | | | | Pos. 9: 2-OMe | (standard) |
| | | | | Ribose | Pos. 13: Phosphate |
| | | | | Pos. 10: 2-OMe | (standard) |
| | | | | Ribose | Pos. 14: Phosphate |
| | | | | Pos. 11: 2-OMe | (standard) |
| | | | | Ribose | Pos. 15: Phosphate |
| | | | | Pos. 12: 2-OMe | (standard) |
| | | | | Ribose | Pos. 16: Phosphate |
| | | | | Pos. 13: 2-OMe | (standard) |
| | | | | Ribose | Pos. 17: Phosphate |
| | | | | Pos. 14: 2- | (standard) |
| | | | | Fluororibose | Pos. 18: Phosphate |
| | | | | Pos. 15: 2-OMe | (standard) |
| | | | | Ribose | Pos. 19: Phosphate |
| | | | | Pos. 16: 2- | (standard) |
| | | | | Fluororibose | Pos. 20: Phosphate |
| | | | | Pos. 17: 2-OMe | (standard) |
| | | | | Ribose | Pos. 21: Phosphorothioate |
| | | | | Pos. 18: 2-OMe | linkage |
| | | | | Ribose | Pos. 22: Phosphorothioate |
| | | | | Pos. 19: 2-OMe | linkage |
| | | | | Ribose | Pos. 23: Phosphate |
| | | | | Pos. 20: 2-OMe | (standard) |
| | | | | Ribose | |
| | | | | Pos. 21: 2-OMe | |
| | | | | Ribose | |
| | | | | Pos. 22: 2-OMe | |
| | | | | Ribose | |
| | | | | Pos. 23: 2-OMe | |
| | | | | Ribose | |
| I-10 | AUAAAA | 29 | 5′: phosphate | Pos. 1: 2-OMe | Pos. 1: Phosphorothioate |
| | UCUACA | | 3′: None | Ribose | linkage |
| | GUCAUA | | | Pos. 2: 2- | Pos. 2: Phosphorothioate |
| | GGA | | | Fluororibose | linkage |
| | | | | Pos. 3: 2-OMe | Pos. 3: Phosphate (standard) |
| | | | | Ribose | Pos. 4: Phosphate (standard) |
| | | | | Pos. 4: 2-OMe | Pos. 5: Phosphate (standard) |
| | | | | Ribose | Pos. 6: Phosphate (standard) |
| | | | | Pos. 5: 2- | Pos. 7: Phosphate (standard) |
| | | | | Fluororibose | Pos. 8: Phosphate (standard) |
| | | | | Pos. 6: 2-OMe | Pos. 9: Phosphate (standard) |
| | | | | Ribose | Pos. 10: Phosphate |
| | | | | Pos. 7: 2-OMe | (standard) |
| | | | | Ribose | Pos. 11: Phosphate |
| | | | | Pos. 8: 2-OMe | (standard) |
| | | | | Ribose | Pos. 12: Phosphate |
| | | | | Pos. 9: 2-OMe | (standard) |
| | | | | Ribose | Pos. 13: Phosphate |
| | | | | Pos. 10: 2-OMe | (standard) |
| | | | | Ribose | Pos. 14: Phosphate |
| | | | | Pos. 11: 2-OMe | (standard) |
| | | | | Ribose | Pos. 15: Phosphate |
| | | | | Pos. 12: 2-OMe | (standard) |
| | | | | Ribose | Pos. 16: Phosphate |
| | | | | Pos. 13: 2- | (standard) |
| | | | | Fluororibose | Pos. 17: Phosphate |
| | | | | Pos. 14: 2-OMe | (standard) |
| | | | | Ribose | Pos. 18: Phosphate |
| | | | | Pos. 15: 2- | (standard) |
| | | | | Fluororibose | Pos. 19: Phosphorothioate |
| | | | | Pos. 16: 2-OMe | linkage |
| | | | | Ribose | Pos. 20: Phosphorothioate |
| | | | | Pos. 17: 2-OMe | linkage |
| | | | | Ribose | Pos. 21: Phosphate |
| | | | | Pos. 18: 2-OMe | (standard) |
| | | | | Ribose | |
| | | | | Pos. 19: 2-OMe | |
| | | | | Ribose | |
| | | | | Pos. 20: 2-OMe | |
| | | | | Ribose | |
| | | | | Pos. 21: 2-OMe | |
| | | | | Ribose | |

Glycan—siRNA

siRNAs functionalized with DBCO at the 3′ end were purchased from WuXi Biologics® or Axolabs® and made by methods well established in the art. siRNAs with DBCO conjugated at the 3′ end were incubated with a 10-fold excess (or 1 equivalent, or 0.75 equivalent) of azide functionalized glycan. Conjugation reactions are performed at 37° C. overnight. Conjugated glycoRNAs were purified by HPLC. HPLC purification of the glycoRNA conjugates was carried out using 200 mM HFIP+16 mM TEA in methanol. Instrument model: Agilent 1260 HPLC; Column: Agilent AdvanceBio Oligonucleotide, 2.1×50 mm, 2.7 μm. The purified glycoRNAs are dried by lyophilization. The glycoRNAs are then resuspended in water to a concentration of 100 μM. GlycoRNAs are then annealed to the complementary sense strand in Annealing Buffer (30 mM Tris, pH 7.5, 100 mM NaCl, 1 mM EDTA). For the annealing reaction, samples are heated to 95° C. and slow cooled to room temperature over approximately 1 hour. The annealed duplex is desalted using a Zeba Spin Desalting Column (Thermo Fisher Scientific®) by centrifugation at 1500×g for 2 minutes.

Similarly, comparison compounds X-1 and X-2 (shown in Table 5B) were synthesized using the general procedures described above, wherein monosaccharides were used in place of the azide functionalized glycans. The conjugated monosaccharides were then annealed to I-1, or a different siRNA of the disclosure, using the general procedures described above.

TABLE 5B
Exemplified monosaccharide modified siRNAs

| Ref # | Monosaccharide | siRNA |
|---|---|---|
| X-1 | 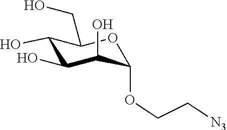 | I-2 |
| | 2-Azidoethyl α-D-mannopyranoside | |
| X-2 | 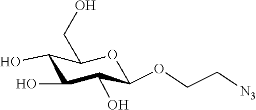 | I-2 |
| | 2-Azidoethyl β-D-glucopyranoside | |

Additionally, comparison compound X-3 was purchased from WuXi Biologics® and made by methods well established in the art. An exemplary siRNA conjugate of X-3 comprises a sense strand that is I-7 (SEQ ID NO: 7). The X-3 conjugate was then annealed to I-4, using the general procedures described above.

TABLE 5C
Exemplified modified siRNA

| Ref # | Compound |
|---|---|
| X-3 | 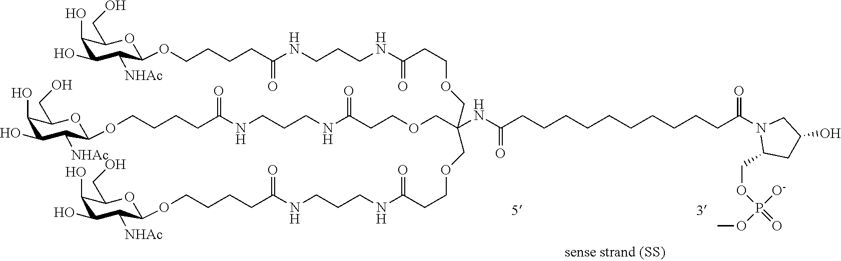 |

### Example 8A: Target mRNA Knock Down In Vitro Using GlycoRNAs in 293T Cells

293T cells are plated 24 hours before the experiment at 200,000 cells in 1 mL of growth media in 12-well plates. 3 μl of lipofectamine is added to 50 μl of serum free media, and glyco-siRNA duplex is added separately to serum free media to 200 nM final concentration. These two mixtures, lipofectamine and diluted duplex, are added together at room temperature and incubated for 10 minutes. Media is aspirated from plated 293 T cells and replaced with 1 ml of fresh media. 100 μl of the lipofectamine-glyco-siRNA mixture is added to each well and incubated overnight. RNA is purified from cells using RNA lysis buffer (Zymo Research Corp.), followed by RNA prep, wash, and elution buffers and spins at 10,000×g for 2 minutes each (Zymo Research Corp.). cDNA is synthesized using 200 ng RNA per sample using the SuperScript™ IV cDNA synthesis system (Life Technologies/Thermo Scientific) with oligo (dT) primers, following manufacturer's instructions using a BioRad thermocycler. cDNA is diluted to 15 ng/μL to have 60 ng per qPCR reaction. Samples are run in duplicate and each sample has a biological replicate. 1× Taqman qPCR probes against β-catenin and β-actin (Assay ID for β-catenin probe set: Hs00355045_m1, endogenous human β-actin control: Hs01060665_g1, both purchased from Applied Bio/Thermo Scientific) and 1× TaqMan gene expression master mix are used to amplify cDNA. Samples are first incubated for 30 minutes at 50° C. then 10 minutes at 95° C. followed by 40 cycles of 30 seconds of 95° C. and 1 minute at 60° C. Beta catenin Ct values are normalized by β-actin Ct values to report relative abundance (% beta catenin mRNA).

### Example 8B: Target mRNA Knockdown In Vitro Using GlycoRNAs in Primary B Cells

Human primary immune cells (Stem Cell Technologies) were thawed and cultured at 37° C. overnight before glyco-siRNA treatment. Glyco-siRNA conjugates made with HPRT1 interfering siRNA (SEQ ID NOS: 8 and 9) were dosed to primary immune cells in a 96-well plate (1×105 cells per well) at different doses. 48 hours following incubation, the plate was washed with cold 1×PBS plus 150 mM NaCl, 3 times. Total RNA extraction was executed using KingFisher Flex system and Thermo Fisher MagMAX™ mirVana™ Total RNA Isolation Kit (Cat. No. A27828) following the provider's guidance. The purified RNA was eluted in 30 μl water. 10 μl eluant was used for reverse transcription using the Roche High-capacity cDNA Reverse Transcription kit (Cat. No. 43-688-14). The QPCR method used was as described below in Example 9.

TABLE 6
Primary B cell HPRT1 mRNA % KD (relative to PBS control)

| | Modified | 100 nM | 500 nM |
|---|---|---|---|
| Glycan | Glycan | conc | conc |
| siRNA | siRNA | 120.99 | 91.14 |
| Glucose | X-2 | 114.50 | 109.45 |
| G-2 | A-2 | 128.63 | 95.28 |
| G-6 | A-6 | 99.06 | 78.81 |
| G-9 | P-3 | 83.31 | 72.85 |
| G-17 | P-11 | 117.91 | 92.58 |
| G-18 | P-12 | 126.52 | 96.42 |
| G-20 | P-14 | 127.45 | 91.97 |
| H-33 | O-1 | 112.69 | 83.36 |
| J-3 | N-11 | 105.81 | 70.14 |
| J-4 | N-9 | 75.39 | 63.47 |

### Example 9: Target mRNA Knockdown In Vitro Using GlycoRNAs in Primary Human Hepatocytes

1×10⁵ primary human hepatocytes from healthy donors are obtained from Lonza Bioscience. The cells are thawed in INVITROGRO HT medium and are cultured in INVITROGRO HI medium (BioIVT). Cells are plated in 96 well flat bottom plates and incubated with titrations of Cy5 labelled duplexed glycoRNAs for 24 hours in serum-free INVITROGRO HT media. After incubation, media is removed via aspiration and cells are washed once in PBS. Dry pellets are frozen at −80° C. until RNA extraction. Total RNA was isolated from cells using RNeasy micro spin columns (QIAGEN) following manufacturer's instructions. Total RNA is eluted in water (30 μL total volume) and an aliquot is quantified on a NanoDrop™ (Thermo Scientific). cDNA is synthesized using 100 ng RNA per sample using the SuperScript™ IV cDNA synthesis system (Life Technologies/Thermo Scientific) with oligo (dT) primers, following manufacturer's instructions using a BioRad thermocycler. Gene expression is assessed using multiplexed TaqMan probes against β-catenin and β-actin (Assay ID for β-catenin probe set: Hs00355045_ml, endogenous human β-actin control: Hs01060665_g1, both purchased from Applied Bio/Thermo Scientific). 10 ng of sample cDNA is plated per well in 96 well optically clear PCR plates in biological and technical replicates (Applied Bio/Thermo Scientific), and 20× TaqMan probes and 2× TaqMan gene expression master mix are added following manufacturer's instructions for 20 μL total reaction volume per well (Applied Bio/Thermo Scientific). Samples are amplified on a QuantStudio 6 Pro Real Time PCR System using the following amplification parameters: Stage 1:50° C. for 2 minutes. Stage 2:95° C. for 10 minutes. Stage 3:95° C. for 15 seconds, 60° C. for 1 minutes. Repeat 40×. Gene expression of β-catenin is calculated using the ΔΔCT method relative to beta actin expression and untreated control cells, where a value of less than 1 indicates siRNA-mediated knock down of β-catenin.

### Example 10: HepG2 Transfection Protocol

HepG2 cells are plated 24 hours before the experiment at 200,000 cells per well in 1 mL of growth media in 12-well plates. 3 μl of lipofectamine is added to 100 μl of serum free media, and glyco-siRNA duplex is added separately to serum free media to 200 nM final concentration. These two mixtures, lipofectamine and diluted duplex, are added together at room temperature and incubated for 10 minutes. Media is aspirated from plated HepG2 cells and replaced with 1 ml of fresh media. 100 μl of the lipofectamine-glyco-siRNA mixture is added to each well and incubated overnight. RNA is purified from cells using RNA lysis buffer (Zymo Research Corp.), followed by RNA prep, wash, and elution buffers and spins at 10,000 g for 2 minutes each (Zymo Research Corp.). cDNA is synthesized using 200 ng RNA per sample using the SuperScript™ IV cDNA synthesis system (Life Technologies/Thermo Scientific) with oligo (dT) primers, following manufacturer's instructions using a BioRad thermocycler. cDNA is diluted to 15 ng/μL to have 60 ng per qPCR reaction. Samples are run in duplicate and each sample had a biological replicate. 1× Taqman qPCR probes against β-catenin and β-actin (Assay ID for β-catenin probe set: Hs00355045_m1, endogenous human β-actin control: Hs01060665_g1, both purchased from Applied Bio/Thermo Scientific) and 1× TaqMan gene expression master mix were used to amplify cDNA. Samples are first incubated for 30 minutes at 50° C. then 10 minutes at 95° C. followed by 40 cycles of 30 seconds of 95° C. and 1 minutes at 60° C. Beta catenin Ct values are normalized by those of Ct values of β-actin to report relative abundance (% beta catenin mRNA).

### Example 11: Glyco-siRNA Internalization Imaging Assays

On Day 1, cells are washed with an excess of 10 mL 1×PBS, then are incubated with 10 mL of ACCUTASE® (Sigma) for 10 minutes at 37° C. Cell are collected, spun down at 300×g for 5 minutes, and resuspended in 4 mL OptiMEM® (Gibco, Thermo Fischer Scientific, Inc.) to a total of 400,000 cells for 200 wells. Cell Mask Green Plasma Membrane Stain (ThermoFisher Scientific, Inc.) at 1:5,000 and Hoechst stain at 1:20,000 are added to the diluted cells and the cells are incubated for 5 minutes at 37° C. Cells are then washed with 6 mL OptiMEM® (Gibco, Thermo Fischer Scientific, Inc.) and spun down at 300 g for 5 minutes. The media is discarded, and cells are resuspended in complete media (Dulbecco's Modified Eagle Medium (DMEM)+10% FBS+1% penicillin G and streptomycin (PEN/STREP), Gibco/Life Technologies) to a concentration of 1×10⁴ cells/mL. Cells are seeded at 2000 cells/well in 20 μL of complete media in 384-well imaging plates (CORNING®). Cells are incubated in standard tissue culture incubators at 37° C., 5% CO₂ overnight. On Day 2, the cells are dosed with 15 nM, 2 nM, and 0 nM of Cy5-labelled duplexed glycoRNAs. The plates are then live-cell imaged every 30 minutes for 4 hours, while maintaining incubation at 37° C., 5% CO₂, on the Opera Phenix High Content Screening System with a 40× water objective in the DAPI, FITC, and Cy5 channels. The images collected are analyzed on the Harmony High-Content Imaging and Analysis Software (Perkin Elmer, Inc.). Generally, nuclei are identified and filtered by size, shape, and intensity in the DAPI channel; cells are then identified from selected nuclei and filtered by size, shape, and intensity in the FITC channel; and signal from the glyco-siRNAs is identified by the Cy5 signal within the selected cells as either spots or intensity, as deemed appropriate for the cell type. All associated metrics (count, intensity, and area) for all three channels are calculated and analyzed with a custom R script. This procedure can be executed on at least 8 cell lines: HepG2 cells, A549 cells, SK-N-DZ cells, Huh7 cells, THP-1 cells, Raji cells, PANC-1 cells and Jurkat cells.

### Example 12A: In Vivo Biodistribution of Injected Glyco-siRNA

BALB/c mice are dosed with each test article either through injection via tail vein or subcutaneous injection with about 0.1-10 mg/kg (per glyco-RNA) in a total volume of about 5-10 mL/kg. In an exemplary experiment, each test article is dosed in 6 mice, and an additional 3 mice are dosed with PBS control. At a first time point (e.g., 4 hours or 24 hours post injection), 3 animals dosed with each test article are whole-body imaged for Cy5 bioluminescence signal using an IVIS Spectrum In Vivo Imaging System (PerkinElmer). Similarly, at a second time point (e.g., 48 hours or 72 hours post injection), the remaining 3 animals are whole-body imaged for Cy5 bioluminescence signal using an IVIS Spectrum In Vivo Imaging System (PerkinElmer). Directly following whole body imaging at each time point, animals are immediately euthanized by CO₂ inhalation, and organs including, but not limited to, liver, spleen, lung, heart and kidneys are harvested and subjected to bioluminescence imaging (BLI) analysis within 10 minutes of animal sacrifice. Organs were collected after imaging and snap frozen using liquid nitrogen. BLI images are detected in the auto-exposure mode. The BLI signals are quantitated using Living Image 4.7 software (Perkin Elmer) following the manufacturer's instruction. After BLI analysis the weights of the collected organs are measured.

Snap frozen tissues are cooled in liquid nitrogen and then homogenized using the GENOMAX® Homogenizer (SPEX EW-41019-48) for 1 minute 45 seconds at 1500 RPM. Powderized tissues are stored in −80° C. freezer until ready for test article extraction. Test articles are extracted and cleaned using a 1.8× bead cleanup with AMPURE® XP (Beckman Coulter A63881) following manufacturer protocols using the KINGFISHER™ Apex.

Single stranded cDNA is prepared using Thermo's SuperScript IV Reverse Transcription kit (oligo(dT)) according to the manufacturer's instructions. cDNA is diluted to 15 ng/μL to have 60 ng per qPCR reaction. Samples are run in duplicate and each sample had a biological replicate. 1× Taqman qPCR probes against β-catenin and β-actin (Assay ID for β-catenin probe set: Hs00355045_m1, endogenous human β-actin control: Hs01060665_g1, both purchased from Applied Bio/Thermo Scientific) and 1× TaqMan gene expression master mix were used to amplify cDNA. Samples are first incubated for 30 minutes at 50° C. then 10 minutes at 95° C. followed by 40 cycles of 30 seconds of 95° C. and 1 minute at 60° C. Beta catenin Ct values are normalized by those of Ct values of β-actin to report relative abundance (% beta catenin mRNA).

All in vivo experiments in this study are performed under the approved animal care guidelines. Time points, dosages and organs/tissues to be harvested can be modified as needed.

### Example 12B: In Vivo Biodistribution of Injected Glyco-siRNA

Glyco-siRNA conjugates were prepared as described in Examples 6 and 7, wherein the siRNA was an HPRT1-siRNA (SEQ ID NOS: 8 and 9). Test article dosing solutions were prepared using sterile PBS at 10 ml/kg body weight. Female C56BL/6 mice (6-8 weeks old) were subcutaneously dosed with glyco-siRNA formulations. Animal wellness was observed 4 and 24 hours post dosing. Animals were dosed with each test article through subcutaneous injection with 1 or 10 mg/kg (per glyco-siRNA) in a total volume of 10 mL/kg. Each test article was dosed in 6 mice, and an additional 3 mice were dosed with PBS control. At 24 hours post dose, 3 animals dosed with each test article, and at 10 days post dose, the further 3 animals dosed with each test article were euthanized by CO₂ inhalation, and organs including liver, kidney, spleen, lymph nodes, and muscle (gastrocnemius) were harvested and snap frozen using liquid nitrogen. Snap frozen tissues were cooled in liquid nitrogen and then homogenized using the GENOMAX® 2050 Homogenizer for 1 minute 45 seconds at 1500 RPM. Powderized tissues are stored in −80° C. freezer until ready for test article extraction.

Stem Loop-RT qPCR

The organ samples were analyzed by stem loop qPCR to determine the amount of glyco-siRNA test article delivered to each tissue. The tissue lysate [10 mg/ml in PBST (1×PBS, 0.25% Triton X-100)) was prepared by Qiagen TissueLyser LT (200 strokes/minute, 2 minutes) with 2×5-mm metal beads in each 2 ml Eppendorf tube. Fresh lymph nodes and isolated splenocytes were briefly sonicated in 300 ul PBST using Q55 Sonicator (Amp 40, 10 seconds). The lysate concentration was measured using Thermo Fisher BCA protein kit (Cat No. PI23227). The prepared lysate were heated at 95° C. for 10 minutes and centrifuged (15,000 rpm, 10 min) before stem-loop RT-qPCR analysis. To prepare the standard curve, 20 nM of glyco-siRNAs used in the study were serial diluted 5-fold for 9 times. The reverse transcription reaction was prepared by using TaqMan MicroRNA Reverse Transcription Kit (Thermo Scientific). A final concentration of 1 mM dNTP, 5 U/μl transcriptase, 0.38 U/μl Rnase inhibitor, 50 nM stem loop RT primer against siRNA targeting F12 (5′-GTCGTATCCAGTGCAGGGTCCGAGGTATTCGCA CTGGATACGAC CAGAAACT CA-3′ (SEQ ID NO: 12)) or HPRT1 (5′-GTCGTATCCAGTGCAGGGTCCGAGGTATTCGCA CTGGATACGACATTCCTATGA-3′ (SEQ ID NO: 13)) was mixed in a 10 μl volume reaction with 1× buffer. Both RT primers were purchased from IDT. 5 μl of the boiled tissue sample was added to each 10 ul of reverse 243 transcription mix. Samples were incubated at 16° C. for 30 minutes then at 42° C. for 30 minutes followed by 85° C. for 5 minutes and the samples are ready to be used for qPCR reaction. A final concentration of 1.5 μM Forward primers from IDT (F12: 5′-GCCGCGCTAAAGCACTTTAT-3′ (SEQ ID NO: 14), HPRT1: 5′-GCCGCGCATAAAATCTACAG-3′ (SEQ ID NO: 15)), 0.7 μM Reverse primers from IDT (5′-GTGCAGGGTCCGAGGT-3′ (SEQ ID NO: 16)), 0.2 μM customized Taqman MGB probe (F12: 5′-GCCGCGC TAAAGCACTTTAT-3′ (SEQ ID NO: 17), and HPRT1: 5′-CTGGATACGAC ATTCCTAT-3′ (SEQ ID NO:18)) from Thermo Scientific were mixed in 1× TaqMan gene expression master mix from Thermo Scientific to a total volume of 8.5 μl. 1.5 μl of cDNA template from the reverse transcription reaction was added to the qPCR mix to a final volume of 10 μl. Samples are first incubated at 95° C. for 10 minutes followed by 40 cycles of 30 seconds of 95° C. and 1 minutes at 60° C. siRNA values were calculated based on the Ct value of standard curve and reported as ng RNA/mg tissue, averaged over the three animals. Biodistribution of siRNA molecules from this experiment are shown in Table 7A. Table 7B shows the results of an analogous experiment with differing glyco-ligands.

TABLE 7A
Biodistribution of Glyco-siRNA Conjugates
in BALB/c Mice After 24 Hours

| Target- | Modified | | | | | | |
|---|---|---|---|---|---|---|---|
| ing | Glycan/ | Dose | | Kid- | | Lymph | |
| moiety | saccharide | (mg/kg) | Liver | ney | Spleen | Node | Muscle |
| siRNA | siRNA | 10 | 0.47 | 3.99 | 0.48 | 41.25 | 0.13 |
| Glucose | X-2 | 10 | 0.84 | 6.74 | 0.65 | 21.50 | 0.24 |
| Tri- | X-3 | 1 | 6.37 | 0.09 | 0.06 | — | — |
| GalNAc | | | | | | | |
| G-29 | A-9 | 1 | 1.99 | 0.16 | 0.07 | — | — |
| G-30 | A-10 | 1 | 2.47 | 0.16 | 0.08 | — | — |
| G-37 | N-6 | 1 | 6.78 | 0.02 | 0.02 | — | — |
| G-38 | N-7 | 1 | 7.37 | 0.04 | 0.04 | — | — |
| G-24 | P-18 | 1 | 0.77 | 0.55 | 0.56 | 5.38 | — |
| G-27 | A-7 | 1 | 0.36 | 0.57 | 0.17 | 6.83 | — |
| H-7 | N-5 | 1 | 0.28 | 1.05 | 0.10 | 6.02 | — |
| H-9 | N-10 | 1 | 0.37 | 1.03 | 0.19 | 5.02 | — |
| G-24 | P-18 | 10 | 3.83 | 9.21 | 3.80 | 85.88 | 0.68 |
| G-27 | A-7 | 10 | 1.82 | 5.49 | 1.31 | 94.60 | — |
| H-7 | N-5 | 10 | 1.34 | 7.04 | 0.59 | 47.48 | — |
| H-9 | N-10 | 10 | 2.11 | 6.23 | 0.86 | 31.13 | — |

** Values recorded as ng RNA/mg tissue

TABLE 7B
Biodistribution of Glyco-siRNA Conjugates
in BALB/c Mice After 24 Hours

| | Modified | | | | |
|---|---|---|---|---|---|
| Targeting | Glycan/ | Dose | | | |
| Moiety | Saccharide | (mg/kg) | Liver | Kidney | Spleen |
| Glucose | X-2 | 1 | 0.14 | 0.44 | 0.03 |
| Tri-GalNAc | X-3 | 1 | 1.18 | 0.06 | 0.01 |
| G-12 | P-6 | 1 | 0.56 | 0.06 | 0.03 |
| G-16 | P-10 | 1 | 2.13 | 0.06 | 0.01 |
| G-37 | N-6 | 1 | 1.92 | 0.02 | 0.01 |
| G-38 | N-7 | 1 | 1.56 | 0.06 | 0.01 |

** Values recorded as ng RNA/mg tissue

RT qPCR to Measure mRNA KD

The organ samples were collected, snap-frozen in liquid nitrogen and saved at −80° C. before analysis. The frozen tissue samples were ground into powder using Genomax® 2050 following vendor's instructions. ˜10 mg tissue powder was transferred into 2 ml Eppendorf tube for RNA extraction. Fresh lymph node and isolated splenocytes were directly subjected to RNA extraction by adding 600 uL RNA lysis buffer (RLT) buffer. Total tissue RNA extraction was executed using QIAshredder (Cat. No. 79656) and QIAgen RNeasy kit (Cat. No. 74106) following the provider's guidance.

cDNA prep (10 ul purified RNA for each sample) was done using the Roche High-capacity cDNA Reverse Transcription kit (Cat. No. 43-688-14). QPCR method methodology used was as described in Example 9.

TABLE 7C
HPRT1-mRNA % Knockdown Relative to PBS Control

| | Modified | | | | | |
|---|---|---|---|---|---|---|
| Targeting | Glycan/ | Dose | | | | Lymph |
| moiety | saccharide | (mg/kg) | Liver | Muscle | Spleen | Node |
| siRNA | siRNA | 10 | 1.00 | 0.96 | 0.91 | 0.97 |
| G-24 | P-18 | 10 | 0.76 | 0.92 | 0.91 | 1.08 |
| G-27 | A-7 | 10 | 0.55 | 1.06 | 0.98 | 0.87 |

### Knockdown Measured Using FXII ELISA

Plasma from the 10-day post dose mice was collected using EDTA tube and saved at −80° C. before analysis. The plasma FXII levels were measured using Innov-Research FXII ELISA kit (Cat. No. IMSF12KT) following the provider's instructions.

TABLE 7D
Knockdown Relative to PBS Control Measured Using FXII ELISA

| | Modified | | |
|---|---|---|---|
| Targeting | Glycan/ | Dose | |
| Moiety | Saccharide | (mg/kg) | % of PBS Control |
| Glucose | X-2 | 0.1 | 83.00 |
| Tri-GalNAc | X-3 | 0.1 | 36.43 |
| G-37 | N-6 | 0.1 | 68.40 |
| G-38 | N-7 | 0.1 | 65.24 |
| Glucose | X-2 | 1 | 77.24 |
| Tri-GalNAc | X-3 | 1 | 3.97 |
| G-37 | N-6 | 1 | 11.24 |
| G-38 | N-7 | 1 | 9.68 |

### Example 12C: In Vivo Uptake in Splenic Macrophages

Glyco-siRNA conjugates were prepared as described in Examples 6 and 7, wherein the siRNA was an HPRT1-siRNA (SEQ ID NOS: 8 and 9). Mice were dosed with glyco-HPRT1 siRNA as described in Example 12B. After being euthanized at 24 hours or 7 days, livers and spleens were collected. Fat from mouse spleens was removed and put in Miltenyi C tubes (Miltenyi 130-093-237) with 2.5 ml Enzyme mix (2.4 mL 1× Buffer S, 50 uL Enzyme D, 15 uL Enzyme A) from Miltenyl Spleen Dissociation Kit (130-095-926). C-Tubes with spleen and buffer were affixed to Miltenyi gentleMACS™ Dissociator (130-093-235) to prepare single cell suspension, which are further filtered with 70 um filter (Miltenyi, 130-098-462). Filters were washed with 5 ml wash buffer (0.5% BSA+2 mM EDTA in Ca/Mg free 1×PBS) and cells were pelleted by centrifugation at 500×g for 3 minutes. Cells were re-suspended in 3 mL ACK buffer (Thermo, A1049201) and incubated for 5 minutes to lyse the red blood cells. Cells were pelleted by centrifugation and resuspended in 5 ml wash buffer followed by passing through a filter and another centrifugation to collect the cell pellets which was further resuspended in 1 ml wash buffer for staining. Single cell suspensions were transferred into 2 ml Eppendorf tubes for immunophenotype staining. Cells were first stained for viability using Invitrogen Live/Dead Aqua at a 1:1000 dilution (cat #L34966) for 30 minutes at room temperature. Following Live/Dead staining, Fc blocking was performed using the Biolegend Fc Block at a 1:200 dilution (cat #B362118) at 4° C. for 5 minutes. Lastly, cells were stained for B cell marker CD19 using the Biolegend BV421 at 1:400 dilution (cat #115538), T Cell marker TCRb using the Biolegend PE/Cy7 at a 1:200 dilution (cat #109222), CD11b using the Biolegend PerCP/Cy5.5 at a 1:200 dilution (cat #101228), and general macrophage marker F4/80 using the Biolegend APC at a 1:200 dilution (cat #123116) for 30 minutes at room temperature. Once stained, cells were placed on ice, filtered, and sorted using the Sony MA900 Cell Sorter. Cells were sorted using a 100 uM nozzle using semi-purity mode. Sorted macrophage fractions were pelleted and placed in −80 freezer for further downstream analysis. T-cells, B-cells and CD11b+ cells were also collected. Sorted macrophages were analyzed for copies of siRNA/cell and HPRT1 KD by RT-qPCR, using methods as described in Example 12B. T-cells, B-cells and CD11b+ cells were also analyzed for KD by RT-qPCR, but no appreciable knockdown in those cell types was observed.

TABLE 7E
HPRT1 siRNA Accumulation

| | Modified | | | |
|---|---|---|---|---|
| Targeting | Glycan/ | Dose | | Copies of |
| moiety | saccharide | (mg/kg) | Days | siRNA/cell |
| siRNA alone | — | 5 | 1 | 170.7 |
| G-24 | P-18 | 5 | 1 | 3193.1 |
| H-65 | N-18 | 5 | 1 | 4292.9 |
| siRNA alone | — | 10 | 7 | 64.6 |
| G-24 | P-18 | 10 | 7 | 939.7 |
| H-65 | N-18 | 10 | 7 | 1380.5 |

TABLE 7F
HPRT1-mRNA % Knockdown Relative to PBS Control

| | Modified | | | Splenic Macrophage |
|---|---|---|---|---|
| Targeting | Glycan/ | Dose | | mRNA % vs |
| moiety | saccharide | (mg/kg) | Days | Control |
| siRNA alone | — | 10 | 7 | 1.03 |
| G-24 | P-18 | 10 | 7 | 0.68 |
| H-65 | N-18 | 10 | 7 | 0.49 |

### Example 13: In Vitro Cytokine Profiling of Glyco-siRNAs Using Luminex in PBMCs and Other Primary Immune Cells

Primary immune cells, whether total PBMCs or purified immune cells such as purified CD19+ B cells or CD3+CD8+ T cells, are plated and dosed with glyco-siRNA test articles for 24 hours in both resting and stimulated conditions. Cell culture supernatants are harvested and cytokine levels are assessed using ProCartaPlex™ bead arrays on Luminex's FlexMAP 3D instrument. Cytokine levels are assessed to determine if glycan binding inhibits/increases expression. Cytokine levels and increased/decreased expression are used to determine if certain signaling pathways are modified as a result of contacting the primary immune cells with glyco-siRNA test articles, and can be used to predict clinical relevance of glyco-siRNA therapies.

### Example 14: In Vitro Glyco-siRNA Internalization Assay Measured by Stem Loop qPCR

Primary B cells, T cells and NK cells were purchased from STEMCELL Technologies Inc. Primary B, T and NK cells were plated 24 hours before the experiment at 100,000 cells per well in 1 mL of Roswell Park Memorial Institute (RPMI) medium with 10% fetal bovine serum (FBS) in 96-well plates. Glyco-siRNAs were incubated with cells for 4 hours. After incubation, media was removed and washed three times in PBS or PBS+150 mM NaCl. Cells were lysed in 100 ul PBST (PBS+0.25% Triton) and frozen at −80° C. To prepare the standard curve, 20 nM of glyco-siRNAs used in the study or synthesized Micro-RNA from IDT (MiR-21-5p: 5′UAGCUUAUCAGACUGAUGUUGA-3′ (SEQ ID NO: 19), MiR155: 5′-UUAAUGCUAAUCGUGAUAGGGGUU-3′ (SEQ ID NO: 20)) were serial diluted 5-fold for 9 times. The reverse transcription reaction was prepared by using TaqMan MicroRNA Reverse Transcription Kit (Thermo Scientific). A final concentration of 1 mM dNTP, 5 U/μl transcriptase, 0.38 U/μl Rnase inhibitor, 50 nM stem loop RT primer (IDT) against siRNA targeting beta catenin (5′-GTCGTATCCAGTGCAGGGTCCGAGGTATTCGCACTGGATACGACCTGTTGG-3′ (SEQ ID NO: 21)) was mixed in a 10 μl volume reaction with 1× buffer. Frozen cell lysates were thawed and heated at 95° C. for 10 minutes and 5 μl of the boiled cell lysate was added to each 10 μl of reverse transcription mix. Samples were incubated first in 16° C. for 30 minutes then 42° C. for 30 minutes followed by 85° C. for 5 minutes and the samples were ready to be used for qPCR reaction. A final concentration of 1.5 uM Forward primers from IDT (beta catenin: 5′-GCCGCGC TACTGTTGGAT-3′ (SEQ ID NO: 22), MIR-21-5p: 5′-GCCGCGCTAGCTTATCAGACTG-3′ (SEQ ID NO: 23), MIR 155: 5′-GCCGCGCTTAATGCTAATCGTGAT-3′ (SEQ ID NO: 24)), 0.7 uM Reverse primers from IDT (5′-GTGCAGGGTCCGAGGT-3′ (SEQ ID NO: 25)), 0.2 uM customized Taqman MGB probe (beta catenin: 5′-CTGGATACGACTTTCGAAT-3′ (SEQ ID NO: 26), MIR-21-5p: 5′-CTGGATACGACTCAACA-3′ (SEQ ID NO: 27), MiR155. 5′-CTGGATACGACAACCCC-3′ (SEQ ID NO: 28)) from Thermo Scientific were mixed in 1× TaqMan gene expression master mix from Thermo Scientific to a total volume of 8.5 ul. 1.5 ul of cDNA template from the reverse transcription reaction is added to the qPCR mix to a final volume for 10 μl. Samples were first incubated at 95° C. for 10 minutes followed by 40 cycles of 30 seconds of 95° C. and 1 minute at 60° C. Relative enrichment of siRNA over endogenous Micro-RNAs were calculated based on the Ct value of standard curve.

Table 8A reports glyco-siRNA uptake in Primary B cells as compared to naked siRNA in B cells from two different donors. Table 8B reports glyco-siRNA uptake in Primary T cells as compared to naked siRNA in T cells from two different donors. Table 8C reports glyco-siRNA uptake in Natural Killer (NK) cells as compared to naked siRNA from a single donor in two replicate studies.

TABLE 8A
Primary B Cell Uptake (Fold Increase vs Naked siRNA)

| | Modified | | |
|---|---|---|---|
| Glycan | Glycan | Donor 1 | Donor 2 |
| PBS | PBS | 0.05 | 0.21 |
| siRNA | siRNA | 1 | 1.06 |
| Glucose | X-2 | 2.72 | 2.17 |
| G-1 | A-1 | 3.57 | 2.27 |
| G-2 | A-2 | 5.05 | 2.56 |
| G-3 | A-3 | 1.99 | 1.82 |
| G-4 | A-4 | 2.67 | 2.43 |
| G-5 | A-5 | 2.45 | 2.21 |
| G-6 | A-6 | 3.85 | 3.35 |
| G-7 | P-1 | 1.35 | 0.86 |
| G-8 | P-2 | 3.67 | 1.04 |
| G-9 | P-3 | 6.18 | 4.09 |
| G-10 | P-4 | 4.63 | 1.46 |
| G-11 | P-5 | 3.02 | 1.14 |
| G-12 | P-6 | 2.66 | 0.70 |
| G-13 | P-7 | 1.24 | 0.52 |
| G-14 | P-8 | 0.78 | 0.45 |
| G-15 | P-9 | 1.60 | 2.27 |
| G-16 | P-10 | 1.83 | 1.83 |
| G-17 | P-11 | 2.55 | 3.49 |
| G-18 | P-12 | 2.76 | 1.81 |
| G-19 | P-13 | 1.83 | 0.77 |
| G-20 | P-14 | 2.98 | 2.09 |
| G-21 | P-15 | 0.59 | 0.80 |
| G-23 | P-17 | 0.72 | 0.31 |
| G-24 | P-18 | 1.34 | 1.99 |
| G-27 | A-7 | 1.43 | 2.03 |
| G-29 | A-9 | 1.64 | 2.08 |
| H-23 | N-3 | 1.93 | 0.32 |
| H-3 | N-4 | 1.06 | 0.36 |
| G-37 | N-6 | 1.39 | 0.45 |
| G-38 | N-7 | 2.25 | 0.42 |

TABLE 8B
Primary T Cell Uptake (Fold Increase vs Naked siRNA)

| Targeting | Modified | | |
|---|---|---|---|
| moiety | Glycan/saccharide | Donor 1 | Donor 2 |
| siRNA | siRNA | 1 | 1 |
| Glucose | X-2 | 2.83 | 1.68 |
| Tri-GalNAc | X-3 | 5.57 | 3.64 |
| G-1 | A-1 | 3.16 | 2.92 |
| G-2 | A-2 | 1.28 | 1.39 |
| G-3 | A-3 | 1.62 | 1.29 |
| G-4 | A-4 | 3.44 | 2.77 |
| G-5 | A-5 | 1.93 | 5.45 |
| G-6 | A-6 | 0.77 | 1.08 |
| G-7 | P-1 | 1.25 | 2.36 |
| G-8 | P-2 | 1.37 | 3.16 |
| G-9 | P-3 | 1.66 | 3.02 |
| G-10 | P-4 | 1.67 | 2.50 |
| G-11 | P-5 | 1.55 | 1.85 |
| G-12 | P-6 | 1.03 | 3.20 |
| G-13 | P-7 | 0.44 | 0.85 |
| G-14 | P-8 | 0.41 | 0.76 |
| G-15 | P-9 | 4.17 | 3.16 |
| G-16 | P-10 | 2.57 | 2.50 |
| G-17 | P-11 | 0.92 | 2.69 |
| G-18 | P-12 | 4.42 | 3.30 |
| G-19 | P-13 | 1.14 | 1.15 |
| G-20 | P-14 | 5.71 | 6.02 |
| G-21 | P-15 | 0.17 | 0.69 |
| G-22 | P-16 | 0.30 | 0.38 |
| G-23 | P-17 | 0.33 | 0.61 |
| G-27 | A-7 | 1.25 | 2.01 |
| G-28 | A-8 | 0.48 | 0.77 |
| G-29 | A-9 | 0.43 | 0.63 |
| G-30 | A-10 | 2.29 | 1.77 |
| G-3 | N-1 | 0.59 | 0.96 |
| H-13 | N-2 | 1.96 | 1.99 |
| H-23 | N-3 | 0.58 | 0.54 |
| H-3 | N-4 | 0.94 | 0.82 |
| H-7 | N-5 | 2.50 | 2.44 |
| J-2 | N-8 | 1.77 | 1.37 |
| J-4 | N-9 | 0.72 | 0.51 |

TABLE 8C
NK Cell Uptake (Fold Increase vs Naked siRNA)

| Targeting | Modified | Donor 1 | Donor 2 |
|---|---|---|---|
| moiety | Glycan/saccharide | repeat 1 | repeat 2 |
| PBS | PBS | 0.00 | 0.00 |
| siRNA | siRNA | 1.00 | 1.00 |
| Glucose | X-2 | 2.16 | 2.60 |
| Tri-GalNAc | X-3 | 3.36 | 5.34 |
| G-1 | A-1 | 2.72 | 3.49 |
| G-2 | A-2 | 3.71 | 3.42 |
| G-3 | A-3 | 0.92 | 1.36 |
| G-4 | A-4 | 2.20 | 1.98 |
| G-5 | A-5 | 2.53 | 3.19 |
| G-6 | A-6 | 2.51 | 6.37 |
| G-7 | P-1 | 1.39 | 2.36 |
| G-8 | P-2 | 1.46 | 3.18 |
| G-9 | P-3 | 2.85 | — |
| G-10 | P-4 | 0.98 | 3.53 |
| G-11 | P-5 | 0.69 | 4.26 |
| G-12 | P-6 | 0.50 | 2.05 |
| G-13 | P-7 | 0.98 | 1.62 |
| G-14 | P-8 | 1.16 | 1.11 |
| G-15 | P-9 | 4.42 | 4.39 |
| G-16 | P-10 | 2.29 | 3.46 |
| G-17 | P-11 | 4.85 | 8.83 |
| G-18 | P-12 | 2.32 | 5.53 |
| G-19 | P-13 | 0.87 | 2.49 |
| G-20 | P-14 | 4.84 | 6.56 |
| G-21 | P-15 | 0.38 | 0.36 |
| G-22 | P-16 | 0.51 | 0.51 |
| G-23 | P-17 | 0.86 | 0.83 |
| G-24 | P-18 | 2.27 | 2.54 |
| G-27 | A-7 | 3.40 | 3.53 |
| G-28 | A-8 | 0.83 | 1.03 |
| G-29 | A-9 | 0.47 | 0.85 |
| G-30 | A-10 | 1.99 | 2.34 |
| G-3 | N-1 | 0.30 | 0.27 |
| H-13 | N-2 | 0.19 | 0.18 |
| H-23 | N-3 | 0.71 | 0.31 |
| H-3 | N-4 | 0.96 | 0.97 |
| G-37 | N-6 | 0.32 | 0.43 |
| G-38 | N-7 | 0.23 | 0.13 |

### Example 15: In Vitro Glycan Array Protein Binding

Glycan arrays were prepared by printing purified glycans (or glucose or negative controls with no saccharide moiety) on a glass substrate at a concentration of 100 μM. Substrates were purchased from Chemily Glycoscience (Glycan array 300) or Zbiotech (Catch All glycan array, Glycosaminoglycan Array and Heparan Sulfate Glycan Array). Commercially available proteins tagged with human fragment crystallizable region (Fc) were used for the glycan array screening: DC-SIGNR (R&D Systems, 162-D2), Siglec7 (R&D Systems, 1138-SL), CD2 (R&D Systems, 1856-CD-050), CD83 (R&D Systems, 2044-CD), KLRF1 (R&D Systems, 1099-NK), CD28 (Sino Biological Inc., 11524-H41H).

Protein samples were prepared with 1% bovine serum albumin (BSA) in tris-sucrose-magnesium chloride (TSM) buffer with additional Ca²⁺ (20 mM tris-HCl, 150 mM sodium chloride, 5 mM calcium chloride, 2 mM magnesium chloride, and 0.05% Tween 20). Proteins were incubated with the glycan array for 1 hour at room temperature at the concentrations indicated in Tables 9A-9F before being washed three times with wash buffer (20 mM tris-HCl, 150 mM sodium chloride, 5 mM calcium chloride, 2 mM magnesium chloride). The arrays were then contacted with Alexa Fluor 488-labeled goat anti-human IgG (H+L) (Invitrogen A-11013) at 5 μg/ml and measured by fluorescence detection. Values in Tables 9A-9F are reported in relative fluorescence units (RFUs).

TABLE 9A
DC-SIGNR Binding at 20 μg/mL Protein

| Glycan or Control | Binding to DC-SIGNR (20 μg/ml)(RFU) |
|---|---|
| K-22 | 1047827 |
| K-23 | 1047468 |
| K-24 | 1015291 |
| K-27 | 862190.8 |
| K-28 | 723698.8 |
| K-29 | 718967.2 |
| K-30 | 631597 |
| K-31 | 543554.7 |
| Glucose | 5989.667 |

TABLE 9B
CD83 Binding at 20 μg/mL Protein

| Glycan or Control | Binding to CD83 (20 μg/ml)(RFU) |
|---|---|
| K-4 | 4060.13 |
| K-10 | 3048.25 |
| K-12 | 3099.5 |
| K-13 | 3962.59 |
| Negative control | 0 |

TABLE 9C
KLRF1 Binding at 20 μg/mL Protein

| Glycan or Control | Binding to KLRF1 (20 μg/ml)(RFU) |
|---|---|
| K-4 | 4556.62 |
| K-10 | 800.75 |
| K-12 | 898.83 |
| K-13 | 4243.75 |
| Negative control | 0 |

TABLE 9D
CD28 Binding at 20 μg/mL Protein

| Glycan or Control | Binding to CD28 (20 μg/ml)(RFU) |
|---|---|
| K-4 | 2812.88 |
| K-10 | 1713.25 |
| K-12 | 1800.25 |
| K-13 | 3364 |
| Negative control | 0 |

TABLE 9E
CD2 Binding at 20 μg/mL Protein

| Glycan or Control | Binding to CD2 (20 μg/ml)(RFU) |
|---|---|
| K-4 | 6572.63 |
| K-10 | 1713.25 |
| K-12 | 1800.25 |
| K-13 | 3364 |
| Negative control | 0 |

TABLE 9F
Siglec-7 Binding at 10 μg/mL Protein

| Glycan or Control | Binding to Siglec-7 (10 μg/ml)(RFU) |
|---|---|
| K-51 | 28368.5 |
| K-52 | 30639 |
| K-53 | 32177.5 |
| Negative control | 4.25 |

The assay protocols described in Example 15 can be used to test the binding affinity of other glycans, including those described herein including but not limited to those described in any one of Tables 1A-1F, and other proteins (e.g., receptors, Lectins, Siglecs), including but not limited to those described in Table 3 herein.

### Example 16: In Vivo Biodistribution of Glyco-siRNA in Tumor-Bearing Mice

2×10⁵ B16F10 cells were injected into C57BL/6J mice and 5 mg/kg glyco-siRNAs were dosed after 7 days. Each test article was dosed in 3 mice and an additional 3 mice were dosed with PBS control. After 48 hours, animals were immediately euthanized by CO₂ inhalation after body weight measurement. Organs were collected after imaging and snap frozen using liquid nitrogen. Snap frozen tissues were cooled liquid nitrogen and then homogenized using the GENOMAX® 2050 Homogenizer (Antylia Scientific Co.) for 1 minute 45 seconds at 1500 RPM. Powderized tissues were stored in −80° C. freezer until ready for test article extraction.

The organ samples were analyzed by stem loop qPCR as described in Example 12B. The test article glyco-siRNAs demonstrated increased uptake in tumor cells as compared to untargeted, naked siRNA controls. The biodistribution results from this experiment are shown in Table 10.

TABLE 10
Biodistribution of Glyco-siRNA Conjugates
in Tumor-bearing Mice After 24 Hours

| | Modified | | | |
|---|---|---|---|---|
| Targeting | Glycan/ | Dose | | |
| Moiety | Saccharide | (mg/kg) | Liver | Tumor |
| siRNA | siRNA | 5 | 0.05 | 0.04 |
| P-18 | G-24 | 5 | 0.25 | 0.10 |
| P-19 | H-65 | 5 | 0.31 | 0.12 |

** Values recorded as ng RNA/mg tissue

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

We claim:

1. A pharmaceutical composition comprising a glyco-ligand, wherein the glyco-ligand comprises one or more glycan moieties operably linked to one or more sites on a synthetic scaffold domain comprising a synthetic short interfering RNA (siRNA), wherein the one or more glycan moieties comprises:
(a) at least one glycan selected from H-3, K-22, K-23, K-24, K-27, K-28, K-29, K-30, and K-31
(b) at least one glycan selected from M-5, M-6 and M-7;
(c) at least one glycan selected from M-2, M-3, and M-4;
(d) at least one glycan selected from H-65, H-14, H-10 and H-40;
(e) at least one glycan selected from H-33 and M-1;
(f) at least on glycan selected from K-4, K-10, K-12, and K-13;
(g) at least one glycan selected from K-51, K-52, K-53, H-7 and H-8;
(h) glycan H-9;
(i) glycan H-6; or
(j) at least one glycan selected from H-23 and H-64.

2. The pharmaceutical composition of claim 1, wherein the one or more glycan moieties comprises at least one glycan selected from M-5, M-6 and M-7.

3. The pharmaceutical composition of claim 1, wherein the one or more glycan moieties comprises at least one glycan selected from M-2, M-3, and M-4.

4. The pharmaceutical composition of claim 1, wherein the one or more glycan moieties comprises at least one glycan selected from H-65, H-14, H-10 and H-40.

5. The pharmaceutical composition of claim 1, wherein the one or more glycan moieties comprises at least one glycan selected from H-33 and M-1.

6. The pharmaceutical composition of claim 1, wherein the one or more glycan moieties comprises at least one glycan selected from K-4, K-10, K-12, and K-13.

7. The pharmaceutical composition of claim 1, wherein the one or more glycan moieties comprises at least one glycan selected from H-3, K-22, K-23, K-24, K-27, K-28, K-29, K-30, and K-31.

8. The pharmaceutical composition of claim 1, wherein the one or more glycan moieties comprises at least one glycan selected from K-51, K-52, K-53, H-7 and H-8.

9. The pharmaceutical composition of claim 1, wherein the one or more glycan moieties comprises H-9.

10. The pharmaceutical composition of claim 1, wherein the one or more glycan moieties comprises H-6.

11. The pharmaceutical composition of claim 1, wherein the one or more glycan moieties comprises at least one glycan selected from H-23 and H-64.

12. The pharmaceutical composition of any one of claims 1-11, wherein the glyco-ligand comprises a linker selected from: 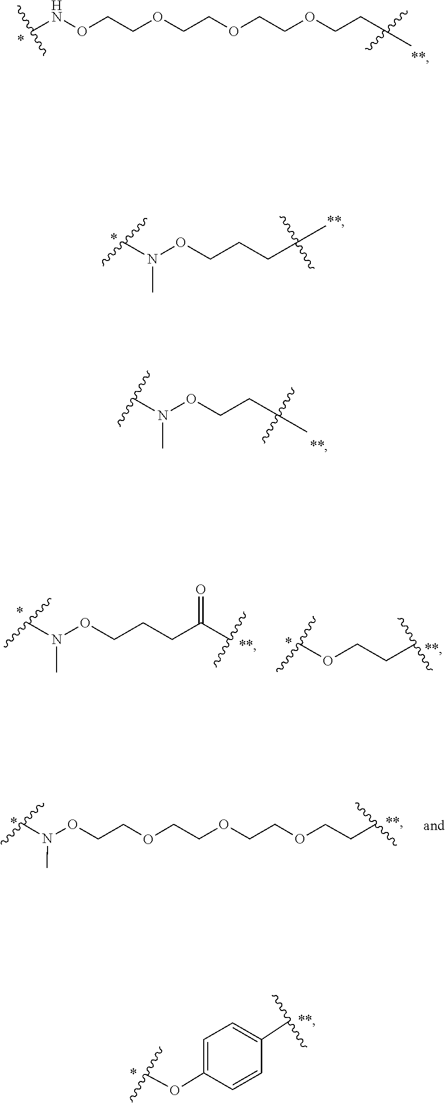
wherein * indicates the point of attachment to the glycan (e.g., at the non-reducing end terminal monosaccharide of the glycan) and ** indicates the point of attachment to the synthetic scaffold domain.

13. The pharmaceutical composition of any one of claims 1-12, wherein the glyco-ligand comprises a linker formed by a click chemistry reaction.

14. The pharmaceutical composition of any one of claims 1-13, wherein the siRNA comprises one or more phosphate linkage modifications selected from phosphorothioate linkage (PS), phosphorodithioate linkage (PS2), phosphoramidate linkage, phosphorodiamidate linkage, thiophosphoramidate linkage, mesyl phosphoramidate linkage, methylphosphonate linkage (MP), methoxypropylphosphonate linkage (MOP), 5′-(E)-vinylphosphonate linkage (5′-(E)-VP), 5′-Methyl Phosphonate linkage (5′-MP), (S)-5′-C-methyl with phosphate linkage, 5′-phosphorothioate linkage (5′-PS), and a peptide nucleic acid linkage (PNA).

15. The pharmaceutical composition of any one of claims 1-14, wherein the siRNA comprises one or more ribose modifications selected from 2′-O-methyl (2′-OMe), 2′-O-methoxyethyl (2′-O-MOE), 2′-deoxy, 2′-deoxy-2′-fluoro (2′-F), 2′-arabino-fluoro (2′-Ara-F), 2′-O-benzyl, 2′-O-methyl-4-pyridine (2′-O—CH2Py (4)), Locked nucleic acid (LNA), (S)-cET-BNA, tricyclo-DNA (tcDNA), phosphorodiamidate morpholino oligomer (PMO), hexose nucleic acid (HNA), Unlocked Nucleic Acid (UNA), threose nucleic acid (TNA), 4′-deoxy-4′thioribonucleic acid, and glycol nucleic acid (GNA).

16. The pharmaceutical composition of any one of claims 1-15, wherein the siRNA comprises one or more modified bases selected from pseudouridine (ψ), 2′thiouridine (s2U), N6′-methyladenosine (m⁶A), 5′methylcytidine (m⁵C), 5′-fluoro-2′-deoxyuridine, N-ethylpiperidine 7′-EAA triazole modified adenine, N-ethylpiperidine 6′-triazole modified adenine, 6′-phenylpyrrolo-cytosine (PhpC), 2′,4′-difluorotoluyl ribonucleoside (rF), and 5′-nitroindole.

17. The pharmaceutical composition of any one of claims 1-13, wherein the siRNA comprises one or more modifications to one or more nucleotides selected from 2-OMe modification, a fluorine modification, a phosphorothioate modification or any combinations thereof.

18. A method of treating a disease or condition comprising administering to a subject in need thereof a therapeutically effective amount of the pharmaceutical composition of any one of claims 1-17.

19. The use of the pharmaceutical composition of any one of claims 1-17 for the manufacture of a medicament for the treatment of a disease or a condition.

20. Use of the pharmaceutical composition of any one of claims 1-17 for the treatment of a disease or a condition in a subject in need thereof.

21. The pharmaceutical composition of claim 13, wherein the click chemistry reaction is a bioorthogonal click chemistry reaction.
